SEQUENCE LISTING (53 sequences)

SEQ ID NO: 1 - protein, 543 aa, Artificial Sequence
  sequence of H0 Metschnikowia species Xyt1p
MGYEEKLVAPALKFKNFLDKTPNIHNVYVIAAISCTSGMMFGFDISSMSVFVDQQPYLKMFDNPSSVIQGFITASMSLGSFFGSLTSTFISEPFGRRASLFICGILWVIGAAVQSSSQNRAQLICGRIIAGWGIGFGSSVAPVYGSEMAPRKIRGTIGGIFQFSVTVGIFIMFLIGYGCSFIQGKASFRIPWGVQMVPGLILLIGLFFIPESPRWLAKQGYWEDAEIIVANVQAKGNRNDANVQIEMSEIKDQLMLDEHLKEFTYADLFTKKYRQRTITAIFAQIWQQLTGMNVMMYYIVYIFQMAGYSGNTNLVPSLIQYIINMAVTVPALFCLDLLGRRTILLAGAAFMMAWQFGVAGILATYSEPAYISDTVRITIPDDHKSAAKGVIACCYLFVCSFAFSWGVGIWVYCSEVWGDSQSRQRGAALATSANWIFNFAIAMFTPSSFKNITWKTYIIYATFCACMFIHVFFFFPETKGKRLEEIGQLWDEGVPAWRSAKWQPTVPLASDAELAHKMDVAHAEHADLLATHSPSSDEKTGTV

SEQ ID NO: 2 - protein, 524 aa, Artificial Sequence
  sequence of H0 Metschnikowia species Gxf1p
MSQDELHTKSGVETPINDSLLEEKHDVTPLAALPEKSFKDYISISIFCLFVAFGGFVFGFDTGTISGFVNMSDFKTRFGEMNAQGEYYLSNVRTGLMVSIFNVGCAVGGIFLCKIADVYGRRIGLMFSMVVYVVGIIIQIASTTKWYQYFIGRLIAGLAVGTVSVISPLFISEVAPKQLRGTLVCCFQLCITLGIFLGYCTTYGTKTYTDSRQWRIPLGICFAWALFLVAGMLNMPESPRYLVEKSRIDDARKSIARSNKVSEEDPAVYTEVQLIQAGIDREALAGSATWMELVTGKPKIFRRVIMGVMLQSLQQLTGDNYFFYYGTTIFKAVGLQDSFQTSIILGIVNFASTFVGIYAIERMGRRLCLLTGSACMFVCFIIYSLIGTQHLYKNGFSNEPSNTYKPSGNAMIFITCLYIFFFASTWAGGVYCIVSESYPLRIRSKAMSVATAANWMWGFLISFFTPFITSAIHFYYGFVFTGCLAFSFFYVYFFVVETKGLSLEEVDILYASGTLPWKSSGWVP

SEQ ID NO: 3 - protein, 454 aa, Artificial Sequence
  sequence of H0 Metschnikowia species ?Gxf1p (variant of Gxf1p with shorter N-terminus)
MSDFKTRFGEMNAQGEYYLSNVRTGLMVSIFNVGCAVGGIFLCKIADVYGRRIGLMFSMVVYVVGIIIQIASTTKWYQYFIGRLIAGLAVGTVSVISPLFISEVAPKQLRGTLVCCFQLCITLGIFLGYCTTYGTKTYTDSRQWRIPLGICFAWALFLVAGMLNMPESPRYLVEKSRIDDARKSIARSNKVSEEDPAVYTEVQLIQAGIDREALAGSATWMELVTGKPKIFRRVIMGVMLQSLQQLTGDNYFFYYGTTIFKAVGLQDSFQTSIILGIVNFASTFVGIYAIERMGRRLCLLTGSACMFVCFIIYSLIGTQHLYKNGFSNEPSNTYKPSGNAMIFITCLYIFFFASTWAGGVYCIVSESYPLRIRSKAMSVATAANWMWGFLISFFTPFITSAIHFYYGFVFTGCLAFSFFYVYFFVVETKGLSLEEVDILYASGTLPWKSSGWVP

SEQ ID NO: 4 - protein, 548 aa, Artificial Sequence
  sequence of H0 Metschnikowia species Gxf2p/Gal2p
MSAEQEQQVSGTSATIDGLASLKQEKTAEEEDAFKPKPATAYFFISFLCGLVAFGGYVFGFDTGTISGFVNMDDYLMRFGQQHADGTYYLSNVRTGLIVSIFNIGCAVGGLALSKVGDIWGRRIGIMVAMIIYMVGIIIQIASQDKWYQYFIGRLITGLGVGTTSVLSPLFISESAPKHLRGTLVCCFQLMVTLGIFLGYCTTYGTKNYTDSRQWRIPLGLCFAWALLLISGMVFMPESPRFLIERQRFDEAKASVAKSNQVSTEDPAVYTEVELIQAGIDREALAGSAGWKELITGKPKMLQRVILGMMLQSIQQLTGNNYFFYYGTTIFKAVGMSDSFQTSIVLGIVNFASTFVGIWAIERMGRRSCLLVGSACMSVCFLIYSILGSVNLYIDGYENTPSNTRKPTGNAMIFITCLFIFFFASTWAGGVYSIVSETYPLRIRSKGMAVATAANWMWGFLISFFTPFITSAIHFYYGFVFTGCLIFSFFYVFFFVRETKGLSLEEVDELYATDLPPWKTAGWTPPSAEDMAHTTGFAEAAKPTNKHV

SEQ ID NO: 5 - protein, 502 aa, Artificial Sequence
  sequence of H0 Metschnikowia species delta-Gxs1p/Delta-Hgt12p (variant of Gxs1p/Hgt12p with shorter N-terminus)
MGIFVGVFAALGGVLFGYDTGTISGVMAMPWVKEHFPKDRVAFSASESSLIVSILSAGTFFGAILAPLLTDTLGRRWCIIISSLVVFNLGAALQTAATDIPLLIVGRVIAGLGVGLISSTIPLYQSEALPKWIRGAVVSCYQWAITIGIFLAAVINQGTHKINSPASYRIPLGIQMAWGLILGVGMFFLPETPRFYISKGQNAKAAVSLARLRKLPQDHPELLEELEDIQAAYEFETVHGKSSWSQVFTNKNKQLKKLATGVCLQAFQQLTGVNFIFYFGTTFFNSVGLDGFTTSLATNIVNVGSTIPGILGVEIFGRRKVLLTGAAGMCLSQFIVAIVGVATDSKAANQVLIAFCCIFIAFFAATWGPTAWVVCGEIFPLRTRAKSIAMCAASNWLLNWAIAYATPYLVDSDKGNLGTNVFFIWGSCNFFCLVFAYFMIYETKGLSLEQVDELYEKVASARKSPGFVPSEHAFREHADVETAMPDNFNLKAEAISVEDASV

SEQ ID NO: 6 - skipped

SEQ ID NO: 7 - protein, 526 aa, Artificial Sequence
  sequence of H0 Metschnikowia species Gxs1p/Hgt12
MGLESNKLIRKYINVGEKRAGSSGMGIFVGVFAALGGVLFGYDTGTISGVMAMPWVKEHFPKDRVAFSASESSLIVSILSAGTFFGAILAPLLTDTLGRRWCIIISSLVVFNLGAALQTAATDIPLLIVGRVIAGLGVGLISSTIPLYQSEALPKWIRGAVVSCYQWAITIGIFLAAVINQGTHKINSPASYRIPLGIQMAWGLILGVGMFFLPETPRFYISKGQNAKAAVSLARLRKLPQDHPELLEELEDIQAAYEFETVHGKSSWSQVFTNKNKQLKKLATGVCLQAFQQLTGVNFIFYFGTTFFNSVGLDGFTTSLATNIVNVGSTIPGILGVEIFGRRKVLLTGAAGMCLSQFIVAIVGVATDSKAANQVLIAFCCIFIAFFAATWGPTAWVVCGEIFPLRTRAKSIAMCAASNWLLNWAIAYATPYLVDSDKGNLGTNVFFIWGSCNFFCLVFAYFMIYETKGLSLEQVDELYEKVASARKSPGFVPSEHAFREHADVETAMPDNFNLKAEAISVEDASV

SEQ ID NO: 8 - protein, 548 aa, Artificial Sequence
  sequence of H0 Metschnikowia species Hxt5p
MSIFEGKDGKGVSSTESLSNDVRYDNMEKVDQDVLRHNFNFDKEFEELEIEAAQVNDKPSFVDRILSLEYKLHFENKNHMVWLLGAFAAAAGLLSGLDQSIISGASIGMNKALNLTEREASLVSSLMPLGAMAGSMIMTPLNEWFGRKSSLIISCIWYTIGSALCAGARDHHMMYAGRFILGVGVGIEGGCVGIYISESVPANVRGSIVSMYQFNIALGEVLGYAVAAIFYTVHGGWRFMVGSSLVFSTILFAGLFFLPESPRWLVHKGRNGMAYDVWKRLRDINDESAKLEFLEMRQAAYQERERRSQESLFSSWGELFTIARNRRALTYSVIMITLGQLTGVNAVMYYMSTLMGAIGFNEKDSVFMSLVGGGSLLIGTIPAILWMDRFGRRVWGYNLVGFFVGLVLVGVGYRFNPVTQKAASEGVYLTGLIVYFLFFGSYSTLTWVIPSESFDLRTRSLGMTICSTFLYLWSFTVTYNFTKMSAAFTYTGLTLGFYGGIAFLGLIYQVCFMPETKDKTLEEIDDIFNRSAFSIARENISNLKKGIW

SEQ ID NO: 9 - protein, 543 aa, Artificial Sequence
  sequence of H0 Metschnikowia species Xyt1p with S75L mutation
MGYEEKLVAPALKFKNFLDKTPNIHNVYVIAAISCTSGMMFGFDISSMSVFVDQQPYLKMFDNPSSVIQGFITALMSLGSFFGSLTSTFISEPFGRRASLFICGILWVIGAAVQSSSQNRAQLICGRIIAGWGIGFGSSVAPVYGSEMAPRKIRGTIGGIFQFSVTVGIFIMFLIGYGCSFIQGKASFRIPWGVQMVPGLILLIGLFFIPESPRWLAKQGYWEDAEIIVANVQAKGNRNDANVQIEMSEIKDQLMLDEHLKEFTYADLFTKKYRQRTITAIFAQIWQQLTGMNVMMYYIVYIFQMAGYSGNTNLVPSLIQYIINMAVTVPALFCLDLLGRRTILLAGAAFMMAWQFGVAGILATYSEPAYISDTVRITIPDDHKSAAKGVIACCYLFVCSFAFSWGVGIWVYCSEVWGDSQSRQRGAALATSANWIFNFAIAMFTPSSFKNITWKTYIIYATFCACMFIHVFFFFPETKGKRLEEIGQLWDEGVPAWRSAKWQPTVPLASDAELAHKMDVAHAEHADLLATHSPSSDEKTGTV

SEQ ID NO: 10 - protein, 524 aa, Artificial Sequence
  sequence of H0 Metschnikowia species Hxt2.6p
MSSTTDTLEKRDTEPFTSDAPVTVHDYIAEERPWWKVPHLRVLTWSVFVITLTSTNNGYDGSMLNGLQSLDIWQEDLGHPAGQKLGALANGVLFGNLAAVPFASYFCDRFGRRPVICFGQILTIVGAVLQGLSNSYGFFLGSRIVLGFGAMIATIPSPTLISEIAYPTHRETSTFAYNVCWYLGAIIASWVTYGTRDLQSKACWSIPSYLQAALPFFQVCMIWFVPESPRFLVAKGKIDQARAVLSKYHTGDSTDPRDVALVDFELHEIESALEQEKLNTRSSYFDFFKKRNFRKRGFLCVMVGVAMQLSGNGLVSYYLSKVLDSIGITETKRQLEINGCLMIYNFVICVSLMSVCRMFKRRVLFLTCFSGMTVCYTIWTILSALNEQRHFEDKGLANGVLAMIFFYYFFYNVGINGLPFLYITEILPYSHRAKGLNLFQFSQFLTQIYNGYVNPIAMDAISWKYYIVYCCILFVELVIVFFTFPETSGYTLEEVAQVFGDEAPGLHNRQLDVAKESLEHVEHV

SEQ ID NO: 11 - protein, 556 aa, Artificial Sequence
  sequence of H0 Metschnikowia species Qup2p
MGFRNLKRRLSNVGDSMSVHSVKEEEDFSRVEIPDEIYNYKIVLVALTAASAAIIIGYDAGFIGGTVSLTAFKSEFGLDKMSATAASAIEANVVSVFQAGAYFGCLFFYPIGEIWGRKIGLLLSGFLLTFGAAISLISNSSRGLGAIYAGRVLTGLGIGGCSSLAPIYVSEIAPAAIRGKLVGCWEVSWQVGGIVGYWINYGVLQTLPISSQQWIIPFAVQLIPSGLFWGLCLLIPESPRFLVSKGKIDKARKNLAYLRGLSEDHPYSVFELENISKAIEENFEQTGRGFFDPLKALFFSKKMLYRLLLSTSMFMMQNGYGINAVTYYSPTIFKSLGVQGSNAGLLSTGIFGLLKGAASVFWVFFLVDTFGRRFCLCYLSLPCSICMWYIGAYIKIANPSAKLAAGDTATTPAGTAAKAMLYIWTIFYGITWNGTTWVICAEIFPQSVRTAAQAVNASSNWFWAFMIGHFTGQALENIGYGYYFLFAACSAIFPVVVWFVYPETKGVPLEAVEYLFEVRPWKAHSYALEKYQIEYNEGEFHQHKPEVLLQGSENSD

SEQ ID NO: 12 - protein, 543 aa, Artificial Sequence; identical to SEQ ID NO: 1
  sequence of H0 Metschnikowia species Aps1p/Hgt19p

SEQ ID NO: 13 - DNA, 1632 bp, Artificial Sequence
  sequence of H0 Metschnikowia species XYT1
atgggttacgaggaaaagcttgtagcgcccgcgttgaaattcaaaaactttcttgacaaaacccccaatattcacaatgtctatgtcattgccgccatctcctgtacatcaggtatgatgtttggatttgatatctcgtcgatgtctgtctttgtcgaccagcagccatacttgaagatgtttgacaaccctagttccgtgattcaaggtttcattaccgcgctgatgagtttgggctcgtttttcggctcgctcacatccacgttcatctctgagccttttggtcgtcgtgcatcgttgttcatttgtggtattctttgggtaattggagcagcggttcaaagttcgtcgcagaacagggcccaattgatttgtgggcgtatcattgcaggatggggcattggctttgggtcatcggtggctcctgtttacgggtccgagatggctccgagaaagatcagaggcacgattggtggaatcttccagttctccgtcaccgtgggtatctttatcatgttcttgattgggtacggatgctctttcattcaaggaaaggcctctttccggatcccctggggtgtgcaaatggttcccggccttatcctcttgattggacttttctttattcctgaatctccccgttggttggccaaacagggctactgggaagacgccgaaatcattgtggccaatgtgcaggccaagggtaaccgtaacgacgccaacgtgcagattgaaatgtcggagattaaggatcaattgatgcttgacgagcacttgaaggagtttacgtacgctgaccttttcacgaagaagtaccgccagcgcacgatcacggcgatctttgcccagatctggcaacagttgaccggtatgaatgtgatgatgtactacattgtgtacattttccagatggcaggctacagcggcaacacgaacttggtgcccagtttgatccagtacatcatcaacatggcggtcacggtgccggcgcttttctgcttggatctcttgggccgtcgtaccattttgctcgcgggtgccgcgttcatgatggcgtggcaattcggcgtggcgggcattttggccacttactcagaaccggcatatatctctgacactgtgcgtatcacgatccccgacgaccacaagtctgctgcaaaaggtgtgattgcatgctgctatttgtttgtgtgctcgtttgcattctcgtggggtgtcggtatttgggtgtactgttccgaggtttggggtgactcccagtcgagacaaagaggcgccgctcttgcgacgtcggccaactggatcttcaacttcgccattgccatgttcacgccgtcctcattcaagaatatcacgtggaagacgtatatcatctacgccacgttctgtgcgtgcatgttcatacacgtgtttttctttttcccagaaacaaagggcaagcgtttggaggagataggccagctttgggacgaaggagtcccagcatggaggtcagccaagtggcagccaacagtgccgctcgcgtccgacgcagagcttgcacacaagatggatgttgcgcacgcggagcacgcggacttattggccacgcactcgccatcttcagacgagaagacgggcacggtctaa

SEQ ID NO: 14 - DNA, 1635 bp, Artificial Sequence
  sequence of H0 Metschnikowia species GXF1
atgtctcaagacgaacttcatacaaagtctggtgttgaaacaccaatcaacgattcgcttctcgaggagaagcacgatgtcaccccactcgcggcattgcccgagaagtccttcaaggactacatttccatttccattttctgtttgtttgtggcatttggtggttttgttttcggtttcgacaccggtacgatttccggtttcgtcaacatgtccgacttcaagaccagatttggtgagatgaatgcccagggcgaatactacttgtccaatgttagaactggtttgatggtttctattttcaacgtcggttgcgccgttggtggtatcttcctttgtaagattgccgatgtttatggcagaagaattggtcttatgttttccatggtggtttatgtcgttggtatcattattcagattgcctccaccaccaaatggtaccaatacttcattggccgtcttattgctggcttggctgtgggtactgtttccgtcatctcgccacttttcatttccgaggttgctcctaaacagctcagaggtacgcttgtgtgctgcttccagttgtgtatcaccttgggtatctttttgggttactgcacgacctacggtacaaagacttacactgactccagacagtggagaatcccattgggtatctgtttcgcgtgggctttgtttttggtggccggtatgttgaacatgcccgagtctcctagatacttggttgagaaatcgagaatcgacgatgccagaaagtccattgccagatccaacaaggtttccgaggaagaccccgccgtgtacaccgaggtgcagcttatccaggctggtattgacagagaggcccttgccggcagcgccacatggatggagcttgtgactggtaagcccaaaatcttcagaagagtcatcatgggtgtcatgcttcagtccttgcaacaattgactggtgacaactactttttctactacggaaccacgattttcaaggctgttggcttgcaggactctttccagacgtcgattatcttgggtattgtcaactttgcctcgacttttgtcggtatttacgccattgagagaatgggcagaagattgtgtttgttgaccggatctgcgtgcatgtttgtgtgtttcatcatctactcgctcattggtacgcagcacttgtacaagaacggcttctctaacgaaccttccaacacatacaagccttccggtaacgccatgatcttcatcacgtgtctttacattttcttctttgcctcgacctgggccggtggtgtttactgtatcgtgtccgagtcttacccattgagaatcagatccaaggccatgtctgtcgccaccgccgccaactggatgtggggtttcttgatctcgttcttcacgcctttcatcacctccgccatccacttttactacggttttgttttcactggctgcttggcgttctccttcttctacgtctacttctttgtcgtggagaccaagggtctttccttggaggaggttgacattttgtacgcttccggtacgcttccatggaagtcctctggctgggtgcctcctaccgcggacgaaatggcccacaacgccttcgacaacaagccaactgacgaacaagtctaa

SEQ ID NO: 15 - DNA, 1425 bp, Artificial Sequence
  sequence of H0 Metschnikowia species delta-GXF1 (variant of GXF1 with shorter N-terminus)
atgtccgacttcaagaccagatttggtgagatgaatgcccagggcgaatactacttgtccaatgttagaactggtttgatggtttctattttcaacgtcggttgcgccgttggtggtatcttcctttgtaagattgccgatgtttatggcagaagaattggtcttatgttttccatggtggtttatgtcgttggtatcattattcagattgcctccaccaccaaatggtaccaatacttcattggccgtcttattgctggcttggctgtgggtactgtttccgtcatctcgccacttttcatttccgaggttgctcctaaacagctcagaggtacgcttgtgtgctgcttccagttgtgtatcaccttgggtatctttttgggttactgcacgacctacggtacaaagacttacactgactccagacagtggagaatcccattgggtatctgtttcgcgtgggctttgtttttggtggccggtatgttgaacatgcccgagtctcctagatacttggttgagaaatcgagaatcgacgatgccagaaagtccattgccagatccaacaaggtttccgaggaagaccccgccgtgtacaccgaggtgcagcttatccaggctggtattgacagagaggcccttgccggcagcgccacatggatggagcttgtgactggtaagcccaaaatcttcagaagagtcatcatgggtgtcatgcttcagtccttgcaacaattgactggtgacaactactttttctactacggaaccacgattttcaaggctgttggcttgcaggactctttccagacgtcgattatcttgggtattgtcaactttgcctcgacttttgtcggtatttacgccattgagagaatgggcagaagattgtgtttgttgaccggatctgcgtgcatgtttgtgtgtttcatcatctactcgctcattggtacgcagcacttgtacaagaacggcttctctaacgaaccttccaacacatacaagccttccggtaacgccatgatcttcatcacgtgtctttacattttcttctttgcctcgacctgggccggtggtgtttactgtatcgtgtccgagtcttacccattgagaatcagatccaaggccatgtctgtcgccaccgccgccaactggatgtggggtttcttgatctcgttcttcacgcctttcatcacctccgccatccacttttactacggttttgttttcactggctgcttggcgttctccttcttctacgtctacttctttgtcgtggagaccaagggtctttccttggaggaggttgacattttgtacgcttccggtacgcttccatggaagtcctctggctgggtgcctcctaccgcggacgaaatggcccacaacgccttcgacaacaagccaactgacgaacaagtctaa

SEQ ID NO: 16 - DNA, 1647 bp, Artificial Sequence
  sequence of H0 Metschnikowia species GXF2/GAL2
atgagtgccgaacaggaacaacaagtatcgggcacatctgccacgatagatgggctggcgtccttgaagcaagaaaaaaccgccgaggaggaagacgccttcaagcctaagcccgccacggcgtactttttcatttcgttcctctgtggcttggtcgcctttggcggctacgttttcggtttcgataccggtacgatttccgggtttgttaacatggacgactatttgatgagattcggccagcagcacgctgatggcacgtattacctttccaacgtgagaaccggtttgatcgtgtcgatcttcaacattggctgtgccgttggtggtcttgcgctttcgaaagtcggtgacatttggggcagaagaattggtattatggttgctatgatcatctacatggtgggaatcatcatccagatcgcttcacaggataaatggtaccagtacttcattggccgtttgatcaccggattgggtgtcggcaccacgtccgtgcttagtcctcttttcatctccgagtcggctccgaagcatttgagaggcacccttgtgtgttgtttccagctcatggtcaccttgggtatctttttgggctactgcacgacctacggtaccaagaactacactgactcgcgccagtggcggattcccttgggtctttgcttcgcatgggctcttttgttgatctcgggaatggttttcatgcctgaatccccacgtttcttgattgagcgccagagattcgacgaggccaaggcttccgtggccaaatcgaaccaggtttcgaccgaggaccccgccgtgtacactgaagtcgagttgatccaggccggtattgaccgtgaggcattggccggatccgctggctggaaagagcttatcacgggtaagcccaagatgttgcagcgtgtgattttgggaatgatgctccagtcgatccagcagcttaccggtaacaactactttttctactatggtaccacgatcttcaaggccgtgggcatgtcggactcgttccagacctcgattgttttgggtattgtcaacttcgcctccacttttgtcggaatctgggccatcgaacgcatgggccgcagatcttgtttgcttgttggttccgcgtgcatgagtgtgtgtttcttgatctactccatcttgggttccgtcaacctttacatcgacggctacgagaacacgccttccaacacgcgtaagcctaccggtaacgccatgattttcatcacgtgtttgttcatcttcttcttcgcctccacctgggccggtggtgtgtacagtattgtgtctgaaacatacccattgagaatccgctctaaaggtatggccgtggccaccgctgccaactggatgtggggtttcttgatttcgttcttcacgcctttcatcacctcggccatccacttctactacgggtttgtgttcacagggtgtcttattttctccttcttctacgtgttcttctttgttagggaaaccaagggtctctcgttggaagaggtggatgagttatatgccactgacctcccaccatggaagaccgcgggctggacgcctccttctgctgaggatatggcccacaccaccgggtttgccgaggccgcaaagcctacgaacaaacacgtttaa

SEQ ID NO: 17 - DNA, 1509 bp, Artificial Sequence
  sequence of H0 Metschnikowia species delta-GXS1 /delta-HGT12 (variant of GXS1 /HGT12 with shorter N-terminus)
atgggcattttcgttggcgttttcgccgcgcttggcggtgttctctttggctacgataccggtaccatctctggtgtgatggccatgccttgggtcaaggaacatttcccaaaagaccgtgttgcatttagtgcttccgagtcgtcgttgattgtgtctattttatctgcaggaactttctttggagccattcttgctccgctcttgaccgatacattgggtagacgctggtgtattatcatctcttcgctcgttgtgttcaatttgggtgctgctttgcagacggctgccacggatatcccgctcttgattgttggtcgtgtcattgccggtttaggggttggtttgatttcgctgacgattccattgtaccagtccgaagcgcttcccaaatggattagaggtgctgttgtctcgtgctaccaatgggccattactattggtatctttttggctgccgtgatcaaccagggcactcacaagatcaacagccctgcgtcgtacagaattccattgggtattcagatggcatggggtcttatcttgggtgtcggcatgttcttcttgcccgagacgcctcgtttctacatttccaagggccagaatgcgaaggctgctgtttcattggcgcgtttgagaaagcttccgcaagatcacccggagttgttggaggaattggaagatatccaggcggcatacgagtttgagactgtccatggcaagtcttcatggctgcaggttttcaccaacaagaacaaacaattgaagaagttggccacgggcgtgtgcttgcaggcgttccaacaattgactggtgtgaacttcattttctactttggcacgactttcttcaacagtgttgggcttgacggattcaccacctccttggccaccaacattgtcaatgttggctcgacgatccctggtattttgggtgttgagattttcggcagaagaaaagtgttgttgaccggcgctgctggtatgtgtctttcgcaattcattgttgccattgttggtgtagccaccgactccaaggctgcgaaccaagttcttattgccttctgctgcattttcattgcgttctttgcagccacctggggccccaccgcatgggttgtttgtggcgagattttccccttgagaaccagagccaagtcgattgccatgtgcgctgcttcgaactggttgttgaactgggcaattgcatacgccacgccatacttggttgactccgataagggtaacttgggcaccaatgtgtttttcatttggggaagctgtaacttcttctgccttgtgtttgcctacttcatgatttacgagaccaagggtctttccttggagcaggttgatgagctttacgagaaggttgccagcgccagaaagtcgcctggcttcgtgccaagcgagcacgctttcagagagcacgccgatgtggagaccgccatgccagacaacttcaacttgaaggcggaggcgatttctgtcgaggatgcctctgtttaa

SEQ ID NO: 18 - skipped

SEQ ID NO: 19 - DNA, 1647 bp, Artificial Sequence
  sequence of H0 Metschnikowia species GXS1/HGT12
atgagcatctttgaaggcaaagacgggaagggggtatcctccaccgagtcgctttccaatgacgtcagatatgacaacatggagaaagttgatcaggatgttcttagacacaacttcaactttgacaaagaattcgaggagctcgaaatcgaggcggcgcaagtcaacgacaaaccttcttttgtcgacaggattttatccctcgaatacaagcttcatttcgaaaacaagaaccacatggtgtggctcttgggcgctttcgcagccgccgcaggcttattgtctggcttggatcagtccattatttctggtgcatccattggaatgaacaaagcattgaacttgactgaacgtgaagcctcattggtgtcttcgcttatgcctttaggcgccatggcaggctccatgattatgacacctcttaatgagtggttcggaagaaaatcatcgttgattatttcttgtatttggtataccatcggatccgctttgtgcgctggcgccagagatcaccacatgatgtacgctggcagatttattcttggtgtcggtgtgggtatagaaggtgggtgtgtgggcatttacatttccgagtctgtcccagccaatgtgcgtggtagtatcgtgtcgatgtaccagttcaatattgctttgggtgaagttctagggtatgctgttgctgccattttctacactgttcatggtggatggaggttcatggtggggtcttctttagtattctctactatattgtttgccggattgtttttcttgcccgagtcacctcgttggttggtgcacaaaggcagaaacggaatggcatacgatgtgtggaagagattgagagacataaacgatgaaagcgcaaagttggaatttttggagatgagacaggctgcttatcaagagagagaaagacgctcgcaagagtctttgttctccagctggggcgaattattcaccatcgctagaaacagaagagcacttacttactctgtcataatgatcactttgggtcaattgactggtgtcaatgccgtcatgtactacatgtcgactttgatgggtgcaattggtttcaacgagaaagactctgtgttcatgtcccttgtgggaggcggttctttgcttataggtaccattcctgccattttgtggatggaccgtttcggcagaagagtttggggttataatcttgttggtttcttcgttggtttggtgctcgttggtgttggctaccgtttcaatcccgtcactcaaaaggcggcttcagaaggtgtgtacttgacgggtctcattgtctatttcttgttctttggttcctactcgaccttaacttgggtcattccatccgagtcttttgatttgagaacaagatctttgggtatgacaatctgttccactttcctttacttgtggtctttcaccgtcacctacaacttcaccaagatgtccgccgccttcacatacactgggttgacacttggtttctacggtggcattgcgttccttggtttgatttaccaggtctgcttcatgcccgagacgaaggacaagactttggaagaaattgacgatatcttcaatcgttctgcgttctctatcgcgcgcgagaacatctccaacttgaagaagggtatttggtaa

SEQ ID NO: 20 - DNA, 1647 bp, Artificial Sequence; identical to SEQ ID NO: 19
  sequence of H0 Metschnikowia species HXT5

SEQ ID NO: 21 - DNA, 1632 bp, Artificial Sequence
  sequence of H0 Metschnikowia species XYT1 codon optimized for expression in S. cerevisiae
atgggatacgaagagaaattagtggcccccgctttgaaatttaagaactttttggataagaccccaaatatacataacgtttacgtaattgcggcgatctcgtgtacctcaggtatgatgttcggtttcgatatatcgtcgatgtccgtgttcgtggaccaacagccgtatttaaaaatgtttgataaccctagcagcgtgatacaagggtttataactgcgttgatgtctttggggagctttttcggatcgctaacgtccacttttatttcagaaccttttggtagacgtgcctctttgttcatatgcgggatcctttgggtaattggggcggcagttcaaagttcttctcagaaccgtgcgcagcttatttgtggccgaattattgcagggtggggcatcggattcggttctagcgttgcgccggtatacggttcagaaatggccccacgcaaaattagaggaacaatcggaggtatttttcaattttctgtcacggtcggaatattcataatgttcctgattggctacggctgctcatttatacaaggcaaggccagttttagaattccgtggggagttcaaatggtaccaggtctcattctgttgatcggactattcttcattcctgaatccccaagatggttagccaaacaaggctactgggaagacgctgagatcatcgtagcaaacgttcaagctaagggtaacaggaacgatgctaatgtgcaaattgaaatgtccgagataaaagatcagttaatgcttgacgagcatttaaaggagtttacttatgccgatttgtttaccaaaaaataccggcaaaggacgataacagctatatttgcccaaatatggcaacagctgacaggtatgaatgtcatgatgtactacatcgtatatatatttcaaatggcaggttattcaggtaatactaatttagttccttcactcattcagtatattataaatatggctgttacggtccccgcattgttctgtcttgatctgcttggcaggaggacaattttattagctggcgccgcttttatgatggcctggcaatttggtgttgctggcattttagctacttattcagagccagcctatatttcagataccgtgagaattacaattccagatgaccataaaagtgccgctaagggtgtcatcgcttgctgctatttgtttgtttgttccttcgccttttcctggggtgtaggtatctgggtttattgttcagaagtgtggggtgatagtcaatccagacaaagaggtgctgcattggcaacttctgctaattggatcttcaatttcgcaattgcaatgtttacaccttcttctttcaaaaatatcacttggaagacttatatcatttatgctacattttgtgcttgtatgttcattcatgtttttttttttttccctgaaacaaagggtaagagactagaagaaattggacagctatgggatgaaggtgtcccagcatggagatctgcaaaatggcaacccactgtcccactagcaagtgacgctgaattagctcacaaaatggatgttgcacacgctgaacacgcagacttattggcaacccattctccaagtagtgacgaaaaaactggtaccgtttaa

SEQ ID NO: 22 - DNA, 1575 bp, Artificial Sequence
  sequence of H0 Metschnikowia species HXT2.6
atgctgagcactaccgataccctcgaaaaaagggacaccgagcctttcacttcagatgctcctgtcacagtccatgactatatcgcagaggagcgtccgtggtggaaagtgccgcatttgcgtgtattgacttggtctgttttcgtgatcaccctcacctccaccaacaacgggtatgatggcctgatgttgaatggattgcaatccttggacatttggcaggaggatttgggtcaccctgcgggccagaaattgggtgccttggccaacggtgttttgtttggtaaccttgctgctgtgccttttgcttcgtatttctgcgatcgttttggtagaaggccggtcatttgtttcggacagatcttgacaattgttggtgctgtattacaaggtttgtccaacagctatggattttttttgggttcgagaattgtgttgggttttggtgctatgatagccactattccgctgccaacattgatttccgaaatcgcctaccctacgcatagagaaacttccactttcgcctacaacgtgtgctggtatttgggagccattatcgcctcctgggtcacatacggcaccagagatttacagagcaaggcttgctggtcaattccttcttatctccaggccgccttacctttctttcaagtgtgcatgatttggtttgtgccagagtctcccagattcctcgttgccaagggcaagatcgaccaagcaagggctgttttgtctaaataccatacaggagactcgactgaccccagagacgttgcgttggttgactttgagctccatgagattgagagtgcattggagcaggaaaaattgaacactcgctcgtcatactttgactttttcaagaagagaaactttagaaagagaggcttcttgtgtgtcatggtcggtgttgcaatgcagctttctggaaacggcttagtgtcctattacttgtcgaaagtgctagactcgattggaatcactgaaaccaagagacagctcgagatcaatggctgcttgatgatctataactttgtcatctgcgtctcgttgatgagtgtttgccgtatgttcaaaagaagagtattatttctcacgtgtttctcaggaatgacggtttgctacacgatatggacgattttgtcagcgcttaatgaacagagacactttgaggataaaggcttggccaatggcgtgttggcaatgatcttcttctactattttttctacaacgttggcatcaatggattgccattcctatacatcaccgagatcttgccttactcacacagagcaaaaggcttgaatttattccaattctcgcaatttctcacgcaaatctacaatggctatgtgaacccaatcgccatggacgcaatcagctggaagtattacattgtgtactgctgtattctcttcgtggagttggtgattgtgtttttcacgttcccagaaacttcgggatacactttggaggaggtcgcccaggtatttggtgatgaggctcccgggctccacaacagacaattggatgttgcgaaagaatcactcgagcatgttgagcatgtttga

SEQ ID NO: 23 - DNA, 1605 bp, Artificial Sequence
  sequence of H0 Metschnikowia species HXT2.6 codon optimized for expression in S. cerevisiae
atgagccagtctaaagaaaagtccaacgttattaccaccgtcttgtctgaagaattgccagttaagtactccgaagaaatctccgattacgtttaccatgatcaacattggtggaagtacaaccacttcagaaaattgcattggtacatcttcgttctgactttgacttctaccaacaatggttacgatggctctatgttgaacggtctacaatctttgtctacttggaaagatgctatgggtaatcctgaaggttacattttgggtgctttggctaatggtactattttcggtggtgttttggctgttgcttttgcttcttgggcttgtgatagatttggtagaaagttgactacctgcttcggttctatcgttactgttattggtgctatattgcaaggtgcctctactaattacgcattctttttcgtttcccgtatggttattggttttggtttcggtctagcttctgttgcttctccaactttgattgctgaattgtctttcccaacttacagaccaacttgtactgccttgtacaatgttttttggtacttgggtgctgttattgctgcatgggttacttatggtactagaactatcgtttctgcctactcttggagaattccatcttacttgcaaggtttgttgccattggttcaagtttgtttggtttggtgggttccagaatctccaagattcttggtttctaagggtaagattgaaaaggccagggaattcttgattaagttccatactggtaacgacacccaagaacaagctactagattggtcgaatttgagttgaaagaaattgaagccgccttggagatggaaaagattaactctaattctaagtacaccgacttcatcaccatcaagactttcagaaagagaatcttcttggttgctttcactgcttgtatgactcaattgtctggtaacggtttggtgtcttactacttgtccaaggttttgatctccattggtattaccggtgagaaagaacaattgcaaatcaacggttgcctgatgatctacaacttggttttgtctttagctgttgccttcacctgttacttgtttagaagaaaggccctgttcatcttctcttgctcattcatgttgttgtcctacgttatttggaccattctgtccgctatcaatcaacagagaaacttcgaacaaaaaggtctaggtcaaggtgtcttggctatgatttttatctactacttggcctacaacatcggtttgaatggtttgccatacttgtacgttaccgaaatcttgccatatactcatagagctaagggcatcaacttgtattccttggttattaacatcaccctgatctataacggtttcgttaacgctattgctatggatgctatttcctggaagtactacatcgtttactgctgcattattgccgttgaattggttgttgttatcttcacctacgttgaaactttcggttacaccttggaagaagttgctagagtttttgaaggtactgattctttggccatggacattaacttgaacggtacagtttccaacgaaaagatcgatatcgttcactctgaaagaggttcctctgcttaa

SEQ ID NO: 24 - DNA, 1692 bp, Artificial Sequence
  sequence of H0 Metschnikowia species QUP2
atgggctttcgcaacttaaagcgcaggctctcaaatgttggcgactccatgtcagtgcactctgtgaaagaggaggaagacttctcccgcgtggaaatcccggatgaaatctacaactataagatcgtccttgtggctttaacagcggcgtcggctgccatcatcatcggctacgatgcaggcttcattggtggcacggtttcgttgacggcgttcaaactggaatttggcttggacaaaatgtctgcgacggcggcttctgctatcgaagccaacgttgtttccgtgttccaggccggcgcctactttgggtgtcttttcttctatccgattggcgagatttggggccgtaaaatcggtcttcttctttccggctttcttttgacgtttggtgctgctatttctttgatttcgaactcgtctcgtggccttggtgccatatatgctggaagagtactaacaggtttggggattggcggatgtctgagtttggccccaatctacgtttctgaaatcgcgcctgcagcaatcagaggcaagcttgtgggctgctgggaagtgtcatggcaggtgggcggcattgttggctactggatcaattacggagtcttgcagactcttccgattagctcacaacaatggatcatcccgtttgctgtacaattgatcccatcggggcttttctggggcctttgtcttttgattccagagctgccacgttttcttgtatcgaagggaaagatcgataaggcgcgcaaaaacttagcgtacttgcgtggacttagcgaggaccacccctattctgtttttgagttggagaacattagtaaggccattgaagagaacttcgagcaaacaggaaggggttttttcgacccattgaaagctttgtttttcagcaaaaaaatgctttaccgccttctcttgtccacgtcaatgttcatgatgcagaatggctatggaatcaatgctgtgacatactactcgcccacgatcttcaaatccttaggcgttcagggctcaaacgccggtttgctctcaacaggaattttcggtcttcttaaaggtgccgcttcggtgttctgggtctttttcttggttgacacattcggccgccggttttgtctttgctacctctctctcccctgctcgatctgcatgtggtatattggcgcatacatcaagattgccaacccttcagcgaagcttgctgcaggagacacagccaccaccccagcaggaactgcagcgaaagcgatgctttacatatggacgattttctacggcattacgtggaatggtacgacctgggtgatctgcgcggagattttcccccagtcggtgagaacagccgcgcaggccgtcaacgcttcttctaattggttctgggctttcatgatcggccacttcactggccaggcgctcgagaatattgggtacggatactacttcttgtttgcggcgtgctctgcaatcttccctgtggtagtctggtttgtgtaccccgaaacaaagggtgtgcctttggaggccgtggagtatttgttcgaggtgcgtccttggaaagcgcactcatatgctttggagaagtaccagattgagtacaacgagggtgaattccaccaacataagcccgaagtactcttacaagggtctgaaaactcggacacgagcgagaaaagcctcgcctga

SEQ ID NO: 25 - DNA, 2049 bp, Artificial Sequence
  sequence of H0 Metschnikowia species QUP2 codon optimized for expression in S. cerevisiae
atgggtttcagaaacttgaagagaagattgtctaacgttggtgactccatgtctgttcactctgttaaggaagaagaagacttctccagagttgaaatcccagatgaaatctacaactacaagatcgtcttggttgctttgactgctgcttctgctgctatcatcatcggttacgatgctggtttcattggtggtactgtttctttgactgctttcaagtctgaattcggtttggacaagatgtctgctactgctgcttctgctatcgaaatgggtttcagaaacttgaagaggcgtttgtctaatgttggtgattccatgtctgttcactccgtcaaagaagaagaggatttctccagagttgaaatcccagacgaaatctacaactacaagatcgttttggttgctttgactgctgcttctgctgctattatcattggttatgatgctggtttcatcggtggtactgtttctttgacagctttcaagtctgaattcggtttggataagatgtctgctacagctgcttcagctattgaagctaatgttgtctctgtttttcaagctggtgcttactttggttgcctgtttttttacccaattggtgaaatttggggtcgtaagattggtttgttgttgtctggtttcttgttgacttttggtgctgccatttccttgatctctaattcttctagaggtttgggtgctatctatgctggtagagttttgactggtttaggtattggtggttgttcttctttagctcccatctacgttagtgaaattgctccagctgcaattagaggtaagttagttggttgttgggaagtttcttggcaagttggtggtatcgttggttattggattaactatggtgtcttgcaaaccctgccaatctcttctcaacaatggattattccattcgccgttcaattgattccatctggtttgttttggggtttgtgcttgttgattccagaatctccaagattcttggtgtccaaaggtaagattgataaggccagaaagaacttggcttacttgagaggtttgtctgaagatcatccatactccgtttttgagttggagaacatttccaaggccatcgaagaaaactttgaacaaacaggtagaggtttcttcgacccattgaaggctttgtttttcagcaagaaaatgctgtacaggctgctgttgtctacttctatgtttatgatgcaaaacggctacggtattaacgctgttacttattactctcccaccatctttaagtccttgggtgttcaaggttctaatgccggtttgttatctactggtattttcggtttgttgaaaggtgccgcttctgttttttgggttttcttcttggttgataccttcggtagaagattctgtttgtgctatttgtctttgccatgctctatctgcatgtggtatattggtgcctacattaagattgctaacccatctgctaaattggctgctggtgatactgctactactccagctggtactgctgctaaagctatgttgtatatttggaccatcttctacggtatcacttggaatggtactacctgggttatttgcgctgaaatttttccacaatctgttagaacagctgctcaagctgttaatgcttcttctaattggttttgggccttcatgattggtcattttactggtcaagctttggaaaacattggttacggttactactttttgttcgctgcttgttccgctattttcccagttgtagtttggttcgtttacccagaaacaaaaggtgttccattggaagctgttgaatacttgtttgaagttagaccatggaaggctcattcttacgctttagaaaagtaccagatcgagtacaacgaaggtgaattccatcaacataagccagaagttttgttgcagggttctgaaaactctgatacctctgaaaagtctttggcctgaaacgaaggtgaattccaccaacataagccagaagttttgttgcaaggttctgaaaactctgacacttctgaaaagtctttggcttaa

SEQ ID NO: 26 - DNA, 1887 bp, Artificial Sequence
  sequence of H0 Metschnikowia species APS1/HGT19
atgtcagaaaagcctgttgtgtcgcacagcatcgacacgacgctgtctacgtcatcgaaacaagtctatgacggtaactcgcttcttaagaccctgaatgagcgcgatggcgaacgcggcaatatcttgtcgcagtacactgaggaacaggccatgcaaatgggccgcaactatgcgttgaagcacaatttagatgcgacactctttggaaaggcggccgcggtcgcaagaaacccatacgagttcaattcgatgagttttttgaccgaagaggaaaaagtcgcgcttaacacggagcagaccaagaaatggcacatcccaagaaagttggtggaggtgattgcattggggtccatggccgctgcggtgcagggtatggatgagtcggtggtgaatggtgcaacgcttttctaccccacggcaatgggtatcacagatatcaagaatgccgatttgattgaaggtttgatcaacggtgcgccctatctttgctgcgccatcatgtgctggacatctgattactggaacaggaagttgggccgtaagtggaccattttctggacatgtgccatttctgcaatcacatgtatctggcaaggtctcgtcaatttgaaatggtaccatttgttcattgcgcgtttctgcttgggtttcggtatcggtgtcaagtctgccaccgtgcctgcgtatgctgccgaaaccaccccggccaaaatcagaggctcgttggtcatgctttggcagttcttcaccgctgtcggaatcatgcttggttacgtggcgtctttggcattctattacattggtgacaatggcatttctggcggcttgaactggagattgatgctaggatctgcatgtcttccagctatcgttgtgttagtccaagttccgtttgttccagaatcccctcgttggctcatgggtaaggaaagacacgctgaagcatatgattcgctccggcaattgcggttcagtgaaatcgaggcggcccgtgactgtttctaccagtacgtgttgttgaaagaggagggctcttatggaacgcagccattcttcagcagaatcaaggagatgttcaccgtgagaagaaacagaaatggtgcattgggcgcgtggatcgtcatgttcatgcagcagttctgtggaatcaacgtcattgcttactactcgtcgtcgatcttcgtggagtcgaatctttctgagatcaaggccatgttggcgtcttgggggttcggtatgatcaatttcttgtttgcaattccagcgttctacaccattgacacgtttggccgacgcaacttgttgctcactactttccctcttatggcggtattcttactcatggccggattcgggttctggatcccgttcgagacaaacccacacggccgtttggcggtgatcactattggtatctatttgtttgcatgtgtctactctgcgggcgagggaccagttcccttcacatactctgccgaagcattcccgttgtatatccgtgacttgggtatgggctttgccacggccacgtgttggttcttcaacttcattttggcattttcctggcctagaatgaagaatgcattcaagcctcaaggtgcctttggctggtatgccgcctggaacattgttggcttcttcttagtgttatggttcttgcccgagacaaagggcttgacgttggaggaattggacgaagtgtttgatgtgcctttgagaaaacacgcgcactaccgtaccaaagaattagtatacaacttgcgcaaatacttcttgaggcagaaccctaagccattgccgccactttatgcacaccaaagaatggctgttaccaacccagaatggttggaaaagaccgaggtcacgcacgaggagaatatctag

SEQ ID NO: 27 - DNA, 1887 bp, Artificial Sequence
  sequence of  H0 Metschnikowia species APS1/HGT19 codon optimized for expression in S. cerevisiae
atgtctgaaaagccagttgtttctcactctatcgacaccacctcttctacctcttctaagcaagtctacgacggtaactctttgttgaagacctctaacgaaagagacggtgaaagaggtaacatcttgtctcaatacactgaagaacaagcaatgcaaatgggtagaaactacgctttgaagcacaacttggacgctaccttgttcggtaaggctgctgctgtcgctagaaacccatacgagttcaactctatgtctttcttgaccgaagaagaaaaggtcgctttgaacaccgaacaaaccaagaagtggcacatcccaagaaagttggttgaagttattgctttgggttctatggctgctgctgttcaaggtatggacgaatctgttgttaacggtgctaccttgttctacccaaccgctatgggtatcaccgacatcaagaacgctgacttgattgaaggtttgattaacggtgccccatacttgtgttgtgctattatgtgttggacctctgactactggaacagaaagttgggtagaaagtggaccattttctggacctgtgctatttctgctatcacctgtatctggcaaggtttggtcaacttgaagtggtatcacttgttcattgctagattctgtttgggtttcggtatcggtgtcaagtctgctaccgttccagcctacgctgctgaaaccaccccagccaagattagaggttctttggttatgttgtggcaattcttcaccgctgtcggtattatgttgggttacgttgcttctttggctttctactacattggtgacaacggtatttctggtggtttgaactggagattgatgttgggttctgcttgtttgccagccatcgttgttttggtccaagttccattcgttccagaatctccaagatggttgatgggtaaggaaagacacgctgaagcctacgactctttgagacaattgagattctctgaaatcgaagccgctagagactgtttctaccaatacgttttgttgaaggaagaaggttcttacggtactcaaccattcttctctagaatcaaggaaatgttcaccgttagaagaaacagaaacggtgctttgggtgcttggattgttatgtttatgcaacaattctgtggtatcaacgtcattgcttactactcttcttctatcttcgttgaatctaacttgtctgaaatcaaggctatgttggcttcttggggtttcggtatgattaacttcttgttcgctattccagccttctacaccattgacaccttcggtagaagaaacttgttgttgactactttcccattgatggctgttttcttgttgatggctggtttcggtttctggattccattcgaaaccaacccacacggtagattggctgttatcactattggtatctacttgttcgcttgtgtctactctgctggtgaaggtccagttccattcacctactctgctgaagccttcccattgtacatcagagacttgggtatgggtttcgctaccgctacctgttggttcttcaacttcattttggctttctcttggccaagaatgaagaacgctttcaagcctcaaggtgctttcggttggtacgctgcttggaacattgttggtttcttcttggttttgtggttcttgccagaaactaagggtttgactttggaagaattggacgaagttttcgacgttccattgagaaagcacgctcactacagaactaaggaattggtttacaacttgagaaagtacttcttgagacaaaacccaaagccattgccaccattgtacgctcaccaaagaatggctgttaccaacccagaatggttggaaaagaccgaagtcacccacgaagaaaacatctaa

SEQ ID NO: 28 - protein, 318 aa, Artificial Sequence
  sequence of H0 Metschnikowia species Xyl1p
MATIKLNSGYDMPQVGFGCWKVTNSTCADTIYNAIKVGYRLFDGAEDYGNEKEVGEGINRAIDEGLVARDELFVVSKLWNNFHHPDNVEKALDKTLGDLNVEYLDLFLIHFPIAFKFVPFEEKYPPGFYCGEGDKFIYEDVPLLDTWRALEKFVKKGKIRSIGISNFSGALIQDLLRGAEIPPAVLQIEHHPYLQQPRLIEYVQSKGIAITAYSSFGPQSFVELDHPKVKECVTLFEHEDIVSIAKAHDKSAGQVLLRWATQRGLAVIPKSNKTERLLLNLNVNDFDLSEAELEQIAKLDVGLRFNNPWDWDKIPIFH

SEQ ID NO: 29 - protein, 362 aa, Artificial Sequence
  sequence of H0 Metschnikowia species Xyl2p
MPANPSLVLNKVNDITFENYEVPLLTDPNDVLVQVKKTGICGSDIHYYTHGRIGDFVLTKPMVLGHESAGVVVEVGKGVTDLKVGDKVAIEPGVPSRTSDEYKSGHYNLCPHMCFAATPNSNPDEPNPPGTLCKYYKSPADFLVKLPEHVSLELGAMVEPLTVGVHASRLGRVTFGDHVVVFGAGPVGILAAAVARKFGAASVTIVDIFDSKLELAKSIGAATHTFNSMTEGVLSEALPAGVRPDVVLECTGAEICVQQGVLALKAGGRHVQVGNAGSYLKFPITEFVTKELTLFGSFRYGYNDYKTSVAILDENYKNGKENALVDFEALITHRFPFKNAIEAYDAVRAGDGAVKCIIDGPE

SEQ ID NO: 30 - protein, 609 aa, Artificial Sequence
  sequence of H0 Metschnikowia species Xks1p
MTYSSSSGLFLGFDLSTQQLKIIVTNENLKALGTYHVEFDAQFKEKYAIKKGVLSDEKTGEILSPVHMWLEAIDHVFGLMKKDNFPFGKVKGISGSGMQHGSVFWSKSASSSLKNMAEYSSLTEALADAFACDTSPNWQDHSTGKEIKDFEKVVGGPDKLAEITGSRAHYRFTGLQIRKLAVRSENDVYQKTDRISLVSSFVASVLLGRITTIEEADACGMNLYNVTESKLDEDLLAIAAGVHPKLDNKSKRETDEGVKELKRKIGEIKPVSYQTSGSIAPYFVEKYGFSPDSKIVSFTGDNLATIISLPLRKNDVLVSLGTSTTVLLVTESYAPSSQYHLFKHPTIKNAYMGMICYSNGALARERVRDAINEKYGVAGDSWDKFNEILDRSGDFNNKLGVYFPIGEIVPNAPAQTKRMEMNSHEDVKEIEKWDLENDVTSIVESQTVSCRVRAGPMLSGSGDSNEGTPENENRKVKTLIDDLHSKFGEIYTDGKPQSYESLTSRPRNIYFVGGASRNKSIIHKMASIMGATEGNFQVEIPNACALGGAYKASWSLECESRQKWVHFNDYLNEKYDFDDVDEFKVDDKWLNYIPAIGLLSKLESNLDQN

SEQ ID NO: 31 - protein, 677 aa, Artificial Sequence
  sequence of H0 Metschnikowia species Tkl1p
MSDIDQLAISTIRLLAVDAVAKANSGHPGAPLGLAPAAHAVWKEMKFNPKNPDWVNRDRFVLSNGHACALLYAMLHLYGFDMSLDDLKQFRQLNSKTPGHPEKFEIPGAEVTTGPLGQGISNAVGLAIAQKQFAATFNKDDFAISDSYTYAFLGDGCLMEGVASEASSLAGHLQLNNLIAFWDDNKISIDGSTEVAFTEDVLKRYEAYGWDTLTIEKGDTDLEGVAQAIKTAKASKKPTLIRLTTIIGYGSLQQGTHGVHGAPLKPDDIKQLKEKFGFDPTKSFVVPQEVYDYYGTLVKKNQELESEWNKTVESYIQKFPEEGAVLARRLKGELPEDWAKCLPTYTADDKPLATRKLSEMALIKILDVVPELIGGSADLTGSNLTRAPDMVDFQPPQTGLGNYAGRYIRYGVREHGMGAIMNGIAGFGAGFRNYGGTFLNFVSYAAGAVRLSALSHLPVIWVATHDSIGLGEDGPTHQPIETLAHFRATPNISVWRPADGNEVSAAYKSAIESTSTPHILALTRQNLPQLAGSSVEKASTGGYTVYQTTDKPAVIIVASGSEVAISIDAAKKLEGEGIKANVVSLVDFHTFDKQPLDYRLSVLPDGVPIMSVEVMSSFGWSKYSHEQFGLNRFGASGKAEDLYKFFDFTPEGVADRAAKTVQFYKGKDLLSPLNRAF

SEQ ID NO: 32 - protein, 323 aa, Artificial Sequence
  sequence of H0 Metschnikowia species Tal1p
MSNSLESLKATGTVIVTDTGEFDSIAKYTPQDATTNPSLILAASKKAEYAKVIDVAIKYAEDKGSNPKEKAAIALDRLLVEFGKEILSIVPGRVSTEVDARLSFDKDATVKKALEIIELYKSIGISKDRVLIKIASTWEGIQAAKELEAKHDIHCNLTLLFSFVQAVACAEAKVTLISPFVGRILDWYKASTGKEYDAESDPGVVSVRQIYNYYKKYGYNTIVMGASFRNTGEIKALAGCDYLTVAPKLLEELMNSSEEVPKVLDAASASSASEEKVSYIDDESEFRFLLNEDAMATEKLAQGIRGFAKDAQTLLAELENRFK

SEQ ID NO: 33 - DNA, 957 bp, Artificial Sequence
  sequence of H0 Metschnikowia species XYL1
atggctactatcaaattgaactctggatacgacatgccccaagtgggttttgggtgctggaaagtaactaacagtacatgtgctgatacgatctacaacgcgatcaaagttggctacagattatttgatggcgctgaagattacgggaacgagaaagaggtgggcgaaggaatcaacagggccattgacgaaggcttggtggcacgtgacgagttgttcgtggtgtccaagctctggaacaacttccatcatccagacaacgtcgagaaggcgttggacaagactttgggcgacttgaatgtcgagtacttggacttgttcttgatccatttcccaattgcgttcaaattcgtgccctttgaggagaaatacccgcccggcttctactgtggagaaggcgataagtttatctacgaggatgtgcctttgcttgacacgtggcgggcattggagaagtttgtgaagaagggtaagatcagatccatcggaatctcgaacttttccggcgcgttgatccaggacttgctcaggggcgccgagatcccccctgccgtgttgcagattgagcaccacccatacttgcagcagcccagattgattgagtatgtgcagtccaagggtattgccatcacagcctactcctcttttggcccacagtcgtttgtggagttggaccaccccaaggtcaaggagtgtgtcacgcttttcgagcacgaagacattgtttccatcgctaaagctcacgacaagtccgcgggccaggtattattgaggtgggccacgcaaaggggtcttgccgtgattccaaagtcaaacaaaaccgagcgtttgttgctgaatttgaatgtgaacgattttgatctctctgaagcagaattggagcaaatcgcaaagttggacgtgggcttgcgcttcaacaacccttgggactgggacaagattccaatcttccattaa

SEQ ID NO: 34 - DNA, 1089 bp, Artificial Sequence
  sequence of H0 Metschnikowia species XYL2
atgcctgctaacccatccttggttttgaacaaagtgaacgacatcacgttcgagaactacgaggttccgttactcacagaccccaacgatgtattggttcaggtgaaaaagactggaatctgtggatctgacatccactactacacccacggcagaattggcgacttcgtgttgacaaagccaatggttttgggccacgaatccgccggtgtggtcgtggaggtcggcaaaggtgtcactgacttgaaggttggtgataaggttgccattgagcccggagtgccttctcgcaccagtgacgagtacaagagtggccactacaacttgtgcccacacatgtgttttgccgccacgcccaactctaaccccgacgagccaaacccgccagggactttgtgcaaatattacaagtccccagcggacttcttggtgaaattgcctgagcacgtctcccttgagttgggcgctatggtcgagcctttgactgtcggtgtgcacgcctcgcgtttgggccgtgtcacttttggtgaccacgttgtggttttcggtgctggcccagtcggtatccttgcggctgccgtggccagaaagtttggcgctgccagcgtgactatcgtcgacatcttcgacagcaaattggaattggccaagtccattggcgcggccactcacacattcaactcaatgactgagggtgttctttcggaggctttgcccgcgggcgtgagacctgacgttgtattggagtgcactggagcagagatctgtgtgcagcaaggtgtacttgcgttgaaggctggtggccgccacgtgcaagttggaaatgccggctcctatctcaaattccccatcaccgaatttgttaccaaggagttgactctctttggatccttccgttacggttacaacgactacaagacgtcggtcgccatcttggacgagaattacaagaacgggaaggagaatgcgttggtggactttgaagccttgattactcaccgtttccccttcaagaatgccattgaggcttacgacgcggtgcgcgctggcgacggagctgtcaagtgtatcattgacggcccagagtaa

SEQ ID NO: 35 - DNA, 1830 bp, Artificial Sequence
  sequence of H0 Metschnikowia species XKS1
atgacttatagttccagctctggcctctttttgggcttcgacttgtcgacgcagcagcttaaaatcattgtgacaaacgagaacttgaaggcgcttggtacctaccatgttgagtttgatgctcaattcaaagagaaatacgcgatcaaaaagggtgttttgtcagatgaaaaaacgggcgagattttatcacccgtgcacatgtggctagaggcaattgaccatgtctttgggttgatgaaaaaagacaatttccccttcggaaaagtgaaaggcataagcggttcagggatgcagcacggatcggtcttttggtcgaagtctgcttcttcatccttaaagaatatggccgaatattcctctttaacagaagccttggctgatgcctttgcgtgtgatacttctcccaactggcaggaccattcgacagggaaagaaatcaaagactttgagaaagtcgttggaggcccggacaaattggcggaaattacaggctcaagagctcactacaggttcactgggttgcagattcggaagttggcagtgagatctgagaatgacgtttaccagaaaaccgatagaatatctttggtgtcgagttttgttgcgtccgttcttttgggcaggatcaccacaattgaggaggcggacgcttgcggaatgaatttatacaatgtgaccgagtctaagcttgatgaagatttgttagcaatcgctgcaggggtgcatccaaagctcgataacaaatccaaaagggaaacagacgagggtgtcaaagaactaaagcgaaagattggtgagatcaaacccgtgagttatcagacttcgggctcaatcgcaccatattttgtcgagaaatacggcttctctccagattcgaagattgtttcgtttacgggtgataatcttgcgaccatcatctctttgcctttgagaaaaaacgacgtcttggtgtcactaggcacatccaccaccgtacttttggtgaccgagagctacgcgccttcttcgcagtatcatcttttcaagcatcctacaattaagaatgcttacatgggaatgatttgctacagtaatggcgcgctagcaagagaaagagttcgtgacgccatcaatgagaagtatggtgtggcaggggattcttgggacaagttcaatgagatcttggatcgctcaggcgacttcaacaataagttgggtgtttactttcccatcggtgaaattgtgcccaatgctccggcccagacaaagagaatggaaatgaactcgcatgaggatgtgaaagagatcgaaaagtgggatttggaaaacgatgtcacttctattgttgagtcacaaaccgttagttgccgagtgagagcgggcccaatgctttctggatcgggtgactcgaatgaaggaacgcccgaaaatgaaaataggaaagtcaaaacactcatcgacgatttacactctaagttcggcgaaatttacacagacgggaaacctcagagctacgagtctttgacttcgaggccgcggaacatctactttgtcggaggggcttcaagaaacaagagtatcatacacaagatggcttcgatcatgggtgctaccgaaggaaactttcaggttgagattccgaatgcgtgtgctcttggcggcgcctacaaggcaagctggagccttgagtgtgagagcagacaaaagtgggtgcacttcaatgattacctcaatgagaagtacgatttcgatgatgtggatgagttcaaagtggacgacaaatggctcaactatattccggcgattggcttgttgtcgaaattggaaagcaaccttgaccagaactaa

SEQ ID NO: 36 - DNA, 2034 bp, Artificial Sequence
  sequence of H0 Metschnikowia species TKL1
atgtccgacatcgatcaattggctatttctaccatccgtttgttggcggtcgacgccgtggccaaggccaactctggtcaccccggtgccccattgggtctcgcccctgccgcccacgccgtttggaaggagatgaaattcaacccaaagaaccccgactgggtcaacagagaccgttttgtgttgtcgaacggtcacgcttgcgctttgttatacgccatgttgcacctttacggcttcgacatgtcgcttgacgacttgaagcagttccgtcagttgaactcgaaaacacccggacatcccgagaagtttgaaatcccaggtgccgaggtcaccacgggccccttgggtcagggtatctccaacgccgtgggtttggccattgcacagaagcaattcgctgccacgttcaacaaggacgatttcgccatctctgactcgtacacctacgccttcttgggtgacggatgtttgatggagggtgtcgcctcggaagcatcttctttggctggccacctccaattgaacaacttgattgcgttctgggacgacaacaagatctcgatcgatggatccactgaagtggccttcaccgaggacgtgttgaagcgttacgaggcttacggttgggacacgctcacgattgagaagggtgacactgacttggagggcgtcgctcaggcgatcaagactgccaaggcgctgaagaagcctactttgatccgtttgaccaccatcatcggctacggctcgctccagcagggtacccacggtgttcacggtgctccattgaagccagatgacatcaagcagttgaaggagaagtttggcttcgacccaaccaagtcgtttgtcgtgcctcaggaagtttacgactactacggcacactcgtaaagaagaaccaggagttggagtccgagtggaacaagaccgtcgagtcctacatccagaaattcccagaggagggcgctgtcttggcgcgcagactcaagggtgagttgcctgaggactgggccaagtgcttgcctacttacaccgctgatgacaagccgttggccacgagaaagttgtctgagatggctctcatcaagatcttggatgtcgttccagagcttattggtggctctgccgacttgaccggctcgaacttgacccgtgcccctgacatggttgacttccagccccctcagaccggcttgggtaactacgctggtagatacatccgttacggtgtgcgtgagcacggtatgggtgccatcatgaacggtatcgccggttttggtgctggtttccgtaactacggcggtaccttcttgaacttcgtctcgtacgccgccggtgctgtgcgtttgtcggctctttctcacttgcctgtgatctgggttgctacgcatgactcgattggtttgggtgaggacggtcctacccaccagcctattgagaccttggcccacttcagagctacccctaacatctctgtgtggagacctgctgacggtaacgaggtgtcagctgcttacaagtctgccattgagtctacctctaccccacacatcttggccttgaccagacagaacttgcctcaattggctggttcttctgtggagaaggcctctaccggtggttacaccgtgtaccagaccactgacaagcctgccgtcatcatcgtggcttctggttccgaggtggccatctctattgacgccgccaagaagttggagggtgagggcatcaaggccaacgttgtttccttggttgacttccacactttcgacaagcagcctttggactaccgtttatctgttttgccagatggcgtgccaatcatgtccgttgaggtgatgtcctcgttcggctggtccaagtattctcacgagcagttcggcttgaacagattcggtgcctccggcaaggccgaagacctttacaagttcttcgacttcacgccagaaggcgttgctgacagagccgccaagaccgtgcagttctacaagggcaaggacctcctttcgcctttgaacagagccttctaa

SEQ ID NO: 37 - DNA, 972 bp, Artificial Sequence
  sequence of H0 Metschnikowia species TAL1
atgtctaactctttggaatccttgaaagctaccggcaccgtgatcgtcaccgacactggtgagttcgactcgattgccaagtacaccccacaagatgccaccaccaacccttcgttgattttagccgcctcgaaaaaggctgagtacgccaaggtgattgatgttgctattaaatacgccgaggacaagggcagcaaccctaaggagaaggccgccattgccttggacagattgttggtggagttcggtaaggaaatcttgctgattgtgcctggcagagtgtctaccgaggttgacgccagattgtcgtttgacaaggacgccaccgtcaagaaggcgcttgagatcatcgaattgtacaagtccattggcatctcgaaggacagagtgttgatcaagatcgcttccacctgggaaggtatccaggccgccaaggagttggaggccaagcacgacatccactgtaacttgacgcttttgttcagtttcgtgcaggcggtggcgtgtgccgaggccaaggtcactttgatctcgcctttcgtcggcagaatcttggactggtacaaggcctccaccggcaaggagtacgatgccgagtccgaccctggtgttgtgtctgtcagacagatctacaactactacaagaagtacggctacaacacgattgtcatgggcgcgtctttcagaaacactggcgagatcaaggccttggctggctgcgactacttgactgtggcccctaagttgttggaggagttgatgaactcttccgaggaggtgcctaaggtgttggacgctgcctcggccagctccgcgtctgaggagaaggtttcctacattgacgacgagagcgagttcagattcttgttgaacgaggacgccatggccaccgagaagttggcccagggtatcagaggctttgccaaggacgcccagaccttgttggccgagttggagaacagattcaagtag

SEQ ID NO: 38 - DNA, 49 bp, Artificial Sequence
  Primer Y10
gaaaaaactggtaccgtttaatcagtactgacaataaaaagattcttgt

SEQ ID NO: 39 - DNA, 49 bp, Artificial Sequence
  Primer Y10R
taatttctcttcgtatcccatggttgtttatgttcggatgtgatgtgag

SEQ ID NO: 40 - DNA, 60 bp, Artificial Sequence
  Primer Y15
acgccgccatccagtgtcgaaaacgagctttgtcttgtaaagagtcttcggtcattttta

SEQ ID NO: 41 - DNA, 60 bp, Artificial Sequence
  Primer Y15R
gcggccgcataggccactagtggatctgatcaatacatacaagcatctcacaatcacaag

SEQ ID NO: 42 - DNA, 60 bp, Artificial Sequence
  Primer Y33
tttttcacccacaacaaataatatcaaaagatgggttacgaggaaaagcttgtagcgccc

SEQ ID NO: 43 - DNA, 60 bp, Artificial Sequence
  Primer Y33R
acgagaacacccagctaaacgcggtgcgcgttagaccgtgcccgtcttctcgtctgaaga

SEQ ID NO: 44 - DNA, 66 bp, Artificial Sequence
  Primer Y41
cagagcagattgtactgagagtgcaccaggcgcgccccatccagtgtcgaaccatcattaaaagat

SEQ ID NO: 45 - DNA, 68 bp, Artificial Sequence
  Primer Y41R
ctccttacgcatctgtgcggtatttcacaccgcactagacaatacatacaagcatctcacaatcacaa

SEQ ID NO: 46 - DNA, 38 bp, Artificial Sequence
  Primer Y53
tcagtactgacaataaaaagattcttgttttcaagaac

SEQ ID NO: 47 - DNA, 28 bp, Artificial Sequence
  Primer Y53R
ctcacatcacatccgaacataaacaacc

SEQ ID NO: 48 - DNA, 22 bp, Artificial Sequence
  Primer Y83
tatcccgtcacttccacattcg

SEQ ID NO: 49 - DNA, 32 bp, Artificial Sequence
  Primer Y83R
tattgatatagtgtttaagcgaatgacagaag

SEQ ID NO: 50 - DNA, 62 bp, Artificial Sequence
  Primer Y96i
atagaaagcaaatagttatataatttttcatggacgtaggtctagagatctgtttagcttgc

SEQ ID NO: 51 - DNA, 66 bp, Artificial Sequence
  Primer Y95Ri
aatgcaaaagcggctcctaaacagaaattcttcagtcaatacatacaagcatctcacaatcacaag

SEQ ID NO: 52 - DNA, 58 bp, Artificial Sequence
  Primer Y93Ri
tcgtctatatcaaaactgcatgtttctctacgtctaattaagggttctcgagagctcg

SEQ ID NO: 53 - DNA, 56 bp, Artificial Sequence
  Primer Y91i
acttcaatagacttcaatagaaagcaaatagttatatgccctgaggatgtatctgg

(19) United States
(12) Patent Application Publication
(10) Pub. No.: US 2018/0195051 A1
(43) Pub. Date: Jul. 12, 2018

(54) METHODS AND ORGANISM WITH INCREASED ETHANOL PRODUCTION

(71) Applicant: Creatus Biosciences Inc., Vancouver (CA)

(72) Inventors: Zongli Luo, Vancouver (CA); Hendrik Jurgens Jansen van Vuuren, Lions Bay (CA); Allan George DeBono, Vancouver (CA); Andrew Taplin Ferguson, Vancouver (CA)

(21) Appl. No.: 15/849,122

(22) Filed: Dec. 20, 2017

Related U.S. Application Data
(60) Provisional application No. 62/437,596, filed on Dec. 21, 2016.

(51) Int. Cl. C12N 9/04; C12N 9/10; C12N 9/16; C12P 7/06; C12N 9/12

(52) U.S. Cl. CPC C12N 9/0006; C12N 9/1022; C12Y 202/01002; C12N 9/16; C12Y 207/01017; C12P 7/06; C12Y 101/01307; C12Y 101/01175; C12N 9/1205; C12Y 301/03041

(57) ABSTRACT

Provided herein are non-naturally occurring microbial organisms having increased xylose metabolism and increased production of ethanol using xylose as a substrate, as well as methods to make and use these microbial organisms to produce ethanol.

### CROSS-REFERENCE TO RELATED APPLICATION

This application claims the benefit of priority of U.S. Provisional Application No. 62/437,596, filed on Dec. 21, 2016, the content of which is herein incorporated by reference in its entirety.

### FIELD

The present invention relates to the field of molecular biology and microbiology. Provided herein are non-naturally occurring microbial organisms having increased xylose metabolism and increased production of bioderived ethanol using xylose as a substrate, as well as methods to produce ethanol using these microbial organisms.

### REFERENCE TO SEQUENCE LISTING

The instant application contains a Sequence Listing which has been submitted in ASCII format via EFS-Web and is hereby incorporated by reference in its entirety. Said ASCII copy, created on Dec. 19, 2017, is named 14305-006-999 Sequence_Listing.txt and is 118,163 bytes in size.

### BACKGROUND

Ethanol has a number of uses and is most commonly used as a fuel additive. As a fuel additive, ethanol is a low value product with much of the cost of its production attributed to the cost of raw materials. It would be desirable, therefore, to develop ethanologens and fermentation processes for the production of ethanol from readily available, inexpensive starting materials, such as lignocellulose. Xylose is an abundant sugar present in lignocellulosic biomass, a renewable feedstock for producing bioderived ethanol. Most microorganisms are able to ferment glucose but few have been reported to utilize xylose efficiently and even fewer ferment this pentose to ethanol. Ethanol production from xylose is limited by the absence or low natural xylose uptake and/or metabolism in microbial organisms. Therefore, methods to confer or enhance xylose uptake and/or metabolism in microbial organisms to increase the production of bioderived ethanol from xylose represent unmet needs. The non-naturally occurring microbial organisms and methods provided herein meet these needs and provide other related advantages.

### SUMMARY OF THE INVENTION

Provided herein are non-naturally occurring microbial organisms having a xylose-ethanol pathway and at least one exogenous nucleic acid encoding an enzyme or protein expressed in a sufficient amount to confer or enhance xylose uptake or metabolism, wherein the enzyme or protein has an amino acid sequence that is at least 89% identical to a Metschnikowia enzyme or protein such as a xylose transporter, a xylose reductase, a xylose dehydrogenase, or a xylulokinase. The Metschnikowia species can be H0 Metschnikowia species.

In some embodiments, the non-naturally occurring microbial organism can have at least two exogenous nucleic acids encoding two enzymes or proteins selected from the group consisting of a xylose transporter, a xylose reductase, a xylose dehydrogenase and a xylulokinase. In some embodiments, the non-naturally occurring microbial organism can have at least three exogenous nucleic acids encoding three enzymes or proteins selected from the group consisting of a xylose transporter, a xylose reductase, a xylose dehydrogenase and a xylulokinase. In some embodiments, the non-naturally occurring microbial organism can have at least four exogenous nucleic acids encoding the four enzymes or proteins of a xylose transporter, a xylose reductase, a xylose dehydrogenase and a xylulokinase.

In some embodiments, the non-naturally occurring microbial organism provided herein can have at least one exogenous nucleic acid encoding an enzyme or protein having an amino acid sequence that is at least 89% identical with a Metschnikowia xylose transporter such as Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, or Aps1p/Hgt19p. In some embodiments, the non-naturally occurring microbial organism provided herein can have at least one exogenous nucleic acid encoding an enzyme or protein that is a Metschnikowia xylose transporter such as Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, or Aps1p/Hgt19p. The xylose transporter can be Xyt1p. The xylose transporter can be Gxf1p. The xylose transporter can be Aps1p/Hgt19p. The Metschnikowia species can be H0 Metschnikowia species.

In some embodiments, the non-naturally occurring microbial organisms provided herein can have at least one exogenous nucleic acid encoding an enzyme or protein that has an amino acid sequence that is at least 89% identical to a Metschnikowia enzyme or protein such as a xylose transporter, a xylose reductase, a xylose dehydrogenase, or a xylulokinase, wherein the xylose reductase is Xyl1p, the xylose dehydrogenase is Xyl2p, and the xylulokinase is Xks1p. The Metschnikowia species can be H0 Metschnikowia species.

In some embodiments, the non-naturally occurring microbial organism can further include at least one exogenous nucleic acid encoding a transketolase, a transaldolase, or both. The transketolase can be Tkl1p. The transaldolase can be Tal1p.

In some embodiments, the exogenous nucleic acid can be codon-optimized to produce the enzyme or protein to confer or enhance xylose uptake or metabolism in the host microbial organism. The exogenous nucleic acid can be a heterologous nucleic acid. The exogenous nucleic acid can be included in an expression vector. The exogenous nucleic acid can also be integrated into the genome of the microbial organism.

In some embodiments, the non-naturally occurring microbial organism provided herein can further include one or more gene disruptions occurring in genes encoding an enzyme or protein that reduces xylose metabolism or enhances ethanol metabolism. In some embodiments, the microbial organism can have a disruption in the gene encoding phosphatase Pho13p.

In some embodiments, the non-naturally occurring microbial organism provided herein can include one or more gene disruptions occurring in genes encoding an enzyme or protein such as an ethanol dehydrogenase, an acetaldehyde dehydrogenase, or an acetate coA-transferase. In some embodiments, the microbial organism can have a gene disruption occurring in genes encoding an acetaldehyde dehydrogenase. The ethanol dehydrogenase can be Adh2p. The acetaldehyde dehydrogenase can be Ald2p. The acetate coA-transferase can be Acs1p.

Provided herein are non-naturally occurring microbial organisms having a xylose-ethanol pathway and at least one exogenous nucleic acid encoding an enzyme or protein expressed in a sufficient amount to confer or enhance xylose uptake or metabolism. The microbial organism can be in a substantially anaerobic culture medium. The non-naturally occurring microbial organism can be a species of bacteria or yeast.

In some embodiments, the microbial organism can be a species of a yeast such as Saccharomyces cerevisiae, Schizosaccharomyces pombe, Candida albicans, Candida tropicalis, Debaryomyces hansenii, Kluyveromyces lactis, Kluyveromyces marxianus, Aspergillus terreus, Aspergillus niger, Chlamydomonas reinhardtii, Pichia pastoris, Rhizopus arrhizus, Rhizobus oryzae, Trichoderma reesei, or Yarrowia lipolytica.

In some embodiments, the microbial organism can be a species of a bacteria such as Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, or Pseudomonas putida.

Provided herein is also a method for producing ethanol including culturing the microbial organism described herein under conditions and for a sufficient period of time to produce ethanol. The microbial organism can be cultured in medium having xylose and a co-substrate such as cellobiose, hemicellulose, glycerol, galactose, glucose, or a combination thereof. The microbial organism can be cultured in substantially anaerobic culturing conditions. The microbial organism can be cultured in batch cultivation, fed-batch cultivation or continuous cultivation.

In some embodiments, the method can further include separating ethanol from other components in the culture. The separation method can include extraction, continuous liquid-liquid extraction, pervaporation, membrane filtration, membrane separation, reverse osmosis, electrodialysis, distillation, crystallization, centrifugation, extractive filtration, ion exchange chromatography, absorption chromatography, or ultrafiltration.

In some embodiments, the methods provided herein has conversion efficiency of ethanol from xylose of at least 0.25 g ethanol/g xylose. In some embodiments, the methods provided herein produces ethanol at a rate of at least 0.5 g/L/h.

Also provided herein is a bioderived ethanol produced using methods described herein. The bioderived ethanol can have glycerol, acetate, glyceraldehyde, acetaldehyde or a combination thereof as impurities.

Also provided herein is a composition having the bioderived ethanol described herein. The composition can be culture medium. The composition can be the culture medium with the microbial organism removed.

### BRIEF DESCRIPTION OF THE DRAWINGS

FIG. 1 shows that the expression of H0 XYT1 in S. cerevisiae increased the xylose transport from about 10% to about 74% (48 hours).

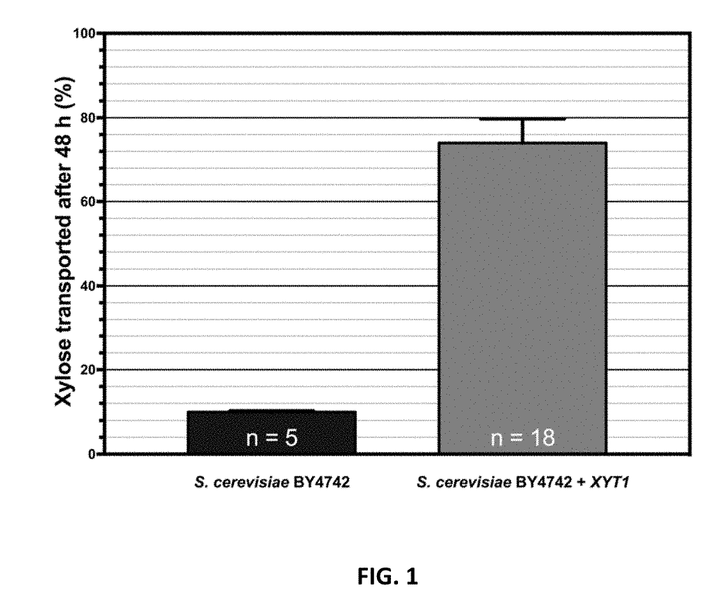

FIG. 2 shows a xylose metabolic pathway. As shown, xylose is reduced to xylitol by an NAD[P]H+ linked xylose reductase (XR, e.g., Xyl1p), and the xylitol is oxidized to xylulose by an NAD+ linked xylitol dehydrogenase (XDH, e.g., Xyl2p). D-xylulokinase (e.g., Xks1p) phosphorylates D-xylulose to form D-xyluose-5-phosphate (X5P). Transketolase (e.g., Tkl1p) catalyzes conversion of xylulose-5-phosphate and ribose-5-phosphate to sedoheptulose-7-phosphate and glyceraldehyde-3-phosphate, and transaldolase (e.g., Tal1p) converts sedoheptulose 7-phosphate and glyceraldehyde 3-phosphate to erythrose 4-phosphate and fructose 6-phosphate. Erythrose 4-phosphate is recycled to the pentose phosphate pathway (PPP), and fructose 6-phosphate can be metabolized further to produce ethanol via glycolysis pathway.

FIG. 3 shows the xylose-ethanol pathway as well as ethanol metabolic pathway. As shown, increased xylose metabolism can lead to increased ethanol production. In the meantime, the endogenous ethanol metabolic pathways can convert ethanol to acetyl-CoA through alcohol dehydrogenase (ADH), which catalyzes the conversion of alcohol to acetaldehyde, aldehyde dehydrogenase (ALD), which catalyzes the conversion of acetaldehyde to acetate, acetate coA-transferase (ACS), which catalyzes the conversion of acetate to acetyl-CoA. Reduction of the endogenous ethanol metabolism can increase the ethanol production from xylose by microbial organism.

### DETAILED DESCRIPTION

Provided herein are non-naturally occurring microbial organisms having enhanced xylose uptake and/or metabolism, and a xylose-ethanol pathway that converts xylose to ethanol. Some Metschnikowia species, e.g. the H0 Metschnikowia species, have a robust xylose uptake and metabolism machinery, and can consume and metabolize xylose as its sole carbon source. Heterologous expression of enzymes or proteins constituting the xylose uptake and metabolism machinery of these Metschnikowia species in a host microbial organism (e.g. S. cerevisiae) can confer or enhance xylose uptake and/or metabolism in the host microbial organism, allowing it to consume xylose and to produce ethanol from xylose through the xylose-ethanol pathway.

As used herein, the term “non-naturally occurring,” when used in reference to a microbial organism or microorganism described herein is intended to mean that the microbial organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. Exemplary metabolic polypeptides include enzymes or proteins within an ethanol biosynthetic pathway.

As used herein, the terms “microbial,” “microbial organism” or “microorganism” are intended to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

As used herein, the term “isolated” when used in reference to a microbial organism is intended to mean an organism that is substantially free of at least one component as the referenced microbial organism is found in nature. The term includes a microbial organism that is removed from some or all components as it is found in its natural environment. The term also includes a microbial organism that is removed from some or all components as the microbial organism is found in non-naturally occurring environments. Therefore, an isolated microbial organism is partly or completely separated from other substances as it is found in nature or as it is grown, stored or subsisted in non-naturally occurring environments. Specific examples of isolated microbial organisms include partially pure microbes, substantially pure microbes and microbes cultured in a medium that is non-naturally occurring.

As used herein, the terms “exogenous” is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism. Therefore, the term “endogenous” refers to a referenced molecule or activity that is present in the host. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism. Accordingly, exogenous expression of an encoding nucleic acid of the invention can utilize either or both a heterologous or homologous encoding nucleic acid.

It is understood that when more than one exogenous nucleic acid is included in a microbial organism that the more than one exogenous nucleic acids refers to the referenced encoding nucleic acid or biosynthetic activity, as discussed above. It is also understood that a microbial organism can have one or multiple copies of the same exogenous nucleic acid. It is further understood that the more than one exogenous nucleic acids can be introduced into the host microbial organism on separate nucleic acid molecules, on polycistronic nucleic acid molecules, or a combination thereof, and still be considered as more than one exogenous nucleic acid. For example, as disclosed herein a microbial organism can be engineered to express two or more exogenous nucleic acids encoding a desired pathway enzyme or protein. In the case where two exogenous nucleic acids encoding a desired activity are introduced into a host microbial organism, it is understood that the two exogenous nucleic acids can be introduced as a single nucleic acid, for example, on a single plasmid, on separate plasmids, can be integrated into the host chromosome at a single site or multiple sites, and still be considered as two exogenous nucleic acids. Similarly, it is understood that more than two exogenous nucleic acids can be introduced into a host organism in any desired combination, for example, on a single plasmid, on separate plasmids, can be integrated into the host chromosome at a single site or multiple sites, and still be considered as two or more exogenous nucleic acids, for example three exogenous nucleic acids. Thus, the number of referenced exogenous nucleic acids or biosynthetic activities refers to the number of encoding nucleic acids or the number of biosynthetic activities, not the number of separate nucleic acids introduced into the host organism.

As used herein, the term “xylose” refers to a five carbon monosaccharide with a formyl functional group having the chemical formula of C5Hio05, a Molar mass of 150.13 g/mol, and one IUPAC name of (3R,4S,5R)-oxane-2,3,4,5-tetrol. Xylose is also known in the art as D-xylose, D-xylopyranose, xyloside, d-(+)-xylose, xylopyranose, wood sugar, xylomed and D-xylopentose.

As used herein, the term “xylose-ethanol pathway” when used in connection with a microbial organism refers to a metabolic pathway that uses xylose as the substrate to produce ethanol. The xylose-ethanol pathway can include multiple enzymes or proteins catalyzing different steps in the production of ethanol from xylose, including, (1) a xylose transporter that allows the uptake of xylose, (2) enzymes or proteins of the xylose metabolic pathway including a xylose reductase, a xylitol dehydrogenase, a xylulokinase, a transketolase and a transaldolase that together convert xylose to xylulose-5-phosphate. Xylulose-5-phosphate is combined with ribose-5-phosphate to yield glyceraldehyde-3-phosphate and sedoheptulose-7-phosphate which are subsequently converted to erythrose-4-phosphate and fructose-6-phosphate, and (3) enzymes or proteins of the glycolysis and fermentation pathways that further metabolize glyceraldehyde-3-phosphate and fructose-6-phosphate to produce ethanol. The xylose-ethanol pathway can also include enzymes or proteins that recycle glyceraldehyde-3-phosphate and xylulose-5-phosphate back into PPP.

These enzymes or proteins can be endogenous or exogenous to the microbial organism. The xylose-ethanol pathway is an “endogenous” pathway to the host microbial organism if the xylose-ethanol pathway includes only endogenous enzymes or proteins. The xylose-ethanol pathway is an “exogenous” pathway to the host microbial organism if the xylose-ethanol pathway includes one or more exogenous enzymes or proteins. An exogenous xylose-ethanol pathway can also include endogenous enzymes or proteins.

As used herein, the term “Metschnikowia species” refers to any species of yeast that falls within the Metschnikowia genus. Exemplary Metschnikowia species include, but are not limited to, Metschnikowia pulcherrima, Metschnikowia fructicola, Metschnikowia chrysoperlae, Metschnikowia reukaufii, Metschnikowia andauensis, Metschnikowia sinensis, Metschnikowia shanxiensis, Metschnikowia zizyphicola, Metschnikowia bicuspidata, Metschnikowia lunata, Metschnikowia zobellii, Metschnikowia australis, Metschnikowia agaveae, Metschnikowia gruessii, Metschnikowia hawaiiensis, Metschnikowia krissii, Metschnikowia sp. strain NS—O-85, Metschnikowia sp. strain NS—O-89 and the unique Metschnikowia species described herein Metschnikowia sp. H0, alternatively referred to as the “H0 Metschnikowia species.” The Metschnikowia species described herein, i.e., the H0 Metschnikowia species, is a newly discovered species, which is designated Accession No. 081116-01, was deposited at International Depositary Authority of Canada (“IDAC”), an International Depositary Authority, at the address of 1015 Arlington Street, Winnipeg, Manitoba, Canada R3E 3R2, on Nov. 8, 2016, under the terms of the Budapest Treaty.

As used herein, the term “attenuate,” or grammatical equivalents thereof, is intended to mean to weaken, reduce or diminish the activity or amount of an enzyme or protein. Attenuation of the activity or amount of an enzyme or protein can mimic complete disruption if the attenuation causes the activity or amount to fall below a critical level required for a given pathway to function. However, the attenuation of the activity or amount of an enzyme or protein that mimics complete disruption for one pathway, can still be sufficient for a separate pathway to continue to function. For example, attenuation of an endogenous enzyme or protein can be sufficient to mimic the complete disruption of the same enzyme or protein for production of a particular product, but the remaining activity or amount of enzyme or protein can still be sufficient to maintain other pathways or reactions, such as a pathway that is critical for the host microbial organism to survive, reproduce or grow. Attenuation of an enzyme or protein can also be weakening, reducing or diminishing the activity or amount of the enzyme or protein in an amount that is sufficient to increase yield of ethanol, but does not necessarily mimic complete disruption of the enzyme or protein.

As used herein, the term “genetic modification,” “gene disruption,” or grammatical equivalents thereof, is intended to mean a genetic alteration that renders the encoded gene product functionally inactive, or active but attenuated. The genetic alteration can be, for example, deletion of the entire gene, deletion of a regulatory sequence required for transcription or translation, deletion of a portion of the gene that results in a truncated gene product, or by any of the various mutation strategies that inactivate or attenuate the encoded gene product well known in the art. One particularly useful method of gene disruption is complete gene deletion because it reduces or eliminates the occurrence of genetic reversions in the non-naturally occurring microorganisms of the invention. A gene disruption also includes a null mutation, which refers to a mutation within a gene or a region containing a gene that results in the gene not being transcribed into RNA and/or translated into a functional gene product. Such a null mutation can arise from many types of mutations including, for example, inactivating point mutations, deletion of a portion of a gene, entire gene deletions, or deletion of chromosomal segments.

As used herein, the term “overexpression” or grammatical equivalents thereof, is intended to mean the expression of a gene product (e.g., ribonucleic acids (RNA), protein or enzyme) in an amount that is greater than is normal for a host microbial organism, or at a time or location within the host microbial organism that is different from that of wild-type expression.

The non-naturally occurring microbal organisms provided herein can contain stable genetic alterations, which refers to microorganisms that can be cultured for greater than five generations without loss of the alteration. Generally, stable genetic alterations include modifications that persist greater than 10 generations, particularly stable modifications will persist more than about 25 generations, and more particularly, stable genetic modifications will be greater than 50 generations, including indefinitely.

In the case of gene disruptions, a particularly useful stable genetic alteration is a gene deletion. The use of a gene deletion to introduce a stable genetic alteration is particularly useful to reduce the likelihood of a reversion to a phenotype prior to the genetic alteration. For example, stable growth-coupled production of a biochemical can be achieved, for example, by deletion of a gene encoding an enzyme catalyzing one or more reactions within a set of metabolic modifications. The stability of growth-coupled production of a biochemical can be further enhanced through multiple deletions, significantly reducing the likelihood of multiple compensatory reversions occurring for each disrupted activity.

Those skilled in the art will understand that the genetic alterations, including metabolic modifications exemplified herein, are described with reference to a suitable host organism such as S. cerevisiae and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the S. cerevisiae metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

An ortholog is a gene or genes that are related by vertical descent and are responsible for substantially the same or identical functions in different organisms. For example, mouse epoxide hydrolase and human epoxide hydrolase can be considered orthologs for the biological function of hydrolysis of epoxides. Genes are related by vertical descent when, for example, they share sequence similarity of sufficient amount to indicate they are homologous, or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Genes that are orthologous can encode proteins with sequence similarity of about 25% to 100% amino acid sequence identity. Genes encoding proteins sharing an amino acid similarity less that 25% can also be considered to have arisen by vertical descent if their three-dimensional structure also shows similarities. Members of the serine protease family of enzymes, including tissue plasminogen activator and elastase, are considered to have arisen by vertical descent from a common ancestor.

Orthologs include genes or their encoded gene products that through, for example, evolution, have diverged in structure or overall activity. For example, where one species encodes a gene product exhibiting two functions and where such functions have been separated into distinct genes in a second species, the three genes and their corresponding products are considered to be orthologs. For the production of a biochemical product, those skilled in the art will understand that the orthologous gene harboring the metabolic activity to be introduced or disrupted is to be chosen for construction of the non-naturally occurring microorganism. An example of orthologs exhibiting separable activities is where distinct activities have been separated into distinct gene products between two or more species or within a single species. A specific example is the separation of elastase proteolysis and plasminogen proteolysis, two types of serine protease activity, into distinct molecules as plasminogen activator and elastase. A second example is the separation of mycoplasma 5′-3′ exonuclease and Drosophila DNA polymerase III activity. The DNA polymerase from the first species can be considered an ortholog to either or both of the exonuclease or the polymerase from the second species and vice versa.

In contrast, paralogs are homologs related by, for example, duplication followed by evolutionary divergence and have similar or common, but not identical functions. Paralogs can originate or derive from, for example, the same species or from a different species. For example, microsomal epoxide hydrolase (epoxide hydrolase I) and soluble epoxide hydrolase (epoxide hydrolase II) can be considered paralogs because they represent two distinct enzymes or proteins, co-evolved from a common ancestor, that catalyze distinct reactions and have distinct functions in the same species. Paralogs are proteins from the same species with significant sequence similarity to each other suggesting that they are homologous, or related through co-evolution from a common ancestor. Groups of paralogous protein families include HipA homologs, luciferase genes, peptidases, and others.

A nonorthologous gene displacement is a nonorthologous gene from one species that can substitute for a referenced gene function in a different species. Substitution includes, for example, being able to perform substantially the same or a similar function in the species of origin compared to the referenced function in the different species. Although generally, a nonorthologous gene displacement will be identifiable as structurally related to a known gene encoding the referenced function, less structurally related but functionally similar genes and their corresponding gene products nevertheless will still fall within the meaning of the term as it is used herein. Functional similarity requires, for example, at least some structural similarity in the active site or binding region of a nonorthologous gene product compared to a gene encoding the function sought to be substituted. Therefore, a nonorthologous gene includes, for example, a paralog or an unrelated gene.

Therefore, in identifying and constructing the non-naturally occurring microbial organisms of the invention having biosynthetic capability, those skilled in the art will understand with applying the teaching and guidance provided herein to a particular species that the identification of metabolic modifications can include identification and inclusion or inactivation of orthologs. To the extent that paralogs and/or nonorthologous gene displacements are present in the referenced microorganism that encode an enzyme catalyzing a similar or substantially similar metabolic reaction, those skilled in the art also can utilize these evolutionally related genes. Similarly for a gene disruption, evolutionally related genes can also be disrupted or deleted in a host microbial organism to reduce or eliminate functional redundancy of enzymatic activities targeted for disruption.

Orthologs, paralogs and nonorthologous gene displacements can be determined by methods well known to those skilled in the art. For example, inspection of nucleic acid or amino acid sequences for two polypeptides will reveal sequence identity and similarities between the compared sequences. Based on such similarities, one skilled in the art can determine if the similarity is sufficiently high to indicate the proteins are related through evolution from a common ancestor. Algorithms well known to those skilled in the art, such as Align, BLAST, Clustal W and others compare and determine a raw sequence similarity or identity, and also determine the presence or significance of gaps in the sequence which can be assigned a weight or score. Such algorithms also are known in the art and are similarly applicable for determining nucleotide sequence similarity or identity. Parameters for sufficient similarity to determine relatedness are computed based on well known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 25% to 100% sequence identity. Proteins that are unrelated can have an identity which is essentially the same as would be expected to occur by chance, if a database of sufficient size is scanned (about 5%). Sequences between 5% and 24% may or may not represent sufficient homology to conclude that the compared sequences are related. Additional statistical analysis to determine the significance of such matches given the size of the data set can be carried out to determine the relevance of these sequences.

Exemplary parameters for determining relatedness of two or more sequences using the BLAST algorithm, for example, can be as set forth below. Briefly, amino acid sequence alignments can be performed using BLASTP version 2.0.8 (Jan. 5, 1999) and the following parameters: Matrix: 0 BLOSUM62; gap open: 11; gap extension: 1; x_dropoff: 50; expect: 10.0; wordsize: 3; filter: on. Nucleic acid sequence alignments can be performed using BLASTN version 2.0.6 (Sep. 16, 1998) and the following parameters: Match: 1; mismatch: −2; gap open: 5; gap extension: 2; x_dropoff: 50; expect: 10.0; wordsize: 11; filter: off. Those skilled in the art will know what modifications can be made to the above parameters to either increase or decrease the stringency of the comparison, for example, and determine the relatedness of two or more sequences.

As used herein, the term “medium,” “culture medium,” “growth medium” or grammatical equivalents thereof refers to a liquid or solid (e.g., gelatinous) substance containing nutrients that supports the growth of a cell, including any microbial organism species described herein. Nutrients that support growth include: a substrate that supplies carbon, such as, but are not limited to, xylose, cellobiose, hemicelluloses, glycerol, galactose and glucose; salts that provide essential elements including magnesium, nitrogen, phosphorus, and sulfur; and a source for amino acids, such as peptone or tryptone; and a source for vitamin content, such as yeast extract. Examples of medium include yeast extract peptone (YEP) medium and yeast nitrogen base (YNB) medium having a carbon source such as, but not limited to xylose, glucose, cellobiose, galactose, or glycerol, or a combination thereof. The formulations of YEP and YNB medium are well known in the art. For example, YEP medium having 4% xylose includes, but is not limited to, yeast extract 1.0 g, peptone 2.0 g, xylose 4.0 g, and 100 ml water. As another example, YNB medium having 2% glucose and 2% xylose includes, but is not limited to, biotin calcium pantothenate 400 μg, folic acid 2 μg, inositol 2000 μg, niacin 400 μg, paminobenzoic acid 200 μg, pyridoxine hydrochloride 400 μg, riboflavin 200 μg, thiamine hydrochloride 400 μg, boric acid 500 μg, copper sulfate 40 μg, potassium iodide 100 μg, ferric chloride 200 μg, manganese sulfate 400 μg, sodium molybdate 200 μg, zinc sulfate 400 μg, potassium phosphate monobasic 1 g, magnesium sulfate 500 mg, sodium chloride 100 mg, calcium chloride 100 mg, 20 g glucose, 20 g, xylose and 1 L water. The amount of the carbon source in the medium can be readily determined by a person skilled in the art. When more than one substrate that supplies carbon is present in the medium, these are referred to as “co-substrates.” Medium can also include substances other than nutrients needed for growth, such as a substance that only allows select cells to grow (e.g., antibiotic or antifungal), which are generally found in selective medium, or a substance that allows for differentiation of one microbial organism over another when grown on the same medium, which are generally found in differential or indicator medium. Such substances are well known to a person skilled in the art.

As used herein, the term “aerobic” when used in reference to a culture or growth condition is intended to mean that the free oxygen (O₂) is available in the culture or growth condition. The term “anaerobic” when used in reference to a culture or growth condition is intended to mean that the culture or growth condition lacks free oxygen (O₂). The term “substantially anaerobic” when used in reference to a culture or growth condition is intended to mean that the amount of dissolved oxygen in a liquid medium is less than about 10% of saturation. The term also is intended to include sealed chambers maintained with an atmosphere of less than about 1% oxygen that include liquid or solid medium.

As used herein, the term “bioderived” means derived from or synthesized by a biological organism and can be considered a renewable resource since it can be generated by a biological organism. Such a biological organism, in particular the microbial organism disclosed herein, can utilize feedstock or biomass, such as, sugars (e.g., xylose, glucose, fructose, galactose, sucrose, and arabinose), carbohydrates obtained from an agricultural, plant, bacterial, or animal source, and glycerol.

As used herein, the term “biobased” means a product is composed, in whole or in part, of a bioderived compound. A biobased or bioderived product is in contrast to a petroleum derived product, wherein such a product is derived from or synthesized from petroleum or a petrochemical feedstock.

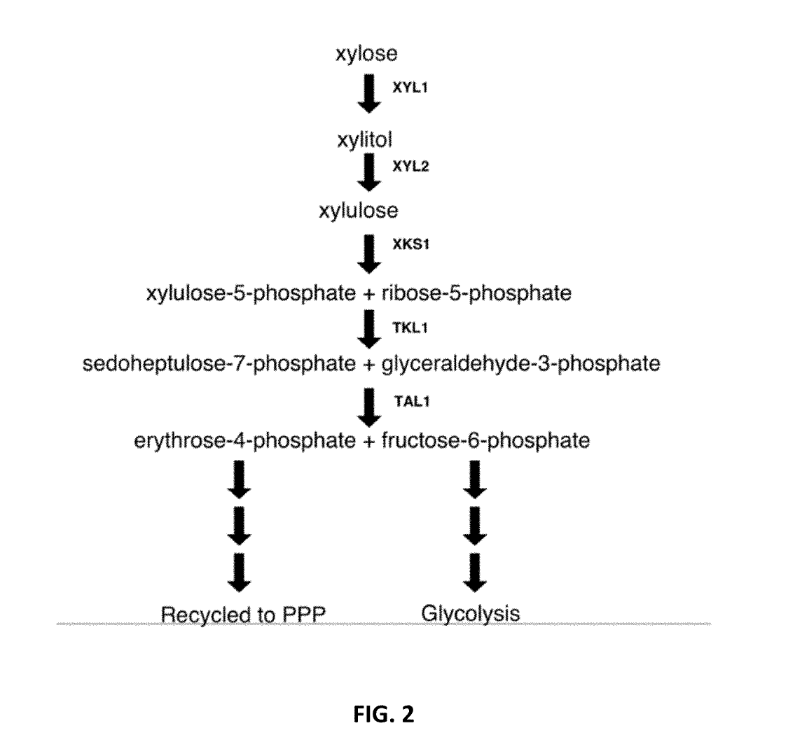

Provided herein are non-naturally occurring microbial organisms having a xylose-ethanol pathway that produces ethanol from xylose. As shown in FIG. 2, a complete xylose-ethanol pathway include enzymes or proteins catalyzing different steps of the production of ethanol from xylose, including, (1) a xylose transporter (e.g. Xyt1), (2) enzymes or proteins of the xylose metabolic pathway including xylose reductase (e.g. Xyl1p), xylose dehydrogenase (e.g. Xyl2p), xylulokinase (e.g. Xks1p), transketolase (e.g. Tkl1p), and transaldolase (e.g. Tal1p) that together convert xylose to erythrose 4-phosphate and fructose 6-phosphate, which can feed into the PPP and the glycolysis pathways, and (3) enzymes or proteins of the glycolysis and fermentation pathways that further metabolize fructose 6-phosphate to produce ethanol. For example, in Saccharomyces, an exemplary model of fermentation, glucose is converted to fructose-6-phosphate by hexokinases (e.g. Hxk1p, Hxk2p) and phosphoglucoisomerase (e.g. Pgi1p), which is further converted to ethanol. Starting from xylose as the carbon source for ethanol production, these two steps can be skipped. In an exemplary xylose-ethanol pathway, fructose-6-phosphate generated from the xylose metabolic pathway and from PPP is converted to fructose-1,6-bisphosphate by phosphofructokinases (e.g. Pfk1p, Pfk2p). Fructose 1,6-bisphosphate aldolase (Fba1p) converts fructose-1,6-bisphosphate into glyceraldehyde-3-phosphate (G3P) and dihydroxy-acetone-phosphate (DHAP). DHAP is then converted to G3P by triosephosphate isomerase (e.g. Tpi1p), ultimately, shunting carbon into ethanol. The G3P is converted to 3-phospho-D-glyceroyl-phosphate by glyceraldehyde-3-phosphate dehydrogenases (e.g. Tdh1p, Tdh2p, Tdh3p). The resulting molecule is phosphorylated by 3-phosphoglycerate kinase (e.g. Pgk1p) yielding 3-phosphoglycerate. The phosphate of this molecule is shifted to carbon 2 by phosphoglycerate mutase (e.g. Gpm1p) yielding 2-phosphoglycerate. Enolases (e.g. Eno1p, Eno2p) convert 2-phospoglycerate to phosphoenolpyruvate. Pyruvate kinases (e.g. Cdc19p, Pyk2p) generate pyruvate from phosphoenolpyruvate, concomitantly yielding ATP. Pyruvate is decarboxylated by pyruvate decarboxylases (e.g. Pdc1p, Pdc5p, Pdc6p) to yield acetaldehyde. The acetaldehyde is reduced by alcohol dehydrogenases (e.g. Adh1p, Adh2p, Adh3p, Adh4p, Adh5p) to ethanol. Adh1p is the isoform responsible for most acetaldehyde to ethanol production. The xylose-ethanol pathway can also include enzymes or proteins that recycle erythrose 4-phosphate to PPP.

In some embodiments, the non-naturally occurring microbial organisms provided herein are generated from host organisms having a complete endogenous xylose-ethanol pathway and naturally producing ethanol from xylose. These microbial organisms can be engineered to have enhanced xylose uptake by increasing either the activity and/or level of its xylose transporter. These microbial organisms can also be engineered to have enhanced xylose metabolism by increasing either the activity and/or level of the enzymes or proteins of the endogenous xylose metabolic pathway. In some embodiments, additional copies of genes encoding the endogenous enzymes or proteins can be expressed in the microbial organisms. In some embodiments, one or more heterologous genes encoding an enzyme or protein having similar or same activity as the enzyme(s) of the endogenous pathway can be expressed. The microbial organisms can also be engineered to have a xylose-ethanol pathway that is different from its endogenous pathway(s) by heterologously expressing enzymes or proteins required by the pathway.

In some embodiments, the non-naturally occurring microbial organisms provided herein are generated from host organisms that lack one or more enzymes or proteins required for the xylose-ethanol pathway and do not naturally produce ethanol from xylose. These microbial organisms can be engineered to have a xylose-ethanol pathway by heterologously expressing any missing enzymes or proteins from the endogenous xylose-ethanol pathway. These microbial organisms can be engineered to have enhanced xylose uptake by increasing either the activity or level of its xylose transporter. It is understood that in addition to heterologously expressing the missing enzymes or proteins, the microbial organisms can also be engineered to have increased activity and/or level of its endogenous enzymes or proteins to further enhance the activity of its xylose-ethanol pathways.

The disclosures provide non-naturally occurring microbial organisms provided herein have a xylose-ethanol pathway and at least one exogenous nucleic acid encoding an enzyme or protein expressed in a sufficient amount to confer and/or enhance xylose uptake and/or xylose metabolism, wherein the enzyme or protein has an amino acid sequence that is at least 30% identical to a Metschnikowia enzyme or protein selected from the group consisting of a xylose transporter, a xylose reductase, a xylose dehydrogenase and a xylulokinase. The microbial organisms provided herein can further have an exogenous nucleic acid encoding a transketolase, a transaldolase, or both. In some embodiments, the microbial organisms have at least two exogenous nucleic acids encoding two enzymes or proteins expressed in a sufficient amount to confer or enhance xylose uptake and/or xylose metabolism. In some embodiments, the microbial organisms have at least three exogenous nucleic acids encoding three enzymes or proteins expressed in a sufficient amount to confer or enhance xylose uptake and/or xylose metabolism. In some embodiments, the microbial organisms have at least four exogenous nucleic acids encoding four enzymes or proteins expressed in a sufficient amount to confer or enhance xylose uptake and/or xylose metabolism. In some embodiments, the microbial organisms have at least five exogenous nucleic acids encoding five enzymes or proteins expressed in a sufficient amount to confer or enhance xylose uptake and/or xylose metabolism. In some embodiments, the microbial organisms have at least six exogenous nucleic acids encoding six enzymes or proteins expressed in a sufficient amount to confer or enhance xylose uptake and/or xylose metabolism. In some embodiments, the microbial organisms have at least seven exogenous nucleic acids encoding seven enzymes or proteins expressed in a sufficient amount to confer or enhance xylose uptake and/or xylose metabolism. In some embodiments, the microbial organisms have at least eight exogenous nucleic acids encoding eight enzymes or proteins expressed in a sufficient amount to confer or enhance xylose uptake and/or xylose metabolism. In some embodiments, the microbial organisms have at least nine, at least ten, at least eleven, or at least twelve exogenous nucleic acids encoding nine, ten, eleven, or twelve enzymes or proteins expressed in a sufficient amount to confer or enhance xylose uptake and/or xylose metabolism.

In some embodiments, the microbial organisms provided herein have at least one exogenous nucleic acid encoding an enzyme or protein expressed in a sufficient amount to confer and/or enhance xylose uptake and/or xylose metabolism, wherein the enzyme or protein has an amino acid sequence that is at least 30% identical to a Metschnikowia enzyme or protein selected from the group consisting of a xylose transporter, a xylose reductase, a xylose dehydrogenase and a xylulokinase. The enzyme or protein can have an amino acid sequence that is at least 35% identical to the Metschnikowia enzyme or protein. The enzyme or protein can have an amino acid sequence that is at least 40% identical to the Metschnikowia enzyme or protein. The enzyme or protein can have an amino acid sequence that is at least 45% identical to the Metschnikowia enzyme or protein. The enzyme or protein can have an amino acid sequence that is at least 50% identical to the Metschnikowia enzyme or protein. The enzyme or protein can have an amino acid sequence that is at least 55% identical to the Metschnikowia enzyme or protein. The enzyme or protein can have an amino acid sequence that is at least 60% identical to the Metschnikowia enzyme or protein. The enzyme or protein can have an amino acid sequence that is at least 65% identical to the Metschnikowia enzyme or protein. The enzyme or protein can have an amino acid sequence that is at least 70% identical to the Metschnikowia enzyme or protein. The enzyme or protein can have an amino acid sequence that is at least 71% identical to the Metschnikowia enzyme or protein. The enzyme or protein can have an amino acid sequence that is at least 72% identical to the Metschnikowia enzyme or protein. The enzyme or protein can have an amino acid sequence that is at least 73% identical to the Metschnikowia enzyme or protein. The enzyme or protein can have an amino acid sequence that is at least 74% identical to the Metschnikowia enzyme or protein. The enzyme or protein can have an amino acid sequence that is at least 75% identical to the Metschnikowia enzyme or protein. The enzyme or protein can have an amino acid sequence that is at least 76% identical to the Metschnikowia enzyme or protein. The enzyme or protein can have an amino acid sequence that is at least 77% identical to the Metschnikowia enzyme or protein. The enzyme or protein can have an amino acid sequence that is at least 78% identical to the Metschnikowia enzyme or protein. The enzyme or protein can have an amino acid sequence that is at least 79% identical to the Metschnikowia enzyme or protein. The enzyme or protein can have an amino acid sequence that is at least 80% identical to the Metschnikowia enzyme or protein. The enzyme or protein can have an amino acid sequence that is at least 81% identical to the Metschnikowia enzyme or protein. The enzyme or protein can have an amino acid sequence that is at least 82% identical to the Metschnikowia enzyme or protein. The enzyme or protein can have an amino acid sequence that is at least 83% identical to the Metschnikowia enzyme or protein. The enzyme or protein can have an amino acid sequence that is at least 84% identical to the Metschnikowia enzyme or protein. The enzyme or protein can have an amino acid sequence that is at least 85% identical to the Metschnikowia enzyme or protein. The enzyme or protein can have an amino acid sequence that is at least 86% identical to the Metschnikowia enzyme or protein. The enzyme or protein can have an amino acid sequence that is at least 87% identical to the Metschnikowia enzyme or protein. The enzyme or protein can have an amino acid sequence that is at least 88% identical to the Metschnikowia enzyme or protein. The enzyme or protein can have an amino acid sequence that is at least 89% identical to the Metschnikowia enzyme or protein. The enzyme or protein can have an amino acid sequence that is at least 90% identical to the Metschnikowia enzyme or protein. The enzyme or protein can have an amino acid sequence that is at least 91% identical to the Metschnikowia enzyme or protein. The enzyme or protein can have an amino acid sequence that is at least 92% identical to the Metschnikowia enzyme or protein. The enzyme or protein can have an amino acid sequence that is at least 93% identical to the Metschnikowia enzyme or protein. The enzyme or protein can have an amino acid sequence that is at least 94% identical to the Metschnikowia enzyme or protein. The enzyme or protein can have an amino acid sequence that is at least 95% identical to the Metschnikowia enzyme or protein. The enzyme or protein can have an amino acid sequence that is at least 96% identical to the Metschnikowia enzyme or protein. The enzyme or protein can have an amino acid sequence that is at least 97% identical to the Metschnikowia enzyme or protein. The enzyme or protein can have an amino acid sequence that is at least 98% identical to the Metschnikowia enzyme or protein. The enzyme or protein can have an amino acid sequence that is at least 99% identical to the Metschnikowia enzyme or protein. In some embodiments, the enzyme or protein is a Metschnikowia enzyme or protein.

The Metschnikowia enzyme or protein can be an enzyme or protein from an H0 Metschnikowia species. In some embodiments, the microbial organisms provided herein have at least one exogenous nucleic acid encoding an enzyme or protein expressed in a sufficient amount to confer or enhance xylose uptake and/or xylose metabolism, wherein the enzyme or protein has an amino acid sequence that is at least 30% identical to the enzyme or protein from the H0 Metschnikowia species selected from the group consisting of a xylose transporter, a xylose reductase, a xylose dehydrogenase and a xylulokinase. The enzyme or protein can have an amino acid sequence that is at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to an enzyme or protein from the H0 Metschnikowia species. Exemplary sequences are provided below of enzyme or protein from the H0 Metschnikowia species are provided below.

| SEQ | | |
|---|---|---|
| ID NO: | Description | SEQUENCES |
| 1 | Amino acid | MGYEEKLVAPALKFKNFLDKTPNIHNVYVIAAISCTSGMMFGFDISSM |
| | sequence of H0 | SVFVDQQPYLKMFDNPSSVIQGFITASMSLGSFFGSLTSTFISEPFGR |
| | Metschnikowia | RASLFICGILWVIGAAVQSSSQNRAQLICGRIIAGWGIGFGSSVAPVY |
| | species Xyt1p | GSEMAPRKIRGTIGGIFQFSVTVGIFIMFLIGYGCSFIQGKASFRIPW |
| | | GVQMVPGLILLIGLFFIPESPRWLAKQGYWEDAEIIVANVQAKGNRND |
| | | ANVQIEMSEIKDQLMLDEHLKEFTYADLFTKKYRQRTITAIFAQIWQQ |
| | | LTGMNVMMYYIVYIFQMAGYSGNTNLVPSLIQYIINMAVTVPALFCLD |
| | | LLGRRTILLAGAAFMMAWQFGVAGILATYSEPAYISDTVRITIPDDHK |
| | | SAAKGVIACCYLFVCSFAFSWGVGIWVYCSEVWGDSQSRQRGAALATS |
| | | ANWIFNFAIAMFTPSSFKNITWKTYIIYATFCACMFIHVFFFFPETKG |
| | | KRLEEIGQLWDEGVPAWRSAKWQPTVPLASDAELAHKMDVAHAEHADL |
| | | LATHSPSSDEKTGTV |
| 2 | Amino acid | MSQDELHTKSGVETPINDSLLEEKHDVTPLAALPEKSFKDYISISIFC |
| | sequence of H0 | LFVAFGGFVFGFDTGTISGFVNMSDFKTRFGEMNAQGEYYLSNVRTGL |
| | Metschnikowia | MVSIFNVGCAVGGIFLCKIADVYGRRIGLMFSMVVYVVGIIIQIASTT |
| | species Gxf1p | KWYQYFIGRLIAGLAVGTVSVISPLFISEVAPKQLRGTLVCCFQLCIT |
| | | LGIFLGYCTTYGTKTYTDSRQWRIPLGICFAWALFLVAGMLNMPESPR |
| | | YLVEKSRIDDARKSIARSNKVSEEDPAVYTEVQLIQAGIDREALAGSA |
| | | TWMELVTGKPKIFRRVIMGVMLQSLQQLTGDNYFFYYGTTIFKAVGLQ |
| | | DSFQTSIILGIVNFASTFVGIYAIERMGRRLCLLTGSACMFVCFIIYS |
| | | LIGTQHLYKNGFSNEPSNTYKPSGNAMIFITCLYIFFFASTWAGGVYC |
| | | IVSESYPLRIRSKAMSVATAANWMWGFLISFFTPFITSAIHFYYGFVF |
| | | TGCLAFSFFYVYFFVVETKGLSLEEVDILYASGTLPWKSSGWVP |
| 3 | Amino acid | MSDFKTRFGEMNAQGEYYLSNVRTGLMVSIFNVGCAVGGIFLCKIADV |
| | sequence of H0 | YGRRIGLMFSMVVYVVGIIIQIASTTKWYQYFIGRLIAGLAVGTVSVI |
| | Metschnikowia | SPLFISEVAPKQLRGTLVCCFQLCITLGIFLGYCTTYGTKTYTDSRQW |
| | species ΔGxf1p | RIPLGICFAWALFLVAGMLNMPESPRYLVEKSRIDDARKSIARSNKVS |
| | (variant of Gxf1p | EEDPAVYIEVQLIQAGIDREALAGSATWMELVTGKPKIFRRVIMGVML |
| | with shorter N- | QSLQQLTGDNYFFYYGTTIFKAVGLQDSFQTSIILGIVNFASTFVGIY |
| | terminus) | AIERMGRRLCLLTGSACMFVCFIIYSLIGTQHLYKNGFSNEPSNTYKP |
| | | SGNAMIFITCLYIFFFASTWAGGVYCIVSESYPLRIRSKAMSVATAAN |
| | | WMWGFLISFFTPFITSAIHFYYGFVFTGCLAFSFFYVYFFVVETKGLS |
| | | LEEVDILYASGTLPWKSSGWVP |
| 4 | Amino acid | MSAEQEQQVSGTSATIDGLASLKQEKTAEEEDAFKPKPATAYFFISFL |
| | sequence of H0 | CGLVAFGGYVFGFDTGTISGFVNMDDYLMRFGQQHADGTYYLSNVRTG |
| | Metschnikowia | LIVSIFNIGCAVGGLALSKVGDIWGRRIGIMVAMIIYMVGIIIQIASQ |
| | species | DKWYQYFIGRLITGLGVGTTSVLSPLFISESAPKHLRGTLVCCFQLMV |
| | Gxf2p/Gal2p | TLGIFLGYCTTYGTKNYTDSRQWRIPLGLCFAWALLLISGMVFMPESP |
| | | RFLIERQRFDEAKASVAKSNQVSIEDPAVYTEVELIQAGIDREALAGS |
| | | AGWKELITGKPKMLQRVILGMMLQSIQQLTGNNYFFYYGTTIFKAVGM |
| | | SDSFQTSIVLGIVNFASTFVGIWAIERMGRRSCLLVGSACMSVCFLIY |
| | | SILGSVNLYIDGYENTPSNTRKPTGNAMIFITCLFIFFFASTWAGGVY |
| | | SIVSETYPLRIRSKGMAVATAANWMWGFLISFFTPFITSAIHFYYGFV |
| | | FTGCLIFSFFYVFFFVRETKGLSLEEVDELYATDLPPWKTAGWTPPSA |
| | | EDMAHTTGFAEAAKPTNKHV |
| 5 | Amino acid | MGIFVGVFAALGGVLFGYDTGTISGVMAMPWVKEHFPKDRVAFSASES |
| | sequence of H0 | SLIVSILSAGTFFGAILAPLLTDTLGRRWCIIISSLVVFNLGAALQTA |
| | Metschnikowia | ATDIPLLIVGRVIAGLGVGLISSTIPLYQSEALPKWIRGAVVSCYQWA |
| | species | ITIGIFLAAVINQGTHKINSPASYRIPLGIQMAWGLILGVGMFFLPET |
| | ΔGxs1p/ΔHgt12p | PRFYISKGQNAKAAVSLARLRKLPQDHPELLEELEDIQAAYEFETVHG |
| | (variant of | KSSWSQVFTNKNKQLKKLATGVCLQAFQQLTGVNFIFYFGTTFFNSVG |
| | Gxs1p/Hgt12p | LDGFTTSLATNIVNVGSTIPGILGVEIFGRRKVLLTGAAGMCLSQFIV |
| | with shorter N- | AIVGVATDSKAANQVLIAFCCIFIAFFAATWGPTAWVVCGEIFPLRTR |
| | terminus) | AKSIANICAASNWLLNWAIAYATPYLVDSDKGNLGTNVFFIWGSCNFF |
| | | CLVFAYFMIYETKGLSLEQVDELYEKVASARKSPGFVPSEHAFREHAD |
| | | VETAMPDNFNLKAEAISVEDASV |
| 6 | NOT USED | |
| 7 | Amino acid | MGLESNKLIRKYINVGEKRAGSSGMGIFVGVFAALGGVLFGYDTGTIS |
| | sequence of H0 | GVMAMPWVKEHFPKDRVAFSASESSLIVSILSAGTFFGAILAPLLTDT |
| | Metschnikowia | LGRRWCIIISSLVVFNLGAALQTAATDIPLLIVGRVIAGLGVGLISST |
| | species | IPLYQSEALPKWIRGAVVSCYQWAITIGIFLAAVINQGTHKINSPASY |
| | Gxs1p/Hgt12 | RIPLGIQMAWGLILGVGMFFLPETPRFYISKGQNAKAAVSLARLRKLP |
| | | QDHPELLEELEDIQAAYEFETVHGKSSWSQVFTNKNKQLKKLATGVCL |
| | | QAFQQLTGVNFIFYFGTTFFNSVGLDGFTTSLATNIVNVGSTIPGILG |
| | | VEIFGRRKVLLTGAAGMCLSQFIVAIVGVATDSKAANQVLIAFCCIFI |
| | | AFFAATWGPTAWVVCGEIFPLRTRAKSIAMCAASNWLLNWAIAYATPY |
| | | LVDSDKGNLGTNVFFIWGSCNFFCLVFAYFMIYETKGLSLEQVDELYE |
| | | KVASARKSPGFVPSEHAFREHADVETAMPDNFNLKAEAISVEDASV |
| 8 | Amino acid | MSIFEGKDGKGVSSTESLSNDVRYDNMEKVDQDVLRHNFNFDKEFEEL |
| | sequence of H0 | EIEAAQVNDKPSFVDRILSLEYKLHFENKNHMVWLLGAFAAAAGLLSG |
| | Metschnikowia | LDQSIISGASIGMNKALNLIEREASLVSSLMPLGAMAGSMIMTPLNEW |
| | species Hxt5p | FGRKSSLITSCIWYTIGSALCAGARDHHMMYAGRFILGVGVGIEGGCV |
| | | GIYISESVPANVRGSIVSMYQFNIALGEVLGYAVAAIFYTVHGGWRFM |
| | | VGSSLVFSTILFAGLFFLPESPRWLVHKGRNGMAYDVWKRLRDINDES |
| | | AKLEFLEMRQAAYQERERRSQESLFSSWGELFTIARNRRALTYSVIMI |
| | | TLGQLTGVNAVMYYMSTLMGAIGFNEKDSVFMSLVGGGSLLIGTIPAI |
| | | LWMDRFGRRVWGYNLVGFFVGLVLVGVGYRFNPVTQKAASEGVYLTGL |
| | | IVYFLFFGSYSTLTWVIPSESFDLRTRSLGMTICSTFLYLWSFTVTYN |
| | | FTKMSAAFTYTGLTLGFYGGIAFLGLIYQVCFMPETKDKTLEEIDDIF |
| | | NRSAFSIARENISNLKKGIW |
| 9 | Amino acid | MGYEEKLVAPALKFKNFLDKTPNIHNVYVIAAISCTSGMMFGFDISSM |
| | sequence of H0 | SVFVDQQPYLKMFDNPSSVIQGFITALMSLGSFFGSLTSTFISEPFGR |
| | Metschnikowia | RASLFICGILWVIGAAVQSSSQNRAQLICGRIIAGWGIGFGSSVAPVY |
| | species Xyt1p | GSEMAPRKIRGTIGGIFQFSVTVGIFIMFLIGYGCSFIQGKASFRIPW |
| | with S75L | GVQMVPGLILLIGLFFIPESPRWLAKQGYWEDAEIIVANVQAKGNRND |
| | mutation. | ANVQIEMSEIKDQLMLDEHLKEFTYADLFTKKYRQRTITAIFAQIWQQ |
| | | LTGMNVMMYYIVYIFQMAGYSGNTNLVPSLIQYIINMAVTVPALFCLD |
| | | LLGRRTILLAGAAFMMAWQFGVAGILATYSEPAYISDTVRITIPDDHK |
| | | SAAKGVIACCYLFVCSFAFSWGVGIWVYCSEVWGDSQSRQRGAALATS |
| | | ANWIFNFAIAMFTPSSFKNITWKTYIIYATFCACMFIHVFFFFPETKG |
| | | KRLEEIGQLWDEGVPAWRSAKWQPTVPLASDAELAHKMDVAHAEHADL |
| | | LATHSPSSDEKTGTV |
| 10 | Amino acid | MSSTTDTLEKRDIEPFTSDAPVTVHDYIAEERPWWKVPHLRVLTWSVF |
| | sequence of H0 | VITLTSTNNGYDGSMLNGLQSLDIWQEDLGHPAGQKLGALANGVLFGN |
| | Metschnikowia | LAAVPFASYFCDRFGRRPVICFGQILTIVGAVLQGLSNSYGFFLGSRI |
| | species Hxt2.6p | VLGFGAMIATIPSPTLISEIAYPTHRETSTFAYNVCWYLGAIIASWVT |
| | | YGTRDLQSKACWSIPSYLQAALPFFQVCMIWFVPESPRFLVAKGKIDQ |
| | | ARAVLSKYHTGDSTDPRDVALVDFELHEIESALEQEKLNTRSSYFDFF |
| | | KKRNFRKRGFLCVMVGVAMQLSGNGLVSYYLSKVLDSIGITETKRQLE |
| | | INGCLMIYNFVICVSLMSVCRMFKRRVLFLTCFSGMTVCYTIWTILSA |
| | | LNEQRHFEDKGLANGVLAMIFFYYFFYNVGINGLPFLYITEILPYSHR |
| | | AKGLNLFQFSQFLTQIYNGYVNPIAMDAISWKYYIVYCCILFVELVIV |
| | | FFTFPETSGYTLEEVAQVFGDEAPGLHNRQLDVAKESLEHVEHV |
| 11 | Amino acid | MGFRNLKRRLSNVGDSMSVHSVKEEEDFSRVEIPDEIYNYKIVLVALT |
| | sequence of H0 | AASAAIIIGYDAGFIGGTVSLTAFKSEFGLDKMSATAASAIEANVVSV |
| | Metschnikowia | FQAGAYFGCLFFYPIGEIWGRKIGLLLSGFLLTFGAAISLISNSSRGL |
| | species Qup2p | GAIYAGRVLTGLGIGGCSSLAPIYVSEIAPAAIRGKLVGCWEVSWQVG |
| | | GIVGYWINYGVLQTLPISSQQWIIPFAVQLIPSGLFWGLCLLIPESPR |
| | | FLVSKGKIDKARKNLAYLRGLSEDHPYSVFELENISKAIEENFEQTGR |
| | | GFFDPLKALFFSKKMLYRLLLSTSMFMMQNGYGINAVTYYSPTIFKSL |
| | | GVQGSNAGLLSTGIFGLLKGAASVFWVFFLVDTFGRRFCLCYLSLPCS |
| | | ICMWYIGAYIKIANPSAKLAAGDTATTPAGTAAKAMLYIWTIFYGITW |
| | | NGTTWVICAEIFPQSVRTAAQAVNASSNWFWAFMIGHFTGQALENIGY |
| | | GYYFLFAACSAIFPVVVWFVYPETKGVPLEAVEYLFEVRPWKAHSYAL |
| | | EKYQIEYNEGEFHQHKPEVLLQGSENSD |
| 12 | Amino acid | MGYEEKLVAPALKFKNFLDKTPNIHNVYVIAAISCTSGMMFGFDISSM |
| | sequence of H0 | SVFVDQQPYLKMFDNPSSVIQGFITASMSLGSFFGSLTSTFISEPFGR |
| | Metschnikowia | RASLFICGILWVIGAAVQSSSQNRAQLICGRIIAGWGIGFGSSVAPVY |
| | species | GSEMAPRKIRGTIGGIFQFSVTVGIFIMFLIGYGCSFIQGKASFRIPW |
| | Aps1p/Hgt19p | GVQMVPGLILLIGLFFIPESPRWLAKQGYWEDAEIIVANVQAKGNRND |
| | | ANVQIEMSEIKDQLMLDEHLKEFTYADLFTKKYRQRTITAIFAQIWQQ |
| | | LTGMNVMMYYIVYIFQMAGYSGNTNLVPSLIQYIINMAVTVPALFCLD |
| | | LLGRRTILLAGAAFMMAWQFGVAGILATYSEPAYISDTVRITIPDDHK |
| | | SAAKGVIACCYLFVCSFAFSWGVGIWVYCSEVWGDSQSRQRGAALATS |
| | | ANWIFNFAIAMFTPSSFKNITWKTYIIYATFCACMFIHVFFFFPETKG |
| | | KRLEEIGQLWDEGVPAWRSAKWQPTVPLASDAELAHKMDVAHAEHADL |
| | | LATHSPSSDEKTGTV |
| 13 | Nucleic acid | ATGGGTTACGAGGAAAAGCTTGTAGCGCCCGCGTTGAAATTCAAAAAC |
| | sequence of H0 | TTTCTTGACAAAACCCCCAATATTCACAATGTCTATGTCATTGCCGCC |
| | Metschnikowia | ATCTCCTGTACATCAGGTATGATGTTTGGATTTGATATCTCGTCGATG |
| | species XYT1 | TCTGTCTTTGTCGACCAGCAGCCATACTTGAAGATGTTTGACAACCCT |
| | | AGTTCCGTGATTCAAGGTTTCATTACCGCGCTGATGAGTTTGGGCTCG |
| | | TTTTTCGGCTCGCTCACATCCACGTTCATCTCTGAGCCTTTTGGTCGT |
| | | CGTGCATCGTTGTTCATTTGTGGTATTCTTTGGGTAATTGGAGCAGCG |
| | | GTTCAAAGTTCGTCGCAGAACAGGGCCCAATTGATTTGTGGGCGTATC |
| | | ATTGCAGGATGGGGCATTGGCTTTGGGTCATCGGTGGCTCCTGTTTAC |
| | | GGGTCCGAGATGGCTCCGAGAAAGATCAGAGGCACGATTGGTGGAATC |
| | | TTCCAGTTCTCCGTCACCGTGGGTATCTTTATCATGTTCTTGATTGGG |
| | | TACGGATGCTCTTTCATTCAAGGAAAGGCCTCTTTCCGGATCCCCTGG |
| | | GGTGTGCAAATGGTTCCCGGCCTTATCCTCTTGATTGGACTTTTCTTT |
| | | ATTCCTGAATCTCCCCGTTGGTTGGCCAAACAGGGCTACTGGGAAGAC |
| | | GCCGAAATCATTGTGGCCAATGTGCAGGCCAAGGGTAACCGTAACGAC |
| | | GCCAACGTGCAGATTGAAATGTCGGAGATTAAGGATCAATTGATGCTT |
| | | GACGAGCACTTGAAGGAGTTTACGTACGCTGACCTTTTCACGAAGAAG |
| | | TACCGCCAGCGCACGATCACGGCGATCTTTGCCCAGATCTGGCAACAG |
| | | TTGACCGGTATGAATGTGATGATGTACTACATTGTGTACATTTTCCAG |
| | | ATGGCAGGCTACAGCGGCAACACGAACTTGGTGCCCAGTTTGATCCAG |
| | | TACATCATCAACATGGCGGTCACGGTGCCGGCGCTTTTCTGCTTGGAT |
| | | CTCTTGGGCCGTCGTACCATTTTGCTCGCGGGTGCCGCGTTCATGATG |
| | | GCGTGGCAATTCGGCGTGGCGGGCATTTTGGCCACTTACTCAGAACCG |
| | | GCATATATCTCTGACACTGTGCGTATCACGATCCCCGACGACCACAAG |
| | | TCTGCTGCAAAAGGTGTGATTGCATGCTGCTATTTGTTTGTGTGCTCG |
| | | TTTGCATTCTCGTGGGGTGTCGGTATTTGGGTGTACTGTTCCGAGGTT |
| | | TGGGGTGACTCCCAGTCGAGACAAAGAGGCGCCGCTCTTGCGACGTCG |
| | | GCCAACTGGATCTTCAACTTCGCCATTGCCATGTTCACGCCGTCCTCA |
| | | TTCAAGAATATCACGTGGAAGACGTATATCATCTACGCCACGTTCTGT |
| | | GCGTGCATGTTCATACACGTGTTTTTCTTTTTCCCAGAAACAAAGGGC |
| | | AAGCGTTTGGAGGAGATAGGCCAGCTTTGGGACGAAGGAGTCCCAGCA |
| | | TGGAGGTCAGCCAAGTGGCAGCCAACAGTGCCGCTCGCGTCCGACGCA |
| | | GAGCTTGCACACAAGATGGATGTTGCGCACGCGGAGCACGCGGACTTA |
| | | TTGGCCACGCACTCGCCATCTTCAGACGAGAAGACGGGCACGGTCTAA |
| 14 | Nucleic acid | ATGTCTCAAGACGAACTTCATACAAAGTCTGGTGTTGAAACACCAATC |
| | sequence of H0 | AACGATTCGCTTCTCGAGGAGAAGCACGATGTCACCCCACTCGCGGCA |
| | Metschnikowia | TTGCCCGAGAAGTCCTTCAAGGACTACATTTCCATTTCCATTTTCTGT |
| | species GXF1 | TTGTTTGTGGCATTTGGTGGTTTTGTTTTCGGTTTCGACACCGGTACG |
| | | ATTTCCGGTTTCGTCAACATGTCCGACTTCAAGACCAGATTTGGTGAG |
| | | ATGAATGCCCAGGGCGAATACTACTTGTCCAATGTTAGAACTGGTTTG |
| | | ATGGTTTCTATTTTCAACGTCGGTTGCGCCGTTGGTGGTATCTTCCTT |
| | | TGTAAGATTGCCGATGTTTATGGCAGAAGAATTGGTCTTATGTTTTCC |
| | | ATGGTGGTTTATGTCGTTGGTATCATTATTCAGATTGCCTCCACCACC |
| | | AAATGGTACCAATACTTCATTGGCCGTCTTATTGCTGGCTTGGCTGTG |
| | | GGTACTGTTTCCGTCATCTCGCCACTTTTCATTTCCGAGGTTGCTCCT |
| | | AAACAGCTCAGAGGTACGCTTGTGTGCTGCTTCCAGTTGTGTATCACC |
| | | TTGGGTATCTTTTTGGGTTACTGCACGACCTACGGTACAAAGACTTAC |
| | | ACTGACTCCAGACAGTGGAGAATCCCATTGGGTATCTGTTTCGCGTGG |
| | | GCTTTGTTTTTGGTGGCCGGTATGTTGAACATGCCCGAGTCTCCTAGA |
| | | TACTTGGTTGAGAAATCGAGAATCGACGATGCCAGAAAGTCCATTGCC |
| | | AGATCCAACAAGGTTTCCGAGGAAGACCCCGCCGTGTACACCGAGGTG |
| | | CAGCTTATCCAGGCTGGTATTGACAGAGAGGCCCTTGCCGGCAGCGCC |
| | | ACATGGATGGAGCTTGTGACTGGTAAGCCCAAAATCTTCAGAAGAGTC |
| | | ATCATGGGTGTCATGCTTCAGTCCTTGCAACAATTGACTGGTGACAAC |
| | | TACTTTTTCTACTACGGAACCACGATTTTCAAGGCTGTTGGCTTGCAG |
| | | GACTCTTTCCAGACGTCGATTATCTTGGGTATTGTCAACTTTGCCTCG |
| | | ACTTTTGTCGGTATTTACGCCATTGAGAGAATGGGCAGAAGATTGTGT |
| | | TTGTTGACCGGATCTGCGTGCATGTTTGTGTGTTTCATCATCTACTCG |
| | | CTCATTGGTACGCAGCACTTGTACAAGAACGGCTTCTCTAACGAACCT |
| | | TCCAACACATACAAGCCTTCCGGTAACGCCATGATCTTCATCACGTGT |
| | | CTTTACATTTTCTTCTTTGCCTCGACCTGGGCCGGTGGTGTTTACTGT |
| | | ATCGTGTCCGAGTCTTACCCATTGAGAATCAGATCCAAGGCCATGTCT |
| | | GTCGCCACCGCCGCCAACTGGATGTGGGGTTTCTTGATCTCGTTCTTC |
| | | ACGCCTTTCATCACCTCCGCCATCCACTTTTACTACGGTTTTGTTTTC |
| | | ACTGGCTGCTTGGCGTTCTCCTTCTTCTACGTCTACTTCTTTGTCGTG |
| | | GAGACCAAGGGTCTTTCCTTGGAGGAGGTTGACATTTTGTACGCTTCC |
| | | GGTACGCTTCCATGGAAGTCCTCTGGCTGGGTGCCTCCTACCGCGGAC |
| | | GAAATGGCCCACAACGCCTTCGACAACAAGCCAACTGACGAACAAGTC |
| | | TAA |
| 15 | Nucleic acid | ATGTCCGACTTCAAGACCAGATTTGGTGAGATGAATGCCCAGGGCGAA |
| | sequence of H0 | TACTACTTGTCCAATGTTAGAACTGGTTTGATGGTTTCTATTTTCAAC |
| | Metschnikowia | GTCGGTTGCGCCGTTGGTGGTATCTTCCTTTGTAAGATTGCCGATGTT |
| | species ΔGXF1 | TATGGCAGAAGAATTGGTCTTATGTTTTCCATGGTGGTTTATGTCGTT |
| | (variant of GXF1 | GGTATCATTATTCAGATTGCCTCCACCACCAAATGGTACCAATACTTC |
| | with shorter N- | ATTGGCCGTCTTATTGCTGGCTTGGCTGTGGGTACTGTTTCCGTCATC |
| | terminus) | TCGCCACTTTTCATTTCCGAGGTTGCTCCTAAACAGCTCAGAGGTACG |
| | | CTTGTGTGCTGCTTCCAGTTGTGTATCACCTTGGGTATCTTTTTGGGT |
| | | TACTGCACGACCTACGGTACAAAGACTTACACTGACTCCAGACAGTGG |
| | | AGAATCCCATTGGGTATCTGTTTCGCGTGGGCTTTGTTTTTGGTGGCC |
| | | GGTATGTTGAACATGCCCGAGTCTCCTAGATACTTGGTTGAGAAATCG |
| | | AGAATCGACGATGCCAGAAAGTCCATTGCCAGATCCAACAAGGTTTCC |
| | | GAGGAAGACCCCGCCGTGTACACCGAGGTGCAGCTTATCCAGGCTGGT |
| | | ATTGACAGAGAGGCCCTTGCCGGCAGCGCCACATGGATGGAGCTTGTG |
| | | ACTGGTAAGCCCAAAATCTTCAGAAGAGTCATCATGGGTGTCATGCTT |
| | | CAGTCCTTGCAACAATTGACTGGTGACAACTACTTTTTCTACTACGGA |
| | | ACCACGATTTTCAAGGCTGTTGGCTTGCAGGACTCTTTCCAGACGTCG |
| | | ATTATCTTGGGTATTGTCAACTTTGCCTCGACTTTTGTCGGTATTTAC |
| | | GCCATTGAGAGAATGGGCAGAAGATTGTGTTTGTTGACCGGATCTGCG |
| | | TGCATGTTTGTGTGTTTCATCATCTACTCGCTCATTGGTACGCAGCAC |
| | | TTGTACAAGAACGGCTTCTCTAACGAACCTTCCAACACATACAAGCCT |
| | | TCCGGTAACGCCATGATCTTCATCACGTGTCTTTACATTTTCTTCTTT |
| | | GCCTCGACCTGGGCCGGTGGTGTTTACTGTATCGTGTCCGAGTCTTAC |
| | | CCATTGAGAATCAGATCCAAGGCCATGTCTGTCGCCACCGCCGCCAAC |
| | | TGGATGTGGGGTTTCTTGATCTCGTTCTTCACGCCTTTCATCACCTCC |
| | | GCCATCCACTTTTACTACGGTTTTGTTTTCACTGGCTGCTTGGCGTTC |
| | | TCCTTCTTCTACGTCTACTTCTTTGTCGTGGAGACCAAGGGTCTTTCC |
| | | TTGGAGGAGGTTGACATTTTGTACGCTTCCGGTACGCTTCCATGGAAG |
| | | TCCTCTGGCTGGGTGCCTCCTACCGCGGACGAAATGGCCCACAACGCC |
| | | TTCGACAACAAGCCAACTGACGAACAAGTCTAA |
| 16 | Nucleic acid | ATGAGTGCCGAACAGGAACAACAAGTATCGGGCACATCTGCCACGATA |
| | sequence of H0 | GATGGGCTGGCGTCCTTGAAGCAAGAAAAAACCGCCGAGGAGGAAGAC |
| | Metschnikowia | GCCTTCAAGCCTAAGCCCGCCACGGCGTACTTTTTCATTTCGTTCCTC |
| | species | TGTGGCTTGGTCGCCTTTGGCGGCTACGTTTTCGGTTTCGATACCGGT |
| | GXF2/GAL2 | ACGATTTCCGGGTTTGTTAACATGGACGACTATTTGATGAGATTCGGC |
| | | CAGCAGCACGCTGATGGCACGTATTACCTTTCCAACGTGAGAACCGGT |
| | | TTGATCGTGTCGATCTTCAACATTGGCTGTGCCGTTGGTGGTCTTGCG |
| | | CTTTCGAAAGTCGGTGACATTTGGGGCAGAAGAATTGGTATTATGGTT |
| | | GCTATGATCATCTACATGGTGGGAATCATCATCCAGATCGCTTCACAG |
| | | GATAAATGGTACCAGTACTTCATTGGCCGTTTGATCACCGGATTGGGT |
| | | GTCGGCACCACGTCCGTGCTTAGTCCTCTTTTCATCTCCGAGTCGGCT |
| | | CCGAAGCATTTGAGAGGCACCCTTGTGTGTTGTTTCCAGCTCATGGTC |
| | | ACCTTGGGTATCTTTTTGGGCTACTGCACGACCTACGGTACCAAGAAC |
| | | TACACTGACTCGCGCCAGTGGCGGATTCCCTTGGGTCTTTGCTTCGCA |
| | | TGGGCTCTTTTGTTGATCTCGGGAATGGTTTTCATGCCTGAATCCCCA |
| | | CGTTTCTTGATTGAGCGCCAGAGATTCGACGAGGCCAAGGCTTCCGTG |
| | | GCCAAATCGAACCAGGTTTCGACCGAGGACCCCGCCGTGTACACTGAA |
| | | GTCGAGTTGATCCAGGCCGGTATTGACCGTGAGGCATTGGCCGGATCC |
| | | GCTGGCTGGAAAGAGCTTATCACGGGTAAGCCCAAGATGTTGCAGCGT |
| | | GTGATTTTGGGAATGATGCTCCAGTCGATCCAGCAGCTTACCGGTAAC |
| | | AACTACTTTTTCTACTATGGTACCACGATCTTCAAGGCCGTGGGCATG |
| | | TCGGACTCGTTCCAGACCTCGATTGTTTTGGGTATTGTCAACTTCGCC |
| | | TCCACTTTTGTCGGAATCTGGGCCATCGAACGCATGGGCCGCAGATCT |
| | | TGTTTGCTTGTTGGTTCCGCGTGCATGAGTGTGTGTTTCTTGATCTAC |
| | | TCCATCTTGGGTTCCGTCAACCTTTACATCGACGGCTACGAGAACACG |
| | | CCTTCCAACACGCGTAAGCCTACCGGTAACGCCATGATTTTCATCACG |
| | | TGTTTGTTCATCTTCTTCTTCGCCTCCACCTGGGCCGGTGGTGTGTAC |
| | | AGTATTGTGTCTGAAACATACCCATTGAGAATCCGCTCTAAAGGTATG |
| | | GCCGTGGCCACCGCTGCCAACTGGATGTGGGGTTTCTTGATTTCGTTC |
| | | TTCACGCCTTTCATCACCTCGGCCATCCACTTCTACTACGGGTTTGTG |
| | | TTCACAGGGTGTCTTATTTTCTCCTTCTTCTACGTGTTCTTCTTTGTT |
| | | AGGGAAACCAAGGGTCTCTCGTTGGAAGAGGTGGATGAGTTATATGCC |
| | | ACTGACCTCCCACCATGGAAGACCGCGGGCTGGACGCCTCCTTCTGCT |
| | | GAGGATATGGCCCACACCACCGGGTTTGCCGAGGCCGCAAAGCCTACG |
| | | AACAAACACGTTTAA |
| 17 | Nucleic acid | ATGGGCATTTTCGTTGGCGTTTTCGCCGCGCTTGGCGGTGTTCTCTTT |
| | sequence of H0 | GGCTACGATACCGGTACCATCTCTGGTGTGATGGCCATGCCTTGGGTC |
| | Metschnikowia | AAGGAACATTTCCCAAAAGACCGTGTTGCATTTAGTGCTTCCGAGTCG |
| | species ΔGXS1/ | TCGTTGATTGTGTCTATTTTATCTGCAGGAACTTTCTTTGGAGCCATT |
| | ΔHGT12 (variant | CTTGCTCCGCTCTTGACCGATACATTGGGTAGACGCTGGTGTATTATC |
| | of GXS1/HGT12 | ATCTCTTCGCTCGTTGTGTTCAATTTGGGTGCTGCTTTGCAGACGGCT |
| | with shorter N- | GCCACGGATATCCCGCTCTTGATTGTTGGTCGTGTCATTGCCGGTTTA |
| | terminus) | GGGGTTGGTTTGATTTCGCTGACGATTCCATTGTACCAGTCCGAAGCG |
| | | CTTCCCAAATGGATTAGAGGTGCTGTTGTCTCGTGCTACCAATGGGCC |
| | | ATTACTATTGGTATCTTTTTGGCTGCCGTGATCAACCAGGGCACTCAC |
| | | AAGATCAACAGCCCTGCGTCGTACAGAATTCCATTGGGTATTCAGATG |
| | | GCATGGGGTCTTATCTTGGGTGTCGGCATGTTCTTCTTGCCCGAGACG |
| | | CCTCGTTTCTACATTTCCAAGGGCCAGAATGCGAAGGCTGCTGTTTCA |
| | | TTGGCGCGTTTGAGAAAGCTTCCGCAAGATCACCCGGAGTTGTTGGAG |
| | | GAATTGGAAGATATCCAGGCGGCATACGAGTTTGAGACTGTCCATGGC |
| | | AAGTCTTCATGGCTGCAGGTTTTCACCAACAAGAACAAACAATTGAAG |
| | | AAGTTGGCCACGGGCGTGTGCTTGCAGGCGTTCCAACAATTGACTGGT |
| | | GTGAACTTCATTTTCTACTTTGGCACGACTTTCTTCAACAGTGTTGGG |
| | | CTTGACGGATTCACCACCTCCTTGGCCACCAACATTGTCAATGTTGGC |
| | | TCGACGATCCCTGGTATTTTGGGTGTTGAGATTTTCGGCAGAAGAAAA |
| | | GTGTTGTTGACCGGCGCTGCTGGTATGTGTCTTTCGCAATTCATTGTT |
| | | GCCATTGTTGGTGTAGCCACCGACTCCAAGGCTGCGAACCAAGTTCTT |
| | | ATTGCCTTCTGCTGCATTTTCATTGCGTTCTTTGCAGCCACCTGGGGC |
| | | CCCACCGCATGGGTTGTTTGTGGCGAGATTTTCCCCTTGAGAACCAGA |
| | | GCCAAGTCGATTGCCATGTGCGCTGCTTCGAACTGGTTGTTGAACTGG |
| | | GCAATTGCATACGCCACGCCATACTTGGTTGACTCCGATAAGGGTAAC |
| | | TTGGGCACCAATGTGTTTTTCATTTGGGGAAGCTGTAACTTCTTCTGC |
| | | CTTGTGTTTGCCTACTTCATGATTTACGAGACCAAGGGTCTTTCCTTG |
| | | GAGCAGGTTGATGAGCTTTACGAGAAGGTTGCCAGCGCCAGAAAGTCG |
| | | CCTGGCTTCGTGCCAAGCGAGCACGCTTTCAGAGAGCACGCCGATGTG |
| | | GAGACCGCCATGCCAGACAACTTCAACTTGAAGGCGGAGGCGATTTCT |
| | | GTCGAGGATGCCTCTGTTTAA |
| 18 | NOT USED | |
| 19 | Nucleic acid | ATGAGCATCTTTGAAGGCAAAGACGGGAAGGGGGTATCCTCCACCGAG |
| | sequence of H0 | TCGCTTTCCAATGACGTCAGATATGACAACATGGAGAAAGTTGATCAG |
| | Metschnikowia | GATGTTCTTAGACACAACTTCAACTTTGACAAAGAATTCGAGGAGCTC |
| | species | GAAATCGAGGCGGCGCAAGTCAACGACAAACCTTCTTTTGTCGACAGG |
| | GXS1/HGT12 | ATTTTATCCCTCGAATACAAGCTTCATTTCGAAAACAAGAACCACATG |
| | | GTGTGGCTCTTGGGCGCTTTCGCAGCCGCCGCAGGCTTATTGTCTGGC |
| | | TTGGATCAGTCCATTATTTCTGGTGCATCCATTGGAATGAACAAAGCA |
| | | TTGAACTTGACTGAACGTGAAGCCTCATTGGTGTCTTCGCTTATGCCT |
| | | TTAGGCGCCATGGCAGGCTCCATGATTATGACACCTCTTAATGAGTGG |
| | | TTCGGAAGAAAATCATCGTTGATTATTTCTTGTATTTGGTATACCATC |
| | | GGATCCGCTTTGTGCGCTGGCGCCAGAGATCACCACATGATGTACGCT |
| | | GGCAGATTTATTCTTGGTGTCGGTGTGGGTATAGAAGGTGGGTGTGTG |
| | | GGCATTTACATTTCCGAGTCTGTCCCAGCCAATGTGCGTGGTAGTATC |
| | | GTGTCGATGTACCAGTTCAATATTGCTTTGGGTGAAGTTCTAGGGTAT |
| | | GCTGTTGCTGCCATTTTCTACACTGTTCATGGTGGATGGAGGTTCATG |
| | | GTGGGGTCTTCTTTAGTATTCTCTACTATATTGTTTGCCGGATTGTTT |
| | | TTCTTGCCCGAGTCACCTCGTTGGTTGGTGCACAAAGGCAGAAACGGA |
| | | ATGGCATACGATGTGTGGAAGAGATTGAGAGACATAAACGATGAAAGC |
| | | GCAAAGTTGGAATTTTTGGAGATGAGACAGGCTGCTTATCAAGAGAGA |
| | | GAAAGACGCTCGCAAGAGTCTTTGTTCTCCAGCTGGGGCGAATTATTC |
| | | ACCATCGCTAGAAACAGAAGAGCACTTACTTACTCTGTCATAATGATC |
| | | ACTTTGGGTCAATTGACTGGTGTCAATGCCGTCATGTACTACATGTCG |
| | | ACTTTGATGGGTGCAATTGGTTTCAACGAGAAAGACTCTGTGTTCATG |
| | | TCCCTTGTGGGAGGCGGTTCTTTGCTTATAGGTACCATTCCTGCCATT |
| | | TTGTGGATGGACCGTTTCGGCAGAAGAGTTTGGGGTTATAATCTTGTT |
| | | GGTTTCTTCGTTGGTTTGGTGCTCGTTGGTGTTGGCTACCGTTTCAAT |
| | | CCCGTCACTCAAAAGGCGGCTTCAGAAGGTGTGTACTTGACGGGTCTC |
| | | ATTGTCTATTTCTTGTTCTTTGGTTCCTACTCGACCTTAACTTGGGTC |
| | | ATTCCATCCGAGTCTTTTGATTTGAGAACAAGATCTTTGGGTATGACA |
| | | ATCTGTTCCACTTTCCTTTACTTGTGGTCTTTCACCGTCACCTACAAC |
| | | TTCACCAAGATGTCCGCCGCCTTCACATACACTGGGTTGACACTTGGT |
| | | TTCTACGGTGGCATTGCGTTCCTTGGTTTGATTTACCAGGTCTGCTTC |
| | | ATGCCCGAGACGAAGGACAAGACTTTGGAAGAAATTGACGATATCTTC |
| | | AATCGTTCTGCGTTCTCTATCGCGCGCGAGAACATCTCCAACTTGAAG |
| | | AAGGGTATTTGGTAA |
| 20 | Nucleic acid | ATGAGCATCTTTGAAGGCAAAGACGGGAAGGGGGTATCCTCCACCGAG |
| | sequence of H0 | TCGCTTTCCAATGACGTCAGATATGACAACATGGAGAAAGTTGATCAG |
| | Metschnikowia | GATGTTCTTAGACACAACTTCAACTTTGACAAAGAATTCGAGGAGCTC |
| | species HXT5 | GAAATCGAGGCGGCGCAAGTCAACGACAAACCTTCTTTTGTCGACAGG |
| | | ATTTTATCCCTCGAATACAAGCTTCATTTCGAAAACAAGAACCACATG |
| | | GTGTGGCTCTTGGGCGCTTTCGCAGCCGCCGCAGGCTTATTGTCTGGC |
| | | TTGGATCAGTCCATTATTTCTGGTGCATCCATTGGAATGAACAAAGCA |
| | | TTGAACTTGACTGAACGTGAAGCCTCATTGGTGTCTTCGCTTATGCCT |
| | | TTAGGCGCCATGGCAGGCTCCATGATTATGACACCTCTTAATGAGTGG |
| | | TTCGGAAGAAAATCATCGTTGATTATTTCTTGTATTTGGTATACCATC |
| | | GGATCCGCTTTGTGCGCTGGCGCCAGAGATCACCACATGATGTACGCT |
| | | GGCAGATTTATTCTTGGTGTCGGTGTGGGTATAGAAGGTGGGTGTGTG |
| | | GGCATTTACATTTCCGAGTCTGTCCCAGCCAATGTGCGTGGTAGTATC |
| | | GTGTCGATGTACCAGTTCAATATTGCTTTGGGTGAAGTTCTAGGGTAT |
| | | GCTGTTGCTGCCATTTTCTACACTGTTCATGGTGGATGGAGGTTCATG |
| | | GTGGGGTCTTCTTTAGTATTCTCTACTATATTGTTTGCCGGATTGTTT |
| | | TTCTTGCCCGAGTCACCTCGTTGGTTGGTGCACAAAGGCAGAAACGGA |
| | | ATGGCATACGATGTGTGGAAGAGATTGAGAGACATAAACGATGAAAGC |
| | | GCAAAGTTGGAATTTTTGGAGATGAGACAGGCTGCTTATCAAGAGAGA |
| | | GAAAGACGCTCGCAAGAGTCTTTGTTCTCCAGCTGGGGCGAATTATTC |
| | | ACCATCGCTAGAAACAGAAGAGCACTTACTTACTCTGTCATAATGATC |
| | | ACTTTGGGTCAATTGACTGGTGTCAATGCCGTCATGTACTACATGTCG |
| | | ACTTTGATGGGTGCAATTGGTTTCAACGAGAAAGACTCTGTGTTCATG |
| | | TCCCTTGTGGGAGGCGGTTCTTTGCTTATAGGTACCATTCCTGCCATT |
| | | TTGTGGATGGACCGTTTCGGCAGAAGAGTTTGGGGTTATAATCTTGTT |
| | | GGTTTCTTCGTTGGTTTGGTGCTCGTTGGTGTTGGCTACCGTTTCAAT |
| | | CCCGTCACTCAAAAGGCGGCTTCAGAAGGTGTGTACTTGACGGGTCTC |
| | | ATTGTCTATTTCTTGTTCTTTGGTTCCTACTCGACCTTAACTTGGGTC |
| | | ATTCCATCCGAGTCTTTTGATTTGAGAACAAGATCTTTGGGTATGACA |
| | | ATCTGTTCCACTTTCCTTTACTTGTGGTCTTTCACCGTCACCTACAAC |
| | | TTCACCAAGATGTCCGCCGCCTTCACATACACTGGGTTGACACTTGGT |
| | | TTCTACGGTGGCATTGCGTTCCTTGGTTTGATTTACCAGGTCTGCTTC |
| | | ATGCCCGAGACGAAGGACAAGACTTTGGAAGAAATTGACGATATCTTC |
| | | AATCGTTCTGCGTTCTCTATCGCGCGCGAGAACATCTCCAACTTGAAG |
| | | AAGGGTATTTGGTAA |
| 21 | Nucleic acid | ATGGGATACGAAGAGAAATTAGTGGCCCCCGCTTTGAAATTTAAGAAC |
| | sequence of H0 | TTTTTGGATAAGACCCCAAATATACATAACGTTTACGTAATTGCGGCG |
| | Metschnikowia | ATCTCGTGTACCTCAGGTATGATGTTCGGTTTCGATATATCGTCGATG |
| | species XYT1 | TCCGTGTTCGTGGACCAACAGCCGTATTTAAAAATGTTTGATAACCCT |
| | codon optimized | AGCAGCGTGATACAAGGGTTTATAACTGCGTTGATGTCTTTGGGGAGC |
| | for expression in | TTTTTCGGATCGCTAACGTCCACTTTTATTTCAGAACCTTTTGGTAGA |
| | S. cerevisiae | CGTGCCTCTTTGTTCATATGCGGGATCCTTTGGGTAATTGGGGCGGCA |
| | | GTTCAAAGTTCTTCTCAGAACCGTGCGCAGCTTATTTGTGGCCGAATT |
| | | ATTGCAGGGTGGGGCATCGGATTCGGTTCTAGCGTTGCGCCGGTATAC |
| | | GGTTCAGAAATGGCCCCACGCAAAATTAGAGGAACAATCGGAGGTATT |
| | | TTTCAATTTTCTGTCACGGTCGGAATATTCATAATGTTCCTGATTGGC |
| | | TACGGCTGCTCATTTATACAAGGCAAGGCCAGTTTTAGAATTCCGTGG |
| | | GGAGTTCAAATGGTACCAGGTCTCATTCTGTTGATCGGACTATTCTTC |
| | | ATTCCTGAATCCCCAAGATGGTTAGCCAAACAAGGCTACTGGGAAGAC |
| | | GCTGAGATCATCGTAGCAAACGTTCAAGCTAAGGGTAACAGGAACGAT |
| | | GCTAATGTGCAAATTGAAATGTCCGAGATAAAAGATCAGTTAATGCTT |
| | | GACGAGCATTTAAAGGAGTTTACTTATGCCGATTTGTTTACCAAAAAA |
| | | TACCGGCAAAGGACGATAACAGCTATATTTGCCCAAATATGGCAACAG |
| | | CTGACAGGTATGAATGTCATGATGTACTACATCGTATATATATTTCAA |
| | | ATGGCAGGTTATTCAGGTAATACTAATTTAGTTCCTTCACTCATTCAG |
| | | TATATTATAAATATGGCTGTTACGGTCCCCGCATTGTTCTGTCTTGAT |
| | | CTGCTTGGCAGGAGGACAATTTTATTAGCTGGCGCCGCTTTTATGATG |
| | | GCCTGGCAATTTGGTGTTGCTGGCATTTTAGCTACTTATTCAGAGCCA |
| | | GCCTATATTTCAGATACCGTGAGAATTACAATTCCAGATGACCATAAA |
| | | AGTGCCGCTAAGGGTGTCATCGCTTGCTGCTATTTGTTTGTTTGTTCC |
| | | TTCGCCTTTTCCTGGGGTGTAGGTATCTGGGTTTATTGTTCAGAAGTG |
| | | TGGGGTGATAGTCAATCCAGACAAAGAGGTGCTGCATTGGCAACTTCT |
| | | GCTAATTGGATCTTCAATTTCGCAATTGCAATGTTTACACCTTCTTCT |
| | | TTCAAAAATATCACTTGGAAGACTTATATCATTTATGCTACATTTTGT |
| | | GCTTGTATGTTCATTCATGTTTTTTTTTTTTTCCCTGAAACAAAGGGT |
| | | AAGAGACTAGAAGAAATTGGACAGCTATGGGATGAAGGTGTCCCAGCA |
| | | TGGAGATCTGCAAAATGGCAACCCACTGTCCCACTAGCAAGTGACGCT |
| | | GAATTAGCTCACAAAATGGATGTTGCACACGCTGAACACGCAGACTTA |
| | | TTGGCAACCCATTCTCCAAGTAGTGACGAAAAAACTGGTACCGTTTAA |
| 22 | Nucleic acid | ATGCTGAGCACTACCGATACCCTCGAAAAAAGGGACACCGAGCCTTTC |
| | sequence of H0 | ACTTCAGATGCTCCTGTCACAGTCCATGACTATATCGCAGAGGAGCGT |
| | Metschnikowia | CCGTGGTGGAAAGTGCCGCATTTGCGTGTATTGACTTGGTCTGTTTTC |
| | species HXT2.6 | GTGATCACCCTCACCTCCACCAACAACGGGTATGATGGCCTGATGTTG |
| | | AATGGATTGCAATCCTTGGACATTTGGCAGGAGGATTTGGGTCACCCT |
| | | GCGGGCCAGAAATTGGGTGCCTTGGCCAACGGTGTTTTGTTTGGTAAC |
| | | CTTGCTGCTGTGCCTTTTGCTTCGTATTTCTGCGATCGTTTTGGTAGA |
| | | AGGCCGGTCATTTGTTTCGGACAGATCTTGACAATTGTTGGTGCTGTA |
| | | TTACAAGGTTTGTCCAACAGCTATGGATTTTTTTTGGGTTCGAGAATT |
| | | GTGTTGGGTTTTGGTGCTATGATAGCCACTATTCCGCTGCCAACATTG |
| | | ATTTCCGAAATCGCCTACCCTACGCATAGAGAAACTTCCACTTTCGCC |
| | | TACAACGTGTGCTGGTATTTGGGAGCCATTATCGCCTCCTGGGTCACA |
| | | TACGGCACCAGAGATTTACAGAGCAAGGCTTGCTGGTCAATTCCTTCT |
| | | TATCTCCAGGCCGCCTTACCTTTCTTTCAAGTGTGCATGATTTGGTTT |
| | | GTGCCAGAGTCTCCCAGATTCCTCGTTGCCAAGGGCAAGATCGACCAA |
| | | GCAAGGGCTGTTTTGTCTAAATACCATACAGGAGACTCGACTGACCCC |
| | | AGAGACGTTGCGTTGGTTGACTTTGAGCTCCATGAGATTGAGAGTGCA |
| | | TTGGAGCAGGAAAAATTGAACACTCGCTCGTCATACTTTGACTTTTTC |
| | | AAGAAGAGAAACTTTAGAAAGAGAGGCTTCTTGTGTGTCATGGTCGGT |
| | | GTTGCAATGCAGCTTTCTGGAAACGGCTTAGTGTCCTATTACTTGTCG |
| | | AAAGTGCTAGACTCGATTGGAATCACTGAAACCAAGAGACAGCTCGAG |
| | | ATCAATGGCTGCTTGATGATCTATAACTTTGTCATCTGCGTCTCGTTG |
| | | ATGAGTGTTTGCCGTATGTTCAAAAGAAGAGTATTATTTCTCACGTGT |
| | | TTCTCAGGAATGACGGTTTGCTACACGATATGGACGATTTTGTCAGCG |
| | | CTTAATGAACAGAGACACTTTGAGGATAAAGGCTTGGCCAATGGCGTG |
| | | TTGGCAATGATCTTCTTCTACTATTTTTTCTACAACGTTGGCATCAAT |
| | | GGATTGCCATTCCTATACATCACCGAGATCTTGCCTTACTCACACAGA |
| | | GCAAAAGGCTTGAATTTATTCCAATTCTCGCAATTTCTCACGCAAATC |
| | | TACAATGGCTATGTGAACCCAATCGCCATGGACGCAATCAGCTGGAAG |
| | | TATTACATTGTGTACTGCTGTATTCTCTTCGTGGAGTTGGTGATTGTG |
| | | TTTTTCACGTTCCCAGAAACTTCGGGATACACTTTGGAGGAGGTCGCC |
| | | CAGGTATTTGGTGATGAGGCTCCCGGGCTCCACAACAGACAATTGGAT |
| | | GTTGCGAAAGAATCACTCGAGCATGTTGAGCATGTTTGA |
| 23 | Nucleic acid | ATGAGCCAGTCTAAAGAAAAGTCCAACGTTATTACCACCGTCTTGTCT |
| | sequence of H0 | GAAGAATTGCCAGTTAAGTACTCCGAAGAAATCTCCGATTACGTTTAC |
| | Metschnikowia | CATGATCAACATTGGTGGAAGTACAACCACTTCAGAAAATTGCATTGG |
| | species HXT2.6 | TACATCTTCGTTCTGACTTTGACTTCTACCAACAATGGTTACGATGGC |
| | codon optimized | TCTATGTTGAACGGTCTACAATCTTTGTCTACTTGGAAAGATGCTATG |
| | for expression in | GGTAATCCTGAAGGTTACATTTTGGGTGCTTTGGCTAATGGTACTATT |
| | S. cerevisiae | TTCGGTGGTGTTTTGGCTGTTGCTTTTGCTTCTTGGGCTTGTGATAGA |
| | | TTTGGTAGAAAGTTGACTACCTGCTTCGGTTCTATCGTTACTGTTATT |
| | | GGTGCTATATTGCAAGGTGCCTCTACTAATTACGCATTCTTTTTCGTT |
| | | TCCCGTATGGTTATTGGTTTTGGTTTCGGTCTAGCTTCTGTTGCTTCT |
| | | CCAACTTTGATTGCTGAATTGTCTTTCCCAACTTACAGACCAACTTGT |
| | | ACTGCCTTGTACAATGTTTTTTGGTACTTGGGTGCTGTTATTGCTGCA |
| | | TGGGTTACTTATGGTACTAGAACTATCGTTTCTGCCTACTCTTGGAGA |
| | | ATTCCATCTTACTTGCAAGGTTTGTTGCCATTGGTTCAAGTTTGTTTG |
| | | GTTTGGTGGGTTCCAGAATCTCCAAGATTCTTGGTTTCTAAGGGTAAG |
| | | ATTGAAAAGGCCAGGGAATTCTTGATTAAGTTCCATACTGGTAACGAC |
| | | ACCCAAGAACAAGCTACTAGATTGGTCGAATTTGAGTTGAAAGAAATT |
| | | GAAGCCGCCTTGGAGATGGAAAAGATTAACTCTAATTCTAAGTACACC |
| | | GACTTCATCACCATCAAGACTTTCAGAAAGAGAATCTTCTTGGTTGCT |
| | | TTCACTGCTTGTATGACTCAATTGTCTGGTAACGGTTTGGTGTCTTAC |
| | | TACTTGTCCAAGGTTTTGATCTCCATTGGTATTACCGGTGAGAAAGAA |
| | | CAATTGCAAATCAACGGTTGCCTGATGATCTACAACTTGGTTTTGTCT |
| | | TTAGCTGTTGCCTTCACCTGTTACTTGTTTAGAAGAAAGGCCCTGTTC |
| | | ATCTTCTCTTGCTCATTCATGTTGTTGTCCTACGTTATTTGGACCATT |
| | | CTGTCCGCTATCAATCAACAGAGAAACTTCGAACAAAAAGGTCTAGGT |
| | | CAAGGTGTCTTGGCTATGATTTTTATCTACTACTTGGCCTACAACATC |
| | | GGTTTGAATGGTTTGCCATACTTGTACGTTACCGAAATCTTGCCATAT |
| | | ACTCATAGAGCTAAGGGCATCAACTTGTATTCCTTGGTTATTAACATC |
| | | ACCCTGATCTATAACGGTTTCGTTAACGCTATTGCTATGGATGCTATT |
| | | TCCTGGAAGTACTACATCGTTTACTGCTGCATTATTGCCGTTGAATTG |
| | | GTTGTTGTTATCTTCACCTACGTTGAAACTTTCGGTTACACCTTGGAA |
| | | GAAGTTGCTAGAGTTTTTGAAGGTACTGATTCTTTGGCCATGGACATT |
| | | AACTTGAACGGTACAGTTTCCAACGAAAAGATCGATATCGTTCACTCT |
| | | GAAAGAGGTTCCTCTGCTTAA |
| 24 | Nucleic acid | ATGGGCTTTCGCAACTTAAAGCGCAGGCTCTCAAATGTTGGCGACTCC |
| | sequence of H0 | ATGTCAGTGCACTCTGTGAAAGAGGAGGAAGACTTCTCCCGCGTGGAA |
| | Metschnikowia | ATCCCGGATGAAATCTACAACTATAAGATCGTCCTTGTGGCTTTAACA |
| | species QUP2 | GCGGCGTCGGCTGCCATCATCATCGGCTACGATGCAGGCTTCATTGGT |
| | | GGCACGGTTTCGTTGACGGCGTTCAAACTGGAATTTGGCTTGGACAAA |
| | | ATGTCTGCGACGGCGGCTTCTGCTATCGAAGCCAACGTTGTTTCCGTG |
| | | TTCCAGGCCGGCGCCTACTTTGGGTGTCTTTTCTTCTATCCGATTGGC |
| | | GAGATTTGGGGCCGTAAAATCGGTCTTCTTCTTTCCGGCTTTCTTTTG |
| | | ACGTTTGGTGCTGCTATTTCTTTGATTTCGAACTCGTCTCGTGGCCTT |
| | | GGTGCCATATATGCTGGAAGAGTACTAACAGGTTTGGGGATTGGCGGA |
| | | TGTCTGAGTTTGGCCCCAATCTACGTTTCTGAAATCGCGCCTGCAGCA |
| | | ATCAGAGGCAAGCTTGTGGGCTGCTGGGAAGTGTCATGGCAGGTGGGC |
| | | GGCATTGTTGGCTACTGGATCAATTACGGAGTCTTGCAGACTCTTCCG |
| | | ATTAGCTCACAACAATGGATCATCCCGTTTGCTGTACAATTGATCCCA |
| | | TCGGGGCTTTTCTGGGGCCTTTGTCTTTTGATTCCAGAGCTGCCACGT |
| | | TTTCTTGTATCGAAGGGAAAGATCGATAAGGCGCGCAAAAACTTAGCG |
| | | TACTTGCGTGGACTTAGCGAGGACCACCCCTATTCTGTTTTTGAGTTG |
| | | GAGAACATTAGTAAGGCCATTGAAGAGAACTTCGAGCAAACAGGAAGG |
| | | GGTTTTTTCGACCCATTGAAAGCTTTGTTTTTCAGCAAAAAAATGCTT |
| | | TACCGCCTTCTCTTGTCCACGTCAATGTTCATGATGCAGAATGGCTAT |
| | | GGAATCAATGCTGTGACATACTACTCGCCCACGATCTTCAAATCCTTA |
| | | GGCGTTCAGGGCTCAAACGCCGGTTTGCTCTCAACAGGAATTTTCGGT |
| | | CTTCTTAAAGGTGCCGCTTCGGTGTTCTGGGTCTTTTTCTTGGTTGAC |
| | | ACATTCGGCCGCCGGTTTTGTCTTTGCTACCTCTCTCTCCCCTGCTCG |
| | | ATCTGCATGTGGTATATTGGCGCATACATCAAGATTGCCAACCCTTCA |
| | | GCGAAGCTTGCTGCAGGAGACACAGCCACCACCCCAGCAGGAACTGCA |
| | | GCGAAAGCGATGCTTTACATATGGACGATTTTCTACGGCATTACGTGG |
| | | AATGGTACGACCTGGGTGATCTGCGCGGAGATTTTCCCCCAGTCGGTG |
| | | AGAACAGCCGCGCAGGCCGTCAACGCTTCTTCTAATTGGTTCTGGGCT |
| | | TTCATGATCGGCCACTTCACTGGCCAGGCGCTCGAGAATATTGGGTAC |
| | | GGATACTACTTCTTGTTTGCGGCGTGCTCTGCAATCTTCCCTGTGGTA |
| | | GTCTGGTTTGTGTACCCCGAAACAAAGGGTGTGCCTTTGGAGGCCGTG |
| | | GAGTATTTGTTCGAGGTGCGTCCTTGGAAAGCGCACTCATATGCTTTG |
| | | GAGAAGTACCAGATTGAGTACAACGAGGGTGAATTCCACCAACATAAG |
| | | CCCGAAGTACTCTTACAAGGGTCTGAAAACTCGGACACGAGCGAGAAA |
| | | AGCCTCGCCTGA |
| 25 | Nucleic acid | ATGGGTTTCAGAAACTTGAAGAGAAGATTGTCTAACGTTGGTGACTCC |
| | sequence of H0 | ATGTCTGTTCACTCTGTTAAGGAAGAAGAAGACTTCTCCAGAGTTGAA |
| | Metschnikowia | ATCCCAGATGAAATCTACAACTACAAGATCGTCTTGGTTGCTTTGACT |
| | species QUP2 | GCTGCTTCTGCTGCTATCATCATCGGTTACGATGCTGGTTTCATTGGT |
| | codon optimized | GGTACTGTTTCTTTGACTGCTTTCAAGTCTGAATTCGGTTTGGACAAG |
| | for expression in | ATGTCTGCTACTGCTGCTTCTGCTATCGAAATGGGTTTCAGAAACTTG |
| | S. cerevisiae | AAGAGGCGTTTGTCTAATGTTGGTGATTCCATGTCTGTTCACTCCGTC |
| | | AAAGAAGAAGAGGATTTCTCCAGAGTTGAAATCCCAGACGAAATCTAC |
| | | AACTACAAGATCGTTTTGGTTGCTTTGACTGCTGCTTCTGCTGCTATT |
| | | ATCATTGGTTATGATGCTGGTTTCATCGGTGGTACTGTTTCTTTGACA |
| | | GCTTTCAAGTCTGAATTCGGTTTGGATAAGATGTCTGCTACAGCTGCT |
| | | TCAGCTATTGAAGCTAATGTTGTCTCTGTTTTTCAAGCTGGTGCTTAC |
| | | TTTGGTTGCCTGTTTTTTTACCCAATTGGTGAAATTTGGGGTCGTAAG |
| | | ATTGGTTTGTTGTTGTCTGGTTTCTTGTTGACTTTTGGTGCTGCCATT |
| | | TCCTTGATCTCTAATTCTTCTAGAGGTTTGGGTGCTATCTATGCTGGT |
| | | AGAGTTTTGACTGGTTTAGGTATTGGTGGTTGTTCTTCTTTAGCTCCC |
| | | ATCTACGTTAGTGAAATTGCTCCAGCTGCAATTAGAGGTAAGTTAGTT |
| | | GGTTGTTGGGAAGTTTCTTGGCAAGTTGGTGGTATCGTTGGTTATTGG |
| | | ATTAACTATGGTGTCTTGCAAACCCTGCCAATCTCTTCTCAACAATGG |
| | | ATTATTCCATTCGCCGTTCAATTGATTCCATCTGGTTTGTTTTGGGGT |
| | | TTGTGCTTGTTGATTCCAGAATCTCCAAGATTCTTGGTGTCCAAAGGT |
| | | AAGATTGATAAGGCCAGAAAGAACTTGGCTTACTTGAGAGGTTTGTCT |
| | | GAAGATCATCCATACTCCGTTTTTGAGTTGGAGAACATTTCCAAGGCC |
| | | ATCGAAGAAAACTTTGAACAAACAGGTAGAGGTTTCTTCGACCCATTG |
| | | AAGGCTTTGTTTTTCAGCAAGAAAATGCTGTACAGGCTGCTGTTGTCT |
| | | ACTTCTATGTTTATGATGCAAAACGGCTACGGTATTAACGCTGTTACT |
| | | TATTACTCTCCCACCATCTTTAAGTCCTTGGGTGTTCAAGGTTCTAAT |
| | | GCCGGTTTGTTATCTACTGGTATTTTCGGTTTGTTGAAAGGTGCCGCT |
| | | TCTGTTTTTTGGGTTTTCTTCTTGGTTGATACCTTCGGTAGAAGATTC |
| | | TGTTTGTGCTATTTGTCTTTGCCATGCTCTATCTGCATGTGGTATATT |
| | | GGTGCCTACATTAAGATTGCTAACCCATCTGCTAAATTGGCTGCTGGT |
| | | GATACTGCTACTACTCCAGCTGGTACTGCTGCTAAAGCTATGTTGTAT |
| | | ATTTGGACCATCTTCTACGGTATCACTTGGAATGGTACTACCTGGGTT |
| | | ATTTGCGCTGAAATTTTTCCACAATCTGTTAGAACAGCTGCTCAAGCT |
| | | GTTAATGCTTCTTCTAATTGGTTTTGGGCCTTCATGATTGGTCATTTT |
| | | ACTGGTCAAGCTTTGGAAAACATTGGTTACGGTTACTACTTTTTGTTC |
| | | GCTGCTTGTTCCGCTATTTTCCCAGTTGTAGTTTGGTTCGTTTACCCA |
| | | GAAACAAAAGGTGTTCCATTGGAAGCTGTTGAATACTTGTTTGAAGTT |
| | | AGACCATGGAAGGCTCATTCTTACGCTTTAGAAAAGTACCAGATCGAG |
| | | TACAACGAAGGTGAATTCCATCAACATAAGCCAGAAGTTTTGTTGCAG |
| | | GGTTCTGAAAACTCTGATACCTCTGAAAAGTCTTTGGCCTGAAACGAA |
| | | GGTGAATTCCACCAACATAAGCCAGAAGTTTTGTTGCAAGGTTCTGAA |
| | | AACTCTGACACTTCTGAAAAGTCTTTGGCTTAA |
| 26 | Nucleic acid | ATGTCAGAAAAGCCTGTTGTGTCGCACAGCATCGACACGACGCTGTCT |
| | sequence of H0 | ACGTCATCGAAACAAGTCTATGACGGTAACTCGCTTCTTAAGACCCTG |
| | Metschnikowia | AATGAGCGCGATGGCGAACGCGGCAATATCTTGTCGCAGTACACTGAG |
| | species | GAACAGGCCATGCAAATGGGCCGCAACTATGCGTTGAAGCACAATTTA |
| | APS1/HGT19 | GATGCGACACTCTTTGGAAAGGCGGCCGCGGTCGCAAGAAACCCATAC |
| | | GAGTTCAATTCGATGAGTTTTTTGACCGAAGAGGAAAAAGTCGCGCTT |
| | | AACACGGAGCAGACCAAGAAATGGCACATCCCAAGAAAGTTGGTGGAG |
| | | GTGATTGCATTGGGGTCCATGGCCGCTGCGGTGCAGGGTATGGATGAG |
| | | TCGGTGGTGAATGGTGCAACGCTTTTCTACCCCACGGCAATGGGTATC |
| | | ACAGATATCAAGAATGCCGATTTGATTGAAGGTTTGATCAACGGTGCG |
| | | CCCTATCTTTGCTGCGCCATCATGTGCTGGACATCTGATTACTGGAAC |
| | | AGGAAGTTGGGCCGTAAGTGGACCATTTTCTGGACATGTGCCATTTCT |
| | | GCAATCACATGTATCTGGCAAGGTCTCGTCAATTTGAAATGGTACCAT |
| | | TTGTTCATTGCGCGTTTCTGCTTGGGTTTCGGTATCGGTGTCAAGTCT |
| | | GCCACCGTGCCTGCGTATGCTGCCGAAACCACCCCGGCCAAAATCAGA |
| | | GGCTCGTTGGTCATGCTTTGGCAGTTCTTCACCGCTGTCGGAATCATG |
| | | CTTGGTTACGTGGCGTCTTTGGCATTCTATTACATTGGTGACAATGGC |
| | | ATTTCTGGCGGCTTGAACTGGAGATTGATGCTAGGATCTGCATGTCTT |
| | | CCAGCTATCGTTGTGTTAGTCCAAGTTCCGTTTGTTCCAGAATCCCCT |
| | | CGTTGGCTCATGGGTAAGGAAAGACACGCTGAAGCATATGATTCGCTC |
| | | CGGCAATTGCGGTTCAGTGAAATCGAGGCGGCCCGTGACTGTTTCTAC |
| | | CAGTACGTGTTGTTGAAAGAGGAGGGCTCTTATGGAACGCAGCCATTC |
| | | TTCAGCAGAATCAAGGAGATGTTCACCGTGAGAAGAAACAGAAATGGT |
| | | GCATTGGGCGCGTGGATCGTCATGTTCATGCAGCAGTTCTGTGGAATC |
| | | AACGTCATTGCTTACTACTCGTCGTCGATCTTCGTGGAGTCGAATCTT |
| | | TCTGAGATCAAGGCCATGTTGGCGTCTTGGGGGTTCGGTATGATCAAT |
| | | TTCTTGTTTGCAATTCCAGCGTTCTACACCATTGACACGTTTGGCCGA |
| | | CGCAACTTGTTGCTCACTACTTTCCCTCTTATGGCGGTATTCTTACTC |
| | | ATGGCCGGATTCGGGTTCTGGATCCCGTTCGAGACAAACCCACACGGC |
| | | CGTTTGGCGGTGATCACTATTGGTATCTATTTGTTTGCATGTGTCTAC |
| | | TCTGCGGGCGAGGGACCAGTTCCCTTCACATACTCTGCCGAAGCATTC |
| | | CCGTTGTATATCCGTGACTTGGGTATGGGCTTTGCCACGGCCACGTGT |
| | | TGGTTCTTCAACTTCATTTTGGCATTTTCCTGGCCTAGAATGAAGAAT |
| | | GCATTCAAGCCTCAAGGTGCCTTTGGCTGGTATGCCGCCTGGAACATT |
| | | GTTGGCTTCTTCTTAGTGTTATGGTTCTTGCCCGAGACAAAGGGCTTG |
| | | ACGTTGGAGGAATTGGACGAAGTGTTTGATGTGCCTTTGAGAAAACAC |
| | | GCGCACTACCGTACCAAAGAATTAGTATACAACTTGCGCAAATACTTC |
| | | TTGAGGCAGAACCCTAAGCCATTGCCGCCACTTTATGCACACCAAAGA |
| | | ATGGCTGTTACCAACCCAGAATGGTTGGAAAAGACCGAGGTCACGCAC |
| | | GAGGAGAATATCTAG |
| 27 | Nucleic acid | ATGTCTGAAAAGCCAGTTGTTTCTCACTCTATCGACACCACCTCTTCT |
| | sequence of H0 | ACCTCTTCTAAGCAAGTCTACGACGGTAACTCTTTGTTGAAGACCTCT |
| | Metschnikowia | AACGAAAGAGACGGTGAAAGAGGTAACATCTTGTCTCAATACACTGAA |
| | species | GAACAAGCAATGCAAATGGGTAGAAACTACGCTTTGAAGCACAACTTG |
| | APS1/HGT19 | GACGCTACCTTGTTCGGTAAGGCTGCTGCTGTCGCTAGAAACCCATAC |
| | codon optimized | GAGTTCAACTCTATGTCTTTCTTGACCGAAGAAGAAAAGGTCGCTTTG |
| | for expression in | AACACCGAACAAACCAAGAAGTGGCACATCCCAAGAAAGTTGGTTGAA |
| | S. cerevisiae | GTTATTGCTTTGGGTTCTATGGCTGCTGCTGTTCAAGGTATGGACGAA |
| | | TCTGTTGTTAACGGTGCTACCTTGTTCTACCCAACCGCTATGGGTATC |
| | | ACCGACATCAAGAACGCTGACTTGATTGAAGGTTTGATTAACGGTGCC |
| | | CCATACTTGTGTTGTGCTATTATGTGTTGGACCTCTGACTACTGGAAC |
| | | AGAAAGTTGGGTAGAAAGTGGACCATTTTCTGGACCTGTGCTATTTCT |
| | | GCTATCACCTGTATCTGGCAAGGTTTGGTCAACTTGAAGTGGTATCAC |
| | | TTGTTCATTGCTAGATTCTGTTTGGGTTTCGGTATCGGTGTCAAGTCT |
| | | GCTACCGTTCCAGCCTACGCTGCTGAAACCACCCCAGCCAAGATTAGA |
| | | GGTTCTTTGGTTATGTTGTGGCAATTCTTCACCGCTGTCGGTATTATG |
| | | TTGGGTTACGTTGCTTCTTTGGCTTTCTACTACATTGGTGACAACGGT |
| | | ATTTCTGGTGGTTTGAACTGGAGATTGATGTTGGGTTCTGCTTGTTTG |
| | | CCAGCCATCGTTGTTTTGGTCCAAGTTCCATTCGTTCCAGAATCTCCA |
| | | AGATGGTTGATGGGTAAGGAAAGACACGCTGAAGCCTACGACTCTTTG |
| | | AGACAATTGAGATTCTCTGAAATCGAAGCCGCTAGAGACTGTTTCTAC |
| | | CAATACGTTTTGTTGAAGGAAGAAGGTTCTTACGGTACTCAACCATTC |
| | | TTCTCTAGAATCAAGGAAATGTTCACCGTTAGAAGAAACAGAAACGGT |
| | | GCTTTGGGTGCTTGGATTGTTATGTTTATGCAACAATTCTGTGGTATC |
| | | AACGTCATTGCTTACTACTCTTCTTCTATCTTCGTTGAATCTAACTTG |
| | | TCTGAAATCAAGGCTATGTTGGCTTCTTGGGGTTTCGGTATGATTAAC |
| | | TTCTTGTTCGCTATTCCAGCCTTCTACACCATTGACACCTTCGGTAGA |
| | | AGAAACTTGTTGTTGACTACTTTCCCATTGATGGCTGTTTTCTTGTTG |
| | | ATGGCTGGTTTCGGTTTCTGGATTCCATTCGAAACCAACCCACACGGT |
| | | AGATTGGCTGTTATCACTATTGGTATCTACTTGTTCGCTTGTGTCTAC |
| | | TCTGCTGGTGAAGGTCCAGTTCCATTCACCTACTCTGCTGAAGCCTTC |
| | | CCATTGTACATCAGAGACTTGGGTATGGGTTTCGCTACCGCTACCTGT |
| | | TGGTTCTTCAACTTCATTTTGGCTTTCTCTTGGCCAAGAATGAAGAAC |
| | | GCTTTCAAGCCTCAAGGTGCTTTCGGTTGGTACGCTGCTTGGAACATT |
| | | GTTGGTTTCTTCTTGGTTTTGTGGTTCTTGCCAGAAACTAAGGGTTTG |
| | | ACTTTGGAAGAATTGGACGAAGTTTTCGACGTTCCATTGAGAAAGCAC |
| | | GCTCACTACAGAACTAAGGAATTGGTTTACAACTTGAGAAAGTACTTC |
| | | TTGAGACAAAACCCAAAGCCATTGCCACCATTGTACGCTCACCAAAGA |
| | | ATGGCTGTTACCAACCCAGAATGGTTGGAAAAGACCGAAGTCACCCAC |
| | | GAAGAAAACATCTAA |
| 28 | Amino acid | MATIKLNSGYDMPQVGFGCWKVTNSTCADTIYNAIKVGYRLFDGAEDY |
| | sequence of H0 | GNEKEVGEGINRAIDEGLVARDELFVVSKLWNNFHHPDNVEKALDKTL |
| | Metschnikowia | GDLNVEYLDLFLIHFPIAFKFVPFEEKYPPGFYCGEGDKFIYEDVPLL |
| | species Xyl1p | DTWRALEKFVKKGKIRSIGISNFSGALIQDLLRGAEIPPAVLQIEHHP |
| | | YLQQPRLIEYVQSKGIAITAYSSFGPQSFVELDHPKVKECVTLFEHED |
| | | IVSIAKAHDKSAGQVLLRWATQRGLAVIPKSNKIERLLLNLNVNDFDL |
| | | SEAELEQIAKLDVGLRFNNPWDWDKIPIFH |
| 29 | Amino acid | MPANPSLVLNKVNDITFENYEVPLLTDPNDVLVQVKKTGICGSDIHYY |
| | sequence of H0 | THGRIGDFVLTKPMVLGHESAGVVVEVGKGVTDLKVGDKVAIEPGVPS |
| | Metschnikowia | RTSDEYKSGHYNLCPHMCFAATPNSNPDEPNPPGTLCKYYKSPADFLV |
| | species Xyl2p | KLPEHVSLELGAMVEPLTVGVHASRLGRVTFGDHVVVFGAGPVGILAA |
| | | AVARKFGAASVTIVDIFDSKLELAKSIGAATHTFNSMTEGVLSEALPA |
| | | GVRPDVVLECTGAEICVQQGVLALKAGGRHVQVGNAGSYLKFPITEFV |
| | | TKELTLFGSFRYGYNDYKTSVAILDENYKNGKENALVDFEALITHRFP |
| | | FKNAIEAYDAVRAGDGAVKCIIDGPE |
| 30 | Amino acid | MTYSSSSGLFLGFDLSTQQLKIIVTNENLKALGTYHVEFDAQFKEKYA |
| | sequence of H0 | IKKGVLSDEKTGEILSPVHMWLEAIDHVFGLMKKDNFPFGKVKGISGS |
| | Metschnikowia | GMQHGSVFWSKSASSSLKNMAEYSSLTEALADAFACDTSPNWQDHSTG |
| | species Xks1p | KEIKDFEKVVGGPDKLAEITGSRAHYRFTGLQIRKLAVRSENDVYQKT |
| | | DRISLVSSFVASVLLGRITTIEEADACGMNLYNVTESKLDEDLLAIAA |
| | | GVHPKLDNKSKRETDEGVKELKRKIGEIKPVSYQTSGSIAPYFVEKYG |
| | | FSPDSKIVSFTGDNLATIISLPLRKNDVLVSLGTSTTVLLVTESYAPS |
| | | SQYHLFKHPTIKNAYMGMICYSNGALARERVRDAINEKYGVAGDSWDK |
| | | FNEILDRSGDFNNKLGVYFPIGEIVPNAPAQTKRMEMNSHEDVKEIEK |
| | | WDLENDVTSIVESQTVSCRVRAGPMLSGSGDSNEGTPENENRKVKTLI |
| | | DDLHSKFGEIYTDGKPQSYESLTSRPRNIYFVGGASRNKSIIHKMASI |
| | | MGATEGNFQVEIPNACALGGAYKASWSLECESRQKWVHFNDYLNEKYD |
| | | FDDVDEFKVDDKWLNYIPAIGLLSKLESNLDQN |
| 31 | Amino acid | MSDIDQLAISTIRLLAVDAVAKANSGHPGAPLGLAPAAHAVWKEMKFN |
| | sequence of H0 | PKNPDWVNRDRFVLSNGHACALLYAMLHLYGFDMSLDDLKQFRQLNSK |
| | Metschnikowia | TPGHPEKFEIPGAEVTTGPLGQGISNAVGLAIAQKQFAATFNKDDFAI |
| | species Tkl1p | SDSYTYAFLGDGCLMEGVASEASSLAGHLQLNNLIAFWDDNKISIDGS |
| | | IEVAFTEDVLKRYEAYGWDTLTIEKGDTDLEGVAQAIKTAKASKKPTL |
| | | IRLTTIIGYGSLQQGTHGVHGAPLKPDDIKQLKEKFGFDPTKSFVVPQ |
| | | EVYDYYGTLVKKNQELESEWNKTVESYIQKFPEEGAVLARRLKGELPE |
| | | DWAKCLPTYTADDKPLATRKLSEMALIKILDVVPELIGGSADLTGSNL |
| | | TRAPDMVDFQPPQTGLGNYAGRYIRYGVREHGMGAIMNGIAGFGAGFR |
| | | NYGGTFLNFVSYAAGAVRLSALSHLPVIWVATHDSIGLGEDGPTHQPI |
| | | ETLAHFRATPNISVWRPADGNEVSAAYKSAIESTSTPHILALTRQNLP |
| | | QLAGSSVEKASTGGYTVYQTTDKPAVIIVASGSEVAISIDAAKKLEGE |
| | | GIKANVVSLVDFHTFDKQPLDYRLSVLPDGVPIMSVEVMSSFGWSKYS |
| | | HEQFGLNRFGASGKAEDLYKFFDFTPEGVADRAAKTVQFYKGKDLLSP |
| | | LNRAF |
| 32 | Amino acid | MSNSLESLKATGTVIVTDTGEFDSIAKYTPQDATTNPSLILAASKKAE |
| | sequence of H0 | YAKVIDVAIKYAEDKGSNPKEKAAIALDRLLVEFGKEILSIVPGRVSI |
| | Metschnikowia | EVDARLSFDKDATVKKALEIIELYKSIGISKDRVLIKIASTWEGIQAA |
| | species Tal1p | KELEAKHDIHCNLTLLFSFVQAVACAEAKVTLISPFVGRILDWYKAST |
| | | GKEYDAESDPGVVSVRQIYNYYKKYGYNTIVMGASFRNTGEIKALAGC |
| | | DYLTVAPKLLEELMNSSEEVPKVLDAASASSASEEKVSYIDDESEFRF |
| | | LLNEDAMATEKLAQGIRGFAKDAQTLLAELENRFK |
| 33 | Nucleic acid | ATGGCTACTATCAAATTGAACTCTGGATACGACATGCCCCAAGTGGGT |
| | sequence of H0 | TTTGGGTGCTGGAAAGTAACTAACAGTACATGTGCTGATACGATCTAC |
| | Metschnikowia | AACGCGATCAAAGTTGGCTACAGATTATTTGATGGCGCTGAAGATTAC |
| | species XYL1 | GGGAACGAGAAAGAGGTGGGCGAAGGAATCAACAGGGCCATTGACGAA |
| | | GGCTTGGTGGCACGTGACGAGTTGTTCGTGGTGTCCAAGCTCTGGAAC |
| | | AACTTCCATCATCCAGACAACGTCGAGAAGGCGTTGGACAAGACTTTG |
| | | GGCGACTTGAATGTCGAGTACTTGGACTTGTTCTTGATCCATTTCCCA |
| | | ATTGCGTTCAAATTCGTGCCCTTTGAGGAGAAATACCCGCCCGGCTTC |
| | | TACTGTGGAGAAGGCGATAAGTTTATCTACGAGGATGTGCCTTTGCTT |
| | | GACACGTGGCGGGCATTGGAGAAGTTTGTGAAGAAGGGTAAGATCAGA |
| | | TCCATCGGAATCTCGAACTTTTCCGGCGCGTTGATCCAGGACTTGCTC |
| | | AGGGGCGCCGAGATCCCCCCTGCCGTGTTGCAGATTGAGCACCACCCA |
| | | TACTTGCAGCAGCCCAGATTGATTGAGTATGTGCAGTCCAAGGGTATT |
| | | GCCATCACAGCCTACTCCTCTTTTGGCCCACAGTCGTTTGTGGAGTTG |
| | | GACCACCCCAAGGTCAAGGAGTGTGTCACGCTTTTCGAGCACGAAGAC |
| | | ATTGTTTCCATCGCTAAAGCTCACGACAAGTCCGCGGGCCAGGTATTA |
| | | TTGAGGTGGGCCACGCAAAGGGGTCTTGCCGTGATTCCAAAGTCAAAC |
| | | AAAACCGAGCGTTTGTTGCTGAATTTGAATGTGAACGATTTTGATCTC |
| | | TCTGAAGCAGAATTGGAGCAAATCGCAAAGTTGGACGTGGGCTTGCGC |
| | | TTCAACAACCCTTGGGACTGGGACAAGATTCCAATCTTCCATTAA |
| 34 | Nucleic acid | ATGCCTGCTAACCCATCCTTGGTTTTGAACAAAGTGAACGACATCACG |
| | sequence of H0 | TTCGAGAACTACGAGGTTCCGTTACTCACAGACCCCAACGATGTATTG |
| | Metschnikowia | GTTCAGGTGAAAAAGACTGGAATCTGTGGATCTGACATCCACTACTAC |
| | species XYL2 | ACCCACGGCAGAATTGGCGACTTCGTGTTGACAAAGCCAATGGTTTTG |
| | | GGCCACGAATCCGCCGGTGTGGTCGTGGAGGTCGGCAAAGGTGTCACT |
| | | GACTTGAAGGTTGGTGATAAGGTTGCCATTGAGCCCGGAGTGCCTTCT |
| | | CGCACCAGTGACGAGTACAAGAGTGGCCACTACAACTTGTGCCCACAC |
| | | ATGTGTTTTGCCGCCACGCCCAACTCTAACCCCGACGAGCCAAACCCG |
| | | CCAGGGACTTTGTGCAAATATTACAAGTCCCCAGCGGACTTCTTGGTG |
| | | AAATTGCCTGAGCACGTCTCCCTTGAGTTGGGCGCTATGGTCGAGCCT |
| | | TTGACTGTCGGTGTGCACGCCTCGCGTTTGGGCCGTGTCACTTTTGGT |
| | | GACCACGTTGTGGTTTTCGGTGCTGGCCCAGTCGGTATCCTTGCGGCT |
| | | GCCGTGGCCAGAAAGTTTGGCGCTGCCAGCGTGACTATCGTCGACATC |
| | | TTCGACAGCAAATTGGAATTGGCCAAGTCCATTGGCGCGGCCACTCAC |
| | | ACATTCAACTCAATGACTGAGGGTGTTCTTTCGGAGGCTTTGCCCGCG |
| | | GGCGTGAGACCTGACGTTGTATTGGAGTGCACTGGAGCAGAGATCTGT |
| | | GTGCAGCAAGGTGTACTTGCGTTGAAGGCTGGTGGCCGCCACGTGCAA |
| | | GTTGGAAATGCCGGCTCCTATCTCAAATTCCCCATCACCGAATTTGTT |
| | | ACCAAGGAGTTGACTCTCTTTGGATCCTTCCGTTACGGTTACAACGAC |
| | | TACAAGACGTCGGTCGCCATCTTGGACGAGAATTACAAGAACGGGAAG |
| | | GAGAATGCGTTGGTGGACTTTGAAGCCTTGATTACTCACCGTTTCCCC |
| | | TTCAAGAATGCCATTGAGGCTTACGACGCGGTGCGCGCTGGCGACGGA |
| | | GCTGTCAAGTGTATCATTGACGGCCCAGAGTAA |
| 35 | Nucleic acid | ATGACTTATAGTTCCAGCTCTGGCCTCTTTTTGGGCTTCGACTTGTCG |
| | sequence of H0 | ACGCAGCAGCTTAAAATCATTGTGACAAACGAGAACTTGAAGGCGCTT |
| | Metschnikowia | GGTACCTACCATGTTGAGTTTGATGCTCAATTCAAAGAGAAATACGCG |
| | species XKS1 | ATCAAAAAGGGTGTTTTGTCAGATGAAAAAACGGGCGAGATTTTATCA |
| | | CCCGTGCACATGTGGCTAGAGGCAATTGACCATGTCTTTGGGTTGATG |
| | | AAAAAAGACAATTTCCCCTTCGGAAAAGTGAAAGGCATAAGCGGTTCA |
| | | GGGATGCAGCACGGATCGGTCTTTTGGTCGAAGTCTGCTTCTTCATCC |
| | | TTAAAGAATATGGCCGAATATTCCTCTTTAACAGAAGCCTTGGCTGAT |
| | | GCCTTTGCGTGTGATACTTCTCCCAACTGGCAGGACCATTCGACAGGG |
| | | AAAGAAATCAAAGACTTTGAGAAAGTCGTTGGAGGCCCGGACAAATTG |
| | | GCGGAAATTACAGGCTCAAGAGCTCACTACAGGTTCACTGGGTTGCAG |
| | | ATTCGGAAGTTGGCAGTGAGATCTGAGAATGACGTTTACCAGAAAACC |
| | | GATAGAATATCTTTGGTGTCGAGTTTTGTTGCGTCCGTTCTTTTGGGC |
| | | AGGATCACCACAATTGAGGAGGCGGACGCTTGCGGAATGAATTTATAC |
| | | AATGTGACCGAGTCTAAGCTTGATGAAGATTTGTTAGCAATCGCTGCA |
| | | GGGGTGCATCCAAAGCTCGATAACAAATCCAAAAGGGAAACAGACGAG |
| | | GGTGTCAAAGAACTAAAGCGAAAGATTGGTGAGATCAAACCCGTGAGT |
| | | TATCAGACTTCGGGCTCAATCGCACCATATTTTGTCGAGAAATACGGC |
| | | TTCTCTCCAGATTCGAAGATTGTTTCGTTTACGGGTGATAATCTTGCG |
| | | ACCATCATCTCTTTGCCTTTGAGAAAAAACGACGTCTTGGTGTCACTA |
| | | GGCACATCCACCACCGTACTTTTGGTGACCGAGAGCTACGCGCCTTCT |
| | | TCGCAGTATCATCTTTTCAAGCATCCTACAATTAAGAATGCTTACATG |
| | | GGAATGATTTGCTACAGTAATGGCGCGCTAGCAAGAGAAAGAGTTCGT |
| | | GACGCCATCAATGAGAAGTATGGTGTGGCAGGGGATTCTTGGGACAAG |
| | | TTCAATGAGATCTTGGATCGCTCAGGCGACTTCAACAATAAGTTGGGT |
| | | GTTTACTTTCCCATCGGTGAAATTGTGCCCAATGCTCCGGCCCAGACA |
| | | AAGAGAATGGAAATGAACTCGCATGAGGATGTGAAAGAGATCGAAAAG |
| | | TGGGATTTGGAAAACGATGTCACTTCTATTGTTGAGTCACAAACCGTT |
| | | AGTTGCCGAGTGAGAGCGGGCCCAATGCTTTCTGGATCGGGTGACTCG |
| | | AATGAAGGAACGCCCGAAAATGAAAATAGGAAAGTCAAAACACTCATC |
| | | GACGATTTACACTCTAAGTTCGGCGAAATTTACACAGACGGGAAACCT |
| | | CAGAGCTACGAGTCTTTGACTTCGAGGCCGCGGAACATCTACTTTGTC |
| | | GGAGGGGCTTCAAGAAACAAGAGTATCATACACAAGATGGCTTCGATC |
| | | ATGGGTGCTACCGAAGGAAACTTTCAGGTTGAGATTCCGAATGCGTGT |
| | | GCTCTTGGCGGCGCCTACAAGGCAAGCTGGAGCCTTGAGTGTGAGAGC |
| | | AGACAAAAGTGGGTGCACTTCAATGATTACCTCAATGAGAAGTACGAT |
| | | TTCGATGATGTGGATGAGTTCAAAGTGGACGACAAATGGCTCAACTAT |
| | | ATTCCGGCGATTGGCTTGTTGTCGAAATTGGAAAGCAACCTTGACCAG |
| | | AACTAA |
| 36 | Nucleic acid | ATGTCCGACATCGATCAATTGGCTATTTCTACCATCCGTTTGTTGGCG |
| | sequence of H0 | GTCGACGCCGTGGCCAAGGCCAACTCTGGTCACCCCGGTGCCCCATTG |
| | Metschnikowia | GGTCTCGCCCCTGCCGCCCACGCCGTTTGGAAGGAGATGAAATTCAAC |
| | species TKL1 | CCAAAGAACCCCGACTGGGTCAACAGAGACCGTTTTGTGTTGTCGAAC |
| | | GGTCACGCTTGCGCTTTGTTATACGCCATGTTGCACCTTTACGGCTTC |
| | | GACATGTCGCTTGACGACTTGAAGCAGTTCCGTCAGTTGAACTCGAAA |
| | | ACACCCGGACATCCCGAGAAGTTTGAAATCCCAGGTGCCGAGGTCACC |
| | | ACGGGCCCCTTGGGTCAGGGTATCTCCAACGCCGTGGGTTTGGCCATT |
| | | GCACAGAAGCAATTCGCTGCCACGTTCAACAAGGACGATTTCGCCATC |
| | | TCTGACTCGTACACCTACGCCTTCTTGGGTGACGGATGTTTGATGGAG |
| | | GGTGTCGCCTCGGAAGCATCTTCTTTGGCTGGCCACCTCCAATTGAAC |
| | | AACTTGATTGCGTTCTGGGACGACAACAAGATCTCGATCGATGGATCC |
| | | ACTGAAGTGGCCTTCACCGAGGACGTGTTGAAGCGTTACGAGGCTTAC |
| | | GGTTGGGACACGCTCACGATTGAGAAGGGTGACACTGACTTGGAGGGC |
| | | GTCGCTCAGGCGATCAAGACTGCCAAGGCGCTGAAGAAGCCTACTTTG |
| | | ATCCGTTTGACCACCATCATCGGCTACGGCTCGCTCCAGCAGGGTACC |
| | | CACGGTGTTCACGGTGCTCCATTGAAGCCAGATGACATCAAGCAGTTG |
| | | AAGGAGAAGTTTGGCTTCGACCCAACCAAGTCGTTTGTCGTGCCTCAG |
| | | GAAGTTTACGACTACTACGGCACACTCGTAAAGAAGAACCAGGAGTTG |
| | | GAGTCCGAGTGGAACAAGACCGTCGAGTCCTACATCCAGAAATTCCCA |
| | | GAGGAGGGCGCTGTCTTGGCGCGCAGACTCAAGGGTGAGTTGCCTGAG |
| | | GACTGGGCCAAGTGCTTGCCTACTTACACCGCTGATGACAAGCCGTTG |
| | | GCCACGAGAAAGTTGTCTGAGATGGCTCTCATCAAGATCTTGGATGTC |
| | | GTTCCAGAGCTTATTGGTGGCTCTGCCGACTTGACCGGCTCGAACTTG |
| | | ACCCGTGCCCCTGACATGGTTGACTTCCAGCCCCCTCAGACCGGCTTG |
| | | GGTAACTACGCTGGTAGATACATCCGTTACGGTGTGCGTGAGCACGGT |
| | | ATGGGTGCCATCATGAACGGTATCGCCGGTTTTGGTGCTGGTTTCCGT |
| | | AACTACGGCGGTACCTTCTTGAACTTCGTCTCGTACGCCGCCGGTGCT |
| | | GTGCGTTTGTCGGCTCTTTCTCACTTGCCTGTGATCTGGGTTGCTACG |
| | | CATGACTCGATTGGTTTGGGTGAGGACGGTCCTACCCACCAGCCTATT |
| | | GAGACCTTGGCCCACTTCAGAGCTACCCCTAACATCTCTGTGTGGAGA |
| | | CCTGCTGACGGTAACGAGGTGTCAGCTGCTTACAAGTCTGCCATTGAG |
| | | TCTACCTCTACCCCACACATCTTGGCCTTGACCAGACAGAACTTGCCT |
| | | CAATTGGCTGGTTCTTCTGTGGAGAAGGCCTCTACCGGTGGTTACACC |
| | | GTGTACCAGACCACTGACAAGCCTGCCGTCATCATCGTGGCTTCTGGT |
| | | TCCGAGGTGGCCATCTCTATTGACGCCGCCAAGAAGTTGGAGGGTGAG |
| | | GGCATCAAGGCCAACGTTGTTTCCTTGGTTGACTTCCACACTTTCGAC |
| | | AAGCAGCCTTTGGACTACCGTTTATCTGTTTTGCCAGATGGCGTGCCA |
| | | ATCATGTCCGTTGAGGTGATGTCCTCGTTCGGCTGGTCCAAGTATTCT |
| | | CACGAGCAGTTCGGCTTGAACAGATTCGGTGCCTCCGGCAAGGCCGAA |
| | | GACCTTTACAAGTTCTTCGACTTCACGCCAGAAGGCGTTGCTGACAGA |
| | | GCCGCCAAGACCGTGCAGTTCTACAAGGGCAAGGACCTCCTTTCGCCT |
| | | TTGAACAGAGCCTTCTAA |
| 37 | Nucleic acid | ATGTCTAACTCTTTGGAATCCTTGAAAGCTACCGGCACCGTGATCGTC |
| | sequence of H0 | ACCGACACTGGTGAGTTCGACTCGATTGCCAAGTACACCCCACAAGAT |
| | Metschnikowia | GCCACCACCAACCCTTCGTTGATTTTAGCCGCCTCGAAAAAGGCTGAG |
| | species TAL1 | TACGCCAAGGTGATTGATGTTGCTATTAAATACGCCGAGGACAAGGGC |
| | | AGCAACCCTAAGGAGAAGGCCGCCATTGCCTTGGACAGATTGTTGGTG |
| | | GAGTTCGGTAAGGAAATCTTGCTGATTGTGCCTGGCAGAGTGTCTACC |
| | | GAGGTTGACGCCAGATTGTCGTTTGACAAGGACGCCACCGTCAAGAAG |
| | | GCGCTTGAGATCATCGAATTGTACAAGTCCATTGGCATCTCGAAGGAC |
| | | AGAGTGTTGATCAAGATCGCTTCCACCTGGGAAGGTATCCAGGCCGCC |
| | | AAGGAGTTGGAGGCCAAGCACGACATCCACTGTAACTTGACGCTTTTG |
| | | TTCAGTTTCGTGCAGGCGGTGGCGTGTGCCGAGGCCAAGGTCACTTTG |
| | | ATCTCGCCTTTCGTCGGCAGAATCTTGGACTGGTACAAGGCCTCCACC |
| | | GGCAAGGAGTACGATGCCGAGTCCGACCCTGGTGTTGTGTCTGTCAGA |
| | | CAGATCTACAACTACTACAAGAAGTACGGCTACAACACGATTGTCATG |
| | | GGCGCGTCTTTCAGAAACACTGGCGAGATCAAGGCCTTGGCTGGCTGC |
| | | GACTACTTGACTGTGGCCCCTAAGTTGTTGGAGGAGTTGATGAACTCT |
| | | TCCGAGGAGGTGCCTAAGGTGTTGGACGCTGCCTCGGCCAGCTCCGCG |
| | | TCTGAGGAGAAGGTTTCCTACATTGACGACGAGAGCGAGTTCAGATTC |
| | | TTGTTGAACGAGGACGCCATGGCCACCGAGAAGTTGGCCCAGGGTATC |
| | | AGAGGCTTTGCCAAGGACGCCCAGACCTTGTTGGCCGAGTTGGAGAAC |
| | | AGATTCAAGTAG |

In some embodiments, the microbial organisms can have at least one exogenous nucleic acid encoding a xylose transporter that is at least 30% identical to a Metschnikowia xylose transporter. The xylose transporter encoded by an exogenous nucleic acid can be at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to a Metschnikowia xylose transporter. The Metschnikowia xylose transporter can be from H0 Metschnikowia species. In some embodiments, the xylose transporter encoded by an exogenous nucleic acid can be have at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to a xylose transporter from H0 Metschnikowia species.

In some embodiments, the microbial organisms can have at least one exogenous nucleic acid encoding a xylose reductase that is at least 30% identical to a Metschnikowia xylose reductase. The xylose reductase encoded by an exogenous nucleic acid can be at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to a Metschnikowia xylose reductase. The Metschnikowia xylose reductase can be from H0 Metschnikowia species. In some embodiments, the xylose reductase encoded by an exogenous nucleic acid can be at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to a xylose reductase from H0 Metschnikowia species.

In some embodiments, the microbial organisms can have at least one exogenous nucleic acid encoding a xylose dehydrogenase that is at least 30% identical to a Metschnikowia xylose dehydrogenase. The xylose dehydrogenase encoded by an exogenous nucleic acid can be at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to a Metschnikowia xylose dehydrogenase. The Metschnikowia xylose dehydrogenase can be from H0 Metschnikowia species. In some embodiments, the xylose dehydrogenase encoded by an exogenous nucleic acid can be at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to a xylose dehydrogenase from H0 Metschnikowia species.

In some embodiments, the microbial organisms can have at least one exogenous nucleic acid encoding a xylulokinase that is at least 30% identical to a Metschnikowia xylulokinase. The xylose transporter encoded by an exogenous nucleic acid can be at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to a Metschnikowia xylulokinase. The Metschnikowia xylose kinase can be from H0 Metschnikowia species. In some embodiments, the xylose kinase encoded by an exogenous nucleic acid can be at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to a xylulokinase from H0 Metschnikowia species.

In some embodiments, the microbial organisms can have at least two exogenous nucleic acids each encoding two enzymes or proteins that is at least 30% identical to a Metschnikowia enzyme or protein. The two enzymes or proteins encoded by the at least two exogenous nucleic acids can each be at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the respective Metschnikowia enzymes or proteins. The Metschnikowia enzymes or proteins can be from H0 Metschnikowia species. In some embodiments, the two Metschnikowia enzymes or proteins encoded by the at least two exogenous nucleic acids can each be at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the respective enzymes or proteins from H0 Metschnikowia species. In some embodiments, the microbial organisms can have at least two exogenous nucleic acids encoding a xylose transporter and a xylose reductase. In some embodiments, the microbial organisms can have at least two exogenous nucleic acids encoding a xylose transporter and a xylose dehydrogenase. In some embodiments, the microbial organisms can have at least two exogenous nucleic acids encoding a xylose transporter and a xylulokinase. In some embodiments, the microbial organisms can have at least two exogenous nucleic acids encoding a xylose reductase and a xylose dehydrogenase. In some embodiments, the microbial organisms can have at least two exogenous nucleic acids encoding a xylose reductase and a xylulokinase. In some embodiments, the microbial organisms can have at least two exogenous nucleic acids encoding a xylose dehydrogenase and a xylulokinase.

In some embodiments, the microbial organisms can have at least three exogenous nucleic acids each encoding an enzyme or protein that is at least 30% identical to a Metschnikowia enzyme or protein. The three enzymes or proteins encoded by the at least three exogenous nucleic acids can each be at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the respective Metschnikowia enzymes or proteins. The Metschnikowia enzymes or proteins can be from H0 Metschnikowia species. In some embodiments, the three Metschnikowia enzymes or proteins encoded by the at least three exogenous nucleic acids can each be at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the respective enzymes or proteins from H0 Metschnikowia species. In some embodiments, the microbial organisms can have at least three exogenous nucleic acids encoding xylose transporter, a xylose reductase, and a xylose dehydrogenase. In some embodiments, the microbial organisms can have at least two exogenous nucleic acids encoding xylose transporter, a xylose reductase, and a xylulokinase. In some embodiments, the microbial organisms can have at least two exogenous nucleic acids encoding xylose transporter, a xylose dehydrogenase and a xylulokinase. In some embodiments, the microbial organisms can have at least two exogenous nucleic acids encoding a xylose reductase, a xylose dehydrogenase and a xylulokinase.

In some embodiments, the microbial organisms can have at least four exogenous nucleic acids each encoding an enzyme or protein that is at least 30% identical to a Metschnikowia enzyme or protein. The four enzymes or proteins encoded by the at least four exogenous nucleic acids can each be at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the respective Metschnikowia enzymes or proteins. The Metschnikowia enzymes or proteins can be from H0 Metschnikowia species. In some embodiments, the four Metschnikowia enzymes or proteins encoded by the at four three exogenous nucleic acids can each be at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the respective enzymes or proteins from H0 Metschnikowia species. In some embodiments, the microbial organisms can have at least four exogenous nucleic acids encoding xylose transporter, a xylose reductase, a xylose dehydrogenase and a xylulokinase.

In some embodiments, provided herein are non-naturally occurring microbial organisms having a xylose-ethanol pathway and at least one exogenous nucleic acid encoding an enzyme or protein expressed in sufficient amount to confer or enhance xylose uptake. Expression of these transporters or their variants in microbial organisms (e.g. S. cerevisiae) can enhance xylose uptake and increase the production of bioderived products from xylose by these microbial organisms. Thus, provided herein is an isolated polypeptide that is a Metschnikowia xylose transporter or a variant thereof; an isolated nucleic acid that encodes a Metschnikowia xylose transporter or a variant thereof; a vector that has an isolated nucleic acid that encodes a Metschnikowia xylose transporter or a variant thereof; as well as a non-naturally occurring microbial organism having a xylose-ethanol pathway and enhanced xylose uptake by expressing at least one exogenous nucleic acid encoding a Metschnikowia xylose transporter or a variant thereof.

Provided herein are non-naturally occurring microbial organisms having a xylose-ethanol pathway enhanced xylose uptake, which have at least one exogenous nucleic acid encoding a xylose transporter, wherein the xylose transporter has an amino acid sequence that is at least 30% identical to a Metschnikowia xylose transporter. Provided herein are also isolated polypeptides that are at least 30% identical to a Metschnikowia xylose transporter. Provided herein are also isolated nucleic acids that encode polypeptides that are at least 30% identical to a Metschnikowia xylose transporter. The Metschnikowia xylose transporters include, for example, transporters such as Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, or Aps1p/Hgt19p from a Metschnikowia species. The Metschnikowia species include, for example, the Metschnikowia sp. H0, Metschnikowia pulcherrima, Metschnikowia fructicola, Metschnikowia chrysoperlae, Metschnikowia reukaufii, Metschnikowia andauensis, Metschnikowia sinensis, Metschnikowia shanxiensis, Metschnikowia zizyphicola, Metschnikowia bicuspidata, Metschnikowia lunata, Metschnikowia zobellii, Metschnikowia australis, Metschnikowia agaveae, Metschnikowia gruessii, Metschnikowia hawaiiensis, Metschnikowia krissii, Metschnikowia sp. strain NS—O-85, and Metschnikowia sp. strain NS—O-89. The Metschnikowia xylose transporter can be a xylose transporter from the H0 Metschnikowia species. A number of xylose transporters were cloned from H0 Metschnikowia species, a species of Metschnikowia. In some embodiments, the xylose transporter can include, for example, Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, or Aps1p/Hgt19p from the H0 Metschnikowia species.

Expression of more than one xylose transporters can further improve xylose uptake. As such, the non-naturally occurring microbial organisms can have a xylose-ethanol pathway and at least one exogenous nucleic acid, or at least two, at least three, at least four, at least five, at least six, at least seven, at least eight, at least nine, at least ten, or at least eleven nucleic acids each encoding a xylose transporter. In some embodiments, the microbial organisms have at least two exogenous nucleic acids each encoding a xylose transporter. In some embodiments, the microbial organisms have at least three exogenous nucleic acids each encoding a xylose transporter. In some embodiments, the microbial organisms have at least four exogenous nucleic acids each encoding a xylose transporter. In some embodiments, the microbial organisms have at least five exogenous nucleic acids each encoding a xylose transporter. In some embodiments, the microbial organisms have at least six exogenous nucleic acids each encoding a xylose transporter. In some embodiments, the microbial organisms have at least seven exogenous nucleic acids each encoding a xylose transporter. In some embodiments, the microbial organisms have at least eight exogenous nucleic acids each encoding a xylose transporter. In some embodiments, the microbial organisms have at least nine exogenous nucleic acids each encoding a xylose transporter. In some embodiments, the microbial organisms have at least ten exogenous nucleic acids each encoding a xylose transporter. In some embodiments, the microbial organisms have at least eleven exogenous nucleic acids each encoding a xylose transporter.

The xylose transporters provided herein can be a Metschnikowia xylose transporter, including such as those from H0 Metschnikowia species having amino acid sequences as shown in sequence listing, as well as their variants that retain their transporter function. For example, provided herein is Xyt1p from H0 Metschnikowia species that has an amino acid sequence of SEQ ID NO:1, as well as variants thereof that retain the transporter function of Xyt1p. The transporter function of Xyt1p includes, but is not limited to, transport of xylose across cell wall and/or cell membrane, which can be determined, for example, by subjecting the variant to a transporter assay as described herin or otherwise known in the art. The xylose transporter function can be determined, for example, by expressing the transporter in a microbial organism and measuring the increase in xylose uptake by the microbial organism. In an exemplary assay, a non-xylose utilizing microbial organism expressing an exogenous transporter can be cultured in a xylose-contaning medium and and the decrease of xylose in the culture medium can be measured by high performance liquid chromatography (HPLC) using Rezex RPM-monosaccharide Pb+2 column (Phenomenex), refractive index detector and water as a mobile phase at 0.6 ml/min. In another exemplary assay, starter cultures for wild type and transgenic microbial organisms expressing various transporters can be grown in YP base medium with controlled amounts of glucose and xylose (%; w/v). Uninoculated medium is used a reference for a given sampling time; the medium indicates 100% of the starting xylose or xylose at time 0 h. At 24 h intervals, samples at volumes of 300-1000 μL can be removed from the culture aseptically and filtered through a 0.2 μm syringe filter, physically separating medium and yeast. The medium can be transferred to glass vials and the xylose content can be examined by HPLC. The amount of xylose remaining in the sampled medium can be determined by comparison with a pre-defined calibration curve, and the remaining sample is normalized to the xylose content in the uninoculated medium, which is counted as containing 100% of the xylose at the initiation of the culture. The non-naturally ocurring microbial organisms expressing an exogenous xylose transporter can consume xylose at a higher rate than their wild type counterparts, and the differences in the decrease rate of xylose in the culture medium between wild type and non-naturally ocurring microbial organisms expressing an exogenous xylose transporter can indicate the transporter function of the exogenous xylose transporter.

In some embodiments, provided herein are also isolated polypeptides that are variants of a Metschnikowia xylose transporter that retains its transporter function. Provided herein are also isolated nucleic acids that encode polypeptides that are variants to a Metschnikowia xylose transporter that retains its transporter function. In some embodiments, provided herein are non-naturally occurring microbial organisms having a xylose-ethanol pathway and an exogenous nucleic acid encoding a xylose transporter, wherein the xylose transporter is a variant of a Metschnikowia xylose transporter that retains its transporter function. In some embodiments, the xylose transporter is a variant of the xylose transporters from the H0 Metschnikowia species as described herein that retains its transporter function.

In some embodiments, provided herein are also isolated polypeptides that are variants of Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, and Aps1p/Hgt19p from a Metschnikowia species that retain the transporter function. Provided herein are also isolated polypeptides that are variants of Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, or Aps1p/Hgt19p from the H0 Metschnikowia species that retain the transporter function. In some embodiments, provided herein are also isolated nucleic acids that encode polypeptides that are variants of Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, or Aps1p/Hgt19p from a Metschnikowia species that retain the transporter function. In some embodiments, provided herein are also isolated nucleic acids that encode polypeptides that are variants of Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, or Aps1p/Hgt19p from the H0 Metschnikowia species that retain the transporter function.

In some embodiments, provided herein are non-naturally occurring microbial organisms having a xylose-ethanol pathway and an exogenous nucleic acid encoding a xylose transporter, wherein the xylose transporter is a variant of Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, or Aps1p/Hgt19p from a Metschnikowia species that retains the transporter function. In some embodiments, provided herein are non-naturally occurring microbial organisms having an exogenous nucleic acid encoding a xylose transporter, wherein the xylose transporter is a variant of Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, or Aps1p/Hgt19p from H0 Metschnikowia species that retains the transporter function.

The xylose transporters described herein can have amino acid sequence of at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is identical, to the amino acid sequences disclosed herein by SEQ ID NO, GenBank and/or GI number. In some embodiments, the xylose transporters described herein can have amino acid sequence of 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity, or be identical, to amino acids described herein by SEQ ID NO, GenBank and/or GI number. In some embodiments, the xylose transporters described herein can have amino acid sequence of 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity, or be identical, to any one of SEQ ID NOs: 1-5 and 7-12.

In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia xylose transporter such as Metschnikowia Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, or Aps1p/Hgt19p. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia xylose transporter such as Metschnikowia Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, or Aps1p/Hgt19p.

In some embodiments, provided herein are non-naturally occurring microbial organisms having a xylose-ethanol pathway and at least one exogenous nucleic acid encoding a xylose transporter, wherein the xylose transporter has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia xylose transporter such as Metschnikowia Xyt1p, Gxf1p, Agxf1p, GXF2/GAL2p, Gxs1p/Hgt12, Hxt5, Hxt2.6, Qup2, or Aps1/Hgt19. In some embodiments, the exogenous nucleic acid encodes a Metschnikowia xylose transporter such as Metschnikowia Xyt1p, Gxf1p, Agxf1p, GXF2/GAL2p, Gxs1p/Hgt12, Hxt5, Hxt2.6, Qup2, or Aps1/Hgt19.

Sequence identity (also known as homology or similarity) refers to sequence similarity between two nucleic acid molecules or between two polypeptides. Identity can be determined by comparing a position in each sequence, which may be aligned for purposes of comparison. When a position in the compared sequence is occupied by the same base or amino acid, then the molecules are identical at that position. A degree of identity between sequences is a function of the number of matching or homologous positions shared by the sequences. The alignment of two sequences to determine their percent sequence identity can be done using software programs known in the art, such as, for example, those described in Ausubel et al., Current Protocols in Molecular Biology, John Wiley and Sons, Baltimore, Md. (1999). Preferably, default parameters are used for the alignment. One alignment program well known in the art that can be used is BLAST set to default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the National Center for Biotechnology Information.

Variants of a specific xylose transporter can also include, for example, amino acid substitutions, deletions, fusions, or truncations when compared to the reference xylose transporter. Variants of the Metschnikowia xylose transporters described herein can also contain conservatively amino acids substitution, meaning that one or more amino acid can be replaced by an amino acid that does not alter the secondary and/or tertiary stricture of the xylose transporter. Such substitutions can include the replacement of an amino acid, by a residue having similar physicochemical properties, such as substituting one aliphatic residue (Ile, Val, Leu, or Ala) for another, or substitutions between basic residues Lys and Arg, acidic residues Glu and Asp, amide residues Gln and Asn, hydroxyl residues Ser and Tyr, or aromatic residues Phe and Tyr. Phenotypically silent amino acid exchanges are described more fully in Bowie et al., Science 247:1306-10 (1990). In addition, variants of Metschnikowia xylose transporters include those having amino acid substitutions, deletions, or additions to the amino acid sequence outside functional regions of the protein so long as the substitution, deletion, or addition does not affect xylose transport function of the resulting polypeptide. Techniques for making these substitutions and deletions are well known in the art and include, for example, site-directed mutagenesis.

In some embodiments, provided herein are non-naturally occurring microbial organisms having a xylose-ethanol pathway and an exogenous nucleic acid encoding a xylose transporter, wherein the xylose transporter has 1 to 50, 1 to 45, 1 to 40, 1 to 35, 1 to 30, 1 to 25, 1 to 20, 1 to 15, 1 to 10, or 1 to 5, amino acid substitutions, deletions or insertions of Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, or Aps1p/Hgt19p from a Metschnikowia species and retains the transporter function. The Metschnikowia species can be the H0 Metschnikowia species. In some embodiments, the xylose transporter has 1 to 10 amino acid substitutions, deletions or insertions of Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, or Aps1p/Hgt19p from a Metschnikowia species and retains the transporter function. The Metschnikowia species can be the H0 Metschnikowia species. In some embodiments, the xylose transporter has 1 to 5 amino acid substitutions, deletions or insertions of Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, or Aps1p/Hgt19p from a Metschnikowia species and retains the transporter function. The Metschnikowia species can be the H0 Metschnikowia species.

The xylose transporters provided herein also include functional fragments of specific Metschnikowia xylose transporters that retain their transporter function. In some embodiments, provided herein is an isolated polypeptide that is a functional fragment of a specific Metschnikowia xylose transporter. In some embodiments, provided herein is an isolated nucleic acid that encodes a polypeptide that is functional fragment of a specific Metschnikowia xylose transporter. In some embodiments, the xylose transporter can be fragments of a xylose transporter such as Metschnikowia Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, or Aps1p/Hgt19p retains the transporter function. In some embodiments, the xylose transporter can be fragments of a xylose transporter such as Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, or Aps1p/Hgt19p from the H0 Metschnikowia species retains the transporter function.

In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a function fragment of a Metschnikowia xylose transporter including such as Metschnikowia Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, or Aps1p/Hgt19p. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to a function fragment of a Metschnikowia xylose transporter including such as Metschnikowia Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, or Aps1p/Hgt19p.

In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to a function fragment of a xylose transporter of H0 Metschnikowia species including such as Metschnikowia Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, or Aps1p/Hgt19p. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to a function fragment of a xylose transporter of H0 Metschnikowia species including such as Metschnikowia Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, or Aps1p/Hgt19p.

In some embodiments, provided herein are non-naturally occurring microbial organisms having a xylose-ethanol pathway and an exogenous nucleic acid encoding a functional fragment of a Metschnikowia xylose transporter that retains its transporter function. In some embodiments, the non-naturally occurring microbial organisms provided herein have at least one exogenous nucleic acid encoding a xylose transporter that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to a function fragment of a Metschnikowia xylose transporter including such as Metschnikowia Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, or Aps1p/Hgt19p. In some embodiments, the non-naturally occurring microbial organisms provided herein can have a xylose-ethanol pathway and at least one exogenous nucleic acid encoding a xylose transporter that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to a function fragment of a xylose transporter of H0 Metschnikowia species such as Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, or Aps1p/Hgt19p.

In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to a Metschnikowia Xyt1p. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Xyt1p. In some embodiments, provided herein are non-naturally occurring microbial organisms having a xylose-ethanol pathway and at least one exogenous nucleic acid encoding a xylose transporter, wherein the xylose transporter has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Xyt1p. In some embodiments, the xylose transporter can be a Metschnikowia Xyt1p. In some embodiments, the xylose transporter can be a variant of a Metschnikowia Xyt1p that retains its transporter function. The xylose transporter can be a functional fragment of a Metschnikowia Xyt1p. In some embodiments, the xylose transporter can have 1 to 50, 1 to 45, 1 to 40, 1 to 35, 1 to 30, 1 to 25, 1 to 20, 1 to 15, 1 to 10, or 1 to 5, amino acid substitutions, deletions or insertions of Xyt1p from a Metschnikowia species. In some embodiments, the xylose transporter has 1 to 10 amino acid substitutions, deletions or insertions of Xyt1p from a Metschnikowia species. In some embodiments, the xylose transporter has 1 to 5 amino acid substitutions, deletions or insertions of Xyt1p from a Metschnikowia species.

The Metschnikowia species can be the H0 Metschnikowia species. In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to Xyt1p of H0 Metschnikowia species. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Xyt1p of H0 Metschnikowia species. In some embodiments, the non-naturally occurring microbial organisms have a xylose-ethanol pathway and at least one exogenous nucleic acid encoding a xylose transporter that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to Xyt1p of H0 Metschnikowia species. In some embodiments, the xylose transporter is Xyt1p of H0 Metschnikowia species. In some embodiments, the xylose transporter can be a variant of Xyt1p of H0 Metschnikowia species that retains its transporter function. In some embodiments, the xylose transporter is a functional fragment of Xyt1p of H0 Metschnikowia species. In some embodiments, the xylose transporter can have 1 to 50, 1 to 45, 1 to 40, 1 to 35, 1 to 30, 1 to 25, 1 to 20, 1 to 15, 1 to 10, or 1 to 5, amino acid substitutions, deletions or insertions of Xyt1p from H0 Metschnikowia species. In some embodiments, the xylose transporter has 1 to 10 amino acid substitutions, deletions or insertions of Xyt1p from H0 Metschnikowia species. In some embodiments, the xylose transporter has 1 to 5 amino acid substitutions, deletions or insertions of Xyt1p from H0 Metschnikowia species. In some embodiments, the xylose transporter has the amino acid sequence of SEQ ID NO: 1. In some embodiments, the amino acid sequence of the xylose transporter is SEQ ID NO: 1. In some embodiments, the nucleic acid has the sequence of SEQ ID NO: 13. In some embodiments, the sequence of the nucleic acid is SEQ ID NO: 13. The nucleic acid encoding Xyt1p from a Metschnikowia species can be codon optimized for heterologous expression. In some embodiments, the nucleic acid encoding Metschnikowia Xyt1p is codon optimized for expression in a yeast host strain. The yeast host strain can be any yeast host strain described herein, such as S. cerevisiae. In some embodiments, the nucleic acid encoding Metschnikowia Xyt1p is codon optimized for expression in a bacterial host strain. The bacterial host strain can be any bacterial host strain described herein, such as E. coli. In some embodiments, the nucleic acid encoding Xyt1p from H0 Metschnikowia species is codon optimized for expression in S. cerevisiae. For example, in some embodiments, the nucleic acid encoding Xyt1p of H0 Metschnikowia species is codon optimized for expression in S. cerevisiae. The nucleic acid can have the sequence of SEQ ID NO: 21.

In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Gxf1p. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Gxf1p. In some embodiments, provided herein are non-naturally occurring microbial organisms having a xylose-ethanol pathway and at least one exogenous nucleic acid encoding a xylose transporter, wherein the xylose transporter has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Gxf1p. In some embodiments, the xylose transporter is a Metschnikowia Gxf1p. In some embodiments, the xylose transporter is a variant of a Metschnikowia Gxf1pthat retains its transporter function. The xylose transporter can be a functional fragment of a Metschnikowia Gxf1p. In some embodiments, the nucleic acid encodes a xylose transporter having 1 to 50, 1 to 45, 1 to 40, 1 to 35, 1 to 30, 1 to 25, 1 to 20, 1 to 15, 1 to 10, or 1 to 5, amino acid substitutions, deletions or insertions of Gxf1p from a Metschnikowia species. In some embodiments, the xylose transporter has 1 to 10 amino acid substitutions, deletions or insertions of Gxf1p from a Metschnikowia species. In some embodiments, the xylose transporter has 1 to 5 amino acid substitutions, deletions or insertions of Gxf1p from a Metschnikowia species.

The Metschnikowia species can be the H0 Metschnikowia species. In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to Gxf1p of H0 Metschnikowia species. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Gxf1pof H0 Metschnikowia species. In some embodiments, the non-naturally occurring microbial organisms have a xylose-ethanol pathway and at least one exogenous nucleic acid encoding a xylose transporter that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to Gxf1p of H0 Metschnikowia species. In some embodiments, the xylose transporter is Gxf1p of H0 Metschnikowia species. In some embodiments, the xylose transporter can be a variant of Gxf1p of H0 Metschnikowia species that retains its transporter function. In some embodiments, the xylose transporter is a functional fragment of Gxf1p of H0 Metschnikowia species. In some embodiments, the xylose transporter can have 1 to 50, 1 to 45, 1 to 40, 1 to 35, 1 to 30, 1 to 25, 1 to 20, 1 to 15, 1 to 10, or 1 to 5, amino acid substitutions, deletions or insertions of Gxf1p from H0 Metschnikowia species. In some embodiments, the xylose transporter has 1 to 10 amino acid substitutions, deletions or insertions of Gxf1p from H0 Metschnikowia species. In some embodiments, the xylose transporter has 1 to 5 amino acid substitutions, deletions or insertions of Gxf1p from H0 Metschnikowia species. In some embodiments, the xylose transporter has the amino acid sequence of SEQ ID NO: 2. In some embodiments, the amino acid sequence of the xylose transporter is SEQ ID NO: 2. In some embodiments, the nucleic acid has the sequence of SEQ ID NO: 14. In some embodiments, the sequence of the nucleic acid is SEQ ID NO: 14. In some embodiments, the nucleic acid encodes a functional fragment of Gxf1p of H0 Metschnikowia species. For example, the fragment of Gxf1p can be a variant of Gxf1p that has a shorter N-terminus, and referred to as ΔGxf1p. In some embodiments, the amino acid sequence of the xylose transporter is SEQ ID NO: 3. In some embodiments, the nucleic acid has the sequence of SEQ ID NO: 15. The nucleic acid encoding Gxf1p from a Metschnikowia species can be codon optimized for heterologous expression. In some embodiments, the nucleic acid encoding Metschnikowia Gxf1p is codon optimized for expression in a yeast host strain. The yeast host strain can be any yeast host strain described herein, such as S. cerevisiae. In some embodiments, the nucleic acid encoding Metschnikowia Gxf1p is codon optimized for expression in a bacterial host strain. The bacterial host strain can be any bacterial host strain described herein, such as E. coli. In some embodiments, the nucleic acid encoding Gxf1p from H0 Metschnikowia species is codon optimized for expression in S. cerevisiae.

In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Gxf2p/Ga12p. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Gxf2p/Ga12p. In some embodiments, provided herein are non-naturally occurring microbial organisms having a xylose-ethanol pathway and at least one exogenous nucleic acid encoding a xylose transporter, wherein the xylose transporter has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Gxf2p/Ga12p. In some embodiments, the xylose transporter is a Metschnikowia Gxf2p/Ga12p. In some embodiments, the xylose transporter is a variant of a Metschnikowia Gxf2p/Ga12p that retains its transporter function. The xylose transporter can be a functional fragment of a Metschnikowia Gxf2p/Ga12p. In some embodiments, the xylose transporter can have 1 to 50, 1 to 45, 1 to 40, 1 to 35, 1 to 30, 1 to 25, 1 to 20, 1 to 15, 1 to 10, or 1 to 5, amino acid substitutions, deletions or insertions of Gxf2p/Ga12p from a Metschnikowia species. In some embodiments, the xylose transporter has 1 to 10 amino acid substitutions, deletions or insertions of Gxf2p/Ga12p from a Metschnikowia species. In some embodiments, the xylose transporter has 1 to 5 amino acid substitutions, deletions or insertions of Gxf2p/Ga12p from a Metschnikowia species.

The Metschnikowia species can be the H0 Metschnikowia species. In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to Gxf2p/Ga12p of H0 Metschnikowia species. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Gxf2p/Ga12p of H0 Metschnikowia species. In some embodiments, the non-naturally occurring microbial organisms have a xylose-ethanol pathway and at least one exogenous nucleic acid encoding a xylose transporter that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to Gxf2p/Ga12p of H0 Metschnikowia species. In some embodiments, the xylose transporter is Gxf2p/Ga12p of H0 Metschnikowia species. In some embodiments, the xylose transporter can be a variant of Gxf2p/Ga12p of H0 Metschnikowia species that retains its transporter function. In some embodiments, the xylose transporter is a functional fragment of Gxf2p/Ga12p of H0 Metschnikowia species. In some embodiments, the xylose transporter can have 1 to 50, 1 to 45, 1 to 40, 1 to 35, 1 to 30, 1 to 25, 1 to 20, 1 to 15, 1 to 10, or 1 to 5, amino acid substitutions, deletions or insertions of Gxf2p/Ga12p from H0 Metschnikowia species. In some embodiments, the xylose transporter has 1 to 10 amino acid substitutions, deletions or insertions of Gxf2p/Ga12p from H0 Metschnikowia species. In some embodiments, the xylose transporter has 1 to 5 amino acid substitutions, deletions or insertions of Gxf2p/Ga12p from H0 Metschnikowia species. In some embodiments, the xylose transporter has the amino acid sequence of SEQ ID NO: 4. In some embodiments, the amino acid sequence of the xylose transporter is SEQ ID NO: 4. In some embodiments, the nucleic acid has the sequence of SEQ ID NO: 16. In some embodiments, the sequence of the nucleic acid is SEQ ID NO: 16. The nucleic acid encoding Gxf2p/Ga12p from a Metschnikowia species can be codon optimized for heterologous expression. In some embodiments, the nucleic acid encoding Metschnikowia Gxf2p/Ga12p is codon optimized for expression in a yeast host strain. The yeast host strain can be any yeast host strain described herein, such as S. cerevisiae. In some embodiments, the nucleic acid encoding Metschnikowia Gxf2p/Ga12p is codon optimized for expression in a bacterial host strain. The bacterial host strain can be any bacterial host strain described herein, such as E. coli. In some embodiments, the nucleic acid encoding Gxf2p/Ga12p from H0 Metschnikowia species is codon optimized for expression in S. cerevisiae.

In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Gxs1p/Hgt12p. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Gxs1p/Hgt12p. In some embodiments, provided herein are non-naturally occurring microbial organisms having a xylose-ethanol pathway and at least one exogenous nucleic acid encoding a xylose transporter, wherein the xylose transporter has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Gxs1p/Hgt12p. In some embodiments, the xylose transporter is a Metschnikowia Gxs1p/Hgt12p. In some embodiments, the xylose transporter is a variant of a Metschnikowia Gxs1p/Hgt12p that retains its transporter function. The xylose transporter can be a functional fragment of a Metschnikowia Gxs1p/Hgt12p. In some embodiments, the xylose transporter can have 1 to 50, 1 to 45, 1 to 40, 1 to 35, 1 to 30, 1 to 25, 1 to 20, 1 to 15, 1 to 10, or 1 to 5, amino acid substitutions, deletions or insertions of Gxs1p/Hgt12p from a Metschnikowia species. In some embodiments, the xylose transporter has 1 to 10 amino acid substitutions, deletions or insertions of Gxs1p/Hgt12p from a Metschnikowia species. In some embodiments, the xylose transporter has 1 to 5 amino acid substitutions, deletions or insertions of Gxs1p/Hgt12p from a Metschnikowia species.

The Metschnikowia species can be the H0 Metschnikowia species. In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to Gxs 1p/Hgt12pof H0 Metschnikowia species. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Gxs1p/Hgt12pof H0 Metschnikowia species. In some embodiments, the non-naturally occurring microbial organisms have a xylose-ethanol pathway and at least one exogenous nucleic acid encoding a xylose transporter that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to Gxs 1p/Hgt12p of H0 Metschnikowia species. In some embodiments, the xylose transporter is Gxs1p/Hgt12p of H0 Metschnikowia species. In some embodiments, the xylose transporter can be a variant of Gxs1p/Hgt12p of H0 Metschnikowia species that retains its transporter function. In some embodiments, the xylose transporter can have 1 to 50, 1 to 45, 1 to 40, 1 to 35, 1 to 30, 1 to 25, 1 to 20, 1 to 15, 1 to 10, or 1 to 5, amino acid substitutions, deletions or insertions of Gxs1p/Hgt12p from H0 Metschnikowia species. In some embodiments, the xylose transporter has 1 to 10 amino acid substitutions, deletions or insertions of Gxs1p/Hgt12p from H0 Metschnikowia species. In some embodiments, the xylose transporter has 1 to 5 amino acid substitutions, deletions or insertions of Gxs1p/Hgt12p from H0 Metschnikowia species. In some embodiments, the xylose transporter has the amino acid sequence of SEQ ID NO: 7. In some embodiments, the amino acid sequence of the xylose transporter is SEQ ID NO: 7. In some embodiments, the nucleic acid has the sequence of SEQ ID NO: 19. In some embodiments, the sequence of the nucleic acid is SEQ ID NO: 19. In some embodiments, the xylose transporter is a functional fragment of Gxs1p/Hgt12p of H0 Metschnikowia species. For example, the fragment of Gxs1p/Hgt12p can be a variant of Gxs1p/Hgt12p that has a shorter N-terminus, and referred to as ΔGxs1p/ΔHgt12p. In some embodiments, the amino acid sequence of the xylose transporter is SEQ ID NO: 5. In some embodiments, the nucleic acid has the sequence of SEQ ID NO: 17. The nucleic acid encoding Gxs1p/Hgt12p from a Metschnikowia species can be codon optimized for heterologous expression. In some embodiments, the nucleic acid encoding Metschnikowia Gxs1p/Hgt12p is codon optimized for expression in a yeast host strain. The yeast host strain can be any yeast host strain described herein, such as S. cerevisiae. In some embodiments, the nucleic acid encoding Metschnikowia Gxs1p/Hgt12p is codon optimized for expression in a bacterial host strain. The bacterial host strain can be any bacterial host strain described herein, such as E. coli. In some embodiments, the nucleic acid encoding Gxs1p/Hgt12p from H0 Metschnikowia species is codon optimized for expression in S. cerevisiae.

In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Hxt5p. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Hxt5p. In some embodiments, provided herein are non-naturally occurring microbial organisms having a xylose-ethanol pathway and at least one exogenous nucleic acid encoding a xylose transporter, wherein the xylose transporter has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Hxt5p. In some embodiments, the xylose transporter is a Metschnikowia Hxt5p. In some embodiments, the xylose transporter is a variant of a Metschnikowia Hxt5p that retains its transporter function. The xylose transporter can be a functional fragment of a Metschnikowia Hxt5p. In some embodiments, the xylose transporter can have 1 to 50, 1 to 45, 1 to 40, 1 to 35, 1 to 30, 1 to 25, 1 to 20, 1 to 15, 1 to 10, or 1 to 5, amino acid substitutions, deletions or insertions of Hxt5p from a Metschnikowia species. In some embodiments, the xylose transporter has 1 to 10 amino acid substitutions, deletions or insertions of Hxt5p from a Metschnikowia species. In some embodiments, the xylose transporter has 1 to 5 amino acid substitutions, deletions or insertions of Hxt5p from a Metschnikowia species.

The Metschnikowia species can be the H0 Metschnikowia species. In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to Hxt5p of H0 Metschnikowia species. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Hxt5p of H0 Metschnikowia species. In some embodiments, the non-naturally occurring microbial organisms have a xylose-ethanol pathway and at least one exogenous nucleic acid encoding a xylose transporter that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to Hxt5p of H0 Metschnikowia species. In some embodiments, the xylose transporter is Hxt5p of H0 Metschnikowia species. In some embodiments, the xylose transporter can be a variant of Hxt5p of H0 Metschnikowia species that retains its transporter function. In some embodiments, the xylose transporter is a functional fragment of Hxt5p of H0 Metschnikowia species. In some embodiments, the xylose transporter can have 1 to 50, 1 to 45, 1 to 40, 1 to 35, 1 to 30, 1 to 25, 1 to 20, 1 to 15, 1 to 10, or 1 to 5, amino acid substitutions, deletions or insertions of Hxt5p from H0 Metschnikowia species. In some embodiments, the xylose transporter has 1 to 10 amino acid substitutions, deletions or insertions of Hxt5p from H0 Metschnikowia species. In some embodiments, the xylose transporter has 1 to 5 amino acid substitutions, deletions or insertions of Hxt5p from H0 Metschnikowia species. In some embodiments, the xylose transporter has the amino acid sequence of SEQ ID NO: 8. In some embodiments, the amino acid sequence of the xylose transporter is SEQ ID NO: 8. In some embodiments, the nucleic acid has the sequence of SEQ ID NO: 20. In some embodiments, the sequence of the nucleic acid is SEQ ID NO: 20. The nucleic acid encoding Hxt5p from a Metschnikowia species can be codon optimized for heterologous expression. In some embodiments, the nucleic acid encoding Metschnikowia Hxt5p is codon optimized for expression in a yeast host strain. The yeast host strain can be any yeast host strain described herein, such as S. cerevisiae. In some embodiments, the nucleic acid encoding Metschnikowia Hxt5p is codon optimized for expression in a bacterial host strain. The bacterial host strain can be any bacterial host strain described herein, such as E. coli. In some embodiments, the nucleic acid encoding Hxt5p from H0 Metschnikowia species is codon optimized for expression in S. cerevisiae.

In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Hxt2.6p. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Hxt2.6p. In some embodiments, provided herein are non-naturally occurring microbial organisms having a xylose-ethanol pathway and at least one exogenous nucleic acid encoding a xylose transporter, wherein the xylose transporter has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Hxt2.6p. In some embodiments, the xylose transporter is a Metschnikowia Hxt2.6p. In some embodiments, the xylose transporter is a variant of a Metschnikowia Hxt2.6p that retains its transporter function. The xylose transporter can be a functional fragment of a Metschnikowia Hxt2.6p. In some embodiments, the xylose transporter can have 1 to 50, 1 to 45, 1 to 40, 1 to 35, 1 to 30, 1 to 25, 1 to 20, 1 to 15, 1 to 10, or 1 to 5, amino acid substitutions, deletions or insertions of Hxt2.6p from a Metschnikowia species. In some embodiments, the xylose transporter has 1 to 10 amino acid substitutions, deletions or insertions of Hxt2.6p from a Metschnikowia species. In some embodiments, the xylose transporter has 1 to 5 amino acid substitutions, deletions or insertions of Hxt2.6p from a Metschnikowia species.

The Metschnikowia species can be the H0 Metschnikowia species. In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to Hxt2.6p of H0 Metschnikowia species. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Hxt2.6p of H0 Metschnikowia species. In some embodiments, the non-naturally occurring microbial organisms have a xylose-ethanol pathway and at least one exogenous nucleic acid encoding a xylose transporter that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to Hxt2.6p of H0 Metschnikowia species. In some embodiments, the xylose transporter is Hxt2.6p of H0 Metschnikowia species. In some embodiments, the xylose transporter can be a variant of Hxt2.6p of H0 Metschnikowia species that retains its transporter function. In some embodiments, the xylose transporter is a functional fragment of Hxt2.6p of H0 Metschnikowia species. In some embodiments, the xylose transporter can have 1 to 50, 1 to 45, 1 to 40, 1 to 35, 1 to 30, 1 to 25, 1 to 20, 1 to 15, 1 to 10, or 1 to 5, amino acid substitutions, deletions or insertions of Hxt2.6p from H0 Metschnikowia species. In some embodiments, the xylose transporter has 1 to 10 amino acid substitutions, deletions or insertions of Hxt2.6p from H0 Metschnikowia species. In some embodiments, the xylose transporter has 1 to 5 amino acid substitutions, deletions or insertions of Hxt2.6p from H0 Metschnikowia species. In some embodiments, the xylose transporter has the amino acid sequence of SEQ ID NO: 10. In some embodiments, the amino acid sequence of the xylose transporter is SEQ ID NO: 10. In some embodiments, the nucleic acid has the sequence of SEQ ID NO: 22. In some embodiments, the sequence of the nucleic acid is SEQ ID NO: 22. The Hxt2.6p from a Metschnikowia species can be codon optimized for heterologous expression. In some embodiments, the nucleic acid encoding Metschnikowia Hxt2.6p is codon optimized for expression in a yeast host strain. The nucleic acid encoding yeast host strain can be any yeast host strain described herein, such as S. cerevisiae. In some embodiments, the nucleic acid encoding Metschnikowia Hxt2.6p is codon optimized for expression in a bacterial host strain. The bacterial host strain can be any bacterial host strain described herein, such as E. coli. In some embodiments, the nucleic acid encoding Hxt2.6p from H0 Metschnikowia species is codon optimized for expression in S. cerevisiae. For example, in some embodiments, the nucleic acid encodes Hxt2.6p of H0 Metschnikowia species that is codon optimized for expression in S. cerevisiae. The nucleic acid can have the sequence of SEQ ID NO: 23.

In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Qup2p. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Qup2p. In some embodiments, provided herein are non-naturally occurring microbial organisms having a xylose-ethanol pathway and at least one exogenous nucleic acid encoding a xylose transporter, wherein the xylose transporter has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Qup2p. In some embodiments, the xylose transporter is a Metschnikowia Qup2p. In some embodiments, the xylose transporter is a variant of a Metschnikowia Qup2p that retains its transporter function. The xylose transporter can be a functional fragment of a Metschnikowia Qup2p. In some embodiments, the xylose transporter can have 1 to 50, 1 to 45, 1 to 40, 1 to 35, 1 to 30, 1 to 25, 1 to 20, 1 to 15, 1 to 10, or 1 to 5, amino acid substitutions, deletions or insertions of Qup2p from a Metschnikowia species. In some embodiments, the xylose transporter has 1 to 10 amino acid substitutions, deletions or insertions of Qup2p from a Metschnikowia species. In some embodiments, the xylose transporter has 1 to 5 amino acid substitutions, deletions or insertions of Qup2p from a Metschnikowia species.

The Metschnikowia species can be the H0 Metschnikowia species. In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to Qup2p of H0 Metschnikowia species. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Qup2p of H0 Metschnikowia species. In some embodiments, the non-naturally occurring microbial organisms have a xylose-ethanol pathway and at least one exogenous nucleic acid encoding a xylose transporter that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to Qup2p of H0 Metschnikowia species. In some embodiments, the xylose transporter can be Qup2p of H0 Metschnikowia species. In some embodiments, the xylose transporter can be a variant of Qup2p of H0 Metschnikowia species that retains its transporter function. In some embodiments, the xylose transporter can be a functional fragment of Qup2p of H0 Metschnikowia species. In some embodiments, the xylose transporter can have 1 to 50, 1 to 45, 1 to 40, 1 to 35, 1 to 30, 1 to 25, 1 to 20, 1 to 15, 1 to 10, or 1 to 5, amino acid substitutions, deletions or insertions of Qup2p from H0 Metschnikowia species. In some embodiments, the xylose transporter has 1 to 10 amino acid substitutions, deletions or insertions of Qup2p from H0 Metschnikowia species. In some embodiments, the xylose transporter has 1 to 5 amino acid substitutions, deletions or insertions of Qup2p from H0 Metschnikowia species. In some embodiments, the xylose transporter has the amino acid sequence of SEQ ID NO: 11. In some embodiments, the amino acid sequence of the xylose transporter is SEQ ID NO: 11. In some embodiments, the nucleic acid has the sequence of SEQ ID NO: 24. In some embodiments, the sequence of the nucleic acid is SEQ ID NO: 24. The Qup2p from a Metschnikowia species can be codon optimized for heterologous expression. In some embodiments, the nucleic acid encoding Metschnikowia Qup2p is codon optimized for expression in a yeast host strain. The yeast host strain can be any yeast host strain described herein, such as S. cerevisiae. In some embodiments, the nucleic acid encoding Metschnikowia Qup2p is codon optimized for expression in a bacterial host strain. The bacterial host strain can be any bacterial host strain described herein, such as E. coli. In some embodiments, the nucleic acid encoding Qup2p from H0 Metschnikowia species is codon optimized for expression in S. cerevisiae. For example, in some embodiments, the nucleic acid encoding Qup2p of H0 Metschnikowia species that is codon optimized for expression in S. cerevisiae. The nucleic acid can have the sequence of SEQ ID NO: 25.

In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Aps1p/Hgt19p. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Aps1p/Hgt19p. In some embodiments, provided herein are non-naturally occurring microbial organisms having a xylose-ethanol pathway and at least one exogenous nucleic acid encoding a xylose transporter, wherein the xylose transporter has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Aps1p/Hgt19p. In some embodiments, the xylose transporter is a Metschnikowia Aps1p/Hgt19p. In some embodiments, the xylose transporter is a variant of a Metschnikowia Aps1p/Hgt19p that retains its transporter function. The xylose transporter can be a functional fragment of a Metschnikowia Aps1p/Hgt19p. In some embodiments, the xylose transporter can have 1 to 50, 1 to 45, 1 to 40, 1 to 35, 1 to 30, 1 to 25, 1 to 20, 1 to 15, 1 to 10, or 1 to 5, amino acid substitutions, deletions or insertions of Aps1p/Hgt19p from a Metschnikowia species. In some embodiments, the xylose transporter has 1 to 10 amino acid substitutions, deletions or insertions of Aps1p/Hgt19p from a Metschnikowia species. In some embodiments, the xylose transporter has 1 to 5 amino acid substitutions, deletions or insertions of Aps1p/Hgt19p from a Metschnikowia species.

The Metschnikowia species can be the Metschnikowia H0 Metschnikowia species. In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to Aps 1p/Hgt19p of H0 Metschnikowia species. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Aps1p/Hgt19p of H0 Metschnikowia species. In some embodiments, the non-naturally occurring microbial organisms have a xylose-ethanol pathway and at least one exogenous nucleic acid encoding a xylose transporter that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to Aps1p/Hgt19p of H0 Metschnikowia species. In some embodiments, the xylose transporter is Aps1p/Hgt19p of H0 Metschnikowia species. In some embodiments, the xylose transporter can be a variant of Aps1p/Hgt19p of H0 Metschnikowia species that retains its transporter function. In some embodiments, the xylose transporter is a functional fragment of Aps1p/Hgt19p of H0 Metschnikowia species. In some embodiments, the xylose transporter can have 1 to 50, 1 to 45, 1 to 40, 1 to 35, 1 to 30, 1 to 25, 1 to 20, 1 to 15, 1 to 10, or 1 to 5, amino acid substitutions, deletions or insertions of Aps1p/Hgt19p from H0 Metschnikowia species. In some embodiments, the xylose transporter has 1 to 10 amino acid substitutions, deletions or insertions of Aps1p/Hgt19p from H0 Metschnikowia species. In some embodiments, the xylose transporter has 1 to 5 amino acid substitutions, deletions or insertions of Aps1p/Hgt19p from H0 Metschnikowia species. In some embodiments, the xylose transporter has the amino acid sequence of SEQ ID NO: 12. In some embodiments, the amino acid sequence of the xylose transporter is SEQ ID NO: 12. In some embodiments, the nucleic acid has the sequence of SEQ ID NO: 26. In some embodiments, the sequence of the nucleic acid is SEQ ID NO: 26. The Aps1p/Hgt19p from a Metschnikowia species can be codon optimized for heterologous expression. In some embodiments, the nucleic acid encoding Metschnikowia Aps1p/Hgt19p is codon optimized for expression in a yeast host strain. The yeast host strain can be any yeast host strain described herein, such as S. cerevisiae. In some embodiments, the nucleic acid encoding Metschnikowia Aps1p/Hgt19p is codon optimized for expression in a bacterial host strain. The bacterial host strain can be any bacterial host strain described herein, such as E. coli. In some embodiments, the nucleic acid encoding Aps1p/Hgt19p from H0 Metschnikowia species is codon optimized for expression in S. cerevisiae. For example, in some embodiments, the nucleic acid encodes Aps1p/Hgt19p of H0 Metschnikowia species that is codon optimized for expression in S. cerevisiae. The nucleic acid can have the sequence of SEQ ID NO: 27.

As provided above, the non-naturally occurring microbial organisms can have a xylose-ethanol pathway and at least one exogenous nucleic acid, or at least two, at least three, at least four, at least five, at least six, at least seven, at least eight, or at least nine nucleic acids encoding a combination of xylose transporters described herein. In some embodiments, the non-naturally occurring microbial organisms express two xylose transporters described herein. In some embodiments, the non-naturally occurring microbial organisms express three xylose transporters described herein. In some embodiments, the non-naturally occurring microbial organisms express four xylose transporters described herein. In some embodiments, the non-naturally occurring microbial organisms express five xylose transporters described herein. In some embodiments, the non-naturally occurring microbial organisms express six xylose transporters described herein. In some embodiments, the non-naturally occurring microbial organisms express seven xylose transporters described herein. In some embodiments, the non-naturally occurring microbial organisms express eight xylose transporters described herein. In some embodiments, the non-naturally occurring microbial organisms express nine xylose transporters described herein. In some embodiments, the non-naturally occurring microbial organisms express ten xylose transporters described herein. In some embodiments, the non-naturally occurring microbial organisms express eleven xylose transporters described herein. In some embodiments, the combination of xylose transporters include two, three, four, five, six, seven, eight, nine, or ten xylose transporters of Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, and Aps1p/Hgt19p from a Metschnikowia species as well as variants thereof. In some embodiments, the combination of xylose transporters include two, three, four, five, six, seven, eight, nine, or ten xylose transporters of Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, and Aps1p/Hgt19p from H0 Metschnikowia species as well as variants thereof.

In some embodiments, provided herein are non-naturally occurring microbial organisms having a xylose-ethanol pathway and at least one exogenous nucleic acid encoding an enzyme or protein of the xylose metabolic pathway expressed in sufficient amount to confer or enhance xylose metabolism. Expression of these enzymes or their variants in microbial organisms (e.g. S. cerevisiae) can enhance xylose metabolism and increase the production of bioderived products from xylose by these microbial organisms. Thus, provided herein is an isolated polypeptide that is an enzyme or protein of Metschnikowia xylose metabolic pathway or a variant thereof; an isolated nucleic acid that encodes an enzyme or protein of Metschnikowia xylose metabolic pathway or a variant thereof; a vector that has an isolated nucleic acid that encodes an enzyme or protein of Metschnikowia xylose metabolic pathway or a variant thereof; as well as a non-naturally occurring microbial organism having a xylose-ethanol pathway and enhanced xylose metabolism by expressing at least one exogenous nucleic acid encoding an enzyme or protein of Metschnikowia xylose metabolic pathway.

Enzymes or proteins of the xylose metabolic pathway include a xylose reductase, a xylose dehydrogenase, a xylulokinase, a transketolase, a transaldolase, or a combination thereof. provided herein are non-naturally occurring microbial organisms having a xylose-ethanol pathway and at least one exogenous nucleic acid encoding an enzyme or protein expressed in sufficient amount to confer or enhance xylose metabolism, wherein enzyme or protein has an amino acid sequence that is at least 30% identical to a Metschnikowia enzyme or protein such as Xyl1p, Xyl2p, Xks1p, Tkl1p, or Tal1p. The Metschnikowia species can be selected from the group consisting of Metschnikowia sp. H0, Metschnikowia pulcherrima, Metschnikowia fructicola, Metschnikowia chrysoperlae, Metschnikowia reukaufii, Metschnikowia andauensis, Metschnikowia sinensis, Metschnikowia shanxiensis, Metschnikowia zizyphicola, Metschnikowia bicuspidata, Metschnikowia lunata, Metschnikowia zobellii, Metschnikowia australis, Metschnikowia agaveae, Metschnikowia gruessii, Metschnikowia hawaiiensis, Metschnikowia krissii, Metschnikowia sp. strain NS—O-85, and Metschnikowia sp. strain NS—O-89. The Metschnikowia enzyme or protein can be from the H0 Metschnikowia species. A number of enzymes or proteins of the xylose metabolic pathway were cloned from H0 Metschnikowia species including XYL1, XYL2, XKS1, TKL1 and TAL1. In some embodiments, the enzyme or protein can be Xyl1p, Xyl2p, Xks1p, Tkl1p, or Tal1p from the H0 Metschnikowia species.

In some embodiments, the non-naturally occurring microbial organisms provided herein are generated from host organisms having a complete xylose metabolic pathway including a xylose reductase, a xylose dehydrogenase, a xylulokinase, a transketolase, and a transaldolase. These microbial organisms can be engineered to have enhanced xylose metabolism by increasing either the activity and/or level of the enzymes or proteins of the xylose metablic pathways. These microbial organisms can also be engineered to have enhanced xylose metabolism by increasing either the activity and/or level of the enzymes or proteins of the endogenous xylose metabolic pathway. In some embodiments, additional copies of genes encoding the endogenous enzymes or proteins can be expressed in the microbial organisms. In some embodiments, one or more heterologous genes encoding an enzyme or protein having similar or same activity as the enzyme(s) of the endogenous pathway can be expressed. The microbial organisms can also be engineered to have an enhanced xylose metablic pathway by heterologously expressing enzymes or proteins of the xylose metablic pathway.

In some embodiments, the non-naturally occurring microbial organisms provided herein are generated from host organisms that lack one or more enzymes or proteins required for the xylose metablic pathway and do not naturally metabolize xylose. These microbial organisms can be engineered to have a xylose metablic pathway by heterologously expressing any missing enzymes or proteins from the endogenous xylose metablic pathway. These microbial organisms can be engineered to have enhanced xylose metabolsim by increasing either the activity or level of enzymes or proteins of the xylose metablic pathway. It is understood that in addition to heterologously expressing the missing enzymes or proteins, the microbial organisms can also be engineered to have increased activity and/or level of its endogenous enzymes or proteins to further enhance the activity of its xylose metabolic pathways.

As such, provided herein are non-naturally occurring microbial organisms having a xylose-ethanol pathway and at least one exogenous nucleic acid, or at least two, at least three, at least four, or at least five nucleic acids each encoding an enzyme or protein of the xylose metablic pathway. Enzymes or proteins of the xylose metabolic pathway include a xylose reductase, a xylose dehydrogenase, a xylulokinase, a transketolase, and a transaldolase. In some embodiments, the microbial organisms have at least two exogenous nucleic acids each encoding an enzyme or protein of the xylose metablic pathway. In some embodiments, the microbial organisms have at least three exogenous nucleic acids each encoding an enzyme or protein of the xylose metablic pathway. In some embodiments, the microbial organisms have at least four exogenous nucleic acids each encoding an enzyme or protein of the xylose metablic pathway. In some embodiments, the microbial organisms have at least five exogenous nucleic acids each encoding an enzyme or protein of the xylose metablic pathway.

The enzymes or proteins of the xylose metablic pathway provided herein can be a Metschnikowia enzyme or protein, including such as those from H0 Metschnikowia species having amino acid sequences as shown in sequence listing, as well as their variants that retain their respective enzymatic function. For example, provided herein is Xyl1p from H0 Metschnikowia species that has an amino acid sequence of SEQ ID NO:28, as well as variants thereof that retain the xylose reductase function of Xyl1p. The xylose reductase function of Xyl1p includes, but is not limited to, the reduction of xylose to xylitol, which can be determined, for example, by subjecting the variant to an in vitro assay as described herin or otherwise known in the art.

In some embodiments, provided herein are also isolated polypeptides that are variants of a Metschnikowia enzymes or proteins of the xylose metablic pathway that retains its enzymatic function. Provided herein are also isolated nucleic acids that encode polypeptides that are variants of Metschnikowia enzymes or proteins of the xylose metablic pathway that retains its enzymatic function. In some embodiments, provided herein are non-naturally occurring microbial organisms having a xylose-ethanol pathway and an exogenous nucleic acid encoding an enzyme or protein of the xylose metablic pathway, wherein the enzyme or protein is a variant of a Metschnikowia enzyme or protein of the xylose metablic pathway that retains its enzymatic function. In some embodiments, the enzyme or protein is a variant of an enzyme or protein of the xylose metablic pathway from the H0 Metschnikowia species that retains its enzymatic function. Various methods to test and confirm the enzymatic activities of xylose reductase, xylose dehydrogenase, xylulokinase, transketolase, and transaldolase are well known in the art. See e.g. Walfridsson, M., et al., Applied microbiology and biotechnology 48.2: 218-224 (1997); Richard. P. et al., FEBS letters 457.1: 135-138(1999). Richard. P. et al., FEMS microbiology letters 190.1: 39-43 (2000); Bruinenberg, P. et al., Microbiology 129.4: 965-971 (1983).

In some embodiments, provided herein are also isolated polypeptides that are variants of Xyl1p, Xyl2p, Xks1p, Tkl1p, or Tal1p from a Metschnikowia species that retain the respective enzymatic function. Provided herein are also isolated polypeptides that are variants of Xyl1p, Xyl2p, Xks1p, Tkl1p, or Tal1p from the H0 Metschnikowia species that retain the respective enzymatic function. In some embodiments, provided herein are also isolated nucleic acids that encode polypeptides that are variants of Xyl1p, Xyl2p, Xks1p, Tkl1p, or Tal1p from a Metschnikowia species that retain the respective enzymatic function. In some embodiments, provided herein are also isolated nucleic acids that encode polypeptides that are variants of Xyl1p, Xyl2p, Xks1p, Tkl1p, or Tal1p from the H0 Metschnikowia species that retain the respective enzymatic function.

In some embodiments, provided herein are non-naturally occurring microbial organisms having a xylose-ethanol pathway and an exogenous nucleic acid encoding enzymes or proteins of the xylose metabolic pathway, wherein the enzymes or proteins of the xylose metabolic pathway is a variant of Xyl1p, Xyl2p, Xks1p, Tkl1p, or Tal1p from a Metschnikowia species that retains its enzymatic function. In some embodiments, provided herein are non-naturally occurring microbial organisms having an exogenous nucleic acid encoding an enzyme or protein of the xylose metabolic pathway, wherein the enzymes or proteins of the xylose metabolic pathway is a variant of Xyl1p, Xyl2p, Xks1p, Tkl1p, or Tal1p from H0 Metschnikowia species that retains its enzymatic function.

Enzymes or proteins of the xylose metabolic pathway described herein can have amino acid sequence of at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical, to the amino acid sequences disclosed herein by SEQ ID NO, GenBank and/or GI number. In some embodiments, the enzymes or proteins of the xylose metabolic pathway described herein can have amino acid sequence of 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity, or be identical, to amino acids described herein by SEQ ID NO, GenBank and/or GI number. In some embodiments, the enzymes or proteins of the xylose metabolic pathway described herein can have amino acid sequence of 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity, or be identical, to any one of SEQ ID NOs: 28-32.

In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia enzyme or protein such as Metschnikowia Xyl1p, Xyl2p, Xks1p, Tkl1p, or Tal1p. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia enzyme or protein such as Metschnikowia Xyl1p, Xyl2p, Xks1p, Tkl1p, or Tal1p.

In some embodiments, provided herein are non-naturally occurring microbial organisms having a xylose-ethanol pathway and at least one exogenous nucleic acid encoding an enzymes or proteins of the xylose metabolic pathway, wherein the enzymes or proteins of the xylose metabolic pathway has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia enzyme or protein such as Metschnikowia Xyl1p, Xyl2p, Xks1p, Tkl1p, or Tal1p. In some embodiments, the exogenous nucleic acid encodes a Metschnikowia enzyme or protein such as Metschnikowia Xyl1p, Xyl2p, Xks1p, Tkl1p, or Tal1p.

Sequence identity (also known as homology or similarity) refers to sequence similarity between two nucleic acid molecules or between two polypeptides. Identity can be determined by comparing a position in each sequence, which may be aligned for purposes of comparison. When a position in the compared sequence is occupied by the same base or amino acid, then the molecules are identical at that position. A degree of identity between sequences is a function of the number of matching or homologous positions shared by the sequences. The alignment of two sequences to determine their percent sequence identity can be done using software programs known in the art, such as, for example, those described in Ausubel et al., Current Protocols in Molecular Biology, John Wiley and Sons, Baltimore, Md. (1999). Preferably, default parameters are used for the alignment. One alignment program well known in the art that can be used is BLAST set to default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the National Center for Biotechnology Information.

Variants of a specific enzyme or protein of the xylose metabolic pathway can also include, for example, amino acid substitutions, deletions, fusions, or truncations when compared to the reference enzyme or protein. Variants of the Metschnikowia enzymes or proteins of the xylose metabolic pathway described herein can also contain conservatively amino acids substitution, meaning that one or more amino acid can be replaced by an amino acid that does not alter the secondary and/or tertiary stricture of the enzyme or protein. Such substitutions can include the replacement of an amino acid, by a residue having similar physicochemical properties, such as substituting one aliphatic residue (Ile, Val, Leu, or Ala) for another, or substitutions between basic residues Lys and Arg, acidic residues Glu and Asp, amide residues Gln and Asn, hydroxyl residues Ser and Tyr, or aromatic residues Phe and Tyr. Phenotypically silent amino acid exchanges are described more fully in Bowie et al., Science 247:1306-10 (1990). In addition, variants of Metschnikowia enzymes or proteins of the xylose metabolic pathway include those having amino acid substitutions, deletions, or additions to the amino acid sequence outside functional regions of the protein so long as the substitution, deletion, or addition does not affect enzymatic function of the resulting polypeptide. Techniques for making these substitutions and deletions are well known in the art and include, for example, site-directed mutagenesis.

In some embodiments, provided herein are non-naturally occurring microbial organisms having a xylose-ethanol pathway and an exogenous nucleic acid encoding an enzyme or protein of the xylose metabolic pathway, wherein the enzyme or protein has 1 to 50, 1 to 45, 1 to 40, 1 to 35, 1 to 30, 1 to 25, 1 to 20, 1 to 15, 1 to 10, or 1 to 5, amino acid substitutions, deletions or insertions of Xyl1p, Xyl2p, Xks1p, Tkl1p, or Tal1p from a Metschnikowia species and retains its enzymatic function. The Metschnikowia species can be the H0 Metschnikowia species. In some embodiments, the enzyme or protein of the xylose metabolic pathway has 1 to 10 amino acid substitutions, deletions or insertions of Xyl1p, Xyl2p, Xks1p, Tkl1p, or Tal1p from a Metschnikowia species and retains the enzymatic function. The Metschnikowia species can be the H0 Metschnikowia species. In some embodiments, the enzyme or protein of the xylose metabolic pathway has 1 to 5 amino acid substitutions, deletions or insertions of Xyl1p, Xyl2p, Xks1p, Tkl1p, or Tal1p from a Metschnikowia species and retains the enzymatic function. The Metschnikowia species can be the H0 Metschnikowia species.

Enzyme or protein of the xylose metabolic pathway provided herein also include functional fragments of specific Metschnikowia enzymes or proteins of the xylose metabolic pathway that retain their enzymatic function. In some embodiments, provided herein is an isolated polypeptide that is a functional fragment of a specific Metschnikowia enzyme or protein of the xylose metabolic pathway. In some embodiments, provided herein is an isolated nucleic acid that encodes a polypeptide that is functional fragment of a specific Metschnikowia enzyme or protein of the xylose metabolic pathway. In some embodiments, the enzyme or protein can be fragments of an enzyme or protein of the xylose metabolic pathway such as Metschnikowia Xyl1p, Xyl2p, Xks1p, Tkl1p, or Tal1p that retains its enzymatic function. In some embodiments, the enzyme or protein can be fragments of an enzyme or protein of the xylose metabolic pathway such as Xyl1p, Xyl2p, Xks1p, Tkl1p, or Tal1p from the H0 Metschnikowia species retains its enzymatic function.

In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a function fragment of a Metschnikowia enzyme or protein of the xylose metabolic pathway including such as Metschnikowia Xyl1p, Xyl2p, Xks1p, Tkl1p, or Tal1p. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a function fragment of a Metschnikowia enzyme or protein of the xylose metabolic pathway including such as Metschnikowia Xyl1p, Xyl2p, Xks1p, Tkl1p, or Tal1p.

In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a function fragment of an enzyme or protein of the xylose metabolic pathway of H0 Metschnikowia species including such as Xyl1p, Xyl2p, Xks1p, Tkl1p, or Tal1p of H0 Metschnikowia species. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a function fragment of an enzyme or protein of the xylose metabolic pathway of H0 Metschnikowia species including such as Metschnikowia Xyl1p, Xyl2p, Xks1p, Tkl1p, or Tal1p of H0 Metschnikowia species.

In some embodiments, provided herein are non-naturally occurring microbial organisms having a xylose-ethanol pathway and an exogenous nucleic acid encoding a functional fragment of a Metschnikowia enzyme or protein of the xylose metabolic pathway that retains its enzymatic function. In some embodiments, the non-naturally occurring microbial organisms provided herein have at least one exogenous nucleic acid encoding an enzyme or protein of the xylose metabolic pathway that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a function fragment of a Metschnikowia enzyme or protein of the xylose metabolic pathway including such as Metschnikowia Xyl1p, Xyl2p, Xks1p, Tkl1p, or Tal1p. In some embodiments, the non-naturally occurring microbial organisms provided herein can have a xylose-ethanol pathway and at least one exogenous nucleic acid encoding an enzyme or protein of the xylose metabolic pathway that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a function fragment of an enzyme or protein of the xylose metabolic pathway of H0 Metschnikowia species such as Xyl1p, Xyl2p, Xks1p, Tkl1p, or Tal1p of H0 Metschnikowia species.

In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Xyl1p. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Xyl1p. In some embodiments, provided herein are non-naturally occurring microbial organisms having a xylose-ethanol pathway and at least one exogenous nucleic acid encoding a xylose reductase, wherein the xylose reductase has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Xyl1p. In some embodiments, the xylose reductase can be a Metschnikowia Xyl1p. In some embodiments, the xylose reductase can be a variant of a Metschnikowia Xyl1p that retains its reductase function. The xylose reductase can be a functional fragment of a Metschnikowia Xyl1p. In some embodiments, the xylose reductase can have 1 to 50, 1 to 45, 1 to 40, 1 to 35, 1 to 30, 1 to 25, 1 to 20, 1 to 15, 1 to 10, or 1 to 5, amino acid substitutions, deletions or insertions of Xyl1p from a Metschnikowia species. In some embodiments, the xylose reductase has 1 to 10 amino acid substitutions, deletions or insertions of Xyl1p from a Metschnikowia species. In some embodiments, the xylose reductase has 1 to 5 amino acid substitutions, deletions or insertions of Xyl1p from a Metschnikowia species.

The Metschnikowia species can be the H0 Metschnikowia species. In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to Xyl1p of H0 Metschnikowia species. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Xyl1p of H0 Metschnikowia species. In some embodiments, the non-naturally occurring microbial organisms have a xylose-ethanol pathway and at least one exogenous nucleic acid encoding a xylose reductase that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to Xyl1p of H0 Metschnikowia species. In some embodiments, the xylose reductase is Xyl1p of H0 Metschnikowia species. In some embodiments, the xylose reductase can be a variant of Xyl1p of H0 Metschnikowia species that retains its reductase function. In some embodiments, the xylose reductase is a functional fragment of Xyl1p of H0 Metschnikowia species. In some embodiments, the xylose reductase can have 1 to 50, 1 to 45, 1 to 40, 1 to 35, 1 to 30, 1 to 25, 1 to 20, 1 to 15, 1 to 10, or 1 to 5, amino acid substitutions, deletions or insertions of Xyl1p from H0 Metschnikowia species. In some embodiments, the xylose reductase has 1 to 10 amino acid substitutions, deletions or insertions of Xyl1p from H0 Metschnikowia species. In some embodiments, the xylose reductase has 1 to 5 amino acid substitutions, deletions or insertions of Xyl1p from H0 Metschnikowia species. In some embodiments, the xylose reductase has the amino acid sequence of SEQ ID NO: 28. In some embodiments, the amino acid sequence of the xylose reductase is SEQ ID NO: 28. In some embodiments, the nucleic acid has the sequence of SEQ ID NO: 33. In some embodiments, the sequence of the nucleic acid is SEQ ID NO: 33. The nucleic acid encoding Xyl1p from a Metschnikowia species can be codon optimized for heterologous expression. In some embodiments, the nucleic acid encoding Metschnikowia Xyl1p is codon optimized for expression in a yeast host strain. The yeast host strain can be any yeast host strain described herein, such as S. cerevisiae. In some embodiments, the nucleic acid encoding Metschnikowia Xyl1p is codon optimized for expression in a bacterial host strain. The bacterial host strain can be any bacterial host strain described herein, such as E. coli. In some embodiments, the nucleic acid encoding Xyl1p from H0 Metschnikowia species is codon optimized for expression in S. cerevisiae. For example, in some embodiments, the nucleic acid encoding Xyl1p of H0 Metschnikowia species is codon optimized for expression in S. cerevisiae.

In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Xyl2p. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Xyl2p. In some embodiments, provided herein are non-naturally occurring microbial organisms having a xylose-ethanol pathway and at least one exogenous nucleic acid encoding a xylose dehydrogenase, wherein the xylose dehydrogenase has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Xyl2p. In some embodiments, the xylose dehydrogenase can be a Metschnikowia Xyl2p. In some embodiments, the xylose dehydrogenase can be a variant of a Metschnikowia Xyl2p that retains its dehydrogenase function. The xylose dehydrogenase can be a functional fragment of a Metschnikowia Xyl2p. In some embodiments, the xylose dehydrogenase can have 1 to 50, 1 to 45, 1 to 40, 1 to 35, 1 to 30, 1 to 25, 1 to 20, 1 to 15, 1 to 10, or 1 to 5, amino acid substitutions, deletions or insertions of Xyl2p from a Metschnikowia species. In some embodiments, the xylose dehydrogenase has 1 to 10 amino acid substitutions, deletions or insertions of Xyl2p from a Metschnikowia species. In some embodiments, the xylose dehydrogenase has 1 to 5 amino acid substitutions, deletions or insertions of Xyl2p from a Metschnikowia species.

The Metschnikowia species can be the H0 Metschnikowia species. In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to Xyl2p of H0 Metschnikowia species. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Xyl2p of H0 Metschnikowia species. In some embodiments, the non-naturally occurring microbial organisms have a xylose-ethanol pathway and at least one exogenous nucleic acid encoding a xylose dehydrogenase that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to Xyl2p of H0 Metschnikowia species. In some embodiments, the xylose dehydrogenase is Xyl2p of H0 Metschnikowia species. In some embodiments, the xylose dehydrogenase can be a variant of Xyl2p of H0 Metschnikowia species that retains its dehydrogenase function. In some embodiments, the xylose dehydrogenase is a functional fragment of Xyl2p of H0 Metschnikowia species. In some embodiments, the xylose dehydrogenase can have 1 to 50, 1 to 45, 1 to 40, 1 to 35, 1 to 30, 1 to 25, 1 to 20, 1 to 15, 1 to 10, or 1 to 5, amino acid substitutions, deletions or insertions of Xyl2p from H0 Metschnikowia species. In some embodiments, the xylose dehydrogenase has 1 to 10 amino acid substitutions, deletions or insertions of Xyl2p from H0 Metschnikowia species. In some embodiments, the xylose dehydrogenase has 1 to 5 amino acid substitutions, deletions or insertions of Xyl2p from H0 Metschnikowia species. In some embodiments, the xylose dehydrogenase has the amino acid sequence of SEQ ID NO: 29. In some embodiments, the amino acid sequence of the xylose dehydrogenase is SEQ ID NO: 29. In some embodiments, the nucleic acid has the sequence of SEQ ID NO: 34. In some embodiments, the sequence of the nucleic acid is SEQ ID NO: 34. The nucleic acid encoding Xyl2p from a Metschnikowia species can be codon optimized for heterologous expression. In some embodiments, the nucleic acid encoding Metschnikowia Xyl2p is codon optimized for expression in a yeast host strain. The yeast host strain can be any yeast host strain described herein, such as S. cerevisiae. In some embodiments, the nucleic acid encoding Metschnikowia Xyl2p is codon optimized for expression in a bacterial host strain. The bacterial host strain can be any bacterial host strain described herein, such as E. coli. In some embodiments, the nucleic acid encoding Xyl2p from H0 Metschnikowia species is codon optimized for expression in S. cerevisiae. For example, in some embodiments, the nucleic acid encoding Xyl2p of H0 Metschnikowia species is codon optimized for expression in S. cerevisiae.

In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Xks1p. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to aMetschnikowia Xks1p. In some embodiments, provided herein are non-naturally occurring microbial organisms having a xylose-ethanol pathway and at least one exogenous nucleic acid encoding a xylulokinase, wherein the xylulokinase has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Xks1p. In some embodiments, the xylulokinase can be aMetschnikowia Xks1p. In some embodiments, the xylulokinase can be a variant of aMetschnikowia Xks1p that retains its xylulokinase function. The xylulokinase can be a functional fragment of aMetschnikowia Xks1p. In some embodiments, the xylulokinase can have 1 to 50, 1 to 45, 1 to 40, 1 to 35, 1 to 30, 1 to 25, 1 to 20, 1 to 15, 1 to 10, or 1 to 5, amino acid substitutions, deletions or insertions of Xks1p from a Metschnikowia species. In some embodiments, the xylulokinase has 1 to 10 amino acid substitutions, deletions or insertions of Xks1p from a Metschnikowia species. In some embodiments, the xylulokinase has 1 to 5 amino acid substitutions, deletions or insertions of Xks1p from a Metschnikowia species.

The Metschnikowia species can be the H0 Metschnikowia species. In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to Xks1p of H0 Metschnikowia species. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Xks1p of H0 Metschnikowia species. In some embodiments, the non-naturally occurring microbial organisms have a xylose-ethanol pathway and at least one exogenous nucleic acid encoding a xylulokinase that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to Xks1p of H0 Metschnikowia species. In some embodiments, the xylulokinase is Xks1p of H0 Metschnikowia species. In some embodiments, the xylulokinase can be a variant of Xks1p of H0 Metschnikowia species that retains its xylulokinase function. In some embodiments, the xylulokinase is a functional fragment of Xks1p of H0 Metschnikowia species. In some embodiments, the xylulokinase can have 1 to 50, 1 to 45, 1 to 40, 1 to 35, 1 to 30, 1 to 25, 1 to 20, 1 to 15, 1 to 10, or 1 to 5, amino acid substitutions, deletions or insertions of Xks1p from H0 Metschnikowia species. In some embodiments, the xylulokinase has 1 to 10 amino acid substitutions, deletions or insertions of Xks1p from H0 Metschnikowia species. In some embodiments, the xylulokinase has 1 to 5 amino acid substitutions, deletions or insertions of Xks1p from H0 Metschnikowia species. In some embodiments, the xylulokinase has the amino acid sequence of SEQ ID NO: 30. In some embodiments, the amino acid sequence of the xylulokinase is SEQ ID NO: 30. In some embodiments, the nucleic acid has the sequence of SEQ ID NO: 35. In some embodiments, the sequence of the nucleic acid is SEQ ID NO: 35. The nucleic acid encoding Xks1p from a Metschnikowia species can be codon optimized for heterologous expression. In some embodiments, the nucleic acid encoding Metschnikowia Xks1p is codon optimized for expression in a yeast host strain. The yeast host strain can be any yeast host strain described herein, such as S. cerevisiae. In some embodiments, the nucleic acid encoding Metschnikowia Xks1p is codon optimized for expression in a bacterial host strain. The bacterial host strain can be any bacterial host strain described herein, such as E. coli. In some embodiments, the nucleic acid encoding Xks1p from H0 Metschnikowia species is codon optimized for expression in S. cerevisiae. For example, in some embodiments, the nucleic acid encoding Xks1p of H0 Metschnikowia species is codon optimized for expression in S. cerevisiae.

In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Tkl1p. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Tkl1p. In some embodiments, provided herein are non-naturally occurring microbial organisms having a xylose-ethanol pathway and at least one exogenous nucleic acid encoding a transketolase, wherein the transketolase has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Tkl1p. In some embodiments, the transketolase can be a Metschnikowia Tkl1p. In some embodiments, the transketolase can be a variant of a Metschnikowia Tkl1p that retains its transketolase function. The transketolase can be a functional fragment of a Metschnikowia Tkl1p. In some embodiments, the transketolase can have 1 to 50, 1 to 45, 1 to 40, 1 to 35, 1 to 30, 1 to 25, 1 to 20, 1 to 15, 1 to 10, or 1 to 5, amino acid substitutions, deletions or insertions of Tkl1p from a Metschnikowia species. In some embodiments, the transketolase has 1 to 10 amino acid substitutions, deletions or insertions of Tkl1p from a Metschnikowia species. In some embodiments, the transketolase has 1 to 5 amino acid substitutions, deletions or insertions of Tkl1p from a Metschnikowia species.

The Metschnikowia species can be the H0 Metschnikowia species. In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to Tkl1p of H0 Metschnikowia species. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Tkl1p of H0 Metschnikowia species. In some embodiments, the non-naturally occurring microbial organisms have a xylose-ethanol pathway and at least one exogenous nucleic acid encoding a transketolase that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to Tkl1p of H0 Metschnikowia species. In some embodiments, the transketolase is Tkl1p of H0 Metschnikowia species. In some embodiments, the transketolase can be a variant of Tkl1p of H0 Metschnikowia species that retains its transketolase function. In some embodiments, the transketolase is a functional fragment of Tkl1p of H0 Metschnikowia species. In some embodiments, the transketolase can have 1 to 50, 1 to 45, 1 to 40, 1 to 35, 1 to 30, 1 to 25, 1 to 20, 1 to 15, 1 to 10, or 1 to 5, amino acid substitutions, deletions or insertions of Tkl1p from H0 Metschnikowia species. In some embodiments, the transketolase has 1 to 10 amino acid substitutions, deletions or insertions of Tkl1p from H0 Metschnikowia species. In some embodiments, the transketolase has 1 to 5 amino acid substitutions, deletions or insertions of Tkl1p from H0 Metschnikowia species. In some embodiments, the transketolase has the amino acid sequence of SEQ ID NO: 31. In some embodiments, the amino acid sequence of the transketolase is SEQ ID NO: 31. In some embodiments, the nucleic acid has the sequence of SEQ ID NO: 36. In some embodiments, the sequence of the nucleic acid is SEQ ID NO: 36. The nucleic acid encoding Tkl1p from a Metschnikowia species can be codon optimized for heterologous expression. In some embodiments, the nucleic acid encoding Metschnikowia Tkl1p is codon optimized for expression in a yeast host strain. The yeast host strain can be any yeast host strain described herein, such as S. cerevisiae. In some embodiments, the nucleic acid encoding Metschnikowia Tkl1p is codon optimized for expression in a bacterial host strain. The bacterial host strain can be any bacterial host strain described herein, such as E. coli. In some embodiments, the nucleic acid encoding Tkl1p from H0 Metschnikowia species is codon optimized for expression in S. cerevisiae. For example, in some embodiments, the nucleic acid encoding Tkl1p of H0 Metschnikowia species is codon optimized for expression in S. cerevisiae.

In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Tal1p. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Tal1p. In some embodiments, provided herein are non-naturally occurring microbial organisms having a xylose-ethanol pathway and at least one exogenous nucleic acid encoding a transaldolase, wherein the transaldolase has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Metschnikowia Tal1p. In some embodiments, the transaldolase can be a Metschnikowia Tal1p. In some embodiments, the transaldolase can be a variant of a Metschnikowia Tal1p that retains its transaldolase function. The transaldolase can be a functional fragment of a Metschnikowia Tal1p. In some embodiments, the transaldolase can have 1 to 50, 1 to 45, 1 to 40, 1 to 35, 1 to 30, 1 to 25, 1 to 20, 1 to 15, 1 to 10, or 1 to 5, amino acid substitutions, deletions or insertions of Tal1p from a Metschnikowia species. In some embodiments, the transaldolase has 1 to 10 amino acid substitutions, deletions or insertions of Tal1p from a Metschnikowia species. In some embodiments, the transaldolase has 1 to 5 amino acid substitutions, deletions or insertions of Tal1p from a Metschnikowia species.

The Metschnikowia species can be the H0 Metschnikowia species. In some embodiments, provided herein is an isolated polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to Tal 1p of H0 Metschnikowia species. In some embodiments, provided herein is an isolated nucleic acid that encodes a polypeptide that has an amino acid sequence that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a Tal1p of H0 Metschnikowia species. In some embodiments, the non-naturally occurring microbial organisms have a xylose-ethanol pathway and at least one exogenous nucleic acid encoding a transaldolase that is at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to Tal1p of H0 Metschnikowia species. In some embodiments, the transaldolase is Tal1p of H0 Metschnikowia species. In some embodiments, the transaldolase can be a variant of Tal1p of H0 Metschnikowia species that retains its transaldolase function. In some embodiments, the transaldolase is a functional fragment of Tal1p of H0 Metschnikowia species. In some embodiments, the transaldolase can have 1 to 50, 1 to 45, 1 to 40, 1 to 35, 1 to 30, 1 to 25, 1 to 20, 1 to 15, 1 to 10, or 1 to 5, amino acid substitutions, deletions or insertions of Tal1p from H0 Metschnikowia species. In some embodiments, the transaldolase has 1 to 10 amino acid substitutions, deletions or insertions of Tal1p from H0 Metschnikowia species. In some embodiments, the transaldolase has 1 to 5 amino acid substitutions, deletions or insertions of Tal1p from H0 Metschnikowia species. In some embodiments, the transaldolase has the amino acid sequence of SEQ ID NO: 32. In some embodiments, the amino acid sequence of the transaldolase is SEQ ID NO: 32. In some embodiments, the nucleic acid has the sequence of SEQ ID NO: 37. In some embodiments, the sequence of the nucleic acid is SEQ ID NO: 37. The nucleic acid encoding Tal1p from a Metschnikowia species can be codon optimized for heterologous expression. In some embodiments, the nucleic acid encoding Metschnikowia Tal1p is codon optimized for expression in a yeast host strain. The yeast host strain can be any yeast host strain described herein, such as S. cerevisiae. In some embodiments, the nucleic acid encoding Metschnikowia Tal1p is codon optimized for expression in a bacterial host strain. The bacterial host strain can be any bacterial host strain described herein, such as E. coli. In some embodiments, the nucleic acid encoding Tal1p from H0 Metschnikowia species is codon optimized for expression in S. cerevisiae. For example, in some embodiments, the nucleic acid encoding Tal1p of H0 Metschnikowia species is codon optimized for expression in S. cerevisiae.

Expression of one or more of xylose transporters can improve xylose uptake. Expression of one or more of enzymes or proteins of the xylose metabolic pathway can improve xylose metabolism. Provided herein are non-naturally occurring microbial organisms having a xylose-ethanol pathway and at least one exogenous nucleic acid encoding an enzyme or protein expressed in a sufficient amount to confer or enhance xylose uptake or metabolism. In some embodiments, the non-naturally occurring microbial organisms can have at least one exogenous nucleic acid encoding a xylose transporter expressed in a sufficient amount to confer or enhance xylose uptake and at least one exogenous nucleic acid encoding an enzyme or protein of the xylose metabolic pathway in a sufficient amount to confer or enhance xylose metabolism. The enzymes or proteins of the xylose metabolic pathway can include one or more of xylose reductase, a xylose dehydrogenase, a xylulokinase, a transketolase, and a transaldolase. In some embodiments, the non-naturally occurring microbial organisms can have at least two exogenous nucleic acids encoding a xylose transporter and a xylose reductase. In some embodiments, the non-naturally occurring microbial organisms can have at least two exogenous nucleic acid encoding a xylose transporter and a xylose dehydrogenase. In some embodiments, the non-naturally occurring microbial organisms can have at least two exogenous nucleic acids encoding a xylose transporter and a xylulokinase. In some embodiments, the non-naturally occurring microbial organisms can have at least three exogenous nucleic acids encoding a xylose transporter, a xylose reductase and a xylose dehydrogenase. In some embodiments, the non-naturally occurring microbial organisms can have at least three exogenous nucleic acids encoding a xylose transporter, a xylose reductase and a xylulokinase. In some embodiments, the non-naturally occurring microbial organisms can have at least three exogenous nucleic acids encoding a xylose transporter, a xylulokinase and a xylose dehydrogenase. In some embodiments, the non-naturally occurring microbial organisms can have at least four exogenous nucleic acids encoding a xylose transporter, a xylose reductase, a xylose dehydrogenase, and a xylulokinase. In some embodiments, the non-naturally occurring microbial organisms can further have at least one exogenous nucleic acid encoding a transketolase. In some embodiments, the non-naturally occurring microbial organisms can further have at least one exogenous nucleic acid encoding a transaldolase. In some embodiments, the non-naturally occurring microbial organisms can further have at least two exogenous nucleic acids encoding a transketolase and a transaldolase.

In some embodiments, the non-naturally occurring microbial organisms can include at least seven exogenous nucleic acids each encoding a Metschnikowia enzyme or protein, including Xyt1p, Gxf1p, Gxf2p/Ga12p, Xyl1p, Xyl2p, Xks1p and Tal1p. The non-naturally occurring microbial organisms can further include at least an exogenous nucleic acids encoding Metschnikowia Tkl1p.

The non-naturally occurring microbial organisms can have at least one exogenous nucleic acid, or at least two, at least three, at least four, at least five, at least six, at least seven, at least eight, at least nine, at least ten, or at least eleven nucleic acids each encoding one, two, three, four, five, six, seven, eight, nine, or ten xylose transporters. The xylose transporter can be any xylose transporter as described herein or their variants that retain their transporter function. The xylose transporters can be Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, or Aps1p/Hgt19p from a Metschnikowia species, or their variants that retain their transporter function. In some embodiments, the xylose transporter can include Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, or Aps1p/Hgt19p from the H0 Metschnikowia species, and their variants that retain their transporter function. The non-naturally occurring microbial organisms can have at least one exogenous nucleic acid, or at least two, at least three, at least four, or at least five nucleic acids each encoding one, two, three, four, or five enzymes or proteins of the xylose metabolic pathway. The enzyme or protein of the xylose metabolic pathway can be any enzyme or protein as described herein or their variants that retain their respective enzymatic function. The enzyme or protein of the xylose metabolic pathway can include xylose reductase, a xylose dehydrogenase, a xylulokinase, a transketolase, and a transaldolase. In some embodiments, the enzyme or protein of the xylose metabolic pathway can include Xyl1p, Xyl2p, Xks1p, Tkl1p, or Tal1p from a Metschnikowia species, and their variants that retain their respective enzymatic function. In some embodiments, the xylose transporter can include Xyl1p, Xyl2p, Xks1p, Tkl1p, or Tal1p from the H0 Metschnikowia species, and their variants that retain their respective enzymatic function. The non-naturally occurring microbial organisms provided herein can include exogenous nucleic acids encoding any combination or permutations of xylose transporters and enzymes or proteins of the xylose metabolic pathway as described herein.

The xylose transporters provided herein include Metschnikowia xylose transporters, including such as those from H0 Metschnikowia species having amino acid sequences as shown in sequence listing, as well as their variants, derivatives, fragments that retain their transporter function. For example, provided herein is Xyt1p from H0 Metschnikowia species that has an amino acid sequence of SEQ ID NO:1, as well as variants, derivatives, and fragments thereof that retain the transporter function of Xyt1p. The transporter function of Xyt1p includes but is not limited to transport of xylose across cell membrane, which can be determined, for example, by subjecting the variant, derivative, or fragment to the transporter assay as described herein or otherwise known in the art. The enzymes or proteins of xylose metabolic pathways can be the enzymes or proteins from a Metschnikowia species including such as those from H0 Metschnikowia species having amino acid sequences as shown in sequence listing, as well as their variants, derivatives, fragments that retain their respective enzymatic function. For example, provided herein is Xyl1p from H0 Metschnikowia species that has an amino acid sequence of SEQ ID NO:28, as well as variants, derivatives, and fragments thereof that retain the xylose reductase function of Xyl1p. The xylose reductase activity of Xyl1p can be determined, for example, by subjecting the variant, derivative, or fragment to the xylose reductase assay as described herein or otherwise known in the art. Various methods to test and confirm the enzymatic activities of xylose reductase, a xylose dehydrogenase, a xylulokinase, a transketolase, and a transaldolase are well known in the art. See e.g Walfridsson, M., et al., Applied microbiology and biotechnology 48.2: 218-224 (1997); Richard. P. et al., FEBS letters 457.1: 135-138(1999). Richard. P. et al., FEMS microbiology letters 190.1: 39-43 (2000); Bruinenberg, P. et al., Microbiology 129.4: 965-971 (1983).

In some embodiments, provided herein are non-naturally occurring microbial organisms having a xylose-ethanol pathway and an exogenous nucleic acid encoding an enzyme or protein expressed in a sufficient amount to confer or enhance xylose uptake or metabolism, wherein said enzyme or protein is selected from the group consisting of a xylose transporter, a xylose reductase, a xylose dehydrogenase and a xylulokinase, and wherein the enzyme or protein is a variant of a Metschnikowia enzyme or protein. In some embodiments, the enzyme or protein is a variant of the enzyme or protein from the H0 Metschnikowia species as described herein. Variants of a specific enzyme or protein can include amino acid substitutions, deletions, fusions, or truncations when compared to the reference enzyme or protein. In some embodiments, provided herein are non-naturally occurring microbial organisms having an exogenous nucleic acid encoding an enzyme or protein that is a variant of Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, Aps1p/Hgt19p, Xyl1p, Xyl2p, Xks1p, Tkl1p, or Tal1p from a Metschnikowia species. In some embodiments, provided herein are non-naturally occurring microbial organisms having an exogenous nucleic acid encoding an enzyme or protein that is a variant of Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, Aps1p/Hgt19p, Xyl1p, Xyl2p, Xks1p, Tkl1p, or Tal1p from H0 Metschnikowia species.

In some embodiments, variants of Metschnikowia xylose transporters and enzymes or proteins of xylose metabolic pathways described herein include covalent modification or aggregative conjugation with other chemical moieties, such as glycosyl groups, polyethylene glycol (PEG) groups, lipids, phosphate, acetyl groups, and the like. In some embodiments, variants of Metschnikowia xylose transporters and enzymes or proteins of xylose metabolic pathways described herein further include, for example, fusion proteins formed of the Metschnikowia enzymes or proteins and another polypeptide. The added polypeptides for constructing the fusion protein include those that facilitate purification or oligomerization of the Metschnikowia enzymes, or those that enhance stability and/or activity of the Metschnikowia enzymes.

The Metschnikowia enzymes or proteins described herein can be fused to heterologous polypeptides to facilitate purification. Many available heterologous peptides (peptide tags) allow selective binding of the fusion protein to a binding partner. Non-limiting examples of peptide tags include 6-His, thioredoxin, hemaglutinin, GST, and the OmpA signal sequence tag. A binding partner that recognizes and binds to the heterologous peptide tags can be any molecule or compound, including metal ions (for example, metal affinity columns), antibodies, antibody fragments, or any protein or peptide that selectively or specifically binds the heterologous peptide to permit purification of the fusion protein.

The Metschnikowia enzymes or proteins can also be modified to facilitate formation of oligomers. For example, the Xyt1p polypeptides can be fused to peptide moieties that promote oligomerization, such as leucine zippers and certain antibody fragment polypeptides, such as Fc polypeptides. Techniques for preparing these fusion proteins are known, and are described, for example, in WO 99/31241 and in Cosman et al., Immunity 14:123-133 (2001). Fusion to an Fc polypeptide offers the additional advantage of facilitating purification by affinity chromatography over Protein A or Protein G columns. Fusion to a leucine-zipper (LZ), for example, a repetitive heptad repeat, often with four or five leucine residues interspersed with other amino acids, is described in Landschulz et al., Science 240:1759-64 (1988).

The xylose transporters and enzymes or proteins of xylose metabolic pathways described herein can be provided in an isolated form, or in a substantially purified form. The polypeptides can be recovered and purified from recombinant cell cultures by known methods, including, for example, ammonium sulfate or ethanol precipitation, anion or cation exchange chromatography, phosphocellulose chromatography, hydrophobic interaction chromatography, affinity chromatography, hydroxylapatite chromatography, and lectin chromatography. In some embodiments, protein chromatography is employed for purification.

The Metschnikowia xylose transporters and enzymes or proteins of xylose metabolic pathways described herein can be recombinantly expressed by suitable hosts. When heterologous expression is desired, the coding sequences of specific Metschnikowia xylose transporters or enzymes or proteins of xylose metabolic pathways can be modified in accordance with the codon usage of the host. The standard genetic code is well known in the art, as reviewed in, for example, Osawa et al., Microbiol Rev. 56(1):229-64 (1992). Yeast species, including but not limited to Saccharomyces cerevisiae, Candida azyma, Candida diversa, Candida magnoliae, Candida rugopelliculosa, Yarrowia hpolytica, and Zygoascus hellenicus, use the standard code. Certain yeast species use alternative codes. For example, “CUG,” standard codon for “Leu,” encodes “Ser” in species such as Candida albicans, Candida cylindracea, Candida melibiosica, Candida parapsilosis, Candida rugose, Pichia stipitis, and Metschnikowia species. The codon table for the H0 Metschnikowia species is provided below. The DNA codon CTG in a foreign gene from a non “CUG” clade species needs to be changed to TTG, CTT, CTC, TTA, or CTA for a functional expression of a protein in the Metschnikowia species. Other codon optimization can result in increase of protein expression of a foreign gene in the Metschnikowia species. The codon table for the H0 Metschnikowia species is provided below. Codon optimization can result in increase protein expression of a foreign gene in the host. Methods of Codon optimization are well known in the art (e.g. Chung et al., BMC Syst Biol. 6:134 (2012); Chin et al., Bioinformatics 30(15):2210-12 (2014)), and various tools are available (e.g. DNA2.0 at https://www.dna20.com/services/genegps; and OPTIMIZER at http://genomes.urv.es/OPTIMIZER).

TABLE
Codon for H0 Metschnikowia species

| Amino Acid | SLC | DNA codons |
|---|---|---|
| Isoleucine | I | ATT ATC ATA |
| Leucine | L | CTT CTC CTA TTA TTG |
| Valine | V | GTT GTC GTA GTG |
| Phenylalanine | F | TTT TTC |
| Methionine | M | ATG |
| Cysteine | C | TGT TGC |
| Alanine | A | GCT GCC GCA GCG |
| Glycine | G | GGT GGC GGA GGG |
| Proline | P | CCT CCC CCA CCG |
| Threonine | T | ACT ACC ACA ACG |
| Serine | S | TCT TCC TCA TCG AGT AGC CTG |
| Tyrosine | Y | TAT TAC |
| Tryptophan | W | TGG |
| Glutamine | Q | CAA CAG |
| Asparagine | N | AAT AAC |
| Histidine | H | CAT CAC |
| Glutamic acid | E | GAA GAG |
| Aspartic acid | D | GAT GAC |
| Lysine | K | AAA AAG |
| Arginine | R | CGT CGC CGA CGG AGA AGG |
| Stop codons | Stop | TAA TAG TGA |

Furthermore, the hosts can simultaneously produce other transporters such that multiple transporters are expressed in the same cell, wherein the different transporters can form oligomers to transport the same sugar. Alternatively, the different transporters can function independently to transport different sugars.

In some embodiments, provided herein are non-naturally occurring microbial organisms having a xylose-ethanol pathway and an exogenous nucleic acid encoding an enzyme or protein selected from a group consisting of a xylose transporter, a xylose reductase, a xylose dehydrogenase, a xylulokinase, a transketolase, and a transaldolase, wherein the enzyme or protein has 1 to 50, 1 to 45, 1 to 40, 1 to 35, 1 to 30, 1 to 25, 1 to 20, 1 to 15, 1 to 10, or 1 to 5, amino acid substitutions, deletions or insertions of Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, Aps1p/Hgt19p, Xyl1p, Xyl2p, Xks1p, Tkl1p, or Tal1p from a Metschnikowia species. In some embodiments, the enzyme or protein has 1 to 10 amino acid substitutions, deletions or insertions of Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, Aps1p/Hgt19p, Xyl1p, Xyl2p, Xks1p, Tkl1p, or Tal1p from a Metschnikowia species. In some embodiments, the xylose transporter has 1 to 5 amino acid substitutions, deletions or insertions of Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, Aps1p/Hgt19p, Xyl1p, Xyl2p, Xks1p, Tkl1p, or Tal1p from a Metschnikowia species. The Metschnikowia species can be the H0 Metschnikowia species.

Variants of Metschnikowia xylose transporters and enzymes or proteins of xylose metabolic pathways can be generated by conventional methods known in the art, such as by introducing mutations at particular locations by oligonucleotide-directed site-directed mutagenesis. Site-directed-mutagenesis is considered an informational approach to protein engineering and can rely on high-resolution crystallographic structures of target proteins for specific amino acid changes (Van Den Burg et al., PNAS 95:2056-60 (1998)). Computational methods for identifying site-specific changes for a variety of protein engineering objectives are also known in the art (Hellinga, Nature Structural Biology 5:525-27 (1998)).

Other techniques known in the art include, but are not limited to, non-informational mutagenesis techniques (referred to generically as “directed evolution”). Directed evolution, in conjunction with high-throughput screening, allows testing of statistically meaningful variations in protein conformation (Arnold, 1998). Directed evolution technology can include diversification methods similar to that described by Crameri et al., Nature 391:288-91 (1998), site-saturation mutagenesis, staggered extension process (StEP) (Zhao et al., Nature Biotechnology 16:258-61 (1998)), and DNA synthesis/reassembly (U.S. Pat. No. 5,965,408).

As disclosed herein, a nucleic acid encoding a xylose transporter and/or an enzyme or protein of the xylose metabolic pathway can be introduced into a host organism. In some cases, it can also be desirable to modify an activity of a biosynthesis pathway enzyme or protein to increase production of a desired product. For example, known mutations that increase the activity of a protein or enzyme can be introduced into an encoding nucleic acid molecule. Additionally, optimization methods can be applied to increase the activity of an enzyme or protein and/or decrease an inhibitory activity, for example, decrease the activity of a negative regulator.

One such optimization method is directed evolution. Directed evolution is a powerful approach that involves the introduction of mutations targeted to a specific gene in order to improve and/or alter the properties of an enzyme or protein. Improved and/or altered enzymes or proteins can be identified through the development and implementation of sensitive high-throughput screening assays that allow the automated screening of many enzyme or protein variants (for example, >10⁴). Iterative rounds of mutagenesis and screening typically are performed to afford an enzyme or protein with optimized properties. Computational algorithms that can help to identify areas of the gene for mutagenesis also have been developed and can significantly reduce the number of enzyme or protein variants that need to be generated and screened. Numerous directed evolution technologies have been developed (for reviews, see Hibbert et al., Biomol. Eng 22:11-19 (2005); Huisman and Lalonde, in Biocatalysis in the pharmaceutical and biotechnology industries pgs. 717-742 (2007), Patel (ed.), CRC Press; Otten and Quax. Biomol. Eng 22:1-9 (2005).; and Sen et al., Appl Biochem. Biotechnol 143:212-223 (2007)) to be effective at creating diverse variant libraries, and these methods have been successfully applied to the improvement of a wide range of properties across many enzyme or protein classes. Enzyme or protein characteristics that have been improved and/or altered by directed evolution technologies include, for example: selectivity/specificity, for conversion of non-natural substrates; temperature stability, for robust high temperature processing; pH stability, for bioprocessing under lower or higher pH conditions; substrate or product tolerance, so that high product titers can be achieved; binding (Kₘ), including broadening substrate binding to include non-natural substrates; inhibition (Kᵢ), to remove inhibition by products, substrates, or key intermediates; activity (kcat), to increases enzymatic reaction rates to achieve desired flux; expression levels, to increase protein yields and overall pathway flux; oxygen stability, for operation of air sensitive enzymes under aerobic conditions; and anaerobic activity, for operation of an aerobic enzyme or protein in the absence of oxygen.

A number of exemplary methods have been developed for the mutagenesis and diversification of genes to target desired properties of specific enzymes or proteins. Such methods are well known to those skilled in the art. Any of these can be used to alter and/or optimize the activity of a xylose transporter and/or an enzyme or protein of the xylose metabolic pathway. Such methods include, but are not limited to EpPCR, which introduces random point mutations by reducing the fidelity of DNA polymerase in PCR reactions (Pritchard et al., J Theor. Biol. 234:497-509 (2005)); Error-prone Rolling Circle Amplification (epRCA), which is similar to epPCR except a whole circular plasmid is used as the template and random 6-mers with exonuclease resistant thiophosphate linkages on the last 2 nucleotides are used to amplify the plasmid followed by transformation into cells in which the plasmid is re-circularized at tandem repeats (Fujii et al., Nucleic Acids Res. 32:e145 (2004); and Fujii et al., Nat. Protoc. 1:2493-2497 (2006)); DNA or Family Shuffling, which typically involves digestion of two or more variant genes with nucleases such as Dnase I or EndoV to generate a pool of random fragments that are reassembled by cycles of annealing and extension in the presence of DNA polymerase to create a library of chimeric genes (Stemmer, Proc Natl Acad Sci USA 91:10747-10751 (1994); and Stemmer, Nature 370:389-391 (1994)); Staggered Extension (StEP), which entails template priming followed by repeated cycles of 2 step PCR with denaturation and very short duration of annealing/extension (as short as 5 sec) (Zhao et al., Nat. Biotechnol. 16:258-261 (1998)); Random Priming Recombination (RPR), in which random sequence primers are used to generate many short DNA fragments complementary to different segments of the template (Shao et al., Nucleic Acids Res 26:681-683 (1998)).

Additional methods include Heteroduplex Recombination, in which linearized plasmid DNA is used to form heteroduplexes that are repaired by mismatch repair (Volkov et al, Nucleic Acids Res. 27:e18 (1999); and Volkov et al., Methods Enzymol. 328:456-463 (2000)); Random Chimeragenesis on Transient Templates (RACHITT), which employs Dnase I fragmentation and size fractionation of single stranded DNA (ssDNA) (Coco et al., Nat. Biotechnol. 19:354-359 (2001)); Recombined Extension on Truncated templates (RETT), which entails template switching of unidirectionally growing strands from primers in the presence of unidirectional ssDNA fragments used as a pool of templates (Lee et al., J. Molec. Catalysis 26:119-129 (2003)); Degenerate Oligonucleotide Gene Shuffling (DOGS), in which degenerate primers are used to control recombination between molecules; (Bergquist and Gibbs, Methods Mol. Biol 352:191-204 (2007); Bergquist et al., Biomol. Eng 22:63-72 (2005); Gibbs et al., Gene 271:13-20 (2001)); Incremental Truncation for the Creation of Hybrid Enzymes (ITCHY), which creates a combinatorial library with 1 base pair deletions of a gene or gene fragment of interest (Ostermeier et al., Proc. Natl. Acad. Sci. USA 96:3562-3567 (1999); and Ostermeier et al., Nat. Biotechnol. 17:1205-1209 (1999)); Thio-Incremental Truncation for the Creation of Hybrid Enzymes (THIO-ITCHY), which is similar to ITCHY except that phosphothioate dNTPs are used to generate truncations (Lutz et al., Nucleic Acids Res 29:E16 (2001)); SCRATCHY, which combines two methods for recombining genes, ITCHY and DNA shuffling (Lutz et al., Proc. Natl. Acad. Sci. USA 98:11248-11253 (2001)); Random Drift Mutagenesis (RNDM), in which mutations made via epPCR are followed by screening/selection for those retaining usable activity (Bergquist et al., Biomol. Eng. 22:63-72 (2005)); Sequence Saturation Mutagenesis (SeSaM), a random mutagenesis method that generates a pool of random length fragments using random incorporation of a phosphothioate nucleotide and cleavage, which is used as a template to extend in the presence of “universal” bases such as inosine, and replication of an inosine-containing complement gives random base incorporation and, consequently, mutagenesis (Wong et al., Biotechnol. 1 3:74-82 (2008); Wong et al., Nucleic Acids Res. 32:e26 (2004); and Wong et al., Anal. Biochem. 341:187-189 (2005)); Synthetic Shuffling, which uses overlapping oligonucleotides designed to encode “all genetic diversity in targets” and allows a very high diversity for the shuffled progeny (Ness et al., Nat. Biotechnol. 20:1251-1255 (2002)); Nucleotide Exchange and Excision Technology NexT, which exploits a combination of dUTP incorporation followed by treatment with uracil DNA glycosylase and then piperidine to perform endpoint DNA fragmentation (Muller et al., Nucleic Acids Res. 33:e117 (2005)).

Further methods include Sequence Homology-Independent Protein Recombination (SHIPREC), in which a linker is used to facilitate fusion between two distantly related or unrelated genes, and a range of chimeras is generated between the two genes, resulting in libraries of single-crossover hybrids (Sieber et al., Nat. Biotechnol. 19:456-460 (2001)); Gene Site Saturation Mutagenesis™ (GSSM™), in which the starting materials include a supercoiled double stranded DNA (dsDNA) plasmid containing an insert and two primers which are degenerate at the desired site of mutations (Kretz et al., Methods Enzymol. 388:3-11 (2004)); Combinatorial Cassette Mutagenesis (CCM), which involves the use of short oligonucleotide cassettes to replace limited regions with a large number of possible amino acid sequence alterations (Reidhaar-Olson et al. Methods Enzymol. 208:564-586 (1991); and Reidhaar-Olson et al. Science 241:53-57 (1988)); Combinatorial Multiple Cassette Mutagenesis (CMCM), which is essentially similar to CCM and uses epPCR at high mutation rate to identify hot spots and hot regions and then extension by CMCM to cover a defined region of protein sequence space (Reetz et al., Angew. Chem. Int. Ed Engl. 40:3589-3591 (2001)); the Mutator Strains technique, in which conditional is mutator plasmids, utilizing the mutD5 gene, which encodes a mutant subunit of DNA polymerase III, to allow increases of 20 to 4000-X in random and natural mutation frequency during selection and block accumulation of deleterious mutations when selection is not required (Selifonova et al., Appl. Environ. Microbiol. 67:3645-3649 (2001)); Low et al., J. Mol. Biol. 260:359-3680 (1996)).

Additional exemplary methods include Look-Through Mutagenesis (LTM), which is a multidimensional mutagenesis method that assesses and optimizes combinatorial mutations of selected amino acids (Rajpal et al., Proc. Natl. Acad. Sci. USA 102:8466-8471 (2005)); Gene Reassembly, which is a DNA shuffling method that can be applied to multiple genes at one time or to create a large library of chimeras (multiple mutations) of a single gene (Tunable GeneReassembly™ (TGR™) Technology supplied by Verenium Corporation), in Silico Protein Design Automation (PDA), which is an optimization algorithm that anchors the structurally defined protein backbone possessing a particular fold, and searches sequence space for amino acid substitutions that can stabilize the fold and overall protein energetics, and generally works most effectively on proteins with known three-dimensional structures (Hayes et al., Proc. Natl. Acad. Sci. USA 99:15926-15931 (2002)); and Iterative Saturation Mutagenesis (ISM), which involves using knowledge of structure/function to choose a likely site for enzyme or protein improvement, performing saturation mutagenesis at chosen site using a mutagenesis method such as Stratagene QuikChange (Stratagene; San Diego Calif.), screening/selecting for desired properties, and, using improved clone(s), starting over at another site and continue repeating until a desired activity is achieved (Reetz et al., Nat. Protoc. 2:891-903 (2007); and Reetz et al., Angew. Chem. Int. Ed Engl. 45:7745-7751 (2006)).

Any of the aforementioned methods for mutagenesis can be used alone or in any combination. Additionally, any one or combination of the directed evolution methods can be used in conjunction with adaptive evolution techniques, as described herein or otherwise known in the art.

Provided herein are also isolated nucleic acids encoding the Metschnikowia xylose transporters or enzyme or protein of xylose metabolic pathway described herein, as well as the variants thereof. Nucleic acids provided herein include those having the nucleic acid sequence provided in the sequence listing; those that hybridize to the nucleic acid sequences provided in the sequence listing, under high stringency hybridization conditions (for example, 42°, 2.5 hr., 6×SCC, 0.1% SDS); and those having substantial nucleic acid sequence identity with the nucleic acid sequence provided in the sequence listing. The nucleic acids provided herein also encompass equivalent substitutions of codons that can be translated to produce the same amino acid sequences. Provided herein are also vectors including the nucleic acids described herein. The vector can be an expression vector suitable for expression in a host microbial organism. The vector can be a 2μ vector. The vector can be an ARS vector.

The nucleic acids provided herein include those encoding xylose transporters or enzymes or proteins of xylose metabolic pathway having an amino acid sequence as described herein, as well as their variants that retain transporter or respective enzymatic activity. The nucleic acids provided herein can be cDNA, chemically synthesized DNA, DNA amplified by PCR, RNA, or combinations thereof. Due to the degeneracy of the genetic code, two DNA sequences can differ and yet encode identical amino acid sequences.

Provided herein are also functional fragments of nucleic acids encoding the Metschnikowia xylose transporters or enzyme or protein of xylose metabolic pathway described herein, include probes and primers. Such probes and primers can be used, for example, in PCR methods to amplify or detect the presence of nucleic acids encoding the Metschnikowia xylose transporters or enzyme or protein of xylose metabolic pathway in vitro, as well as in Southern and Northern blots for analysis. Cells expressing the Metschnikowia xylose transporters or enzyme or protein of xylose metabolic pathway an also be identified by the use of such probes. Methods for the production and use of such primers and probes are known.

Provided herein are also fragments of nucleic acids encoding the Metschnikowia xylose transporters or enzymes or proteins of xylose metabolic pathway that are antisense or sense oligonucleotides having a single-stranded nucleic acid capable of binding to a target mRNA or DNA sequence of a Metschnikowia xylose transporter or enzyme or protein of xylose metabolic pathway.

A nucleic acid encoding a xylose transporter or an enzyme or protein of xylose metabolic pathway described herein can include nucleic acids that hybridize to a nucleic acid disclosed herein by SEQ ID NO, GenBank and/or GI number or a nucleic acid molecule that hybridizes to a nucleic acid molecule that encodes an amino acid sequence disclosed herein by SEQ ID NO, GenBank and/or GI number. Hybridization conditions can include highly stringent, moderately stringent, or low stringency hybridization conditions that are well known to one of skill in the art such as those described herein.

Stringent hybridization refers to conditions under which hybridized polynucleotides are stable. As known to those of skill in the art, the stability of hybridized polynucleotides is reflected in the melting temperature (Tₘ) of the hybrids. In general, the stability of hybridized polynucleotides is a function of the salt concentration, for example, the sodium ion concentration and temperature. A hybridization reaction can be performed under conditions of lower stringency, followed by washes of varying, but higher, stringency. Reference to hybridization stringency relates to such washing conditions. Highly stringent hybridization includes conditions that permit hybridization of only those nucleic acid sequences that form stable hybridized polynucleotides in 0.018M NaCl at 65° C., for example, if a hybrid is not stable in 0.018M NaCl at 65° C., it will not be stable under high stringency conditions, as contemplated herein. High stringency conditions can be provided, for example, by hybridization in 50% formamide, 5X Denhart's solution, 5× SSPE, 0.2% SDS at 42° C., followed by washing in 0.1× SSPE, and 0.1% SDS at 65° C. Hybridization conditions other than highly stringent hybridization conditions can also be used to describe the nucleic acid sequences disclosed herein. For example, the phrase moderately stringent hybridization refers to conditions equivalent to hybridization in 50% formamide, 5× Denhart's solution, 5× SSPE, 0.2% SDS at 42° C., followed by washing in 0.2× SSPE, 0.2% SDS, at 42° C. The phrase low stringency hybridization refers to conditions equivalent to hybridization in 10% formamide, 5× Denhart's solution, 6X SSPE, 0.2% SDS at 22° C., followed by washing in 1×SSPE, 0.2% SDS, at 37° C. Denhart's solution contains 1% Ficoll, 1% polyvinylpyrolidone, and 1% bovine serum albumin (BSA). 20× SSPE (sodium chloride, sodium phosphate, ethylene diamide tetraacetic acid (EDTA)) contains 3M sodium chloride, 0.2M sodium phosphate, and 0.025M (EDTA). Other suitable low, moderate and high stringency hybridization buffers and conditions are well known to those of skill in the art and are described, for example, in Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, Md. (1999).

Nucleic acids encoding a xylose transporter or an enzyme or protein of xylose metabolic pathway provided herein include those having a certain percent sequence identity to a nucleic acid disclosed herein by SEQ ID NO, GenBank and/or GI number. For example, the nucleic acids encoding a xylose transporter can have at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity, or be identical, to a nucleic acid described herein by SEQ ID NO, GenBank and/or GI number. In some embodiments, the nucleic acid molecule can have 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity, or be identical, to a sequence selected from SEQ ID NOs: 10-17, 19-27 and 33-37.

The xylose transporter or enzyme or protein of xylose metabolic pathway provided herein can be isolated by a variety of methods well-known in the art, for example, recombinant expression systems, precipitation, gel filtration, ion-exchange, reverse-phase and affinity chromatography, and the like. Other well-known methods are described in Deutscher et al., Guide to Protein Purification: Methods in Enzymology, Vol. 182 (Academic Press, (1990)). Alternatively, the isolated xylose transporter or enzyme or protein of xylose metabolic pathway provided herein can be obtained using well-known recombinant methods (see, for example, Sambrook et al., supra, 1989; Ausubel et al., supra, 1999). The methods and conditions for biochemical purification of the isolated xylose transporter or enzyme or protein of xylose metabolic pathway provided herein can be chosen by those skilled in the art, and purification monitored, for example, by a functional assay.

One non-limiting example of a method for preparing the xylose transporter or enzyme or protein of xylose metabolic pathway is to express nucleic acids encoding the xylose transporter or enzyme or protein of xylose metabolic pathway in a suitable host cell, such as a bacterial cell, a yeast cell, or other suitable cell, using methods well known in the art, and recovering the expressed xylose transporter or enzyme or protein of xylose metabolic pathway, again using well-known purification methods, as described herein. The xylose transporter or enzyme or protein of xylose metabolic pathway provided herein can be isolated directly from cells that have been transformed with expression vectors as described herein. Recombinantly expressed xylose transporters or enzymes or proteins of xylose metabolic pathway can also be expressed as fusion proteins with appropriate affinity tags, such as glutathione S transferase (GST), poly His, streptavidin, and the like, and affinity purified, if desired. The polypeptide of the xylose transporters or enzymes or proteins of xylose metabolic pathway described herein can retain the affinity tag, if desired, or optionally the affinity tag can be removed from the polypeptide using well known methods to remove an affinity tag, for example, using appropriate enzymatic or chemical cleavage. Thus, provided herein are polypeptide of xylose transporters or enzymes or proteins of xylose metabolic pathway without or optionally with an affinity tag. Accordingly, in some embodiments, provided herein is a host cell expressing a polypeptide of the xylose transporters or enzymes or proteins of xylose metabolic pathway herein. A polypeptide of the xylose transporter or enzyme or protein of xylose metabolic pathway described herein can also be produced by chemical synthesis using a method of polypeptide synthesis well know to one of skill in the art.

In some embodiments, provided herein are methods of constructing a host strain that can include, among other steps, introducing a vector disclosed herein into a host cell that is capable of fermentation. Vectors of the invention can be introduced stably or transiently into a host cell using techniques well known in the art including, but not limited to, conjugation, electroporation, chemical transformation, transduction, transfection, and ultrasound transformation. Additional methods are disclosed herein, any one of which can be used in the method of the invention.

Provided herein are also vectors containing the nucleic acid molecules encoding xylose transporters and/or enzymes or proteins of xylose metabolic pathway, as well as host cells transformed with such vectors. Any of the polynucleotide molecules of the disclosure can be contained in a vector, which generally includes a selectable marker and an origin of replication, for propagation in a host. The vectors can further include suitable transcriptional or translational regulatory sequences, such as those derived from a mammalian, fungal, bacterial, viral, or insect genes, operably linked to the polynucleotide molecule that encode xylose transporter or enzyme or protein of xylose metabolic pathway. Examples of such regulatory sequences include transcriptional promoters, operators, or enhancers, mRNA ribosomal binding sites, and appropriate sequences which control transcription and translation. Nucleotide sequences are operably linked when the regulatory sequence functionally relates to the DNA encoding the target protein. Thus, a promoter nucleotide sequence is operably linked to a xylose transporter or enzyme or protein of xylose metabolic pathway if the promoter nucleotide sequence directs the transcription of sequence of the transporter or enzyme or protein.

Selection of suitable vectors for the cloning of nucleic acid molecules of this disclosure depends upon the host cell in which the vector will be transformed, and, where applicable, the host cell from which the enzyme or protein is to be expressed. Suitable host cells for expression of xylose transporter and/or enzyme or protein of xylose metabolic pathway include prokaryotes and yeasts, which are discussed below. Selection of suitable combinations of vectors and host organisms is a routine matter from a perspective of skill.

The xylose transporter and/or enzyme or protein of xylose metabolic pathway to be expressed in such host cells can also be fusion proteins that include sequences from other proteins. As discussed above, such regions can be included to allow, for example, enhanced functionality, improved stability, or facilitated purification. For example, a nucleic acid sequence encoding a peptide that binds strongly to xylose can be fused in-frame to the transmembrane sequence of a xylose transporter so that the resulting fusion protein binds xylose and transports the sugar across the cell membrane at a higher rate than the wild type transporter.

While generally described herein as a microbial organism that contains a xylose-ethanol pathway and at least one exogenous nucleic acid encoding enzyme or protein expressed in a sufficient amount to confer or enhance xylose uptake or metabolism for ethanol production, it is understood that the disclosure additionally provides a non-naturally occurring microbial organism having at least one exogenous nucleic acid encoding enzyme or protein expressed in a sufficient amount to confer or enhance xylose uptake or metabolism for the production of an intermediate of a xylose-ethanol pathway. For example, as disclosed herein, ethanol can be produced through either PPP or the glycolysis pathway with either glyceraldehyde-3-phosphate or pyruvate as an intermediate. Therefore, the microbial organisms provided herein can also have at least one exogenous nucleic acid encoding enzyme or protein expressed in a sufficient amount to confer or enhance xylose uptake or metabolism, where the microbial organism produces a xylose-ethanol pathway intermediate, for example, xylitol, xylulose, glycerol, succinate, pyruvate, sedoheptulose-7-phosphate, glyceraldehyde-3-phosphate, acetyl-CoA, or acetate from xylose.

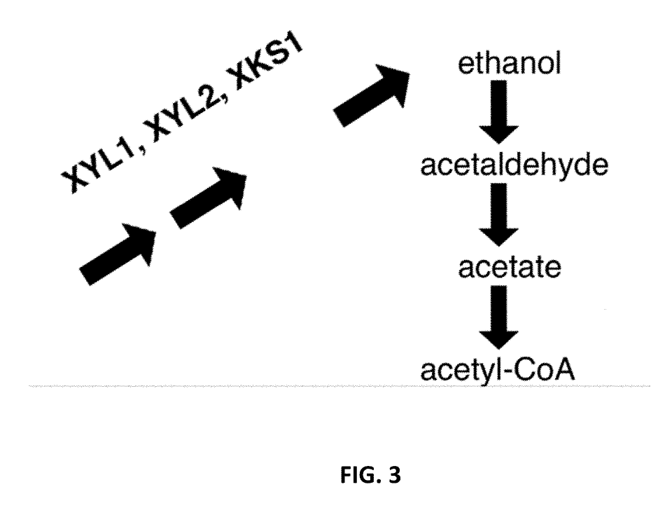

The ethanol production by the non-naturally occurring microbial organism provided herein can be further improved by additional genetic modifications to enhance the xylose metabolism or reduce the ethanol metabolism. As provided in FIG. 2, increased xylose metabolism can increase the PPP activity and glycolysis activity, both can lead to increased production of ethanol. As provided in FIG. 3, reduced ethanol metabolism can reduce the conversion of ethanol to other metabolites, resulting in the increased production of ethanol. Thus, it is understood by those skilled in the art that microbial organisms provided herein can include additional genetic modifications as described herein or otherwise known in the art to further enhance xylose metabolism or reduce ethanol metabolism.

For example, the non-naturally occurring microbial organism provided herein can have attenuated phosphatase Pho13p. (U.S. Pat. No. 7,285,403; Xu et al., Metabolic engineering 34: 88-96 (2016).) In some embodiments, the microbial organism can have a disruption in the gene encoding Pho13p. Pho13p is an alkaline phosphatase, has shown significant hydrolytic activity against a broad spectrum of substrates including p-nitrophenylphosphate, phosphorylated histone II-A and casein. (Tuleva et al., FEMS Microbiol Lett. 161:139-44 (1998).) The deletion of PHO13 (pho13Δ) in S. cerevisiae induced TAL1 upregulation. (Xu et al., 2016.) As shown in FIG. 2, limited transaldolase activity leads to sedoheptulose accumulation, and the TAL1 upregulation can improve xylose metabolism.

A BLAST analysis of Pho13p identified 13 closely related proteins produced by Debaryomyces hansenii, Gibberella zeae, Ustilago maydis, Schizosaccharomyces pombe, Neurospora crassa, Candida albicans, Yarrowia hpolytica, Ashbya gossypii, Kluyveromyces lactis, Candida glabrata, and Saccharomyces cerevisiae. More than 100 similar proteins are known from other organisms. The non-naturally occurring microbial organisms provided herein having attenuated or disrupted PHO13 can have improved xylose fermentation. With any given host microbial organism, the gene sequence encoding Pho13p ortholog can be disrupted using homologous recombination for those species for which a homologous putative translation product is known. Alternatively, the Pho13p ortholog p-nitrophenylphosphatase can be identified from a genomic library using degenerative oligonucleotide probes and sequenced or partially sequence to permit design of a disruption cassette to disrupt the in vivo coding sequence.

The non-naturally occurring microbial organism provided herein can also have attenuated ethanol metabolism. For example, the microbial organisms provided herein can also have attenuated ethanol dehydrogenase, attenuated acetaldehyde dehydrogenase, attenuated acetate coA-transferase, or any combination thereof (See FIG. 3.) In some embodiments, the microbial organisms provided herein can have attenuated ethanol dehydrogenase. Adh2p is an alcohol dehydrogenase that targets ethanol. In some embodiments, the microbial organisms provided herein can have attenuated acetaldehyde dehydrogenase. Ald2p is a dehydrogenase that targets acetaldehyde. In some embodiments, the microbial organisms provided herein can have attenuated acetate coA-transferase. In some embodiments, the microbial organisms provided herein can have attenuated ethanol dehydrogenase and attenuated acetaldehyde dehydrogenase. In some embodiments, the microbial organisms provided herein can have attenuated ethanol dehydrogenase and attenuated acetate coA-transferase. In some embodiments, the microbial organisms provided herein can have attenuated acetaldehyde dehydrogenase and attenuated acetate coA-transferase. In some embodiments, the microbial organisms provided herein can have attenuated ethanol dehydrogenase, attenuated acetaldehyde dehydrogenase and attenuated acetate coA-transferase. The ethanol dehydrogenase can be Adh2p. The acetaldehyde dehydrogenase can be Ald2p. The acetate coA-transferase can be Acs1p.

In some embodiments, the microbial organisms provided herein can have a disruption in the gene encoding ethanol dehydrogenase. In some embodiments, the microbial organisms provided herein can have a disruption in the gene encoding acetaldehyde dehydrogenase. In some embodiments, the microbial organisms provided herein can have a disruption in the gene encoding acetate coA-transferase. In some embodiments, the microbial organisms provided herein can have a disruption in the genes encoding ethanol dehydrogenase and acetaldehyde dehydrogenase. In some embodiments, the microbial organisms provided herein can have a disruption in the genes encoding ethanol dehydrogenase and acetate coA-transferase. In some embodiments, the microbial organisms provided herein can have a disruption in the genes encoding acetaldehyde dehydrogenase and acetate coA-transferase. In some embodiments, the microbial organisms provided herein can have a disruption in the genes encoding ethanol dehydrogenase, acetaldehyde dehydrogenase and acetate coA-transferase. The ethanol dehydrogenase can be Adh2p, The acetaldehyde dehydrogenase can be Ald2p. The acetate coA-transferase can be Acs1p.

It is understood that, if a genetic modification is to be introduced into a host organism to disrupt a gene, such as one encoding ethanol dehydrogenase (e.g. Adh2p), acetaldehyde dehydrogenase (e.g. Ald2p) or acetate coA-transferase (e.g. Acs1p), any homologs, orthologs or paralogs that catalyze similar, yet non-identical metabolic reactions can similarly be disrupted to ensure that a desired metabolic reaction is sufficiently disrupted. Because certain differences exist among metabolic networks between different organisms, those skilled in the art will understand that the actual genes disrupted in a given organism may differ between organisms. However, given the teachings and guidance provided herein, as well as the genetic information known in the art, those skilled in the art also will understand that the methods provided herein can be applied to any suitable host microorganism to identify the cognate metabolic alterations needed to construct an organism in a species of interest that will increase ethanol biosynthesis. In a particular embodiment, the increased production couples biosynthesis of ethanol to growth of the organism, and can obligatorily couple production of ethanol to growth of the organism if desired and as disclosed herein.

The invention provides non naturally occurring microbial organisms having genetic alterations such as gene disruptions that increase production of ethanol, for example, growth-coupled production of ethanol. Product production can be, for example, obligatorily linked to the exponential growth phase of the microorganism by genetically altering the metabolic pathways of the cell, as disclosed herein. The genetic alterations can increase the production of ethanol or even make the ethanol an obligatory product during the growth phase.

Given the teachings and guidance provided herein, those skilled in the art will understand that to introduce a metabolic alteration such as attenuation of an enzyme or protein, it can be necessary to disrupt the catalytic activity of the one or more enzymes involved in the reaction. Alternatively, a metabolic alteration can include disrupting expression of a regulatory protein or cofactor necessary for enzyme activity or maximal activity. Furthermore, genetic loss of a cofactor necessary for an enzymatic reaction can also have the same effect as a disruption of the gene encoding the enzyme or protein. Disruption can occur by a variety of methods including, for example, deletion of an encoding gene or incorporation of a genetic alteration in one or more of the encoding gene sequences. The encoding genes targeted for disruption can be one, some, or all of the genes encoding enzymes involved in the catalytic activity. For example, where a single enzyme is involved in a targeted catalytic activity, disruption can occur by a genetic alteration that reduces or eliminates the catalytic activity of the encoded gene product. Similarly, where the single enzyme is multimeric, including heteromeric, disruption can occur by a genetic alteration that reduces or destroys the function of one or all subunits of the encoded gene products. Destruction of activity can be accomplished by loss of the binding activity of one or more subunits required to form an active complex, by destruction of the catalytic subunit of the multimeric complex or by both. Other functions of multimeric protein association and activity also can be targeted in order to disrupt a metabolic reaction of the invention. Such other functions are well known to those skilled in the art. Similarly, a target enzyme or protein activity can be reduced or eliminated by disrupting expression of a protein or enzyme that modifies and/or activates the target enzyme, for example, a molecule required to convert an apoenzyme to a holoenzyme. Further, some or all of the functions of a single polypeptide or multimeric complex can be disrupted according to the invention in order to reduce or abolish the catalytic activity of one or more enzymes or proteins involved in a reaction or metabolic modification of the invention. Similarly, some or all of enzymes or proteins involved in a reaction or metabolic modification of the invention can be disrupted so long as the targeted reaction is reduced or eliminated.

Given the teachings and guidance provided herein, those skilled in the art also will understand that an enzymatic reaction can be disrupted by reducing or eliminating reactions encoded by a common gene and/or by one or more orthologs of that gene exhibiting similar or substantially the same activity. Reduction of both the common gene and all orthologs can lead to complete abolishment of any catalytic activity of a targeted reaction. However, disruption of either the common gene or one or more orthologs can lead to a reduction in the catalytic activity of the targeted reaction sufficient to promote coupling of growth to product biosynthesis. Exemplified herein are both the common genes encoding catalytic activities for a variety of metabolic modifications as well as their orthologs. Those skilled in the art will understand that disruption of some or all of the genes encoding an enzyme or protein of a targeted metabolic reaction can be practiced in the methods of the invention and incorporated into the non-naturally occurring microbial organisms of the invention in order to achieve the increased production of ethanol or growth-coupled product production.

Given the teachings and guidance provided herein, those skilled in the art also will understand that enzymatic activity or expression can be attenuated using well known methods. Reduction of the activity or amount of an enzyme or protein can mimic complete disruption of a gene if the reduction causes activity of the enzyme or protein to fall below a critical level that is normally required for a pathway to function. Reduction of enzymatic activity by various techniques rather than use of a gene disruption can be important for an organism's viability. Methods of reducing enzymatic activity that result in similar or identical effects of a gene disruption include, but are not limited to: reducing gene transcription or translation; destabilizing mRNA, protein or catalytic RNA; and mutating a gene that affects enzyme or protein activity or kinetics (See, Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, Md. (1999). Natural or imposed regulatory controls can also accomplish enzyme or protein attenuation including: promoter replacement (See, Wang et al., Mol. Biotechnol. 52(2):300-308 (2012)); loss or alteration of transcription factors (Dietrick et al., Annu. Rev. Biochem. 79:563-590 (2010); and Simicevic et al., Mol. Biosyst. 6(3):462-468 (2010)); introduction of inhibitory RNAs or peptides such as siRNA, antisense RNA, RNA or peptide/small-molecule binding aptamers, ribozymes, aptazymes and riboswitches (Wieland et al., Methods 56(3):351-357 (2012); O'Sullivan, Anal. Bioanal. Chem. 372(1):44-48 (2002); and Lee et al., Curr. Opin. Biotechnol. 14(5):505-511 (2003)); and addition of drugs or other chemicals that reduce or disrupt enzymatic activity such as an enzyme or protein inhibitor, an antibiotic or a target-specific drug.

One skilled in the art will also understand and recognize that attenuation of an enzyme or protein can be done at various levels. For example, at the gene level, a mutation causing a partial or complete null phenotype, such as a gene disruption, or a mutation causing epistatic genetic effects that mask the activity of a gene product (Miko, Nature Education 1(1) (2008)), can be used to attenuate an enzyme or protein. At the gene expression level, methods for attenuation include: coupling transcription to an endogenous or exogenous inducer, such as isopropylthio-β-galactoside (IPTG), then adding low amounts of inducer or no inducer during the production phase (Donovan et al., J. Ind. Microbiol. 16(3):145-154 (1996); and Hansen et al., Curr. Microbiol. 36(6):341-347 (1998)); introducing or modifying a positive or a negative regulator of a gene; modify histone acetylation/deacetylation in a eukaryotic chromosomal region where a gene is integrated (Yang et al., Curr. Opin. Genet. Dev. 13(2):143-153 (2003) and Kurdistani et al., Nat. Rev. Mol. Cell Biol. 4(4):276-284 (2003)); introducing a transposition to disrupt a promoter or a regulatory gene (Bleykasten-Brosshans et al., C. R. Biol. 33(8-9):679-686 (2011); and McCue et al., PLoS Genet. 8(2):e1002474 (2012)); flipping the orientation of a transposable element or promoter region so as to modulate gene expression of an adjacent gene (Wang et al., Genetics 120(4):875-885 (1988); Hayes, Annu. Rev. Genet. 37:3-29 (2003); in a diploid organism, deleting one allele resulting in loss of heterozygosity (Daigaku et al., Mutation Research/Fundamental and Molecular Mechanisms of Mutagenesis 600(1-2)177-183 (2006)); introducing nucleic acids that increase RNA degradation (Houseley et al., Cell, 136(4):763-776 (2009); or in bacteria, for example, introduction of a transfer-messenger RNA (tmRNA) tag, which can lead to RNA degradation and ribosomal stalling (Sunohara et al., RNA 10(3):378-386 (2004); and Sunohara et al., J. Biol. Chem. 279:15368-15375 (2004)). At the translational level, attenuation can include: introducing rare codons to limit translation (Angov, Biotechnol. J. 6(6):650-659 (2011)); introducing RNA interference molecules that block translation (Castel et al., Nat. Rev. Genet. 14(2):100-112 (2013); and Kawasaki et al., Curr. Opin. Mol. Ther. 7(2):125-131 (2005); modifying regions outside the coding sequence, such as introducing secondary structure into an untranslated region (UTR) to block translation or reduce efficiency of translation (Ringner et al., PLoS Comput. Biol. 1(7):e72 (2005)); adding RNAase sites for rapid transcript degradation (Pasquinelli, Nat. Rev. Genet. 13(4):271-282 (2012); and Arraiano et al., FEMS Microbiol. Rev. 34(5):883-932 (2010); introducing antisense RNA oligomers or antisense transcripts (Nashizawa et al., Front. Biosci. 17:938-958 (2012)); introducing RNA or peptide aptamers, ribozymes, aptazymes, riboswitches (Wieland et al., Methods 56(3):351-357 (2012); O'Sullivan, Anal. Bioanal. Chem. 372(1):44-48 (2002); and Lee et al., Curr. Opin. Biotechnol. 14(5):505-511 (2003)); or introducing translational regulatory elements involving RNA structure that can prevent or reduce translation that can be controlled by the presence or absence of small molecules (Araujo et al., Comparative and Functional Genomics, Article ID 475731, 8 pages (2012)). At the level of enzyme localization and/or longevity, enzyme or protein attenuation can include: adding a degradation tag for faster protein turnover (Hochstrasser, Annual Rev. Genet. 30:405-439 (1996); and Yuan et al., PLoS One 8(4):e62529 (2013)); or adding a localization tag that results in the enzyme or protein being secreted or localized to a subcellular compartment in a eukaryotic cell, where the enzyme or protein would not be able to react with its normal substrate (Nakai et al. Genomics 14(4):897-911 (1992); and Russell et al., J. Bact. 189(21)7581-7585 (2007)). At the level of post-translational regulation, enzyme attenuation can include: increasing intracellular concentration of known inhibitors; or modifying post-translational modified sites (Mann et al., Nature Biotech. 21:255-261 (2003)). At the level of enzyme or protein activity, enzyme or protein attenuation can include: adding an endogenous or an exogenous inhibitor, such as an enzyme or protein inhibitor, an antibiotic or a target-specific drug, to reduce enzyme or protein activity; limiting availability of essential cofactors, such as vitamin B12, for an enzyme or protein that requires the cofactor; chelating a metal ion that is required for enzyme or protein activity; or introducing a dominant negative mutation. The applicability of a technique for attenuation described above can depend upon whether a given host microbial organism is prokaryotic or eukaryotic, and it is understood that a determination of what is the appropriate technique for a given host can be readily made by one skilled in the art.

In some embodiments, a candidate gene is silenced using Clustered Regularly Interspaced Short Palindromic Repeats (CRISPR)/CAS9 system described, for example, in Wiedenheft et al., Nature 482 (7385): 331-8 (2012). An engineered, programmable, non-naturally occurring CRISPR-Cas system including a Cas9 protein and one or more guide RNAs that target the genomic loci of DNA molecules encoding one or more gene products in a eukaryotic cell is provided, and the Cas9 protein cleaves the genomic loci of the DNA molecules encoding the one or more gene products, altering expression of the one or more gene products. (See e.g. U.S. Pat. No. 8,697,359).

In some embodiments, microaerobic designs can be used based on the growth-coupled formation of the desired product. To examine this, production cones can be constructed for each strategy by first maximizing and, subsequently minimizing the product yields at different rates of biomass formation feasible in the network. If the rightmost boundary of all possible phenotypes of the mutant network is a single point, it implies that there is a unique optimum yield of the product at the maximum biomass formation rate possible in the network. In other cases, the rightmost boundary of the feasible phenotypes is a vertical line, indicating that at the point of maximum biomass the network can make any amount of the product in the calculated range, including the lowest amount at the bottommost point of the vertical line. Such designs are given a low priority.

The ethanol-production strategies identified by the methods disclosed herein such as the OptKnock framework are generally ranked on the basis of their (i) theoretical yields, and (ii) growth-coupled ethanol formation characteristics. Accordingly, the invention also provides a non-naturally occurring microbial organism having a set of metabolic modifications coupling ethanol production to growth of the organism.

Each of the strains can be supplemented with additional deletions if it is determined that the strain designs do not sufficiently increase the production of ethanol and/or couple the formation of the product with biomass formation. Alternatively, some other enzymes or proteins not known to possess significant activity under the growth conditions can become active due to adaptive evolution or random mutagenesis. Such activities can also be knocked out. However, the list of gene deletion sets disclosed herein allows the construction of strains exhibiting high-yield production of ethanol, including growth-coupled production of ethanol.

The non-naturally occurring microbial organisms provided herein can be produced by introducing expressible nucleic acids encoding one or more of xylose transporters, enzymes or proteins of the xylose metabolic pathway, and/or enzymes or proteins of in one or more ethanol biosynthetic pathways. Depending on the host microbial organism chosen for biosynthesis, nucleic acids for some or all of a particular xylose-ethano; biosynthetic pathway can be expressed. For example, if a chosen host is deficient in one or more enzymes or proteins for a desired biosynthetic pathway, then expressible nucleic acids for the deficient enzyme(s) or protein(s) are introduced into the host for subsequent exogenous expression. Alternatively, if the chosen host exhibits endogenous expression of some pathway genes, but is deficient in others, then an encoding nucleic acid is needed for the deficient enzyme(s) or protein(s) to achieve ethanol biosynthesis. Thus, a non-naturally occurring microbial organism provided herein can be produced by introducing exogenous enzyme or protein activities to obtain a desired biosynthetic pathway or a desired biosynthetic pathway can be obtained by introducing one or more exogenous enzyme or protein activities that, together with one or more endogenous enzymes or proteins, produces ethanol from xylose.

A relatively small number of wild type microorganisms can ferment D-xylose. These microorganisms are generally not suitable for large-scale fermentation. This unfavorability can arise, for example, as a result of unfamiliarity with the microorganisms, difficulty obtaining the microorganisms, poor productivity and/or growth on pretreated lignocellulosics or unsatisfactory yield when grown on mixed sugars derived from biomass. (C. Abbas, “Lignocellulosics to ethanol: meeting ethanol demand in the future,” The Alcohol Textbook, 4^{th} Edition. (K. A. Jacques, T. P. Lyons and D. R. Kelsall, eds). Nottingham University Press, Nottingham, UK, 2003, pp. 41-57.; C. Abbas, “Emerging biorefineries and biotechnological applications of nonconventional yeast: now and in the future,” The Alcohol Textbook, 4^{th} Edition. (K. A. Jacques, T. P. Lyons and D. R. Kelsall, eds). Nottingham University Press, Nottingham, United Kingdom, 2003, pp. 171-191).

Host microbial organisms can be selected from, and the non-naturally occurring microbial organisms generated in, for example, bacteria, yeast, fungus or any of a variety of other microorganisms applicable or suitable to fermentation processes. Similarly, exemplary species of yeast or fungi species include any species selected from the order Saccharomycetales, family Saccaromycetaceae, including the genera Saccharomyces, Debaryomyces, Candida, Kluyveromyces and Pichia; the order Saccharomycetales, family Dipodascaceae, including the genus Yarrowia; the order Schizosaccharomycetales, family Schizosaccaromycetaceae, including the genus Schizosaccharomyces; the order Eurotiales, family Trichocomaceae, including the genus Aspergillus; and the order Mucorales, family Mucoraceae, including the genus Rhizopus. Non-limiting species of host yeast or fungi include S. cerevisiae, C. albicans, C. tropicalis, D. hansenii, S. pombe, K. lactis, K marxianus, A. terreus, A. niger, C. reinhardtii, P. pastoris, R. arrhizus, R. oryzae, T reesei, Y. lipolytica, and the like.

The xylose transporters described herein can also be expressed in yeast host cells from genera including Saccharomyces, Pichia, and Kluveromyces. In one embodiment, the yeast host is S. cerevisiae. Yeast vectors can contain an origin of replication sequence from a 2μ yeast plasmid for high copy vectors and a CEN sequence for a low copy number vector. Other sequences on a yeast vector can include an autonomously replicating sequence (ARS), a promoter region, sequences for polyadenylation, sequences for transcription termination, and a selectable marker gene. In some embodiments, vectors are replicable in both yeast and bacteria such as E. coli (termed shuttle vectors). In addition to the above-mentioned features of yeast vectors, a shuttle vector also includes sequences for replication and selection in bacteria such as E. coli.

Exemplary bacteria include any species selected from the order Enterobacteriales, family Enterobacteriaceae, including the genera Escherichia and Klebsiella; the order Aeromonadales, family Succinivibrionaceae, including the genus Anaerobiospirillum; the order Pasteurellales, family Pasteurellaceae, including the genera Actinobacillus and Mannheimia; the order Rhizobiales, family Bradyrhizobiaceae, including the genus Rhizobium; the order Bacillales, family Bacillaceae, including the genus Bacillus; the order Actinomycetales, families Corynebacteriaceae and Streptomycetaceae, including the genus Corynebacterium and the genus Streptomyces, respectively; order Rhodospirillales, family Acetobacteraceae, including the genus Gluconobacter; the order Sphingomonadales, family Sphingomonadaceae, including the genus Zymomonas; the order Lactobacillales, families Lactobacillaceae and Streptococcaceae, including the genus Lactobacillus and the genus Lactococcus, respectively; the order Clostridiales, family Clostridiaceae, genus Clostridium; and the order Pseudomonadales, family Pseudomonadaceae, including the genus Pseudomonas. Non-limiting species of host bacteria include E. coli, K. oxytoca, A. succiniciproducens, A. succinogenes, M. succiniciproducens, R. etli, B. subtilis, C. glutamicum, G. oxydans, Z. mobilis, L. lactis, L. plantarum, S. coelicolor, C. acetobutylicum, P. fluorescens, and P. putida.

Expression vectors for use in prokaryotic hosts generally comprise one or more phenotypic selectable marker genes. Such genes encode, for example, a protein that confers antibiotic resistance or that supplies an auxotrophic requirement. A wide variety of such vectors are readily available from commercial sources. Examples include pSPORT vectors, pGEM vectors (Promega, Madison, Wis.), pPROEX vectors (LTI, Bethesda, Md.), Bluescript vectors (Stratagene), and pQE vectors (Qiagen).

Insect host cell culture systems can also be used for the expression of the xylose transporters described herein. The target xylose transporters can be expressed using a baculovirus expression system, as described, for example, in the review by Luckow and Summers, 1988.

S. cerevisiae is a particularly useful host organism since it is a well characterized microbial organism suitable for genetic engineering. Other particularly useful host organisms include bacteria such as E. coli.

Depending on the xylose-ethanol biosynthetic pathway constituents of a selected host microbial organism, the non-naturally occurring microbial organisms provided herein can include at least one exogenously expressed xylose-ethanol pathway-encoding nucleic acid and up to all encoding nucleic acids for one or more xylose-ethanol biosynthetic pathways. For example, ethanol biosynthesis can be established in a host deficient in a pathway enzyme or protein through exogenous expression of the corresponding encoding nucleic acid. In a host deficient in all enzymes or proteins of a xylose-ethanol pathway, exogenous expression of all enzyme or proteins in the pathway can be included, although it is understood that all enzymes or proteins of a pathway can be expressed even if the host contains at least one of the pathway enzymes or proteins. For example, exogenous expression of all enzymes or proteins in a pathway for production of ethanol from xylose can be included in a host microbial organism.

Given the teachings and guidance provided herein, those skilled in the art will understand that the number of encoding nucleic acids to introduce in an expressible form will, at least, parallel the xylose-ethanol pathway deficiencies of the selected host microbial organism. Therefore, a non-naturally occurring microbial organism of the invention can have one, two, three, four, five, six, seven, eight, nine, ten, to all nucleic acids encoding the enzymes or proteins constituting a xylose-ethanol biosynthetic pathway disclosed herein. In some embodiments, the non-naturally occurring microbial organisms also can include other genetic modifications that facilitate or optimize ethanol biosynthesis or that confer other useful functions onto the host microbial organism.

Generally, a host microbial organism is selected such that it produces the precursor of a xylose-ethanol pathway, either as a naturally produced molecule or as an engineered product that either provides de novo production of a desired precursor or increased production of a precursor naturally produced by the host microbial organism. For example, ethanol is produced naturally in a host organism such as P. stipitis.

In some embodiments, a non-naturally occurring microbial organism provided herein is generated from a host that contains the enzymatic capability to synthesize ethanol. In this specific embodiment, it can be useful to increase the xylose update or xyloe metabolism to drive xylose-ethanol pathway reactions toward ethanol production. Increased synthesis or accumulation can be accomplished by, for example, overexpression of nucleic acids encoding one or more of the above-described xylose transporters or xylose-ethanol pathway enzymes or proteins. Overexpression of the transporter(s), enzyme(s) or protein(s) can occur, for example, through exogenous expression of the endogenous gene or genes, or through exogenous expression of the heterologous gene or genes. Therefore, naturally occurring organisms can be readily generated to be non-naturally occurring microbial organisms provided herein, for example, producing ethanol, through overexpression of one, two, three, four, five, or up to all nucleic acids encoding the xylose-ethanol biosynthetic pathway enzymes or proteins. In addition, a non-naturally occurring organism can be generated by mutagenesis of an endogenous gene that results in an increase in activity of an enzyme or protein in the xylose-ethanol biosynthetic pathway.

In particularly useful embodiments, exogenous expression of the encoding nucleic acids is employed. Exogenous expression confers the ability to custom tailor the expression and/or regulatory elements to the host and application to achieve a desired expression level that is controlled by the user. However, endogenous expression also can be utilized in other embodiments such as by removing a negative regulatory effector or induction of the gene's promoter when linked to an inducible promoter or other regulatory element. Thus, an endogenous gene having a naturally occurring inducible promoter can be up-regulated by providing the appropriate inducing agent, or the regulatory region of an endogenous gene can be engineered to incorporate an inducible regulatory element, thereby allowing the regulation of increased expression of an endogenous gene at a desired time. Similarly, an inducible promoter can be included as a regulatory element for an exogenous gene introduced into a non-naturally occurring microbial organism.

It is understood that, in methods provided herein, any of the one or more exogenous nucleic acids can be introduced into a microbial organism to produce a non-naturally occurring microbial organism as described herein. The nucleic acids can be introduced so as to confer, for example, a xylose-ethanol biosynthetic pathway into the microbial organism. Alternatively, encoding nucleic acids can be introduced to produce an intermediate microbial organism having the biosynthetic capability to catalyze some of the required reactions to confer biosynthetic capability. For example, a non-naturally occurring microbial organism having a xylose-ethanol biosynthetic pathway can comprise at least two exogenous nucleic acids encoding desired enzymes or proteins, such as the combination of a xylose transporter and a xylose reductase, and the like. Thus, it is understood that any combination of two or more enzymes or proteins of a biosynthetic pathway can be included in a non-naturally occurring microbial organism provided herein. Similarly, it is understood that any combination of three or more enzymes or proteins of a biosynthetic pathway can be included in a non-naturally occurring microbial organism of the invention, for example, a xylose transporter, a xylose reductase, and a xylose dehydrogenase and so forth, as desired, so long as the combination of enzymes and/or proteins of the desired biosynthetic pathway results in production of the corresponding desired product. Similarly, any combination of four, five, six or more enzymes or proteins of a biosynthetic pathway as disclosed herein can be included in a non-naturally occurring microbial organism provided herein, as desired, so long as the combination of enzymes and/or proteins of the desired biosynthetic pathway results in production of the corresponding desired product.

Similarly, it is understood by those skilled in the art that a host organism can be selected based on desired characteristics for introduction of one or more gene disruptions to increase production of ethanol. Thus, it is understood that, if a genetic modification is to be introduced into a host organism to disrupt a gene, any homologs, orthologs or paralogs that catalyze similar, yet non-identical metabolic reactions can similarly be disrupted to ensure that a desired metabolic reaction is sufficiently disrupted. Because certain differences exist among metabolic networks between different organisms, those skilled in the art will understand that the actual genes disrupted in a given organism may differ between organisms. However, given the teachings and guidance provided herein, those skilled in the art also will understand that the methods of the invention can be applied to any suitable host microorganism to identify the cognate metabolic alterations needed to construct an organism in a species of interest that will increase ethanol biosynthesis. In a particular embodiment, the increased production couples biosynthesis of ethanol to growth of the organism, and can obligatorily couple production of ethanol to growth of the organism if desired and as disclosed herein.

Modification of nucleic acids encoding xylose transporters and enzymes or proteins of the xylose metabolic pathway described herein to facilitate insertion into a particular vector (for example, by modifying restriction sites), ease of use in a particular expression system or host (for example, using preferred host codons), and the like, are known and are contemplated for use. Genetic engineering methods for the production of xylose transporters and enzymes or proteins of the xylose metabolic pathway include the expression of the polynucleotide molecules in cell free expression systems, in host cells, in tissues, and in animal models, according to known methods.

Methods for constructing and testing the expression of a desired enzyme and/or protein in a non-naturally occurring host can be performed, for example, by recombinant and detection methods well known in the art. Such methods can be found described in, for example, Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, Md. (1999).

Exogenous nucleic acid can be introduced stably or transiently into a host cell using techniques well known in the art including, but not limited to, conjugation, electroporation, chemical transformation, transduction, transfection, and ultrasound transformation. For exogenous expression in E. coli or other prokaryotic cells, some nucleic acid sequences in the genes or cDNAs of eukaryotic nucleic acids can encode targeting signals such as an N-terminal mitochondrial or other targeting signal, which can be removed before transformation into prokaryotic host cells, if desired. For example, removal of a mitochondrial leader sequence led to increased expression in E. coli (Hoffmeister et al., J. Biol. Chem. 280:4329-4338 (2005)). For exogenous expression in yeast or other eukaryotic cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques well known in the art to achieve optimized expression of the proteins. Available tools for codon optimization include “UpGene,” described in Gao et al., Biotechnology progress 20.2 (2004): 443-448; “Codon optimizer,” described in Fuglsang, Protein expression and purification 31.2 (2003): 247-249. As a person of ordinary skill would understand, it would have been a routine practice to use these or any other available tools in the art to codon optimize the specific nucleic acid sequences described herein to express the corresponding gene in a specific host strain.

An expression vector or vectors can be constructed to include one or more nucleic acids encoding xylose transporters and/or and enzymes or proteins of the xylose metabolic pathway operably linked to expression control sequences functional in the host organism. Expression vectors applicable for use in the microbial host organisms of the invention include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to confer or enhance xylose uptake or metabolism, and it is further understood that expression levels can be optimized to obtain sufficient expression using methods well known in the art and as disclosed herein.

Provided herein are also reagents, compositions, and methods that are useful for analysis of xylose transporter activity and for assessing the amount and rate of xylose transport. Provided herein are also reagents, compositions, and methods that are useful for analysis of the activity of enzyme(s) of xylose metabolic pathway and for assessing activity of xylose metabolism.

The polypeptide of xylose transporters or enzymes or proteins of the xylose metabolic pathways of the present disclosure, in whole or in part, can be used to raise polyclonal and monoclonal antibodies that are useful in purifying the desired enzyme or protein, or detecting their expression, as well as a reagent tool for characterizing their molecular actions. Preferably, a peptide containing a unique epitope of the target protein is used in preparation of antibodies, using conventional techniques. Methods for the selection of peptide epitopes and production of antibodies are known. See, for example, Antibodies: A Laboratory Manual, Harlow and Land (eds.), 1988 Cold Spring Harbor Laboratory Press, Cold Spring Harbor, N.Y.; Monoclonal Antibodies, Hybridomas: A New Dimension in Biological Analyses, Kennet et al. (eds.), 1980 Plenum Press, New York.

The non-naturally occurring microbial organisms provided herein have a xylose-ethanol pathway and enhanced xylose uptake or metabolism by expressing xylose transporter or enzymes or proteins of the xylose metabolic pathways described herein.

Given the teachings and guidance provided herein, those skilled in the art will understand that a wide variety of combinations and permutations exist for the non-naturally occurring microbial organisms and methods provided herein, together with other microbial organisms, with the co-culture of other non-naturally occurring microbial organisms having subpathways and with combinations of other chemical and/or biochemical procedures well known in the art to produce a desired product.

Provided herein are also methods to produce ethanol using the non-naturally occurring microbial organisms described herein. Suitable purification and/or assays to test for the production of ethanol can be performed using well known methods. Suitable replicates such as triplicate cultures can be grown for each engineered strain to be tested. For example, product and byproduct formation in the engineered production host can be monitored. The final product and intermediates, and other organic compounds, can be analyzed by methods such as HPLC (High Performance Liquid Chromatography), GC-MS (Gas Chromatography-Mass Spectroscopy) and LC-MS (Liquid Chromatography-Mass Spectroscopy) or other suitable analytical methods using routine procedures well known in the art. The release of product in the fermentation broth can also be tested with the culture supernatant. Byproducts and residual glucose can be quantified by HPLC using, for example, a refractive index detector for glucose and alcohols, and a UV detector for organic acids (Lin et al., Biotechnol. Bioeng. 90:775-779 (2005)), or other suitable assay and detection methods well known in the art. The individual enzyme or protein activities from the exogenous DNA sequences can also be assayed using methods well known in the art.

The ethanol can be separated from other components in the culture using a variety of methods well known in the art. Such separation methods include, for example, extraction procedures as well as methods that include continuous liquid-liquid extraction, pervaporation, membrane filtration, membrane separation, reverse osmosis, electrodialysis, distillation, crystallization, centrifugation, extractive filtration, ion exchange chromatography, size exclusion chromatography, adsorption chromatography, ultrafiltration, activated charcoal adsorption, pH adjustment and precipitation, or a combination of one or more methods enumerated above. All of the above methods are well known in the art.

Provided herein are non-naturally occurring microbial organism having a xylose-ethanol pathway and at least one exogenous nucleic acid encoding an enzyme or protein expressed in a sufficient amount to enhance xylose uptake or metabolism, which produces ethanol from xylose when cultured in medium having xylose. Accordingly, in some embodiments, provided herein a non-naturally occurring microbial organism that produces at least 0.1 g/L/h of ethanol from xylose when cultured. Also provided herein is a non-naturally occurring microbial organism that converts at least 0.1% (w/v) xylose to ethanol when cultured.

As can be understood by a person skilled in the art, the amount of ethanol from xylose produced by the non-naturally occurring microbial organism provided herein can vary depending on the culturing conditions. Accordingly, in some embodiments, the rate of ethanol production by the non-naturally occurring microbial organism provided herein is at least 0.2 g/L/h of ethanol from xylose. The rate of ethanol production can be at least 0.25 g/L/h of ethanol from xylose. The rate of ethanol production can be at least 0.3 g/L/h of ethanol from xylose. The rate of ethanol production can be at least 0.35 g/L/h of ethanol from xylose. The rate of ethanol production can be at least 0.4 g/L/h of ethanol from xylose. The rate of ethanol production can be at least 0.45 g/L/h of ethanol from xylose. The rate of ethanol production can be at least 0.50 g/L/h of ethanol from xylose. The rate of ethanol production can be at least 0.55 g/L/h of ethanol from xylose. The rate of ethanol production can be at least 0.60 g/L/h of ethanol from xylose. The rate of ethanol production can be at least 0.65 g/L/h of ethanol from xylose. The rate of ethanol production can be at least 0.70 g/L/h of ethanol from xylose. The rate of ethanol production can be at least 0.75 g/L/h of ethanol from xylose. The rate of ethanol production can be at least 0.80 g/L/h of ethanol from xylose. The rate of ethanol production can be at least 0.85 g/L/h of ethanol from xylose. The rate of ethanol production can be at least 0.90 g/L/h of ethanol from xylose. The rate of ethanol production can be at least 0.95 g/L/h of ethanol from xylose. The rate of ethanol production can be at least 1.00 g/L/h of ethanol from xylose. The rate of ethanol production can be at least 1.10 g/L/h of ethanol from xylose. The rate of ethanol production can be at least 1.20 g/L/h of ethanol from xylose. The rate of ethanol production can be at least 1.30 g/L/h of ethanol from xylose. The rate of ethanol production can be at least 1.40 g/L/h of ethanol from xylose. The rate of ethanol production can be at least 1.50 g/L/h of ethanol from xylose. The rate of ethanol production can be at least 1.60 g/L/h of ethanol from xylose. The rate of ethanol production can be at least 1.70 g/L/h of ethanol from xylose. The rate of ethanol production can be at least 1.80 g/L/h of ethanol from xylose. The rate of ethanol production can be at least 1.90 g/L/h of ethanol from xylose. The rate of ethanol production can be at least 2.00 g/L/h of ethanol from xylose. The rate of ethanol production can be at least 2.50 g/L/h of ethanol from xylose. The rate of ethanol production can be at least 3.00 g/L/h of ethanol from xylose. The rate of ethanol production can be at least 3.50 g/L/h of ethanol from xylose. The rate of ethanol production can be at least 4.00 g/L/h of ethanol from xylose. The rate of ethanol production can be at least 5.00 g/L/h of ethanol from xylose. The rate of ethanol production can be at least 6.00 g/L/h of ethanol from xylose. The rate of ethanol production can be at least 7.00 g/L/h of ethanol from xylose. The rate of ethanol production can be at least 8.00 g/L/h of ethanol from xylose. The rate of ethanol production can be at least 9.00 g/L/h of ethanol from xylose. The rate of ethanol production can be or at least 10.00 g/L/h of ethanol from xylose.

In some embodiments, the conversion efficiency of the non-naturally occurring microbial organism provided herein to convert xylose to ethanol is at least 0.01 g ethanol/g xylose. The conversion efficiency can be at least 0.02 g ethanol/g xylose. The conversion efficiency can be at least 0.03 g ethanol/g xylose. The conversion efficiency can be at least 0.04 g ethanol/g xylose. The conversion efficiency can be at least 0.05 g ethanol/g xylose. The conversion efficiency can be at least 0.06 g ethanol/g xylose. The conversion efficiency can be at least 0.07 g ethanol/g xylose. The conversion efficiency can be at least 0.08 g ethanol/g xylose. The conversion efficiency can be at least 0.09 g ethanol/g xylose. The conversion efficiency can be at least 0.1 g ethanol/g xylose. The conversion efficiency can be at least 0.15 g ethanol/g xylose. The conversion efficiency can be at least 0.2 g ethanol/g xylose. The conversion efficiency can be at least 0.25 g ethanol/g xylose. The conversion efficiency can be at least 0.3 g ethanol/g xylose. The conversion efficiency can be at least 0.35 g ethanol/g xylose. The conversion efficiency can be at least 0.4 g ethanol/g xylose. The conversion efficiency can be at least 0.45 g ethanol/g xylose. The conversion efficiency can be at least 0.5 g ethanol/g xylose. The conversion efficiency can be 0.51 g ethanol/g xylose.

In some embodiments, the yield of the non-naturally occurring microbial organism provided herein produce ethanol from xylose is at least 0.1% (w/v). The yield can be at least 0.2% (w/v). The yield can be at least 0.3% (w/v). The yield can be at least 0.4% (w/v). The yield can be at least 0.5% (w/v). The yield can be at least 0.6% (w/v). The yield can be at least 0.7% (w/v). The yield can be at least 0.8% (w/v). The yield can be at least 0.9% (w/v). The yield can be at least 1.0% (w/v). The yield can be at least 1.5% (w/v). The yield can be at least 2.0% (w/v). The yield can be at least 2.5% (w/v). The yield can be at least 3.5% (w/v). The yield can be at least 4.0% (w/v). The yield can be at least 4.5% (w/v). The yield can be at least 5.0% (w/v). The yield can be at least 6.0% (w/v). The yield can be at least 7.0% (w/v). The yield can be at least 8.0% (w/v). The yield can be at least 9.0% (w/v). The yield can be at least 10.0% (w/v). The yield can be at least 11.0% (w/v). The yield can be at least 12.0% (w/v). The yield can be at least 13.0% (w/v). The yield can be at least 14.0% (w/v). The yield can be at least 15.0% (w/v).

Culturing conditions that can yield the rate of ethanol from xylose described herein include conditions that vary the amount of aeration of the medium, the temperature of the medium, the amount of time the culture is grown for and the composition of the medium. In some embodiments, the culturing of the non-naturally occurring microbial organism provided herein occurs under aerobic conditions. In some embodiments, the culturing of the non-naturally occurring microbial organism provided herein occures under substantially anaerobic conditions. In some embodiments, the temperature of the medium ranges from 20° C. to 38° C., or alternatively 26° C. to 35° C., or alternatively 28° C. to 32° C., or alternatively at about 30° C. In some embodiments, the culture is grown for 1 day. In some embodiment, the culture is grown for 2 days. In some embodiments, the culture is grown for 3 days. In some embodiments, the culture is grown for 4 days. In some embodiments, the culture is grown for 5 days. In some embodiments, the culture is grown for 6 days. In some embodiments, the culture is grown for 7 or more days. The composition of the medium can be any medium well known in the art for culturing the non-naturally occurring microbial organism provided herein. Exemplary medium includes, but is not limited to, yeast extract peptone (YEP) medium or yeast nitrogen base (YNB) medium. Additionaly, the carbon source in the medium used by the isolated Metschnikowia species can include xylose as the only carbon source, as well as xylose in combination with other carbon sources described herein. The amount of the carbon source in the medium can range from 1% to 20% (e.g., 1% to 20% xylose), or alternatively 2% to 14% (e.g., 2% to 14% xylose), or alternatively 4% to 10% (e.g., 4% to 10% xylose). In some embodiments, the amount of the carbon sourse is 4% (e.g., 4% xylose).

Any of the non-naturally occurring microbial organisms described herein can be cultured to produce and/or secrete ethanol. For example, the microbial organisms provided herein can be cultured for the biosynthetic production ethanol. Accordingly, in some embodiments, provided herein are culture media containing ethanol or an intermediate of the xylose-ethanol pathway, such as a xylitol. In some aspects, the culture medium can also be separated from the non-naturally occurring microbial organisms that produced ethanol or an intermediate of the xylose-ethanol pathway. Methods for separating a microbial organism from culture medium are well known in the art. Exemplary methods include filtration, flocculation, precipitation, centrifugation, sedimentation, and the like.

For the production of ethanol, the microbial organisms provided herein are cultured in a medium with carbon source and other essential nutrients. In some embodiments, the microbial organisms provided herein are cultured in an aerobic culture medium. In some embodiments, the microbial organisms provided herein are cultured in a substantially anaerobic culture medium. As described herein, one exemplary growth condition for achieving biosynthesis of ethanol includes anaerobic culture or fermentation conditions. In certain embodiments, the non-naturally occurring microbial organisms provided herein can be sustained, cultured or fermented under anaerobic or substantially anaerobic conditions. Briefly, an anaerobic condition refers to an environment devoid of oxygen. Substantially anaerobic conditions include, for example, a culture, batch fermentation or continuous fermentation such that the dissolved oxygen concentration in the medium remains between 0 and 10% of saturation. Substantially anaerobic conditions also includes growing or resting cells in liquid medium or on solid agar inside a sealed chamber maintained with an atmosphere of less than 1% oxygen. The percent of oxygen can be maintained by, for example, sparging the culture with an N₂/CO₂ mixture or other suitable non-oxygen gas or gases.

It is sometimes desirable and can be highly desirable to maintain anaerobic conditions in the fermenter to reduce the cost of the overall process. Such conditions can be obtained, for example, by first sparging the medium with nitrogen and then sealing the flasks with a septum and crimp-cap. For strains where growth is not observed anaerobically, microaerobic or substantially anaerobic conditions can be applied by perforating the septum with a small hole for limited aeration. Exemplary anaerobic conditions have been described previously and are well-known in the art. Exemplary aerobic and anaerobic conditions are described, for example, in United States publication 2009/0047719, filed Aug. 10, 2007. Fermentations can be performed in a batch, fed-batch or continuous manner, as disclosed herein. Fermentations can also be conducted in two phases, if desired. The first phase can be aerobic to allow for high growth and therefore high productivity, followed by an anaerobic phase of high yields.

If desired, the pH of the medium can be maintained at a desired pH, in particular neutral pH, such as a pH of around 7 by addition of a base, such as NaOH or other bases, or acid, as needed to maintain the culture medium at a desirable pH. The growth rate can be determined by measuring optical density using a spectrophotometer (600 nm), and the glucose uptake rate by monitoring carbon source depletion over time.

The culture medium for the microbial organisms provided herein can include xylose, either as the sole source of carbon or in combination with one or more co-substrates described herein or known in the art. The culture medium can further include other supplements, such as yeast extract, and/or peptone. The culture medium can further include, for example, any other carbohydrate source which can supply a source of carbon to the non-naturally occurring microorganism. Such sources include, for example: other sugars such as cellobiose, hemicelluloses, glucose, arabinose, galactose, mannose, fructose, sucrose and starch; or glycerol. Thus, the culture medium can include xylose and the co-substrate glucose. The culture medium can include xylose and the co-substrate cellobiose. The culture medium can include xylose and the co-substrate hemicellulose. The culture medium can include xylose and the co-substrate galactose. The culture medium can include xylose and the co-substrate glycerol.

The culture medium can have 1%, 2%, 3%, 4%, 5%, 6%, 7%, 8%, 9%, 10%, 11%, 12%, 13%, 14%, 15%, 16%, 17%, 18%, 19%, 20%, or higher amount of sugar (w/v). In some embodiments, the culture medium can have 2% sugar. In some embodiments, the culture medium can have 4% sugar. In some embodiments, the culture medium can have 10% sugar. In some embodiments, the culture medium can have 1%, 2%, 3%, 4%, 5%, 6%, 7%, 8%, 9%, 10%, 11%, 12%, 13%, 14%, 15%, 16%, 17%, 18%, 19%, 20%, or higher amount of xylose (w/v). The culture medium can have 1% xylose. The culture medium can have 2% xylose. The culture medium can have 3% xylose. The culture medium can have 4% xylose. The culture medium can have 5% xylose. The culture medium can have 6% xylose. The culture medium can have 7% xylose. The culture medium can have 8% xylose. The culture medium can have 9% xylose. The culture medium can have 10% xylose. The culture medium can have 11% xylose. The culture medium can have 12% xylose. The culture medium can have 12% xylose. The culture medium can have 13% xylose. The culture medium can have 14% xylose. The culture medium can have 15% xylose. The culture medium can have 16% xylose. The culture medium can have 17% xylose. The culture medium can have 18% xylose. The culture medium can have 19% xylose. The culture medium can have 20% xylose.

In some embodiments, xylose is not the only carbon source. For example, in some embodiments, the medium includes xylose and a C3 carbon source, a C4 carbon source, a C5 carbon source, a C6 carbon source, or a combination thereof. Accordingly, in some embodiments, the medium includes xylose and a C3 carbon source (e.g., glycerol). In some embodiments, the medium includes xylose and a C4 carbon source (e.g., erythrose or threose). In some embodiments, the medium includes xylose and a C5 carbon source (e.g., arabinose, arabitol, ribose or lyxose). In some embodiments, the medium includes xylose and a C6 carbon source (e.g., glucose, galactose, mannose, allose, altrose, gulose, and idose). Alternatively or additionally, in some embodiments, the medium includes xylose, cellobiose, galactose, glucose, arabitol, sorbitol, glycerol, or a combination thereof. In a specific embodiment, the medium includes xylose and glucose. The amount of the two or more carbon sources in the medium can range independently from 1% to 20% (e.g., 1% to 20% xylose and 1% to 20% glucose), or alternatively 2% to 14% (e.g., 2% to 14% xylose and 2% to 14% glucose), or alternatively 4% to 10% (e.g., 4% to 10% xylose and 4% to 10%). In a specific embodiment, the amount of each of the carbon sourses is 2% (e.g., 2% xylose and 2% glucose)

The culture medium can be a C5-rich medium, with a five carbon sugar (such as xylose) as the primary carbon source. The culture medium can also have a C6 sugar (six-carbon sugar). In some embodiments, the culture medium can have a C6 sugar as the primary carbon source. In some embodiments, the C6 sugar is glucose. The culture can have both a C6 sugar and a C5 sugar as the carbon source can have the C6 sugar and the C5 sugar present at different ratios. In some embodiment, the ratio of the amount of C6 sugar to that of the C5 sugar (the C6: C5 ratio) in the culture medium is between about 10:1 and about 1:10. For example, the C6: C5 ratio in the culture medium can be about 10:1, 9:1, 8:1, 7:1, 6:1, 5:1, 3:1, 2:1, 1:1, 1:2, 1:3, 1:4, 1:5, 1:6, 1:7, 1:8, 1:9 or 1:10. In some embodiments, the C6: C5 ratio in the culture medium is about 3:1. In some embodiments, the C6: C5 ratio in the culture medium is about 1:1. In some embodiments, the C6: C5 ratio in the culture medium is about 1:5. In some embodiments, the C6: C5 ratio in the culture medium is about 1:10. The C5 sugar can be xylose, and the C6 sugar can be glucose. In some embodiment, the ratio of the amount of glucose to that of xylose (the glucose: xylose ratio) in the culture medium is between about 10:1 and about 1:10. For example, the glucose: xylose ratio in the culture medium can be about 10:1, 9:1, 8:1, 7:1, 6:1, 5:1, 3:1, 2:1, 1:1, 1:2, 1:3, 1:4, 1:5, 1:6, 1:7, 1:8, 1:9 or 1:10. In some embodiments, the glucose: xylose ratio in the culture medium is about 3:1. In some embodiments, the glucose: xylose ratio in the culture medium is about 1:1. In some embodiments, the glucose: xylose ratio in the culture medium is about 1:5. In some embodiments, the glucose: xylose ratio in the culture medium is about 1:10.

Other sources of carbohydrate include, for example, renewable feedstocks and biomass. Exemplary types of biomasses that can be used as feedstocks in the methods of the invention include cellulosic biomass, hemicellulosic biomass and lignin feedstocks or portions of feedstocks. Such biomass feedstocks contain, for example, carbohydrate substrates useful as carbon sources such as xylose, glucose, arabinose, galactose, mannose, fructose and starch. Given the teachings and guidance provided herein, those skilled in the art will understand that renewable feedstocks and biomass other than those exemplified above also can be used for culturing the microbial organisms of the invention for the production of ethanol.

Accordingly, given the teachings and guidance provided herein, those skilled in the art will understand that a non-naturally occurring microbial organism can be produced that secretes ethanol or an intermediate of the xylose-ethanol pathway when grown on xylose as a carbon source. All that is required is to engineer in one or more of the required enzyme or protein activities to achieve biosynthesis of ethanol or an intermediate of the xylose-ethanol pathway. Accordingly, provided herein is a non-naturally occurring microbial organism that produces and/or secretes ethanol or an intermediate of the xylose-ethanol pathway when grown on xylose alone or together with another carbohydrate or other carbon source and produces and/or secretes ethanol or an intermediate of the xylose-ethanol pathway.

The non-naturally occurring microbial organisms provided herein are constructed using methods well known in the art as exemplified herein to exogenously express at least one nucleic acid encoding an enzyme or protein expressed in a sufficient amount to confer or enhance xylose uptake or metabolism and increase the production of ethanol from xylose. It is understood that the microbial organisms provided herein are cultured under conditions sufficient to produce ethanol. Following the teachings and guidance provided herein, the non-naturally occurring microbial organisms provided herein can achieve biosynthesis of ethanol resulting in intracellular concentrations between about 0.1-200 mM or more. Generally, the intracellular concentration of ethanol between about 3-150 mM, particularly between about 5-125 mM and more particularly between about 8-100 mM, including about 10 mM, 20 mM, 50 mM, 80 mM, or more. Intracellular concentrations between and above each of these exemplary ranges also can be achieved from the non-naturally occurring microbial organisms provided herein.

In some embodiments, culture conditions include anaerobic or substantially anaerobic growth or maintenance conditions. Exemplary anaerobic conditions have been described previously and are well known in the art. Exemplary anaerobic conditions for fermentation processes are described herein and are described, for example, in U.S. publication 2009/0047719. Any of these conditions can be employed with the non-naturally occurring microbial organisms as well as other anaerobic conditions well known in the art. Under such anaerobic or substantially anaerobic conditions, the producer strains can synthesize the desired product at intracellular concentrations of 5-10 mM or more as well as all other concentrations exemplified herein. It is understood that, even though the above description refers to intracellular concentrations, the producing microbial organisms can produce the desired product intracellularly and/or secrete the product into the culture medium.

Exemplary fermentation processes include, but are not limited to, fed-batch fermentation and batch separation; fed-batch fermentation and continuous separation; and continuous fermentation and continuous separation. In an exemplary fed-batch fermentation protocol, the production organism is grown in a suitably sized bioreactor sparged with an appropriate gas. Under anaerobic conditions, the culture is sparged with an inert gas or combination of gases, for example, nitrogen, N₂/CO₂ mixture, argon, helium, and the like. As the cells grow and utilize the carbon source, additional carbon source(s) and/or other nutrients are fed into the bioreactor at a rate approximately balancing consumption of the carbon source and/or nutrients. The temperature of the bioreactor is maintained at a desired temperature, generally in the range of 22-37 degrees C., but the temperature can be maintained at a higher or lower temperature depending on the growth characteristics of the production organism and/or desired conditions for the fermentation process. Growth continues for a desired period of time to achieve desired characteristics of the culture in the fermenter, for example, cell density, product concentration, and the like. In a fed-batch fermentation process, the time period for the fermentation is generally in the range of several hours to several days, for example, 8 to 24 hours, or 1, 2, 3, 4 or 5 days, or up to a week, depending on the desired culture conditions. The pH can be controlled or not, as desired, in which case a culture in which pH is not controlled will typically decrease to pH 3-6 by the end of the run. In some embodiment, the initial pH can first decrease and then increase during the cultivation period. In one embodiment, the initial pH of the medium is around 6, and during the cultivation period, the pH decreased first to 5.5 and later increased to around 6.5. Upon completion of the cultivation period, the fermenter contents can be passed through a cell separation unit, for example, a centrifuge, filtration unit, and the like, to remove cells and cell debris. In the case where the desired product is expressed intracellularly, the cells can be lysed or disrupted enzymatically or chemically prior to or after separation of cells from the fermentation broth, as desired, in order to release additional product. The fermentation broth can be transferred to a product separations unit. Isolation of product occurs by standard separations procedures employed in the art to separate a desired product from dilute aqueous solutions. Such methods include, but are not limited to, liquid-liquid extraction using a water immiscible organic solvent (e.g., toluene or other suitable solvents, including but not limited to diethyl ether, ethyl acetate, tetrahydrofuran (THF), methylene chloride, chloroform, benzene, pentane, hexane, heptane, petroleum ether, methyl tertiary butyl ether (MTBE), dioxane, dimethylformamide (DMF), dimethyl sulfoxide (DMSO), and the like) to provide an organic solution of the product, if appropriate, standard distillation methods, and the like, depending on the chemical characteristics of the product of the fermentation process.

In an exemplary fully continuous fermentation protocol, the production organism is generally first grown up in batch mode in order to achieve a desired cell density. When the carbon source and/or other nutrients are exhausted, feed medium of the same composition is supplied continuously at a desired rate, usually with relatively high sugar concentration, and fermentation liquid is withdrawn at the same rate. Under such conditions, the product concentration in the bioreactor generally remains constant, as well as the cell density. The temperature of the fermenter is maintained at a desired temperature, as discussed above. During the continuous fermentation phase, it is generally desirable to maintain a suitable pH range for optimized production. The pH can be monitored and maintained using routine methods, including the addition of suitable acids or bases to maintain a desired pH range. The bioreactor is operated continuously for extended periods of time, generally at least one week to several weeks and up to one month, or longer, as appropriate and desired. The fermentation liquid and/or culture is monitored periodically, including sampling up to every day, as desired, to assure consistency of product concentration and/or cell density. In continuous mode, fermenter contents are constantly removed as new feed medium is supplied. The exit stream, containing cells, medium, and product, are generally subjected to a continuous product separations procedure, with or without removing cells and cell debris, as desired. Continuous separations methods employed in the art can be used to separate the product from dilute aqueous solutions, including but not limited to continuous liquid-liquid extraction using a water immiscible organic solvent (e.g., toluene or other suitable solvents, including but not limited to diethyl ether, ethyl acetate, tetrahydrofuran (THF), methylene chloride, chloroform, benzene, pentane, hexane, heptane, petroleum ether, methyl tertiary butyl ether (MTBE), dioxane, dimethylformamide (DMF), dimethyl sulfoxide (DMSO), and the like), standard continuous distillation methods, and the like, or other methods well known in the art.

In addition to the culturing and fermentation conditions disclosed herein, growth condition for achieving biosynthesis of ethanol can include the addition of an osmoprotectant to the culturing conditions. In certain embodiments, the non-naturally occurring microbial organisms provided herein can be sustained, cultured or fermented as described herein in the presence of an osmoprotectant. Briefly, an osmoprotectant refers to a compound that acts as an osmolyte and helps a microbial organism as described herein survive osmotic stress. Osmoprotectants include, but are not limited to, betaines, amino acids, and the sugar trehalose. Non-limiting examples of such are glycine betaine, praline betaine, dimethylthetin, dimethyl slfonioproprionate, 3-dimethylsulfonio-2-methylproprionate, pipecolic acid, dimethylsulfonioacetate, choline, L-carnitine and ectoine. In one aspect, the osmoprotectant is glycine betaine. It is understood to one of ordinary skill in the art that the amount and type of osmoprotectant suitable for protecting a microbial organism described herein from osmotic stress will depend on the microbial organism used. The amount of osmoprotectant in the culturing conditions can be, for example, no more than about 0.1 mM, no more than about 0.5 mM, no more than about 1.0 mM, no more than about 1.5 mM, no more than about 2.0 mM, no more than about 2.5 mM, no more than about 3.0 mM, no more than about 5.0 mM, no more than about 7.0 mM, no more than about 10 mM, no more than about 50 mM, no more than about 100 mM or no more than about 500 mM.

The culture conditions can include, for example, liquid culture procedures as well as fermentation and other large scale culture procedures. As described herein, particularly useful yields of the biosynthetic ethanol can be obtained under anaerobic or substantially anaerobic culture conditions.

The culture conditions described herein can be scaled up and grown continuously for manufacturing of a desired product. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of ethanol. Generally, and as with non-continuous culture procedures, the continuous and/or near-continuous production includes culturing the microbial organisms provided herein in sufficient nutrients and medium to sustain and/or nearly sustain growth in an exponential phase. Continuous culture under such conditions can include, for example, growth or culturing for 1 day, 2, 3, 4, 5, 6 or 7 days or more. Additionally, continuous culture can include longer time periods of 1 week, 2, 3, 4 or 5 or more weeks and up to several months. Alternatively, organisms of the invention can be cultured for hours, if suitable for a particular application. It is to be understood that the continuous and/or near-continuous culture conditions also can include all time intervals in between these exemplary periods. It is further understood that the time of culturing the microbial organism provided herein is for a sufficient period of time to produce a sufficient amount of product for a desired purpose.

Fermentation procedures are well known in the art. Briefly, fermentation for the biosynthetic production of a desired product can be utilized in, for example, fed-batch fermentation and batch separation; fed-batch fermentation and continuous separation, or continuous fermentation and continuous separation. Examples of batch and continuous fermentation procedures are well known in the art.

In addition to the above fermentation procedures using producer strains provided herein using continuous production of substantial quantities of ethanol, the bioderived ethanol also can be, for example, simultaneously subjected to chemical synthesis and/or enzymatic procedures to convert the ethanol to other compounds, or the bioderived ethanol can be separated from the fermentation culture and sequentially subjected to chemical and/or enzymatic conversion to convert ethanol to other compounds, if desired.

To generate better producers, metabolic modeling can be utilized to optimize growth conditions. Modeling can also be used to design gene knockouts that additionally optimize utilization of the pathway (see, for example, U.S. patent publications US 2002/0012939, US 2003/0224363, US 2004/0029149, US 2004/0072723, US 2003/0059792, US 2002/0168654 and US 2004/0009466, and U.S. Pat. No. 7,127,379). Modeling analysis allows reliable predictions of the effects on cell growth of shifting the metabolism towards more efficient production of a desired product.

Provided herein is also bioderived ethanol produced using the non-naturally occurring microbial organisms described herein. In some embodiments, the carbon feedstock and other cellular uptake sources such as phosphate, ammonia, sulfate, chloride and other halogens can be chosen to alter the isotopic distribution of the atoms present in ethanol or any xylose-ethanol pathway intermediate product, such as xylitol. The various carbon feedstock and other uptake sources enumerated above will be referred to herein, collectively, as “uptake sources.” Uptake sources can provide isotopic enrichment for any atom present in the product ethanol or any xylose-ethanol pathway intermediate product, or for side products generated in reactions diverging away from a xylose-ethanol pathway. Isotopic enrichment can be achieved for any target atom including, for example, carbon, hydrogen, oxygen, nitrogen, sulfur, phosphorus, chloride or other halogens.

In some embodiments, the uptake sources can be selected to alter the carbon-12, carbon-13, and carbon-14 ratios. In some embodiments, the uptake sources can be selected to alter the oxygen-16, oxygen-17, and oxygen-18 ratios. In some embodiments, the uptake sources can be selected to alter the hydrogen, deuterium, and tritium ratios. In some embodiments, the uptake sources can be selected to alter the nitrogen-14 and nitrogen-15 ratios. In some embodiments, the uptake sources can be selected to alter the sulfur-32, sulfur-33, sulfur-34, and sulfur-35 ratios. In some embodiments, the uptake sources can be selected to alter the phosphorus-31, phosphorus-32, and phosphorus-33 ratios. In some embodiments, the uptake sources can be selected to alter the chlorine-35, chlorine-36, and chlorine-37 ratios.

In some embodiments, the isotopic ratio of a target atom can be varied to a desired ratio by selecting one or more uptake sources. An uptake source can be derived from a natural source, as found in nature, or from a man-made source, and one skilled in the art can select a natural source, a man-made source, or a combination thereof, to achieve a desired isotopic ratio of a target atom. An example of a man-made uptake source includes, for example, an uptake source that is at least partially derived from a chemical, synthetic reaction. Such isotopically enriched uptake sources can be purchased commercially or prepared in the laboratory and/or optionally mixed with a natural source of the uptake source to achieve a desired isotopic ratio. In some embodiments, a target atom isotopic ratio of an uptake source can be achieved by selecting a desired origin of the uptake source as found in nature. For example, as discussed herein, a natural source can be a biobased derived from or synthesized by a biological organism or a source such as petroleum-based products or the atmosphere. In some such embodiments, a source of carbon, for example, can be selected from a fossil fuel-derived carbon source, which can be relatively depleted of carbon-14, or an environmental or atmospheric carbon source, such as CO₂, which can possess a larger amount of carbon-14 than its petroleum-derived counterpart.

The unstable carbon isotope carbon-14 or radiocarbon makes up for roughly 1 in 10¹² carbon atoms in the earth's atmosphere and has a half-life of about 5700 years. The stock of carbon is replenished in the upper atmosphere by a nuclear reaction involving cosmic rays and ordinary nitrogen (¹⁴N). Fossil fuels contain no carbon-14, as it decayed long ago. Burning of fossil fuels lowers the atmospheric carbon-14 fraction, the so-called “Suess effect”.

Methods of determining the isotopic ratios of atoms in a compound are well known to those skilled in the art. Isotopic enrichment is readily assessed by mass spectrometry using techniques known in the art such as accelerated mass spectrometry (AMS), Stable Isotope Ratio Mass Spectrometry (SIRMS) and Site-Specific Natural Isotopic Fractionation by Nuclear Magnetic Resonance (SNIF-NMR). Such mass spectral techniques can be integrated with separation techniques such as liquid chromatography (LC), high performance liquid chromatography (HPLC) and/or gas chromatography, and the like.

In the case of carbon, ASTM D6866 was developed in the United States as a standardized analytical method for determining the biobased content of solid, liquid, and gaseous samples using radiocarbon dating by the American Society for Testing and Materials (ASTM) International. The standard is based on the use of radiocarbon dating for the determination of a product's biobased content. ASTM D6866 was first published in 2004, and the current active version of the standard is ASTM D6866-11 (effective Apr. 1, 2011). Radiocarbon dating techniques are well known to those skilled in the art, including those described herein.

The biobased content of a compound is estimated by the ratio of carbon-14 (¹⁴C) to carbon-12 (¹²C). Specifically, the Fraction Modern (Fm) is computed from the expression: Fm=(S−B)/(M−B), where B, S and M represent the ¹⁴C/¹²C ratios of the blank, the sample and the modern reference, respectively. Fraction Modern is a measurement of the deviation of the ¹⁴C/¹²C ratio of a sample from “Modern.” Modern is defined as 95% of the radiocarbon concentration (in AD 1950) of National Bureau of Standards (NBS) Oxalic Acid I (i.e., standard reference materials (SRM) 4990b) normalized to δ¹³C_{VPDB}=−19 per mil (Olsson, The use of Oxalic acid as a Standard. in, Radiocarbon Variations and Absolute Chronology, Nobel Symposium, 12th Proc., John Wiley & Sons, New York (1970)). Mass spectrometry results, for example, measured by ASM, are calculated using the internationally agreed upon definition of 0.95 times the specific activity of NBS Oxalic Acid I (SRM 4990b) normalized to δ¹³C_{VPDB}=−19 per mil. This is equivalent to an absolute (AD 1950) ¹⁴C/¹²C ratio of 1.176±0.010×10¹² (Karlen et al., Arkiv Geoftsik, 4:465-471 (1968)). The standard calculations take into account the differential uptake of one isotope with respect to another, for example, the preferential uptake in biological systems of ¹²C over ¹³C over ¹⁴C, and these corrections are reflected as a Fm corrected for δ¹³.

An oxalic acid standard (SRM 4990b or HOx 1) was made from a crop of 1955 sugar beet. Although there were 1000 lbs made, this oxalic acid standard is no longer commercially available. The Oxalic Acid II standard (HOx 2; N.I.S.T designation SRM 4990 C) was made from a crop of 1977 French beet molasses. In the early 1980's, a group of 12 laboratories measured the ratios of the two standards. The ratio of the activity of Oxalic acid II to 1 is 1.2933±0.001 (the weighted mean). The isotopic ratio of HOx II is −17.8 per mil. ASTM D6866-11 suggests use of the available Oxalic Acid II standard SRM 4990 C (Hox2) for the modern standard (see discussion of original vs. currently available oxalic acid standards in Mann, Radiocarbon, 25(2):519-527 (1983)). A Fm=0% represents the entire lack of carbon-14 atoms in a material, thus indicating a fossil (for example, petroleum based) carbon source. A Fm=100%, after correction for the post-1950 injection of carbon-14 into the atmosphere from nuclear bomb testing, indicates an entirely modern carbon source. As described herein, such a “modern” source includes biobased sources.

As described in ASTM D6866, the percent modern carbon (pMC) can be greater than 100% because of the continuing but diminishing effects of the 1950s nuclear testing programs, which resulted in a considerable enrichment of carbon-14 in the atmosphere as described in ASTM D6866-11. Because all sample carbon-14 activities are referenced to a “pre-bomb” standard, and because nearly all new biobased products are produced in a post-bomb environment, all pMC values (after correction for isotopic fraction) must be multiplied by 0.95 (as of 2010) to better reflect the true biobased content of the sample. A biobased content that is greater than 103% suggests that either an analytical error has occurred, or that the source of biobased carbon is more than several years old.

ASTM D6866 quantifies the biobased content relative to the material's total organic content and does not consider the inorganic carbon and other non-carbon containing substances present. For example, a product that is 50% starch-based material and 50% water would be considered to have a Biobased Content=100% (50% organic content that is 100% biobased) based on ASTM D6866. In another example, a product that is 50% starch-based material, 25% petroleum-based, and 25% water would have a Biobased Content=66.7% (75% organic content but only 50% of the product is biobased). In another example, a product that is 50% organic carbon and is a petroleum-based product would be considered to have a Biobased Content=0% (50% organic carbon but from fossil sources). Thus, based on the well-known methods and standards for determining the biobased content of a compound or material, one skilled in the art can readily determine the biobased content and/or prepared downstream products that utilize of the invention having a desired biobased content.

Applications of carbon-14 dating techniques to quantify bio-based content of materials are known in the art (Currie et al., Nuclear Instruments and Methods in Physics Research B, 172:281-287 (2000)). For example, carbon-14 dating has been used to quantify bio-based content in terephthalate-containing materials (Colonna et al., Green Chemistry, 13:2543-2548 (2011)). Notably, polypropylene terephthalate (PPT) polymers derived from renewable 1,3-propanediol and petroleum-derived terephthalic acid resulted in Fm values near 30% (i.e., since 3/11 of the polymeric carbon derives from renewable 1,3-propanediol and 8/11 from the fossil end member terephthalic acid) (Currie et al., supra, 2000). In contrast, polybutylene terephthalate polymer derived from both renewable 1,4-butanediol and renewable terephthalic acid resulted in bio-based content exceeding 90% (Colonna et al., supra, 2011).

Accordingly, in some embodiments, the present invention provides bioderived ethanol or any xylose-ethanol pathway intermediate product that has a carbon-12, carbon-13, and carbon-14 ratio that reflects an atmospheric carbon, also referred to as environmental carbon, uptake source. For example, in some aspects the bioderived ethanol or any xylose-ethanol pathway intermediate product can have an Fm value of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or as much as 100%. In some such embodiments, the uptake source is CO₂. In some embodiments, the present invention provides bioderived ethanol or any xylose-ethanol pathway intermediate product that has a carbon-12, carbon-13, and carbon-14 ratio that reflects petroleum-based carbon uptake source. In this aspect, the bioderived ethanol or any xylose-ethanol pathway intermediate product can have an Fm value of less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 2% or less than 1%. In some embodiments, the present invention provides bioderived ethanol or any xylose-ethanol pathway intermediate product that has a carbon-12, carbon-13, and carbon-14 ratio that is obtained by a combination of an atmospheric carbon uptake source with a petroleum-based uptake source. Using such a combination of uptake sources is one way by which the carbon-12, carbon-13, and carbon-14 ratio can be varied, and the respective ratios would reflect the proportions of the uptake sources.

Further, the present invention relates to the bioderived ethanol or any xylose-ethanol pathway intermediate product as disclosed herein, and to the products derived therefrom, wherein the ethanol or any xylose-ethanol pathway intermediate product has a carbon-12, carbon-13, and carbon-14 isotope ratio of about the same value as the CO₂ that occurs in the environment. For example, in some aspects the invention provides bioderived ethanol or any xylose-ethanol pathway intermediate product having a carbon-12 versus carbon-13 versus carbon-14 isotope ratio of about the same value as the CO₂ that occurs in the environment, or any of the other ratios disclosed herein. It is understood, as disclosed herein, that a product can have a carbon-12 versus carbon-13 versus carbon-14 isotope ratio of about the same value as the CO₂ that occurs in the environment, or any of the ratios disclosed herein, wherein the product is generated from bioderived ethanol or any xylose-ethanol pathway intermediate product as disclosed herein, wherein the bioderived product is chemically modified to generate a final product. Methods of chemically modifying a bioderived product of ethanol or an intermediate product of the xylose-ethanol pathway, to generate a desired product are well known to those skilled in the art, as described herein. The invention further provides biobased products having a carbon-12 versus carbon-13 versus carbon-14 isotope ratio of about the same value as the CO₂ that occurs in the environment, wherein the biobased products are generated directly from or in combination with bioderived ethanol or any xylose-ethanol pathway intermediate product as disclosed herein.

Provided herein are also compositions having the bioderived ethanol or an intermediate of the xylose-ethanol pathway produced by the microbial organisms described herein, and an additional component. The additional component can be a cellular portion, for example, a trace amount of a cellular portion of the culture medium, or can be fermentation broth or culture medium or a purified or partially purified fraction thereof produced in the presence of, a non-naturally occurring microbial organism provided herein. The additional component can be the microbial organisms described herein used to produce ethanol or an intermediate of the xylose-ethanol pathway. The additional component can be the cell lysate of the microbial organism provided herein. The composition can have, for example, a reduced level of a byproduct when produced by the microbial organism disclosed herein. The additional component can be a byproduct, or an impurity, such as glycerol, acetate, glyceraldehyde, acetaldehyde, or a combination thereof. The byproduct can be glycerol. The byproduct can be acetaldehyde. The byproduct can be glyceraldehyde. The byproduct can be acetaldehyde. The impurity can be glycerol. The impurity can be acetate. The impurity can be glyceraldehyde. The impurity can be acetaldehyde.

Provided herein are also biobased products having bioderived ethanol or an intermediate of the xylose-ethanol pathway produced by a non-naturally occurring microorganism described herein or produced using a method described herein. Such manufacturing can include chemically reacting the bioderived compound (e.g. chemical conversion, chemical functionalization, chemical coupling, oxidation, reduction, polymerization, copolymerization and the like) into the final product. In some embodiments, provided herein are biobased products having at least 2%, at least 3%, at least 5%, at least 10%, at least 15%, at least 20%, at least 25%, at least 30%, at least 35%, at least 40%, at least 50%, at least 60%, at least 70%, at least 80%, at least 90%, at least 95%, at least 98% or 100% bioderived ethanol as disclosed herein.

It is understood that modifications which do not substantially affect the activity of the various embodiments of this invention are also provided within the definition of the invention provided herein. Accordingly, the following examples are intended to illustrate but not limit the present invention. Throughout this application various publications have been referenced. The disclosures of these publications in their entireties, including GenBank and GI number publications, are hereby incorporated by reference in this application in order to more fully describe the state of the art to which this invention pertains.

### EXAMPLE I

### Engineering S. cerevisiae with Enhanced Xylose Uptake

S. cerevisiae does not have the functional machinery to efficiently utilize xylose as the carbon source. S. cerevisiae has a fully annotated genome, complete transcriptomic data and hundreds of tools developed for genetic and biochemical manipulation. The xylose transporters from the H0 Metschnikowia species were introduced to S. cerevisiae to increase xylose uptake and to synthesize bioderived product from renewable biomass. S. cerevisiae BY4742 was used as the genetic platform to heterologously over-express xylose transporter from the H0 Metschnikowia species.

The following xylose transporters from the H0 Metschnikowia species were cloned XYT1, GXF1, ΔGXF1 (encoding variant of GXF1 with shorter N-terminus), GXS1/HGT12, ΔGXS1/HGT12 (encoding variant of GXS1/HGT12 with shorter N-terminus), and HXT5, and codon optimized for expression in S. cerevisiae. As shown in FIG. 3, the expression of XYT1 in Saccharomyces, the xylose transferred in 48 hours from the medium increased from about 10% in BY4742 to about 74% in BY4742 expressing Xyt1p.

GXF1, ΔGXF1, GXS1/HGT12, ΔGXS1/HGT12 and HXT5 from H0 Metschnikowia species were synthesized and transformed into BY4742 for xylose transport testing by HPLC. Following the design for XYT1, each of GXF1, ΔGXF1, GXS1/HGT12, ΔGXS1/HGT12 and HXT5 was expressed from the TEF promoter and TEF terminator derived from the plasmid pUG6. All open reading frames (ORFs) were selected for with nourseothricin.

Due to H0 CTG codon usage, all ORFs corresponding to H0 transporters were synthesized by ThermoFisher as double stranded “gene strings.” H0 transporter ORFs were translated with codon translation table provided above. The resulting amino acid sequence was converted back to DNA. The resulting DNA was entered into the ThermoFisher genestrings web interface. The web interface modified the nucleotide sequence such that the amino acid remained as desired but the nucleotides would be altered such to achieve nearly balanced ratio of adenine-thymidine to guanine-cytosine. The synthetic XYT1 ORF contained approximately 25 bp of homology with the TEF promoter at the 5′ terminus and TEF terminator at the 3′ terminus on pUG6 in order to facilitate Gibson assembly, respectively. The ORF was Gibson assembled into the pUG6, linearized with Y10, Y10R primers, deleting the G418 resistance ORF. Using primers Y15 and Y15R, H0 ADH1 promoter-NAT-H0 PGK1 terminator was amplified from pZL29 and assembled into the TEF promoter-XYT1-TEF terminator plasmid. The complete plasmid containing XYT1 is designated DeBONO_E35.3. E35.3 was used as base vector clone all of GXF1, ΔGXF1, GXS1/HGT12, ΔGXS1/HGT12, HXT5, HGT19. The DeBONO_E35.3 vector was amplified with Y53 and Y53R primers to linearize the vector and simultaneously, omitting XYT1, creating fragment Y53. Each of the synthesized, codon optimized transporters were assembled listed was cloned into fY53 by Gibson assembly. The cassettes expressing transporters and NAT resistance were linearized by PCR with primers Y16, Y16R or Y96i and Y95Ri for integration into dubious ORFs at loci YIL100W and YLR123C. The linearized transporters were integrated into said dubious loci using standard Saccharomyces electroporation or chemical transformation methods.

Transgenic yeasts were recovered with 100 μg/mL NAT in solid YPD medium. GXF2/GAL2 was synthesized as described above. GXF2/GAL2 was cloned into a G418 resistance vector with general structure: CCW12 promoter—GXF2/GAL2—H0 DIT] terminator. The promoter-terminator sequences were amplified from vector DeBONO_E54. This vector was linearized with primers Y83 and Y83R to yield fY83. The GXF2/GAL2 genestring was Gibson assembled into fY83. The transporter cassette was linearized by PCR with primers Y91+Y93R for integration into the dubious ORF at locus YLR122C. The linearized cassette was transformed as described above and GXF2/GAL2 transgenics were selected with 200 ug/mL of G418.

Relevant primer sequences used in this example are provided below.

| SEQ | | |
|---|---|---|
| ID NO: | Primer | Sequences |
| 38 | Primer | GAAAAAACTGGTACCGTTTAATCAGTACTGACA |
| | Y10 | ATAAAAAGATTCTTGT |
| 39 | Primer | TAATTTCTCTTCGTATCCCATGGTTGTTTATGT |
| | Y10R | TCGGATGTGATGTGAG |
| 40 | Primer | ACGCCGCCATCCAGTGTCGAAAACGAGCTTTGT |
| | Y15 | CTTGTAAAGAGTCTTCGGTCATTTTTA |
| 41 | Primer | GCGGCCGCATAGGCCACTAGTGGATCTGATCAA |
| | Y15R | TACATACAAGCATCTCACAATCACAAG |
| 42 | Primer | TTTTTCACCCACAACAAATAATATCAAAAGATG |
| | Y33 | GGTTACGAGGAAAAGCTTGTAGCGCCC |
| 43 | Primer | ACGAGAACACCCAGCTAAACGCGGTGCGCGTTA |
| | Y33R | GACCGTGCCCGTCTTCTCGTCTGAAGA |
| 44 | Primer | CAGAGCAGATTGTACTGAGAGTGCACCAGGCGC |
| | Y41 | GCCCCATCCAGTGTCGAACCATCATTAAAAGAT |
| 45 | Primer | CTCCTTACGCATCTGTGCGGTATTTCACACCGC |
| | Y41R | ACTAGACAATACATACAAGCATCTCACAATCAC |
| | | AA |
| 46 | Primer | TCAGTACTGACAATAAAAAGATTCTTGTTTTCA |
| | Y53 | AGAAC |
| 47 | Primer | CTCACATCACATCCGAACATAAACAACC |
| | Y53R | |
| 48 | Primer | TATCCCGTCACTTCCACATTCG |
| | Y83 | |
| 49 | Primer | TATTGATATAGTGTTTAAGCGAATGACAGAAG |
| | Y83R | |
| 50 | Primer | ATAGAAAGCAAATAGTTATATAATTTTTCATGG |
| | Y96i | ACGTAGGTCTAGAGATCTGTTTAGCTTGC |
| 51 | Primer | AATGCAAAAGCGGCTCCTAAACAGAAATTCTTC |
| | Y95Ri | AGTCAATACATACAAGCATCTCACAATCACAAG |
| 52 | Primer | TCGTCTATATCAAAACTGCATGTTTCTCTACGT |
| | Y93Ri | CTAATTAAGGGTTCTCGAGAGCTCG |
| 53 | Primer | ACTTCAATAGACTTCAATAGAAAGCAAATAGTT |
| | Y91i | ATATGCCCTGAGGATGTATCTGG |

### EXAMPLE II

### Engineering S. cerevisiae with Enhanced Xylose Metabolism

A shown in FIG. 2, The xylose metabolism pathway including Xyl1p, Xyl2p and Xks1p converts xylose for use by the pentose metabolism pathway. Briefly, Xyl1p converts xylose to xylitol; Xyl2p converts the latter to xylulose; and Xks1p converts xylulose to xylulose-5-phosphate. This process allows xylose direct entry into the pentose phosphate pathway and eventually the tricarboxylic acid cycle. Each of the H0 gene products synthesized for S. cerevisiae: Xyl1p, Xyl2p, Xks1p.

Each of the xylose metabolism genes were cloned from the H0 Metschnikowia species. Similar to the transporters described above, each gene was synthesized with S. cerevisiae codon optimization. The H0 XYL1, XYL2 and XKS1 ORFs were expressed in S. cerevisiae from the promoters of ScTDH3, ScCPS1, and AgTEF2, respectively.

### EXAMPLE III

### Engineering S. cerevisiae with Further Enhanced Xylose Metabolism

The S. cerevisiae pentose metabolism primarily functions to supply reducing energy and riboses for nucleotide synthesis. Deletion of phosphatase gene PHO13 can induce upregulation of xylose metabolism genes derepression of transaldolase (TAL1). CRISPR-Cas9 was used to delete the PHO13 gene (Xu et al., 2016). CCW12 promoter was also introduced to express ScTAL1 to further enhance the xylose metabolism in the pho13 deletion mutant.

### EXAMPLE IV

### Engineering S. cerevisiae with Reduced Ethanol Metabolism

Additional metabolic modulations can further improve ethanol production in S. cerevisiae. For example, as shown in FIG. 3, genes encoding enzymes or proteins of the ethanol metabolism pathway that catalyze the conversion of ethanol to other products can be attenuated or deleted, such as genes encoding Adh2p, Ald2p, or Acs1p. Briefly, Adh2p converts ethanol to acetaldehyde, Ald2p converts acetaldehyde to acetate, and Acs1p converts acetate to acetyl-CoA. The attenuation or deletion of one or more of these genes can increase the yield of ethanol.

1. A non-naturally occurring microbial organism comprising a xylose-ethanol pathway and at least one exogenous nucleic acid encoding an enzyme or protein expressed in a sufficient amount to confer or enhance xylose uptake or metabolism, wherein said enzyme or protein has an amino acid sequence that is at least 89% identical to a Metschnikowia enzyme or protein selected from the group consisting of a xylose transporter, a xylose reductase, a xylose dehydrogenase and a xylulokinase.

2. The non-naturally occurring microbial organism of claim 1, comprising at least two, three, or four exogenous nucleic acids encoding two, three, or four enzymes or proteins, respectively, selected from the group consisting of a xylose transporter, a xylose reductase, a xylose dehydrogenase and a xylulokinase.

3-4. (canceled)

5. The non-naturally occurring microbial organism of claim 1, wherein said enzyme or protein has an amino acid sequence that is at least 89% identical with a Metschnikowia xylose transporter selected from the group consisting of Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, and Aps1p/Hgt19p.

6. The non-naturally occurring microbial organism of claim 5, wherein said enzyme or protein is a Metschnikowia xylose transporter selected from the group consisting of Xyt1p, Gxf1p, ΔGxf1p, Gxf2p/Ga12p, Gxs1p/Hgt12p, ΔGxs1p/ΔHgt12p, Hxt5p, Hxt2.6p, Qup2p, and Aps1p/Hgt19p.

7-9. (canceled)

10. The non-naturally occurring microbial organism of claim 1, wherein said xylose reductase is Xyl1p, said xylose dehydrogenase is Xyl2p, and said xylulokinase is Xks1p.

11. The non-naturally occurring microbial organism of claim 1, wherein said Metschnikowia species is H0 Metschnikowia species.

12. The non-naturally occurring microbial organism of claim 1, wherein said exogenous nucleic acid is codon-optimized to produce said enzyme or protein in said microbial organism.

13. The non-naturally occurring microbial organism of claim 1, further comprising at least one exogenous nucleic acid encoding a transketolase, a transaldolase, or both.

14. The non-naturally occurring microbial organism of claim 13, wherein said transketolase is Tkl1p and said transaldolase is Tal1p.

15. The non-naturally occurring microbial organism of claim 1, wherein said at least one exogenous nucleic acid is a heterologous nucleic acid.

16. The non-naturally occurring microbial organism of claim 1, comprising an expression vector having said at least one exogenous nucleic acid.

17. The non-naturally occurring microbial organism of claim 1, wherein said at least one exogenous nucleic acid is integrated into the genome of said microbial organism.

18. The non-naturally occurring microbial organism of claim 1, further comprising one or more gene disruptions occurring in genes encoding an enzyme or protein that reduces xylose metabolism or enhances ethanol metabolism.

19. The non-naturally occurring microbial organism of claim 18, comprising a disruption in the gene encoding phosphatase Pho13p.

20. The non-naturally occurring microbial organism of claim 18, comprising one or more gene disruptions occurring in genes encoding an enzyme or protein selected from the group consisting of an ethanol dehydrogenase, an acetaldehyde dehydrogenase, and an acetate coA-transferase.

21. (canceled)

22. The non-naturally occurring microbial organism of claim 20, wherein said ethanol dehydrogenase is Adh2p, said acetaldehyde dehydrogenase is Ald2p, and said acetate coA-transferase is Acs1p.

23. The non-naturally occurring microbial organism of claim 1, wherein said microbial organism is in a substantially anaerobic culture medium.

24. The non-naturally occurring microbial organism of claim 1, wherein the microbial organism is a species of bacteria or yeast.

25. (canceled)

26. The non-naturally occurring microbial organism of claim 25, wherein the yeast is Saccharomyces cerevisiae.

27. (canceled)

28. A method for producing ethanol comprising culturing the microbial organism of claim 1 under conditions and for a sufficient period of time to produce ethanol.

29. The method of claim 28, wherein the conditions comprise culturing the microbial organism in medium comprising xylose and a co-substrate selected from the group consisting of cellobiose, hemicellulose, glycerol, galactose, and glucose, or a combination thereof.

30. (canceled)

31. The method of claim 28, wherein the culturing comprises substantially anaerobic culturing conditions.

32. The method of claim 28, wherein the culturing comprises batch cultivation, fed-batch cultivation or continuous cultivation.

33. The method of claim 28, wherein the method further comprises separating ethanol from other components in the culture.

34. (canceled)

35. The method of claim 28 wherein the conversion efficiency of ethanol from xylose is at least 0.25 g ethanol/g xylose.

36. The method of claim 28 wherein ethanol is produced at a rate of at least 0.5 g/L/h.

37-41. (canceled)
